SEQUENCE LISTING (476 sequences)

SEQ ID NO: 1 - protein, 670 aa, synthetic construct
  REGION 1..670: note = huTfR Extracellular Domain (ECD) Avi-His
CKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEFGGGLNDIFEAQKIEWHEHHHHHHHHH

SEQ ID NO: 2 - protein, 698 aa, synthetic construct
  REGION 1..698: note = Cyno TfR ECD Avi-His
CKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEFGGGLNDIFEAQKIEWHEHHHHHHHHH

SEQ ID NO: 3 - protein, 701 aa, synthetic construct
  REGION 1..701: note = Mouse TfR (muTfR) ECD Avi-His
CKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEFGGGLNDIFEAQKIEWHEHHHHHHHHH

SEQ ID NO: 4 - protein, 221 aa, synthetic construct
  REGION 1..221: note = huTfR Apical Domain-Avi-His
KIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEGGGLNDIFEAQKIEWHEHHHHHHHHH

SEQ ID NO: 5 - protein, 237 aa, synthetic construct
  REGION 1..237: note = huTfR Apical Domain-myc-V5-His
KIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKERAAAGSGSEQKLISEEDLGKPIPNPLLGLDSTHHHHHHHHHH

SEQ ID NO: 6 - protein, 180 aa, synthetic construct
  REGION 1..180: note = HuTfR apical permuted N-term Avi-His
HHHHHHHHGLNDIFEAQKIEWHESSGLPNIPVQTISRAAAEKLFGNMEGDCPSDWKTDSTCRMVTSESKNVKLTVSNDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELS

SEQ ID NO: 7 - protein, 182 aa, synthetic construct
  REGION 1..182: note = Cyno TfR apical permuted N-term Avi-His
HHHHHHHHHHGLNDIFEAQKIEWHESSGLPNIPVQTISRAAAEKLFGNMEGDCPSDWKTDSTCKMVTSENKSVKLTVSNDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLS

SEQ ID NO: 8 - protein, 760 aa, synthetic construct
  REGION 1..760: note = human Full Length TfR Sequence
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 9 - protein, 763 aa, synthetic construct
  REGION 1..763: note = Full length Mouse TfR
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 10 - protein, 119 aa, synthetic construct
  REGION 1..119: note = TfR1 VH
QVQLQQSGPELVKPGASVKMSCKASGYTFTNYYIHWVKQRPGQGLEWIGWIYPGDGSTKYNEKFKGKTTLTADKSSSTAYMLLSSLTSEDSAIYFCARDGSSAPKFTYWGQGTLVTVSA

SEQ ID NO: 11 - protein, 113 aa, synthetic construct
  REGION 1..113: note = TfR1 VL
DIVMTQSPSSLTVTAGEKVTMRCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 12 - protein, 118 aa, synthetic construct
  REGION 1..118: note = TfR2 VH
EVQLQQSGPELVKPGASVKISCKASGYTFTDYNMHWVKQSHGKSLEWIGYIYPYNGGTGYNQKFKSKATLTVDNSSSTAYMELRSLTSEDSAVYYCATLYDGYYFDYWGQGTTLTVSS

SEQ ID NO: 13 - protein, 107 aa, synthetic construct
  REGION 1..107: note = TfR2 VL
DILLTQSPAILSVSPGERVSFSCRASQSIGTSIHWYQQRTNGSPRLLIKYASESISGIPSRFSGSGSGTDFTLSINNVESEDIADYYCQQSNSWPFTFGSGTKLEIK

SEQ ID NO: 14 - protein, 117 aa, synthetic construct
  REGION 1..117: note = TfR3 VH
QVQLRQSGPELVKPGASVRISCKASGYTFTSFYIHWVKQRPGQGLEWIGWIYPGDVNTHYSDKFKGKATLTADKSSSTAYMQLSSLTSEDSAVYFCAREGYGYFAYWGQGTLVTVSA

SEQ ID NO: 15 - protein, 113 aa, synthetic construct
  REGION 1..113: note = TfR3 VL
DIVMTQSPSSLTVTAGEKVTMSCKSSQSLLNSGNQKNYLTWYQQKPGQPPKLLIYWASTRESGVPDRFTGVGSGTDFTLTISSVQAEDLAVYYCQNDYSYPLTFGSGTKLELK

SEQ ID NO: 16 - protein, 116 aa, synthetic construct
  REGION 1..116: note = TfR4 VH
QVQLQQSGAELVRPGTSVKVSCKASGYAFTNYLIEWVKQRPGQGLEWIGVINPGSGGTTYNEKFKGKATLTADKSSSTAYMQLSGLTSDDSAVYFCARRSPYFDYWGQGTTLTVSS

SEQ ID NO: 17 - protein, 106 aa, synthetic construct
  REGION 1..106: note = TfR4 VL
SIVMTQTPKFLLVSAGDRVTITCKASQSVSNDVAWYQQKPGQSPKLLIYYASNRYTGVPDRFTGSGYGTDFTFTISTVQAEDLAVYFCHQDYISPTFGGGTKLEIK

SEQ ID NO: 18 - protein, 119 aa, synthetic construct
  REGION 1..119: note = TfR5 VH
QVQLQQSGPELVKPGASVKMSCKASGYTFTNYYIHWVKQRPGQGLEWIGWIYPGDGNTNYNEKFKGKTTLTADKSSSTAYMLLSSLTSEDSAIYFCARDGSSAPKFTYWGQGTLVTVSA

SEQ ID NO: 19 - protein, 113 aa, synthetic construct
  REGION 1..113: note = TfR5 VL
DIVMTQSPSSLTVTAGEKVTMRCKSSQSLLNSGNQKNYLTWYQQKPGQPPKLLFYWASTRESGVPDRFTGSGSGTDFTLTISSVQAEDLAVYYCQNDYSYPLTFGAGTKLELK

SEQ ID NO: 20 - protein, 120 aa, synthetic construct
  REGION 1..120: note = TfR6 VH
DVQLVESGGGLVQPGGSRKLSCAASGFTFSSFGMHWVRQAPEKGLEWVAYISSGSSAIYYADTVKGRFTVSRDNPKNTLFLQMTSLRSEDTAIYYCATSYVYDPPWFAYWGQGTLVTVSA

SEQ ID NO: 21 - protein, 107 aa, synthetic construct
  REGION 1..107: note = TfR6 VL
DIVMTQSHKFMSTSVGDRVSITCKASQDVSTAVAWYQQKPGQSPKLLIYWASTRPTGVPDRFTGSGSGTDYTLTISSVQAEDLALYSCQQHFSTPWTFGGGTKLEIK

SEQ ID NO: 22 - protein, 116 aa, synthetic construct
  REGION 1..116: note = TfR7 VH
DVQLQESGPGLVKPSQSLSLTCTVTGYSITSDYAWNWLRQFPGNKLEWMGYISYSGYSRHNPSLKSRISITRDTSKNQFFLQLNSVTSEDTATYYCARGDWDFDYWGQGTTLTVSS

SEQ ID NO: 23 - protein, 107 aa, synthetic construct
  REGION 1..107: note = TfR7 VL
DIVMTQSQKFMSTSVGDRVSVTCKASQNVLTNVAWYQQKPGHSPKALIYSSSSRNSGVPDRFTGSGSGTDFTLTISNVQSEDLAEYFCQQYNTYPYTFGGGTKLEIK

SEQ ID NO: 24 - protein, 116 aa, synthetic construct
  REGION 1..116: note = TfR8 VH
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARRSPYFDYWGQGTTLTVSS

SEQ ID NO: 25 - protein, 106 aa, synthetic construct; identical to SEQ ID NO: 17
  REGION 1..106: note = TfR8 VL

SEQ ID NO: 26 - protein, 119 aa, synthetic construct
  REGION 1..119: note = TfR9 VH
EVQLQQSGPELVKPGASVKISCKTSGYTFTEYAMHWVKQSHGKSLEWIGGINPNDGGTSYNQKFKGKATLTVDKSSSTAYMELRSLTSEDSAVYYCSSQRWLLRGGDYWGQGTTLTVSS

SEQ ID NO: 27 - protein, 112 aa, synthetic construct
  REGION 1..112: note = TfR9 VL
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 28 - protein, 117 aa, synthetic construct
  REGION 1..117: note = TfR10 VH
QVQLQQSGPELVKPGASVKMSCKASGYTFTSYYIHWVKQRPGQGLEWIGWIYPGDGNTNYNEKFKGKTTLTADKSSSTAYMLLSSLTSEDSAIYFCARDGSIPFAYWGQGTLVTVSA

SEQ ID NO: 29 - protein, 113 aa, synthetic construct
  REGION 1..113: note = TfR10 VL
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 30 - protein, 115 aa, synthetic construct
  REGION 1..115: note = TfR11 VH
EVQLQQSGTVLARPGASVKMSCKASGYSFTSYWMHWVKQRPGQGLEWIGAIFPGNIDTIYNQKFKDKAKLTAVTSASTAYMELSSLTNEDSAVYFCTRLYYDYVWGAGTTVTVSS

SEQ ID NO: 31 - protein, 107 aa, synthetic construct
  REGION 1..107: note = TfR11 VL
DIKMTQSPSSMYASLGERVTLTCKASQDINSYLSWFQQKPGKSPKTLIYHVNRLVDGVPSRFSGSGSGQDYSLTISSLEYEDMGIYYCLQYDEFPYTFGGGTKLEIK

SEQ ID NO: 32 - protein, 121 aa, synthetic construct
  REGION 1..121: note = TfR12 VH
QVQLQQPGSELVRPGASVKLSCKASGYTFTSYWMHWVKQRPGQGLEWIGNIYPGSGSSNYDEKFKNKATLTVDTSSNTAYMQLSSLTSEDSAVYYCTRSSYYGNFDTMDNWGQGTSVTVSS

SEQ ID NO: 33 - protein, 106 aa, synthetic construct
  REGION 1..106: note = TfR12 VL
QIVLTQSPAIMSASPGEKVTMTCSASSSIRYMNWYQQKPVTSPKRWIYDTSTLASGVPARFSGSGSGTSYSLTISSMEAEDAATYYCHQRSSYPWTFGGGTKLEIK

SEQ ID NO: 34 - protein, 117 aa, synthetic construct
  REGION 1..117: note = TfR13 VH
DVKLVESGGGLVKPGGSLKLSCAASGFTFSSYTMSWVRQTPEKRLEWVATISSGGSYTYFPDSVKGRFTISRDNAKNTLYLQMSSLKSEDTAIYYCTRAHYYAYDYWGQGTTLTVSS

SEQ ID NO: 35 - protein, 108 aa, synthetic construct
  REGION 1..108: note = TfR13 VL
DILLTQSPAILSVSPGERVSFSCRASQSIGTSIHWYQQRTNGSPRLLIKYASGSISGIPSRFSGSGSGTDFTLSINSVESEDIADYYCQQSNSWPTSTFGAGTKLELK

SEQ ID NO: 36 - protein, 122 aa, synthetic construct
  REGION 1..122: note = TfR14 VH
QVQLQQSGPELVKPGASVKISCKASGYAFSSSWMNWVKQRPGQGLEWIGRIYPGDGDTNYNGKFKGKATLTADKSSSTAYMQLSSLTSVDSAVYFCARDPIIDYYYYAMDYWGQGTSVTVSS

SEQ ID NO: 37 - protein, 111 aa, synthetic construct
  REGION 1..111: note = TfR14 VL
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIK

SEQ ID NO: 38 - protein, 121 aa, synthetic construct
  REGION 1..121: note = TfR15 VH
QVQLQQPGSELVRPGASVKLSCKASGYTFTSYWMHWVKQRPGQGLEWIGNIYPGSGSSNYDEKFKSKATLTVDTSSSTAYMQLNSLTSEDSAVYYCTRSSYYGNFDTMDNWGQGTSVTVSS

SEQ ID NO: 39 - protein, 106 aa, synthetic construct; identical to SEQ ID NO: 33
  REGION 1..106: note = TfR15 VL

SEQ ID NO: 40 - protein, 115 aa, synthetic construct
  REGION 1..115: note = TfR16 VH
EVQLQQSGTVLARPGASVKMSCKTSGYSFSSYWMHWVKQRPGQGLEWIGTIYPGNSDTIYNQNFKGKAKLTAVTSASTAYMELSSLTNEDSAVYYCSTIYYFSNWGQGTTLTVSS

SEQ ID NO: 41 - protein, 107 aa, synthetic construct
  REGION 1..107: note = TfR16 VL
DIKMTQSPSPMFASLGERVTITCKASQDINSYLSWFQQKPGKSPKTLIYRANRLLDGVPSRFSGSGSGQDYSLTISSLDYEDVGIYYCLQYDVFPYTFGGGTKLEIK

SEQ ID NO: 42 - protein, 118 aa, synthetic construct
  REGION 1..118: note = TfR17 VH
DVQLQESGPGLVKPSQSLSLTCTVTGYSITSDYAWNWIRQFPGNKLEWMGYISYSGSTSYNPSLKSRISITRDTSKNQFFLQLNSVTTEDTATYYCASWGYRYLEDYWGQGTSVTVSS

SEQ ID NO: 43 - protein, 106 aa, synthetic construct
  REGION 1..106: note = TfR17 VL
QIVLTQSPAIMSASPGEKVTMTCSASSSISSMHWYQQKPGTSPKRWISDTSKLASGIPARFSGSGSGTSYSLTISSMAAEDAAAYYCHQRSSYPWTFGGGTKLEIK

SEQ ID NO: 44 - protein, 116 aa, synthetic construct
  REGION 1..116: note = TfR18 VH
QVQLQQSGAELVRPGTSVKVSCKASGYAFTNYLIEWIKQRPGQGLEWIGVINPGNNDTTYNEMFKAKTTLTADKSSNTAYMQLSSLTSDDSAVYFCARRSPYFDYWGQGTTLTVSS

SEQ ID NO: 45 - protein, 106 aa, synthetic construct
  REGION 1..106: note = TfR18 VL
SIVMTQTPKFLLVSAGDRVTITCKASQSVSNDVAWYQQKPGQSPKLLIYYASNRYTGVPDRFTGSGYGTDFTFTISTVQAEDLAVYFCHQDYSSPTFGGGTKLEIK

SEQ ID NO: 46 - protein, 121 aa, synthetic construct
  REGION 1..121: note = TfR19 VH
QVQLQQPGSELVRPGASVKLSCKASGYTFTSYWTHWVKQRPGQGLEWIGNIYPGSGSSNYDEKFKSKATLTVDTSSSTAYMQLSSLTSEDSAVYFCTRSSYYGNFDTMDNWGQGTSVTVSS

SEQ ID NO: 47 - protein, 106 aa, synthetic construct; identical to SEQ ID NO: 33
  REGION 1..106: note = TfR19 VL

SEQ ID NO: 48 - protein, 116 aa, synthetic construct
  REGION 1..116: note = TfR20 VH
DVQLQESGPGLVKPSQSLSLTCTVTGYSITSDHAWNWIRQFPGNKLEWMGYISYSGSSSYNPSLKSRISITRDTSKNQFFLQLNSVTTEDTATYYCARSGWGMDYWGQGTSVTVSS

SEQ ID NO: 49 - protein, 107 aa, synthetic construct
  REGION 1..107: note = TfR20 VL
DIQMTQSPASLSASVGETVTITCRASEYINSYLAWYQQKQGKSPQLLVFKEKTLAEGVPSRFSGSGSGTQFSLKINSLQPEDFGSYYCQHHYGNPWTFGGGTKLEIK

SEQ ID NO: 50 - protein, 121 aa, synthetic construct; identical to SEQ ID NO: 46
  REGION 1..121: note = TfR21 VH

SEQ ID NO: 51 - protein, 107 aa, synthetic construct; identical to SEQ ID NO: 49
  REGION 1..107: note = TfR21 VL

SEQ ID NO: 52 - protein, 118 aa, synthetic construct
  REGION 1..118: note = TfR22 VH
DVQLQESGPGLVKPSQSLSLTCTVTGYSITSDYAWNWIRQFPGNKLEWMGYISYSGSTRYNPSLKSRISITRDTSKNQFFLQLNSVTTEDTATYYCTSWGYRYLEDYWGQGTSVTVSA

SEQ ID NO: 53 - protein, 106 aa, synthetic construct
  REGION 1..106: note = TfR22 VL
QIVLTQSPAIMSASPGERVTMTCSASSSISSMHWYQQKPGTSPKKWIYDTSKLASGVPARFSGSGSGTSYSLTISSMEAEDAATYYCHQRSSYPWTFGGGTKLEIK

SEQ ID NO: 54 - protein, 118 aa, synthetic construct
  REGION 1..118: note = TfR23 VH
DVQLQESGPGLVKPSQSLSLTCTVTGYSITSDYAWNWIRQFPGNKLEWMGYISYSGSTSYNPSLKSRISITRDTSKNQFFLQLNSVTTEDTATYYCTSWGYRYLEDYWGQGTSVTVSS

SEQ ID NO: 55 - protein, 106 aa, synthetic construct
  REGION 1..106: note = TfR23 VL
QIVLTQSPAIMSASPGERVTMTCSASSSISSLHWYQQKPGTSPKKWIYDTSKLASGVPARFSGSGSGTSYSLAISSMEAEDAATYYCHQRSSYPWTFGGGTKLEIK

SEQ ID NO: 56 - protein, 117 aa, synthetic construct
  REGION 1..117: note = TfR24 VH
QVQLQQSGPELVKPGTSVKMSCKASGYTFTSYYIHWVKQRPGQGLEWIGWIYPGDDNTHYNENFKGKTTLTADKSSSTAYMLLSSLTSEDSAIYFCARDYYGRFAYWGQGTLVTVSA

SEQ ID NO: 57 - protein, 113 aa, synthetic construct
  REGION 1..113: note = TfR24 VL
DIVMTQSPSSLTVTAGERVTMSCKSSQSLLNSGNQKNYLTWFQQKPGQPPKLLIYWASTRESGVPDRFTGSGSGTDFTVTISSVQAEDLAVYYCQNDYGYPLTFGAGTKLELK

SEQ ID NO: 58 - protein, 118 aa, synthetic construct
  REGION 1..118: note = TfR25 VH
QVQLQQSGPELVKPGTSVKMSCKASGYTFTNYYIHWMKQRPGQGLEWIGWIYPGDGNTHYNERFKGKTTLTADKSSSTVYMFLTSLTSEDSAIYFCAKDKDMPGMDYWGQGTSVTVSS

SEQ ID NO: 59 - protein, 113 aa, synthetic construct
  REGION 1..113: note = TfR25 VL
DMVMTQSPSSLTVTAGEKVTMSCKSSQSLFNSENQKNYLTWYQQKPGQPPKLLISWASTRESGVPDRFTGSGSGTDFTLTINSVQAEDLAVYYCQNDYSYPLTFGAGTKLELK

SEQ ID NO: 60 - protein, 118 aa, synthetic construct
  REGION 1..118: note = TfR26 VH
QVQLHQSGPELVKPGASVKMSCKASGYTFTNYYIHWVKQRPGQGLEWIGWIYPGDGVTHYNEKFKGKTTLTADKSSSTAYMLLSSLTSEDSAIYFCARDKDVPGMDYWGQGTSVTVSS

SEQ ID NO: 61 - protein, 113 aa, synthetic construct
  REGION 1..113: note = TfR26 VL
DIVMTQSPSSLTVTSGEKVTMSCKSSQSLLNSENQKNYLTWYQQKPGQPPKLLIYWASTRESGVPDRFTGSGSGPDFTLTISSVQAEDLAVYYCQNDYSYPLTFGAGTKLELK

SEQ ID NO: 62 - protein, 121 aa, synthetic construct
  REGION 1..121: note = TfR27 VH
QVQLQQPGSELVRPGASVKLSCKASGYTFTSYWMHWVKQRPGQGLEWIGNIYPGTGSTNYDEKFKSKATLTVDTSSSTAYMQLSSLTSEDSAVYYCTRSSYYGNFDTMDYWGQGTSVTVSS

SEQ ID NO: 63 - protein, 106 aa, synthetic construct
  REGION 1..106: note = TfR27 VL
QIFLTQSPAIMSASPGERVTMTCSASSSIRYMNWYQQKPGTSPKRWIYDTSELASGVPARFTGSGSGTSYSLTISSMEAEDAATYYCHQRSGYPWTFGGGTKLEIK

SEQ ID NO: 64 - protein, 118 aa, synthetic construct
  REGION 1..118: note = TfR28 VH
DVQLQESGPGLVKPSQSLSLTCTVTGYSITSDYAWNWIRQFPGNKLEWMGYISYSGSTSYNPSLRSRISITRDTSKNQFFLQLNSVTTEDTATYYCAIRGYRYEEDYWGQGTTLTVSS

SEQ ID NO: 65 - protein, 106 aa, synthetic construct
  REGION 1..106: note = TfR28 VL
QIVLTQSPAIMSASPREKVTMTCSASSSISSMHWYQQKPGTSPKRWIYDTSKLASGVPARFSGSGSGTSYSLTISSMEAEDAATYYCHQRSSYPWTFGGGTKLEIK

SEQ ID NO: 66 - protein, 119 aa, synthetic construct
  REGION 1..119: note = TfR29 VH
QVQLQQSGPQLVRPGASVKISCKASGYSFTNYWLHWVKQRPGQGLEWIGMIDPSDSETRLNQKFKDKATLTVDKSSSTAYMQLSSPTSEDSAVYYCARWGGGYYGFAYWGQGTLVTVSA

SEQ ID NO: 67 - protein, 104 aa, synthetic construct
  REGION 1..104: note = TfR29 VL
QIVLTQSPAIMSASPGEKVTMTCSASSSISYMHWYQQKPGTSPKRWIYDTSKLASGVPARFSGSGSGTSYSLTISSMEAEDAATYYCHQRSSYTFGGGTKLEIK

SEQ ID NO: 68 - protein, 119 aa, synthetic construct; identical to SEQ ID NO: 66
  REGION 1..119: note = TfR30 VH

SEQ ID NO: 69 - protein, 107 aa, synthetic construct
  REGION 1..107: note = TfR30 VL
DIVMTQSQKFMSTSVGDRVSITCKASQNVRTAVAWYQQKPGQSPKALIYLASNRHTGVPDRFTGSGSGTDFTLTISNVQSEDLADYFCLQHWNYPWTFGGGTKLEIK

SEQ ID NO: 70 - protein, 118 aa, synthetic construct
  REGION 1..118: note = TfR31 VH
DVQLQESGPGLVKPSQSLSLTCTVTGYSITSDYAWNWIRQFPGNKLEWMGYISYSGRTSYNPSLKSRISFTRDTSKNQFFLQLNSVTTEDTATYYCAGWGYRYLEDYWGQGTSVTVSS

SEQ ID NO: 71 - protein, 106 aa, synthetic construct
  REGION 1..106: note = TfR31 VL
QIVLTQSPAIMSASPGEKVTMTCSASSSISSMHWYQLKPGTSPKKWIYDTSKLASGVPARFSGSGSGTSYSLTISSMEAEDAATYYCHQRSSYPWTFGGGTKLEIK

SEQ ID NO: 72 - protein, 118 aa, synthetic construct
  REGION 1..118: note = TfR32 VH
QVQLQQSGADLVRPGTSVKVSCKASGNAFSNYLIEWVKQRPGQGLEWIGVINPGSGETNYNEKFKGKATLTADKSSSTAYMQLSSLTSDDSAVYFCAIRYDGPLLDHWGQGTSVTVSS

SEQ ID NO: 73 - protein, 106 aa, synthetic construct
  REGION 1..106: note = TfR32 VL
SIVMTQTPKFLLVSAGDRVTITCKASQSVSNDVAWFQQKPGQSPKLLIYYASNRYTGVPDRFTGSGYGTDFTFTIRNVQAEDLAVYFCQQDYISPTFGAGTKLELK

SEQ ID NO: 74 - protein, 5 aa, synthetic construct
  REGION 1..5: note = anti-huTfr HVR-H1
NYYIH

SEQ ID NO: 75 - protein, 17 aa, synthetic construct
  REGION 1..17: note = anti-huTfr HVR-H2
WIYPGDGSTKYNEKFKG

SEQ ID NO: 76 - protein, 10 aa, synthetic construct
  REGION 1..10: note = anti-huTfr HVR-H3
DGSSAPKFTY

SEQ ID NO: 77 - protein, 5 aa, synthetic construct
  REGION 1..5: note = anti-huTfr HVR-H1
DYNMH

SEQ ID NO: 78 - protein, 17 aa, synthetic construct
  REGION 1..17: note = anti-huTfr HVR-H2
YIYPYNGGTGYNQKFKS

SEQ ID NO: 79 - protein, 9 aa, synthetic construct
  REGION 1..9: note = anti-huTfr HVR-H3
LYDGYYFDY

SEQ ID NO: 80 - protein, 5 aa, synthetic construct
  REGION 1..5: note = anti-huTfr HVR-H1
SFYIH

SEQ ID NO: 81 - protein, 17 aa, synthetic construct
  REGION 1..17: note = anti-huTfr HVR-H2
WIYPGDVNTHYSDKFKG

SEQ ID NO: 82 - protein, 8 aa, synthetic construct
  REGION 1..8: note = anti-huTfr HVR-H3
EGYGYFAY

SEQ ID NO: 83 - protein, 5 aa, synthetic construct
  REGION 1..5: note = anti-huTfr HVR-H1
NYLIE

SEQ ID NO: 84 - protein, 17 aa, synthetic construct
  REGION 1..17: note = anti-huTfr HVR-H2
VINPGSGGTTYNEKFKG

SEQ ID NO: 85 - protein, 7 aa, synthetic construct
  REGION 1..7: note = anti-huTfr HVR-H3
RSPYFDY

SEQ ID NO: 86 - protein, 17 aa, synthetic construct
  REGION 1..17: note = anti-huTfr HVR-H2
WIYPGDGNTNYNEKFKG

SEQ ID NO: 87 - protein, 5 aa, synthetic construct
  REGION 1..5: note = anti-huTfr HVR-H1
SFGMH

SEQ ID NO: 88 - protein, 17 aa, synthetic construct
  REGION 1..17: note = anti-huTfr HVR-H2
YISSGSSAIYYADTVKG

SEQ ID NO: 89 - protein, 11 aa, synthetic construct
  REGION 1..11: note = anti-huTfr HVR-H3
SYVYDPPWFAY

SEQ ID NO: 90 - protein, 6 aa, synthetic construct
  REGION 1..6: note = anti-huTfr HVR-H1
SDYAWN

SEQ ID NO: 91 - protein, 16 aa, synthetic construct
  REGION 1..16: note = anti-huTfr HVR-H2
YISYSGYSRHNPSLKS

SEQ ID NO: 92 - protein, 7 aa, synthetic construct
  REGION 1..7: note = anti-huTfr HVR-H3
GDWDFDY

SEQ ID NO: 93 - protein, 17 aa, synthetic construct
  REGION 1..17: note = anti-huTfr HVR-H2
VINPGSGGTNYNEKFKG

SEQ ID NO: 94 - protein, 5 aa, synthetic construct
  REGION 1..5: note = anti-huTfr HVR-H1
EYAMH

SEQ ID NO: 95 - protein, 17 aa, synthetic construct
  REGION 1..17: note = anti-huTfr HVR-H2
GINPNDGGTSYNQKFKG

SEQ ID NO: 96 - protein, 10 aa, synthetic construct
  REGION 1..10: note = anti-huTfr HVR-H3
QRWLLRGGDY

SEQ ID NO: 97 - protein, 5 aa, synthetic construct
  REGION 1..5: note = anti-huTfr HVR-H1
SYYIH

SEQ ID NO: 98 - protein, 8 aa, synthetic construct
  REGION 1..8: note = anti-huTfr HVR-H3
DGSIPFAY

SEQ ID NO: 99 - protein, 5 aa, synthetic construct
  REGION 1..5: note = anti-huTfr HVR-H1
SYWMH

SEQ ID NO: 100 - protein, 17 aa, synthetic construct
  REGION 1..17: note = anti-huTfr HVR-H2
AIFPGNIDTIYNQKFKD

SEQ ID NO: 101 - protein, 6 aa, synthetic construct
  REGION 1..6: note = anti-huTfr HVR-H3
LYYDYV

SEQ ID NO: 102 - protein, 17 aa, synthetic construct
  REGION 1..17: note = anti-huTfr HVR-H2
NIYPGSGSSNYDEKFKN

SEQ ID NO: 103 - protein, 12 aa, synthetic construct
  REGION 1..12: note = anti-huTfr HVR-H3
SSYYGNFDTMDN

SEQ ID NO: 104 - protein, 5 aa, synthetic construct
  REGION 1..5: note = anti-huTfr HVR-H1
SYTMS

SEQ ID NO: 105 - protein, 17 aa, synthetic construct
  REGION 1..17: note = anti-huTfr HVR-H2
TISSGGSYTYFPDSVKG

SEQ ID NO: 106 - protein, 8 aa, synthetic construct
  REGION 1..8: note = anti-huTfr HVR-H3
AHYYAYDY

SEQ ID NO: 107 - protein, 5 aa, synthetic construct
  REGION 1..5: note = anti-huTfr HVR-H1
SSWMN

SEQ ID NO: 108 - protein, 17 aa, synthetic construct
  REGION 1..17: note = anti-huTfr HVR-H2
RIYPGDGDTNYNGKFKG

SEQ ID NO: 109 - protein, 13 aa, synthetic construct
  REGION 1..13: note = anti-huTfr HVR-H3
DPIIDYYYYAMDY

SEQ ID NO: 110 - protein, 17 aa, synthetic construct
  REGION 1..17: note = anti-huTfr HVR-H2
NIYPGSGSSNYDEKFKS

SEQ ID NO: 111 - protein, 17 aa, synthetic construct
  REGION 1..17: note = anti-huTfr HVR-H2
TIYPGNSDTIYNQNFKG

SEQ ID NO: 112 - protein, 6 aa, synthetic construct
  REGION 1..6: note = anti-huTfr HVR-H3
IYYFCN

SEQ ID NO: 113 - protein, 16 aa, synthetic construct
  REGION 1..16: note = anti-huTfr HVR-H2
YISYSGSTSYNPSLKS

SEQ ID NO: 114 - protein, 9 aa, synthetic construct
  REGION 1..9: note = anti-huTfr HVR-H3
WGYRYLEDY

SEQ ID NO: 115 - protein, 17 aa, synthetic construct
  REGION 1..17: note = anti-huTfr HVR-H2
VINPGNNDTTYNEMFKA

SEQ ID NO: 116 - protein, 5 aa, synthetic construct
  REGION 1..5: note = anti-huTfr HVR-H1
SYWTH

SEQ ID NO: 117 - protein, 6 aa, synthetic construct
  REGION 1..6: note = anti-huTfr HVR-H2
SDHAWN

SEQ ID NO: 118 - protein, 16 aa, synthetic construct
  REGION 1..16: note = anti-huTfr HVR-H2
YISYSGSSSYNPSLKS

SEQ ID NO: 119 - protein, 7 aa, synthetic construct
  REGION 1..7: note = anti-huTfr HVR-H3
SGWGMDY

SEQ ID NO: 120 - protein, 16 aa, synthetic construct
  REGION 1..16: note = anti-huTfr HVR-H2
YISYSGSTRYNPSLKS

SEQ ID NO: 121 - protein, 17 aa, synthetic construct
  REGION 1..17: note = anti-huTfr HVR-H2
WIYPGDDNTHYNENFKG

SEQ ID NO: 122 - protein, 8 aa, synthetic construct
  REGION 1..8: note = anti-huTfr HVR-H3
DYYGRFAY

SEQ ID NO: 123 - protein, 17 aa, synthetic construct
  REGION 1..17: note = anti-huTfr HVR-H2
WIYPGDGNTHYNERFKG

SEQ ID NO: 124 - protein, 9 aa, synthetic construct
  REGION 1..9: note = anti-huTfr HVR-H3
DKDMPGMDY

SEQ ID NO: 125 - protein, 17 aa, synthetic construct
  REGION 1..17: note = anti-huTfr HVR-H2
WIYPGDGVTHYNEKFKG

SEQ ID NO: 126 - protein, 9 aa, synthetic construct
  REGION 1..9: note = anti-huTfr HVR-H3
DKDVPGMDY

SEQ ID NO: 127 - protein, 17 aa, synthetic construct
  REGION 1..17: note = anti-huTfr HVR-H2
NIYPGTGSTNYDEKFKS

SEQ ID NO: 128 - protein, 12 aa, synthetic construct
  REGION 1..12: note = anti-huTfr HVR-H3
SSYYGNFDTMDY

SEQ ID NO: 129 - protein, 16 aa, synthetic construct
  REGION 1..16: note = anti-huTfr HVR-H2
YISYSGSTSYNPSLRS

SEQ ID NO: 130 - protein, 9 aa, synthetic construct
  REGION 1..9: note = anti-huTfr HVR-H3
RGYRYEEDY

SEQ ID NO: 131 - protein, 5 aa, synthetic construct
  REGION 1..5: note = anti-huTfr HVR-H1
NYWLH

SEQ ID NO: 132 - protein, 17 aa, synthetic construct
  REGION 1..17: note = anti-huTfr HVR-H2
MIDPSDSETRLNQKFKD

SEQ ID NO: 133 - protein, 10 aa, synthetic construct
  REGION 1..10: note = anti-huTfr HVR-H3
WGGGYYGFAY

SEQ ID NO: 134 - protein, 16 aa, synthetic construct
  REGION 1..16: note = anti-huTfr HVR-H2
YISYSGRTSYNPSLKS

SEQ ID NO: 135 - protein, 17 aa, synthetic construct
  REGION 1..17: note = anti-huTfr HVR-L1
KSSQSLLNSGNQKNYLT

SEQ ID NO: 136 - protein, 7 aa, synthetic construct
  REGION 1..7: note = anti-huTfr HVR-L2
WASTRES

SEQ ID NO: 137 - protein, 9 aa, synthetic construct
  REGION 1..9: note = anti-huTfr HVR-L3
QNDYSYPLT

SEQ ID NO: 138 - protein, 11 aa, synthetic construct
  REGION 1..11: note = anti-huTfr HVR-L1
RASQSIGTSIH

SEQ ID NO: 139 - protein, 7 aa, synthetic construct
  REGION 1..7: note = anti-huTfr HVR-L2
YASESIS

SEQ ID NO: 140 - protein, 9 aa, synthetic construct
  REGION 1..9: note = anti-huTfr HVR-L3
QQSNSWPFT

SEQ ID NO: 141 - protein, 11 aa, synthetic construct
  REGION 1..11: note = anti-huTfr HVR-L1
KASQSVSNDVA

SEQ ID NO: 142 - protein, 7 aa, synthetic construct
  REGION 1..7: note = anti-huTfr HVR-L2
YASNRYT

SEQ ID NO: 143 - protein, 8 aa, synthetic construct
  REGION 1..8: note = anti-huTfr HVR-L3
HQDYISPT

SEQ ID NO: 144 - protein, 7 aa, synthetic construct
  REGION 1..7: note = anti-huTfr HVR-L2
WASTRPT

SEQ ID NO: 145 - protein, 9 aa, synthetic construct
  REGION 1..9: note = anti-huTfr HVR-L3
QQHFSTPWT

SEQ ID NO: 146 - protein, 11 aa, synthetic construct
  REGION 1..11: note = anti-huTfr HVR-L1
KASQNVLTNVA

SEQ ID NO: 147 - protein, 7 aa, synthetic construct
  REGION 1..7: note = anti-huTfr HVR-L2
SSSSRNS

SEQ ID NO: 148 - protein, 9 aa, synthetic construct
  REGION 1..9: note = anti-huTfr HVR-L3
QQYNTYPYT

SEQ ID NO: 149 - protein, 16 aa, synthetic construct
  REGION 1..16: note = anti-huTfr HVR-L1
KSSQSLLDSDGKTYLN

SEQ ID NO: 150 - protein, 7 aa, synthetic construct
  REGION 1..7: note = anti-huTfr HVR-L2
LVSKLDS

SEQ ID NO: 151 - protein, 9 aa, synthetic construct
  REGION 1..9: note = anti-huTfr HVR-L3
WQGTHFPQT

SEQ ID NO: 152 - protein, 11 aa, synthetic construct
  REGION 1..11: note = anti-huTfr HVR-L3
KASQDINSYLS

SEQ ID NO: 153 - protein, 7 aa, synthetic construct
  REGION 1..7: note = anti-huTfr HVR-L2
HVNRLVD

SEQ ID NO: 154 - protein, 9 aa, synthetic construct
  REGION 1..9: note = anti-huTfr HVR-L3
LQYDEFPYT

SEQ ID NO: 155 - protein, 10 aa, synthetic construct
  REGION 1..10: note = anti-huTfr HVR-L1
SASSSIRYMN

SEQ ID NO: 156 - protein, 7 aa, synthetic construct
  REGION 1..7: note = anti-huTfr HVR-L2
DTSTLAS

SEQ ID NO: 157 - protein, 9 aa, synthetic construct
  REGION 1..9: note = anti-huTfr HVR-L3
HQRSSYPWT

SEQ ID NO: 158 - protein, 7 aa, synthetic construct
  REGION 1..7: note = anti-huTfr HVR-L2
YASGSIS

SEQ ID NO: 159 - protein, 10 aa, synthetic construct
  REGION 1..10: note = anti-huTfr HVR-L3
QQSNSWPTST

SEQ ID NO: 160 - protein, 15 aa, synthetic construct
  REGION 1..15: note = anti-huTfr HVR-L1
RASESVDNYGISFMN

SEQ ID NO: 161 - protein, 7 aa, synthetic construct
  REGION 1..7: note = anti-huTfr HVR-L2
AASNQGS

SEQ ID NO: 162 - protein, 9 aa, synthetic construct
  REGION 1..9: note = anti-huTfr HVR-L3
QQSKEVPWT

SEQ ID NO: 163 - protein, 7 aa, synthetic construct
  REGION 1..7: note = anti-huTfr HVR-L2
RANRLLD

SEQ ID NO: 164 - protein, 9 aa, synthetic construct
  REGION 1..9: note = anti-huTfr HVR-L3
LQYDVFPYT

SEQ ID NO: 165 - protein, 10 aa, synthetic construct
  REGION 1..10: note = anti-huTfr HVR-L1
SASSSISSMH

SEQ ID NO: 166 - protein, 7 aa, synthetic construct
  REGION 1..7: note = anti-huTfr HVR-L2
DTSKLAS

SEQ ID NO: 167 - protein, 8 aa, synthetic construct
  REGION 1..8: note = anti-huTfr HVR-L3
HQDYSSPT

SEQ ID NO: 168 - protein, 11 aa, synthetic construct
  REGION 1..11: note = anti-huTfr HVR-L1
RASEYINSYLA

SEQ ID NO: 169 - protein, 7 aa, synthetic construct
  REGION 1..7: note = anti-huTfr HVR-L2
KEKTLAE

SEQ ID NO: 170 - protein, 9 aa, synthetic construct
  REGION 1..9: note = anti-huTfr HVR-L3
QHHYGNPWT

SEQ ID NO: 171 - protein, 10 aa, synthetic construct
  REGION 1..10: note = anti-huTfr HVR-L1
SASSSISSLH

SEQ ID NO: 172 - protein, 9 aa, synthetic construct
  REGION 1..9: note = anti-huTfr HVR-L2
QNDYGYPLT

SEQ ID NO: 173 - protein, 17 aa, synthetic construct
  REGION 1..17: note = anti-huTfr HVR-L1
KSSQSLFNSENQKNYLT

SEQ ID NO: 174 - protein, 17 aa, synthetic construct
  REGION 1..17: note = anti-huTfr HVR-L1
KSSQSLLNSENQKNYLT

SEQ ID NO: 175 - protein, 7 aa, synthetic construct
  REGION 1..7: note = anti-huTfr HVR-L2
DTSELAS

SEQ ID NO: 176 - protein, 9 aa, synthetic construct
  REGION 1..9: note = anti-huTfr HVR-L2
HQRSGYPWT

SEQ ID NO: 177 - protein, 10 aa, synthetic construct
  REGION 1..10: note = anti-huTfr HVR-L1
SASSSISYMH

SEQ ID NO: 178 - protein, 7 aa, synthetic construct
  REGION 1..7: note = anti-huTfr HVR-L3
HQRSSYT

SEQ ID NO: 179 - protein, 11 aa, synthetic construct
  REGION 1..11: note = anti-huTfr HVR-L1
KASQNVRTAVA

SEQ ID NO: 180 - protein, 7 aa, synthetic construct
  REGION 1..7: note = anti-huTfr HVR-L2
LASNRHT

SEQ ID NO: 181 - protein, 9 aa, synthetic construct
  REGION 1..9: note = anti-huTfr HVR-L3
LQHWNYPWT

SEQ ID NO: 182 - protein, 8 aa, synthetic construct
  REGION 1..8: note = anti-huTfr HVR-L3
QQDYISPT

SEQ ID NO: 183 - protein, 20 aa, synthetic construct
  REGION 1..20: note = Linker
GGSEGKSSGSGSESKSTGGS

SEQ ID NO: 184 - protein, 15 aa, synthetic construct
  REGION 1..15: note = Linker
GGGGSGGGGSGGGGS

SEQ ID NO: 185 - protein, 486 aa, synthetic construct
  REGION 1..486: note = Isotype control heavy chain hIgG1 wt hole scFv (T366S_L368A_Y407V)-(G4S)3 linker-VH-20AA linker-VL
  SITE 465: note = wherein X is the amino acid sequence of any one of SEQ ID NOs:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 313, 315, 317, 318, 319, 320, 321, 322, 323, 324, 325, 327, 330, 334, 335, 337, 339, 343, 344, 345, 346, 348, 349, 351, 357, 359, 361, 363, 364, 369, 371, 372, 375, 376, 378, 380, 384, 385, 387, 389, 390, 391, 393, 394, 396, 397, 398, 399, 401, and 402
  SITE 486: note = wherein X is the amino acid sequence of any one of SEQ ID NOs:11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 314, 316, 326, 328, 329, 331, 332, 333, 336, 338, 340, 341, 342, 347, 350, 352, 353, 354, 355, 356, 358, 360, 362, 365, 366, 367, 368, 370, 373, 374, 377, 379, 381, 382, 383, 386, 388, 392, 395, 400, and 403.
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSXGGSEGKSSGSGSESKSTGGSX

SEQ ID NO: 186 - protein, 486 aa, synthetic construct
  REGION 1..485: note = Isotype control heavy chain hIgG1 wt hole scFv (T366S_L368A_Y407V)-(G4S)3 linker-VH-20AA linker-VL with mutations (H435R, Y436F)
  SITE 465: note = wherein X is the amino acid sequence of any one of SEQ ID NOs:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 313, 315, 317, 318, 319, 320, 321, 322, 323, 324, 325, 327, 330, 334, 335, 337, 339, 343, 344, 345, 346, 348, 349, 351, 357, 359, 361, 363, 364, 369, 371, 372, 375, 376, 378, 380, 384, 385, 387, 389, 390, 391, 393, 394, 396, 397, 398, 399, 401, and 402
  SITE 486: note = Wherein X is the amino acid sequence of any one of SEQ ID NO:s 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 314, 316, 326, 328, 329, 331, 332, 333, 336, 338, 340, 341, 342, 347, 350, 352, 353, 354, 355, 356, 358, 360, 362, 365, 366, 367, 368, 370, 373, 374, 377, 379, 381, 382, 383, 386, 388, 392, 395, 400, and 403
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSXGGSEGKSSGSGSESKSTGGSX

SEQ ID NO: 187 - protein, 449 aa, synthetic construct
  REGION 1..449: note = Isotype control heavy chain hIgG1 wt knob (T366W)
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 214 aa, synthetic construct
  REGION 1..214: note = Isotype control light chain
DIQMTQSPSSLSASVGDRVTITCRASQSISSYLNWYQQKPGKAPKLLIYAASSLQSGVPSRFSGSGSGTDFTLTISSLQPEDFATYYCQQADLPAFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - protein, 486 aa, synthetic construct
  REGION 1..485: note = Isotype control heavy chain hIgG1 LALAPS hole scFv (T366S_L368A_Y407V) (L234A_L235A_P331S)-(G4S)3 linker-VH-20AA linker-VL
  SITE 465: note = Wherein X can be the amino acid sequence of any one of SEQ ID NOs: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 313, 315, 317, 318, 319, 320, 321, 322, 323, 324, 325, 327, 330, 334, 335, 337, 339, 343, 344, 345, 346, 348, 349, 351, 357, 359, 361, 363, 364, 369, 371, 372, 375, 376, 378, 380, 384, 385, 387, 389, 390, 391, 393, 394, 396, 397, 398, 399, 401, and 402
  SITE 486: note = wherein X is the amino acid sequence of any one of SEQ ID NOs:11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 314, 316, 326, 328, 329, 331, 332, 333, 336, 338, 340, 341, 342, 347, 350, 352, 353, 354, 355, 356, 358, 360, 362, 365, 366, 367, 368, 370, 373, 374, 377, 379, 381, 382, 383, 386, 388, 392, 395, 400, and 403.
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSXGGSEGKSSGSGSESKSTGGSX

SEQ ID NO: 190 - protein, 486 aa, synthetic construct
  REGION 1..485: note = Isotype control heavy chain hIgG1 LALAPS hole scFv (T366S_L368A_Y407V) (L234A_L235A_P331S)-(G4S)3 linker-VH-20AA linker-VL with mutations (H435R, Y436F)
  SITE 486: note = Wherein X is the amino acid sequence of any one of SEQ ID NOs:11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 314, 316, 326, 328, 329, 331, 332, 333, 336, 338, 340, 341, 342, 347, 350, 352, 353, 354, 355, 356, 358, 360, 362, 365, 366, 367, 368, 370, 373, 374, 377, 379, 381, 382, 383, 386, 388, 392, 395, 400, and 403
  SITE 465: note = wherein X1 is the amino acid sequence of any one of SEQ ID NOs:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 313, 315, 317, 318, 319, 320, 321, 322, 323, 324, 325, 327, 330, 334, 335, 337, 339, 343, 344, 345, 346, 348, 349, 351, 357, 359, 361, 363, 364, 369, 371, 372, 375, 376, 378, 380, 384, 385, 387, 389, 390, 391, 393, 394, 396, 397, 398, 399, 401, and 402; note = wherein X1 is the amino acid sequence of any one of SEQ ID NOs:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 313, 315, 317, 318, 319, 320, 321, 322, 323, 324, 325, 327, 330, 334, 335, 337, 339, 343, 344, 345, 346, 348, 349, 351, 357, 359, 361, 363, 364, 369, 371, 372, 375, 376, 378, 380, 384, 385, 387, 389, 390, 391, 393, 394, 396, 397, 398, 399, 401, and 402
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSXGGSEGKSSGSGSESKSTGGSX

SEQ ID NO: 191 - protein, 449 aa, synthetic construct
  REGION 1..449: note = Isotype control heavy chain hIgG1 LALAPS knob (T366W) (L234A_L235A_P331S)
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 486 aa, synthetic construct
  REGION 1..485: note = Isotype control heavy chain hIgG1 NSLF hole scFv (T366S_L368A_Y407V) (N325S_L328F)-(G4S)3 linker-VH-20AA linker-VL
  SITE 465: note = wherein X1 is the amino acid sequence of any one of SEQ ID NOs:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 313, 315, 317, 318, 319, 320, 321, 322, 323, 324, 325, 327, 330, 334, 335, 337, 339, 343, 344, 345, 346, 348, 349, 351, 357, 359, 361, 363, 364, 369, 371, 372, 375, 376, 378, 380, 384, 385, 387, 389, 390, 391, 393, 394, 396, 397, 398, 399, 401, and 402
  SITE 486: note = Wherein X is the amino acid sequence of any one of SEQ ID NOs:11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 314, 316, 326, 328, 329, 331, 332, 333, 336, 338, 340, 341, 342, 347, 350, 352, 353, 354, 355, 356, 358, 360, 362, 365, 366, 367, 368, 370, 373, 374, 377, 379, 381, 382, 383, 386, 388, 392, 395, 400, and 403.
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSXGGSEGKSSGSGSESKSTGGSX

SEQ ID NO: 193 - protein, 486 aa, synthetic construct
  REGION 1..485: note = Isotype control heavy chain hIgG1 NSLF hole scFv (T366S_L368A_Y407V) (N325S_L328F)-(G4S)3 linker-VH-20AA linker-VL mutations (H435R, Y436F)
  SITE 465: note = Wherein X is the amino acid sequence of any one of SEQ ID NOs:10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 313, 315, 317, 318, 319, 320, 321, 322, 323, 324, 325, 327, 330, 334, 335, 337, 339, 343, 344, 345, 346, 348, 349, 351, 357, 359, 361, 363, 364, 369, 371, 372, 375, 376, 378, 380, 384, 385, 387, 389, 390, 391, 393, 394, 396, 397, 398, 399, 401, and 402
  SITE 486: note = wherein X2 is the amino acid sequence of any one of SEQ ID NOs:11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 314, 316, 326, 328, 329, 331, 332, 333, 336, 338, 340, 341, 342, 347, 350, 352, 353, 354, 355, 356, 358, 360, 362, 365, 366, 367, 368, 370, 373, 374, 377, 379, 381, 382, 383, 386, 388, 392, 395, 400, and 403.
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSXGGSEGKSSGSGSESKSTGGSX

SEQ ID NO: 194 - protein, 449 aa, synthetic construct
  REGION 1..449: note = Isotype control heavy chain hIgG1 NSLF knob (T366W) (N325S_L328F)
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 251 aa, synthetic construct
  REGION 1..251: note = parental TfR9 scFv
EVQLQQSGPELVKPGASVKISCKTSGYTFTEYAMHWVKQSHGKSLEWIGGINPNDGGTSYNQKFKGKATLTVDKSSSTAYMELRSLTSEDSAVYYCSSQRWLLRGGDYWGQGTTLTVSSGGSEGKSSGSGSESKSTGGSDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 196 - protein, 251 aa, synthetic construct
  REGION 1..251: note = TfR9.1A scFv
QVQLVQSGAEVKKPGASVKVSCKASGYTFTEYAMHWVRQAPGQGLEWMGGINPNDGGTSYNQKFKGRVTMTRDTSTSTVYMELSSLRSEDTAVYYCASQRWLLRGGDYWGQGTLVTVSSGGSEGKSSGSGSESKSTGGSDVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHFPQTFGGGTKVEIK

SEQ ID NO: 197 - protein, 251 aa, synthetic construct
  REGION 1..251: note = TfR9.1B scFv
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 198 - protein, 251 aa, synthetic construct
  REGION 1..251: note = TfR9.2A scFv
QVQLVQSGAEVKKPGASVKVSCKASGYTFTEYAMHWVRQAPGQRLEWMGGINPNDGGTSYNQKFKGRVTITRDTSASTAYMELSSLRSEDTAVYYCASQRWLLRGGDYWGQGTLVTVSSGGSEGKSSGSGSESKSTGGSDVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHFPQTFGGGTKVEIK

SEQ ID NO: 199 - protein, 251 aa, synthetic construct
  REGION 1..251: note = TfR9.2B scFv
QVQLVQSGAEVKKPGASVKVSCKASGYTFTEYAMHWVRQAPGQRLEWMGGINPNDGGTSYNQKFKGRVTITVDTSASTAYMELRSLRSEDTAVYYCASQRWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 200 - protein, 251 aa, synthetic construct
  REGION 1..251: note = TfR9.3A scFv
EVQLVQSGAEVKKPGATVKISCKVSGYTFTEYAMHWVQQAPGKGLEWMGGINPNDGGTSYNQKFKGRVTITADTSTDTAYMELSSLRSEDTAVYYCATQRWLLRGGDYWGQGTLVTVSSGGSEGKSSGSGSESKSTGGSDVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHFPQTFGGGTKVEIK

SEQ ID NO: 201 - protein, 251 aa, synthetic construct
  REGION 1..251: note = TfR9.3B scFv
EVQLVQSGAEVKKPGATVKISCKVSGYTFTEYAMHWVQQAPGKGLEWMGGINPNDGGTSYNQKFKGRVTITADKSTDTAYMELRSLRSEDTAVYYCATQRWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 202 - protein, 251 aa, synthetic construct
  REGION 1..251: note = TfR9.4A scFv
QVQLVQSGAEVKKPGASVKVSCKVSGYTLTEYAMHWVRQAPGKGLEWMGGINPNDGGTSYNQKFKGRVTMTEDTSTDTAYMELSSLRSEDTAVYYCATQRWLLRGGDYWGQGTLVTVSSGGSEGKSSGSGSESKSTGGSDVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHFPQTFGGGTKVEIK

SEQ ID NO: 203 - protein, 251 aa, synthetic construct
  REGION 1..251: note = TfR9.4B scFv
QVQLVQSGAEVKKPGASVKVSCKVSGYTLTEYAMHWVRQAPGKGLEWMGGINPNDGGTSYNQKFKGRVTMTEDKSTDTAYMELRSLRSEDTAVYYCATQRWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 204 - protein, 251 aa, synthetic construct
  REGION 1..251: note = TfR9.5A scFv
QVQLVQSGAEVKKPGASVKVSCKASGYTFTEYAMHWVRQAPGQGLEWMGGINPNDGGTSYNQKFKGRVTSTRDTSISTAYMELSRLRSDDTVVYYCASQRWLLRGGDYWGQGTLVTVSSGGSEGKSSGSGSESKSTGGSDVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHFPQTFGGGTKVEIK

SEQ ID NO: 205 - protein, 251 aa, synthetic construct
  REGION 1..251: note = TfR9.5B scFv
QVQLVQSGAEVKKPGASVKVSCKASGYTFTEYAMHWVRQAPGQGLEWMGGINPNDGGTSYNQKFKGRVTLTVDTSISTAYMELSRLRSDDTVVYYCASQRWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 206 - protein, 247 aa, synthetic construct
  REGION 1..247: note = TfR9.5B scFv
QVQLQQPGSELVRPGASVKLSCKASGYTFTSYWMHWVKQRPGQGLEWIGNIYPGSGSSNYDEKFKNKATLTVDTSSNTAYMQLSSLTSEDSAVYYCTRSSYYGNFDTMDNWGQGTSVTVSSGGSEGKSSGSGSESKSTGGSQIVLTQSPAIMSASPGEKVTMTCSASSSIRYMNWYQQKPVTSPKRWIYDTSTLASGVPARFSGSGSGTSYSLTISSMEAEDAATYYCHQRSSYPWTFGGGTKLEIK

SEQ ID NO: 207 - protein, 247 aa, synthetic construct
  REGION 1..247: note = TfR12.1A scFv
QVQLVQSGAEVKKPGASVKVSCKASGYTFTSYWMHWVRQAPGQGLEWMGNIYPGSGSSNYDEKFKNRVTMTRDTSTSTVYMELSSLRSEDTAVYYCARSSYYGNFDTMDNWGQGTTVTVSSGGSEGKSSGSGSESKSTGGSDIQLTQSPSFLSASVGDRVTITCSASSSIRYMNWYQQKPGKAPKLLIYDTSTLASGVPSRFSGSGSGTEFTLTISSLQPEDFATYYCHQRSSYPWTFGGGTKVEIK

SEQ ID NO: 208 - protein, 247 aa, synthetic construct
  REGION 1..247: note = TfR12.1B scFv
QVQLVQSGAEVKKPGASVKVSCKASGYTFTSYWMHWVRQAPGQGLEWMGNIYPGSGSSNYDEKFKNRVTLTVDTSTSTAYMELSSLRSEDTAVYYCARSSYYGNFDTMDNWGQGTTVTVSSGGSEGKSSGSGSESKSTGGSQIQLTQSPSFLSASVGDRVTITCSASSSIRYMNWYQQKPGKAPKRLIYDTSTLASGVPSRFSGSGSGTSYTLTISSLQPEDFATYYCHQRSSYPWTFGGGTKVEIK

SEQ ID NO: 209 - protein, 247 aa, synthetic construct
  REGION 1..247: note = TfR12.2A scFv
QVQLVQSGAEVKKPGASVKVSCKASGYTFTSYWMHWVRQAPGQGLEWMGNIYPGSGSSNYDEKFKNRVTMTRDTSTSTVYMELSSLRSEDTAVYYCARSSYYGNFDTMDNWGQGTTVTVSSGGSEGKSSGSGSESKSTGGSEIVLTQSPATLSLSPGERATLSCSASSSIRYMNWYQQKPGQAPRLLIYDTSTLASGIPARFSGSGSGTDFTLTISSLEPEDFAVYYCHQRSSYPWTFGGGTKVEIK

SEQ ID NO: 210 - protein, 247 aa, synthetic construct
  REGION 1..247: note = TfR12.2B scFv
QVQLVQSGAEVKKPGASVKVSCKASGYTFTSYWMHWVRQAPGQGLEWMGNIYPGSGSSNYDEKFKNRVTLTVDTSTSTVYMELSSLRSEDTAVYYCARSSYYGNFDTMDNWGQGTTVTVSSGGSEGKSSGSGSESKSTGGSQIVLTQSPATLSLSPGERATLSCSASSSIRYMNWYQQKPGQAPRRLIYDTSTLASGIPARFSGSGSGTSFTLTISSLEPEDFAVYYCHQRSSYPWTFGGGTKVEIK

SEQ ID NO: 211 - protein, 247 aa, synthetic construct
  REGION 1..247: note = TfR12.3A scFv
QVQLVQSGAEVKKPGASVKVSCKASGYTFTSYWMHWVRQAPGQGLEWMGNIYPGSGSSNYDEKFKNRVTMTRDTSTSTVYMELSSLRSEDTAVYYCARSSYYGNFDTMDNWGQGTTVTVSSGGSEGKSSGSGSESKSTGGSDIQMTQSPSSLSASVGDRVTITCSASSSIRYMNWYQQKPGKAPKLLIYDTSTLASGVPSRFSGSGSGTDFTLTISSLQPEDFATYYCHQRSSYPWTFGGGTKVEIK

SEQ ID NO: 212 - protein, 247 aa, synthetic construct
  REGION 1..247: note = TfR12.3B scFv
QVQLVQSGAEVKKPGASVKVSCKASGYTFTSYWMHWVRQAPGQGLEWMGNIYPGSGSSNYDEKFKNRVTLTVDTSTSTVYMELSSLRSEDTAVYYCARSSYYGNFDTMDNWGQGTTVTVSSGGSEGKSSGSGSESKSTGGSQIQMTQSPSSLSASVGDRVTITCSASSSIRYMNWYQQKPGKAPKRLIYDTSTLASGVPSRFSGSGSGTDYTLTISSLQPEDFATYYCHQRSSYPWTFGGGTKVEIK

SEQ ID NO: 213 - protein, 247 aa, synthetic construct
  REGION 1..247: note = TfR12.4B scFv
QVQLVQSGAEVKKPGASVKVSCKASGYTFTSYWMHWVRQAPGQGLEWMGNIYPGSGSSNYDEKFKNRVTSTRDTSISTAYMELSRLRSDDTVVYYCARSSYYGNFDTMDNWGQGTTVTVSSGGSEGKSSGSGSESKSTGGSDIQMTQSPSSLSASVGDRVTITCSASSSIRYMNWYQQKPGKAPKLLIYDTSTLASGVPSRFSGSGSGTDFTLTISSLQPEDFATYYCHQRSSYPWTFGGGTKVEIK

SEQ ID NO: 214 - protein, 247 aa, synthetic construct
  REGION 1..247: note = TfR12.4B scFv
QVQLVQSGAEVKKPGASVKVSCKASGYTFTSYWMHWVRQAPGQGLEWMGNIYPGSGSSNYDEKFKNRVTLTVDTSISTAYMELSRLRSDDTVVYYCARSSYYGNFDTMDNWGQGTTVTVSSGGSEGKSSGSGSESKSTGGSQIQMTQSPSSLSASVGDRVTITCSASSSIRYMNWYQQKPGKAPKRLIYDTSTLASGVPSRFSGSGSGTSYTLTISSLQPEDFATYYCHQRSSYPWTFGGGTKVEIK

SEQ ID NO: 215 - protein, 247 aa, synthetic construct
  REGION 1..247: note = TfR12.EB scFv
QVQLVQSGAEVKKPGASVKVSCKASGYTFTSYWMHWVRQAPGQGLEWMGNIYPGSGSSSYAQKFQGRVTMTVDTSTNTVYMELSSLRSEDTAVYYCTRSSYYGNFDTMDNWGQGTMVTVSSGGSEGKSSGSGSESKSTGGSDIQLTQSPSFLSASVGDRVTITCRASSSIRYMNWYQQKPGKAPKRLIYDTSTLQSGVPSRFSGSGSGTEYTLTISSLQPEDFATYYCHQRSSYPWTFGGGTKVEIK

SEQ ID NO: 216 - protein, 247 aa, synthetic construct
  REGION 1..247: note = parental TfR15
QVQLQQPGSELVRPGASVKLSCKASGYTFTSYWMHWVKQRPGQGLEWIGNIYPGSGSSNYDEKFKSKATLTVDTSSSTAYMQLNSLTSEDSAVYYCTRSSYYGNFDTMDNWGQGTSVTVSSGGSEGKSSGSGSESKSTGGSQIVLTQSPAIMSASPGEKVTMTCSASSSIRYMNWYQQKPVTSPKRWIYDTSTLASGVPARFSGSGSGTSYSLTISSMEAEDAATYYCHQRSSYPWTFGGGTKLEIK

SEQ ID NO: 217 - protein, 247 aa, synthetic construct
  REGION 1..247: note = TfR15.WH1
QVQLVQSGAEVKKPGASVKVSCKASGYTFTSYWMHWVRQAPGQGLEWMGIIYPGSGSSNYAQKFQGRVTMTRDTSTSTVYMELSSLRSEDTAVYYCTRSSYYGNFDTMDNWGQGTTVTVSSGGSEGKSSGSGSESKSTGGSDIQLTQSPSFLSASVGDRVTITCRASSSIRYMNWYQQKPGKAPKRLIYDTSTLQSGVPSRFSGSGSGTEFTLTISSLQPEDFATYYCHQRSSYPWTFGGGTKVEIK

SEQ ID NO: 218 - protein, 247 aa, synthetic construct
  REGION 1..247: note = TfR15.WH2
QVQLVQSGAEVKKPGASVKVSCKASGYTFTSYWMHWVRQAPGQGLEWMGIIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 219 - protein, 247 aa, synthetic construct
  REGION 1..247: note = TfR15.WH3
QVQLVQSGAEVKKPGASVKVSCKASGYTFTSYWMHWVRQAPGQGLEWMGIIYPGSGSSNYAQKFQGRVTMTRDTSTSTVYMELSSLRSEDTAVYYCTRSSYYGNFDTMDNWGQGTTVTVSSGGSEGKSSGSGSESKSTGGSDIQMTQSPSSLSASVGDRVTITCRASSSIRYLNWYQQKPGKAPKRLIYDTSSLQSGVPSRFSGSGSGTDFTLTISSLQPEDFATYYCHQRSSYPWTFGGGTKVEIK

SEQ ID NO: 220 - protein, 247 aa, synthetic construct
  REGION 1..247: note = TfR15.WH4
QVQLVQSGAEVKKPGASVKVSCKASGYTFTSYWMHWVRQAPGQGLEWMGNIYPGSGSSSYAQKFQGRVTMTRDTSTSTVYMELSSLRSEDTAVYYCTRSSYYGNFDTMDNWGQGTTVTVSSGGSEGKSSGSGSESKSTGGSDIQLTQSPSFLSASVGDRVTITCRASSSIRYMNWYQQKPGKAPKRLIYDTSTLQSGVPSRFSGSGSGTEFTLTISSLQPEDFATYYCHQRSSYPWTFGGGTKVEIK

SEQ ID NO: 221 - protein, 247 aa, synthetic construct
  REGION 1..247: note = TfR15.WH6
QVQLVQSGAEVKKPGASVKVSCKASGYTFTSYWMHWVRQAPGQGLEWMGNIYPGSGSSSYAQKFQGRVTMTRDTSTSTVYMELSSLRSEDTAVYYCTRSSYYGNFDTMDNWGQGTTVTVSSGGSEGKSSGSGSESKSTGGSDIQMTQSPSSLSASVGDRVTITCRASSSIRYLNWYQQKPGKAPKRLIYDTSSLQSGVPSRFSGSGSGTDFTLTISSLQPEDFATYYCHQRSSYPWTFGGGTKVEIK

SEQ ID NO: 222 - protein, 247 aa, synthetic construct
  REGION 1..247: note = TfR15.WH7
QVQLVQSGAEVKKPGASVKVSCKASGYTFTSYWMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSDIQLTQSPSFLSASVGDRVTITCRASSSIRYMNWYQQKPGKAPKRLIYDTSTLQSGVPSRFSGSGSGTEFTLTISSLQPEDFATYYCHQRSSYPWTFGGGTKVEIK

SEQ ID NO: 223 - protein, 247 aa, synthetic construct
  REGION 1..247: note = TfR15.WH8
QVQLVQSGAEVKKPGASVKVSCKASGYTFTSYWMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 224 - protein, 247 aa, synthetic construct
  REGION 1..247: note = TfR15.WH9
QVQLVQSGAEVKKPGASVKVSCKASGYTFTSYWMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSDIQMTQSPSSLSASVGDRVTITCRASSSIRYLNWYQQKPGKAPKRLIYDTSSLQSGVPSRFSGSGSGTDFTLTISSLQPEDFATYYCHQRSSYPWTFGGGTKVEIK

SEQ ID NO: 225 - protein, 247 aa, synthetic construct
  REGION 1..247: note = TfR15.WH10
QVQLVQSGAEVKKPGASVKVSCKASGYTFTSYWMHWVRQAPGQGLEWMGNIYPGSGSSNYAQKFQGRVTLTVDTSTSTVYMELSSLRSEDTAVYYCTRSSYYGNFDTMDNWGQGTTVTVSSGGSEGKSSGSGSESKSTGGSDIQLTQSPSFLSASVGDRVTITCRASSSIRYMNWYQQKPGKAPKRLIYDTSTLQSGVPSRFSGSGSGTEFTLTISSLQPEDFATYYCHQRSSYPWTFGGGTKVEIK

SEQ ID NO: 226 - protein, 247 aa, synthetic construct
  REGION 1..247: note = TfR15.WH11
QVQLVQSGAEVKKPGASVKVSCKASGYTFTSYWMHWVRQAPGQGLEWMGNIYPGSGSSNYAQKFQGRVTLTVDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 227 - protein, 247 aa, synthetic construct
  REGION 1..247: note = TfR15.WH12
QVQLVQSGAEVKKPGASVKVSCKASGYTFTSYWMHWVRQAPGQGLEWMGNIYPGSGSSNYAQKFQGRVTLTVDTSTSTVYMELSSLRSEDTAVYYCTRSSYYGNFDTMDNWGQGTTVTVSSGGSEGKSSGSGSESKSTGGSDIQMTQSPSSLSASVGDRVTITCRASSSIRYLNWYQQKPGKAPKRLIYDTSSLQSGVPSRFSGSGSGTDFTLTISSLQPEDFATYYCHQRSSYPWTFGGGTKVEIK

SEQ ID NO: 228 - protein, 251 aa, synthetic construct
  REGION 1..251: note = TfR9.1B.1 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLQRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQSFGGGTKVEIK

SEQ ID NO: 229 - protein, 251 aa, synthetic construct
  REGION 1..251: note = TfR9.1B.2 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLLRGGDHWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 230 - protein, 251 aa, synthetic construct
  REGION 1..251: note = TfR9.1B.3 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQSFGGGTKVEIK

SEQ ID NO: 231 - protein, 251 aa, synthetic construct
  REGION 1..251: note = TfR9.1B.4 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKSSQSLLDSDGKIYLNWLQQRPGQSPRRLIYLVSKLDSGVPDRFSGSGSGTDFTLKISRVEAEDVGVYYCWQGTHFPQTFGGGTKVEIK

SEQ ID NO: 232 - protein, 251 aa, synthetic construct
  REGION 1..251: note = TfR9.1B.5 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLLIGGDYWGQGTLVTVSSGGSEGKSSGSGSESKSTGGSDVVMTQSPLSLPVTLGQPASISCKSSQSDLDSDGKTYLNWLQQRPGQSPRRLIYLVSKLDSGVPDRFSGSGSGTDFTLKISRVEAEDVGVYYCWQGTSFPQTFGGGTKVEIK

SEQ ID NO: 233 - protein, 251 aa, synthetic construct
  REGION 1..251: note = TfR9.1B.6 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKTSQSLLDPDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 234 - protein, 251 aa, synthetic construct
  REGION 1..251: note = TfR9.1B.7 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLQRGGDYWGQGTLVTVSSGGSEGKPSGSGSESKSTGGSDVVMTQSPLSLPVTLGQPASISCKSSQSLKDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 235 - protein, 251 aa, synthetic construct
  REGION 1..251: note = TfR9.1B.8 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKSSKSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 236 - protein, 251 aa, synthetic construct
  REGION 1..251: note = TfR9.1B.9 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKSSVSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 237 - protein, 251 aa, synthetic construct
  REGION 1..251: note = TfR9.1B.10 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 238 - protein, 251 aa, synthetic construct
  REGION 1..251: note = TfR9.1B.11 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKSSQSLLDSDGVTYLNWLQQRPGQSPRRLIYLVSKLDSGVPDRFSGSGSGTDFTLKISRVEAEDVGVYYCWQGTHFPQTFGGGTKVEIK

SEQ ID NO: 239 - protein, 251 aa, synthetic construct
  REGION 1..251: note = TfR9.1B.12 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 240 - protein, 251 aa, synthetic construct
  REGION 1..251: note = TfR9.1B.14 anti- tFR scFv Sequence
QVQLVQSGAEVKKPGASVKVSCKASGYTFTEYAMHWVRQAPGQGLEWMGGINPNDGGTHYNQKFKGRVTMTVDTSTSTAYMELSSLRSEDTAVYYCASQRWLLRGGDYWGQGTLVTVSSGGSEGKSSGSGSESKSTGGSDVVMTQSPLSLPVTLGQPASISCKSSRSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 241 - protein, 251 aa, synthetic construct
  REGION 1..251: note = TfR9.1B.15 anti- tFR scFv Sequence
QVQLVQSGAEVKKPGASVKVSCKASGYTFTEYAMHWVRQAPGQGLEWMGGINPNDGGTSYSQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKSSQSLLDKDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 242 - protein, 251 aa, synthetic construct
  REGION 1..251: note = TfR9.1B.16 anti- tFR scFv Sequence
QVQLVQSGAEVKKPGASVKVSCKASGYTFTEYAMHWVRQAPGQGLEWMGGINPNDSGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 243 - protein, 251 aa, synthetic construct
  REGION 1..251: note = TfR9.1B.17 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSNLRSEDTAVYYCASQRWLLKGGDYWGQGTLVTVSSGGSEGKSSGSGSESKSTGGSDVVMTQSPLSLPVTLGQPASISCKSSQSLLDSDGITYLNWLQQRPGQSPRRLIYLVSKLDSGVPDRFSGSGSGTDFTLKISRVEAEDVGVYYCWQGTHFPQTFGGGTKVEIK

SEQ ID NO: 244 - protein, 251 aa, synthetic construct
  REGION 1..251: note = TfR9.1B.18 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKSSTSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 245 - protein, 251 aa, synthetic construct
  REGION 1..251: note = TfR9.1B.19 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLQRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 246 - protein, 251 aa, synthetic construct
  REGION 1..251: note = TfR9.1B.20 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLQRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTKFPQTFGGGTKVEIK

SEQ ID NO: 247 - protein, 251 aa, synthetic construct
  REGION 1..251: note = TfR9.1B.21 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKSSQSLLDSDGRTYLNWLQQRPGQSPRRLIYLVSKLDSGVPDRFSGSGSGTDFTLKISRVEAEDVGVYYCWQGTHFPQTFGGGTKVEIK

SEQ ID NO: 248 - protein, 251 aa, synthetic construct
  REGION 1..251: note = TfR9.1B.22 anti- tFR scFv Sequence
QVQLVQSGAEVKKPGASVKVSCKASGYTFTEYAMHWVRQAPGQGLEWMGGINPKDGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 249 - protein, 251 aa, synthetic construct
  REGION 1..251: note = TfR9.1B.23 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLQRGGDYWGQGTLVTVSSGGSEGKSSGSGSESKSTGGSDVVMTQSPLSLPVTLGQPASISCKSSQSLIDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 250 - protein, 251 aa, synthetic construct
  REGION 1..251: note = TfR9.1B.24 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLMRGGDYWGQGTLVTVSSGGSEGKSSSSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 251 - protein, 251 aa, synthetic construct
  REGION 1..251: note = TfR9.1B.25 anti- tFR scFv Sequence
QVQLVQSGAEVKKPGASVKVSCKASGYTFTEHAMHWVRQAPGQGLEWMGGINPNDGGTSTNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKSSVSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 252 - protein, 251 aa, synthetic construct
  REGION 1..251: note = TfR9.1B.26 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLLRGGDYWGQGTLVTVSSGGSEDKSSGSGSESKSTGGSDVVMTQSPLSLPVTLGQPASISCKSSQSLLDSDGVTYLNWLQQRPGQSPRRLIYLVSKLDSGVPDRFSGSGSGTDFTLKISRVEAEDVGVYYCWQGTHFPQTFGGGTKVEIK

SEQ ID NO: 253 - protein, 251 aa, synthetic construct
  REGION 1..251: note = TfR9.1B.27 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKSSQSLLDEDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 254 - protein, 251 aa, synthetic construct
  REGION 1..251: note = TfR9.1B.28 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLQRGGDYWGQGTLVTVSSGGSEGKSSGSGSESKSTGGSDVVMTQSPLSLPVTLGQPASISCKSSQSLEDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 255 - protein, 251 aa, synthetic construct
  REGION 1..251: note = TfR9.1B.29 anti- tFR scFv Sequence
QVQLVQSGAEVKKPGASVKVSCKASGYAFTEYAMHWVRQAPGQGLEWMGGINPNDGGTSYNQKFKGRVTMTVDTSTSTAYMELSSLRSEDTAVYYCASQRWLERGGNYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 256 - protein, 251 aa, synthetic construct
  REGION 1..251: note = TfR9.1B.30 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLFRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 257 - protein, 251 aa, synthetic construct
  REGION 1..251: note = TfR9.1B.31 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLQRGGDYWGQGTLVTVSSGGSEGKSSGSGSESKSTGGSDVVMTQSPLSLPVTLGQPASISCKSSQSLQDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 258 - protein, 251 aa, synthetic construct
  REGION 1..251: note = TfR9.1B.32 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRSLLRGGDYWGQGTLVTVSSGGSEGKSSGSGSESKSTGGSDVVMTQSPLSLPVTLGQPASISCKPSQSLLDGDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 259 - protein, 251 aa, synthetic construct
  REGION 1..251: note = TfR9.1B.33 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLYRGGDYWGQGTLVTVSSGGSEGKSSGSGSESKSTGGSDVVMTQSPLSLPVTLGQPASISCKSSQSLLDEDGKTYLNWLQQRPGQSPRRLIYQVSKLDSGVPDRFSGSGSGTDFTLKISRVEAEDVGVYYCWQGTHFPQTFGGGTKVEIK

SEQ ID NO: 260 - skipped

SEQ ID NO: 261 - protein, 251 aa, synthetic construct
  REGION 1..251: note = TfR9.1B.34 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSSGGSEGKSSGSGSESKSTGGSDVVMTQSPLSLPVTLGQPASISCKTSQSLLDPDGKTYLNWLQQRPGQSPRRLIYLVSKLDSGVPDRFSGSGSGTDFTLKISRVEAEDVGVYYCWQSTHFPQTFGGGTKVEIK

SEQ ID NO: 262 - protein, 251 aa, synthetic construct
  REGION 1..251: note = TfR9.1B.35 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYHCWQGTHFPQSFGGGTKVEIK

SEQ ID NO: 263 - protein, 251 aa, synthetic construct; identical to SEQ ID NO: 238
  REGION 1..251: note = TfR9.1B.36 anti- tFR scFv Sequence

SEQ ID NO: 264 - protein, 251 aa, synthetic construct
  REGION 1..251: note = TfR9.1B.37 anti- tFR scFv Sequence
QVQLVQSGAEVKKPGASVKVSCKASGYTFTEYAMHWVRQAPGQGLEWMGGINPNDGGTFYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 265 - protein, 251 aa, synthetic construct
  REGION 1..251: note = TfR9.1B.38 anti- tFR scFv Sequence
QVQLVQSGAEVKKPGASVKVSCKASGYTFTEYAMHWVRQAPGQGLEWMGGINPNDGGTSYAQKFKGRVTMTVDTSTSTAYMELSSLRSEDTAVYYCASQRWLNRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 266 - protein, 251 aa, synthetic construct
  REGION 1..251: note = TfR9.1B.39 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 267 - protein, 251 aa, synthetic construct
  REGION 1..251: note = TfR9.1B.40 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLDRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTQFPQTFGGGTKVEIK

SEQ ID NO: 268 - protein, 251 aa, synthetic construct
  REGION 1..251: note = TfR9.1B.41 anti- tFR scFv Sequence
QVQLVQSGAEVKKPGASVKVSCKASGYTFTEYAMHWVRQAPGQGLEWMGGINPNDGGTHYNQKFKGRVTMTVDTSTSTAYMELSSLRSEDTAVYYCASQRWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 269 - protein, 251 aa, synthetic construct
  REGION 1..251: note = TfR9.1B.42 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRELLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 270 - protein, 251 aa, synthetic construct
  REGION 1..251: note = TfR9.1B.43 anti- tFR scFv Sequence
QVQLVQSGAEVKKPGASVKVSCKASGYTFTEYAMHWVRQAPGQGLEWMGGINPNDGGTSYLQKFKGRVTMTVDTSTSTAYMELSSLRSEDTAVYYCASQRWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 271 - protein, 251 aa, synthetic construct
  REGION 1..251: note = TfR9.1B.44 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLLRGGDYWGQGTLVTVSSGGSEGKSSGSGSESKSTGGSDVVMTQSPLSLPVTLGQPASISCKSSQSLLDSIGKTYLNWLQQRPGQSPRRLIYLVSKLDSGVPDRFSGSGSGTDFTLKISRVEAEDVGVYYCWQGTHFPQTFGGGTKVEIK

SEQ ID NO: 272 - protein, 251 aa, synthetic construct
  REGION 1..251: note = TfR9.1B.45 anti- tFR scFv Sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLQRGGDYWGQGTLVTVSSGGSEGKSSGSGSESKSTGGSDVVMTQSPLSLPVTLGQPASISCKSSVSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 273 - protein, 251 aa, synthetic construct
  REGION 1..251: note = TfR9.1B.46 anti- tFR scFv Sequence
QVQLVQSGAEVKKPGASVKVSCKASGYTFTEYAMHWVRQAPGQGLEWMGGINPNDGGTSYLQKFKGRVTMTVDTSTSTAYMELSSLRSEDTAVYYCASQRWLLRGGDYWGQGTLVTVSSGGSEGKSSGSGSESKSTGGSDVVMTQSPLSLPVTLGQPASISCKSSQSLLDSIGKTYLNWLQQRPGQSPRRLIYLVSKLDSGVPDRFSGSGSGTDFTLKISRVEAEDVGVYYCWQGTHFPQTFGGGTKVEIK

SEQ ID NO: 274 - protein, 251 aa, synthetic construct; identical to SEQ ID NO: 266
  REGION 1..251: note = TfR9.1B.39 scFv

SEQ ID NO: 275 - protein, 251 aa, synthetic construct
  REGION 1..251: note = TfR9.1B.39.5 scFv
QVQLVQSGAEVKKPGASVKVSCKASGYTFTEYAMHWVRQAPGQGLEWMGGIDPRSGGTSYNQKFKGRVTMTVDTSTSTAYMELSSLRSEDTAVYYCASQGWLKRGGDYWGQGTLVTVSSGGSEGRSSGSGSESKSTGGSDVVMTQSPLSLPVTLGQPASISCKASQSLLDEEGKTYLNWLQQRPGQSPRRLIYLVSKLDSGVPDRFSGSGSGTDFTLKISRVEAEDVGVYYCWQGTHFPQTFGGGTKVEIK

SEQ ID NO: 276 - protein, 251 aa, synthetic construct
  REGION 1..251: note = TfR9.1B.39.8 scFv
QVQLVQSGAEVKKPGASVKVSCKASGYTFTEYAMHWVRQAPGQGLEWMGGIDPRSGGTSYNQKFKGRVTMTVDTSTSTAYMELSSLRSEDTAVYYCASQGWLQRGGDYWGQGTLVTVSSGGSEGKSSGSGSESKSTGGSDVVMTQSPLSLPVTLGQPASISCKASQSLLDEEGKTYLNWLQQRPGQSPRRLIYLVSKLDSGVPDRFSGSGSGTDFTLKISRVEAEDVGVYYCWQGTHFPQTFGGGTKVEIK

SEQ ID NO: 277 - protein, 251 aa, synthetic construct
  REGION 1..251: note = TfR9.1B.39.36 scFv
QVQLVQSGAEVKKPGASVKVSCKASGYTFTEYAMHWVRQAPGQGLEWMGGIDPNTGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEEGKTYLNWLQQRPGQSPRRLIYLVSKLDSGVPDRFSGSGSGTDFTLKISRVEAEDVGVYYCWQGTHFPQTFGGGTKVEIK

SEQ ID NO: 278 - protein, 251 aa, synthetic construct
  REGION 1..251: note = TfR9.1B.39.37 scFv
QVQLVQSGAEVKKPGASVKVSCKASGYTFTEYAMHWVRQAPGQGLEWMGGIAPGTGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEEGKTYLNWLQQRPGQSPRRLIYLVSKLDSGVPDRFSGSGSGTDFTLKISRVEAEDVGVYYCWQGTHFPQTFGGGTKVEIK

SEQ ID NO: 279 - protein, 251 aa, synthetic construct
  REGION 1..251: note = TfR9.1B.39.38 scFv
QVQLVQSGAEVKKPGASVKVSCKASGYTFTEYAMHWVRQAPGQGLEWMGGIAPNTGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEGGKTYLNWLQQRPGQSPRRLIYLVSKLDSGVPDRFSGSGSGTDFTLKISRVEAEDVGVYYCWQGTHFPQTFGGGTKVEIK

SEQ ID NO: 280 - protein, 251 aa, synthetic construct
  REGION 1..251: note = TfR9.1B.39.39 scFv
QVQLVQSGAEVKKPGASVKVSCKASGYTFTEYAMHWVRQAPGQGLEWMGGINPSFGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEEGKTYLNWLQQRPGQSPRRLIYLVSKLDSGVPDRFSGSGSGTDFTLKISRVEAEDVGVYYCWQGTHFPQTFGGGTKVEIK

SEQ ID NO: 281 - protein, 251 aa, synthetic construct
  REGION 1..251: note = TfR9.1B.39.40 scFv
QVQLVQSGAEVKKPGASVKVSCKASGYTFTEYAMHWVRQAPGQGLEWMGGINPKEGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEDQKTYLNWLQQRPGQSPRRLIYLVSKLDSGVPDRFSGSGSGTDFTLKISRVEAEDVGVYYCWQGTHFPQTFGGGTKVEIK

SEQ ID NO: 282 - protein, 247 aa, synthetic construct
  REGION 1..247: note = TfR15.WH7.1
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSDIQLTQSPSFLSASVGDRVTITCRASSSIRYMNWYQQKPGKAPKRLIYDTSTLQSGVPSRFSGSGSGTEFTLTISSLQPEDFATYYCHQRSSYPWTFGGGTKVEIK

SEQ ID NO: 283 - protein, 247 aa, synthetic construct
  REGION 1..247: note = TfR15.WH8.1
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 284 - protein, 247 aa, synthetic construct
  REGION 1..247: note = TfR15.WH12.1
QVQLVQSGAEVKKPGASVKVSCKASGYTFTSYYMHWVRQAPGQGLEWMGNIYPGSGSSNYAQKFQGRVTLTVDTSTSTVYMELSSLRSEDTAVYYCTRSSYYGNFDTMDNWGQGTTVTVSSGGSEGKSSGSGSESKSTGGSDIQMTQSPSSLSASVGDRVTITCRASSSIRYLNWYQQKPGKAPKRLIYDTSSLQSGVPSRFSGSGSGTDFTLTISSLQPEDFATYYCHQRSSYPWTFGGGTKVEIK

SEQ ID NO: 285 - protein, 4 aa, synthetic construct
  REGION 1..4: note = Linker
GGGG

SEQ ID NO: 286 - protein, 6 aa, synthetic construct
  REGION 1..6: note = Linker
GGGNGG

SEQ ID NO: 287 - protein, 4 aa, synthetic construct
  REGION 1..4: note = Linker
GGGS

SEQ ID NO: 288 - protein, 20 aa, synthetic construct
  REGION 1..20: note = Linker
GGGGSGGGGSGGGGSGGGGS

SEQ ID NO: 289 - protein, 15 aa, synthetic construct
  REGION 1..15: note = Linker
GGSGGGGSGGGGSGG

SEQ ID NO: 290 - protein, 119 aa, synthetic construct
  REGION 1..119: note = BACE 1 CNS Antigen
EVQLVESGGGLVQPGGSLRLSCAASGFTFSGYAIHWVRQAPGKGLEWVGWISPAGGSTDYADSVKGRFTISADTSKNTAYLQMNSLRAEDTAVYYCARGPFSPWVMDYWGQGTLVTVSS

SEQ ID NO: 291 - protein, 108 aa, synthetic construct
  REGION 1..108: note = BACE 1 CNS Antigen
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKR

SEQ ID NO: 292 - protein, 126 aa, synthetic construct
  REGION 1..126: note = BACE 1 CNS Antigen
EVQLVESGGGLVQPGGSLRLSCAASGFNFYYSSIHWVRQAPGKGLEWVASISPYSGYTSYADSVKGRFTISADTSKNTAYLQMNSLRAEDTAVYYCARQPTHYYYYAKGYKAMDYWGQGTLVTVSS

SEQ ID NO: 293 - protein, 108 aa, synthetic construct
  REGION 1..108: note = BACE 1 CNS Antigen
DIQMTQSPSSLSASVGDRVTITCRASQSVSSAVAWYQQKPGKAPKLLIYSASSLYSGVPSRFSGSGSGTDFTLTISSLQPEDFATYYCQQYSYSPFTFGQGTKVEIKR

SEQ ID NO: 294 - protein, 116 aa, synthetic construct
  REGION 1..116: note = Alpha-synuclein CNS Antigen
EVQLLESGGGLVQPGGSLRLSCAASGFTFSRFTMTWVRQAPGKGLEWVSAISGSGGGTSYADSVKGRLTVSRDNSKNTLYLQMNSLRAEDTAVYYCAKNWAPFDYWGQGTLVTVSS

SEQ ID NO: 295 - protein, 108 aa, synthetic construct
  REGION 1..108: note = Alpha-synuclein CNS Antigen
EIVLTQSPGTLSLSPGERATLSCRASQSVSRSYLAWYQQKPGQAPRLLIYGASSRATGIPDRFSGSGSGTDFTLTVSRLEPEDFAVYYCQQYGSSPWTFGQGTKVEIK

SEQ ID NO: 296 - protein, 116 aa, synthetic construct
  REGION 1..116: note = Alpha-synuclein CNS Antigen
EVQLLESGGGLVQTGGSLRLSCAASGFTFSSYAMTWVRQAPGKGLEWVSAIRSNGDRTDYADSVKGRFTISRDNSQNTLYLQMNSLRAEDTAVYYCAKNWAPFDSWGQGTLVTVSS

SEQ ID NO: 297 - protein, 108 aa, synthetic construct
  REGION 1..108: note = Alpha-synuclein CNS Antigen
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 298 - protein, 115 aa, synthetic construct
  REGION 1..115: note = Alpha Beta CNS Antigen
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSS

SEQ ID NO: 299 - protein, 112 aa, synthetic construct
  REGION 1..112: note = Alpha Beta CNS Antigen
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIK

SEQ ID NO: 300 - protein, 121 aa, synthetic construct
  REGION 1..121: note = Tau CNS Antigen
EVQLVESGGGLVQPGGSLKLSCAASGFNFNISAIHWVRQASGKGLEWVGRIRSKSHNYATLYAASLKGRFTLSRDDSRNTAYLQMSSLQTEDMAVYYCTVLSANYDTFDYWGQGTLVTVSS

SEQ ID NO: 301 - protein, 107 aa, synthetic construct
  REGION 1..107: note = Tau CNS Antigen
SYELTQPPSVSVSPGQTARISCFGDTLPKQYTYWYQQKPGQAPVLVIYKDTERPSGIPERFSGSSSGTTVTLTISGVQAEDEADYYCLSADNSATWVFGGGTKVTVL

SEQ ID NO: 302 - protein, 17 aa, synthetic construct
  REGION 1..17: note = anti-huTfr HVR-H2
VINPGSGETNYNEKFKG

SEQ ID NO: 303 - protein, 9 aa, synthetic construct
  REGION 1..8: note = anti-huTfr HVR-H3
RYDGPLLDH

SEQ ID NO: 304 - protein, 5 aa, synthetic construct
  REGION 1..5: note = Linker
GGSGG

SEQ ID NO: 305 - protein, 17 aa, synthetic construct
  REGION 1..17: note = anti-huTfr HVR-H2
NIYPGSGSSSYAQKFQG

SEQ ID NO: 306 - protein, 17 aa, synthetic construct
  REGION 1..17: note = anti-huTfr HVR-H2
IIYPGSGSSNYAQKFQG

SEQ ID NO: 307 - protein, 10 aa, synthetic construct
  REGION 1..10: note = anti-huTfr HVR-L1
RASSSIRYMN

SEQ ID NO: 308 - protein, 7 aa, synthetic construct
  REGION 1..7: note = anti-huTfr HVR-L1
DTSTLQS

SEQ ID NO: 309 - protein, 7 aa, synthetic construct
  REGION 1..7: note = anti-huTfr HVR-L2
DTSNRAT

SEQ ID NO: 310 - protein, 10 aa, synthetic construct
  REGION 1..10: note = anti-huTfr HVR-L3
RASSSIRYLN

SEQ ID NO: 311 - protein, 7 aa, synthetic construct
  REGION 1..7: note = anti-huTfr HVR-L3
DTSSLQS

SEQ ID NO: 312 - protein, 17 aa, synthetic construct
  REGION 1..17: note = anti-huTfr HVR-L1
NIYPGSGSSNYAQKFQG

SEQ ID NO: 313 - protein, 119 aa, synthetic construct
  REGION 1..119: note = TfR9.1A scFv VH
QVQLVQSGAEVKKPGASVKVSCKASGYTFTEYAMHWVRQAPGQGLEWMGGINPNDGGTSYNQKFKGRVTMTRDTSTSTVYMELSSLRSEDTAVYYCASQRWLLRGGDYWGQGTLVTVSS

SEQ ID NO: 314 - protein, 112 aa, synthetic construct
  REGION 1..112: note = TfR9.1A scFv VL
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHFPQTFGGGTKVEIK

SEQ ID NO: 315 - protein, 119 aa, synthetic construct
  REGION 1..119: note = TfR9.1B scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLLRGGDYWGQGTLVTVSS

SEQ ID NO: 316 - protein, 112 aa, synthetic construct
  REGION 1..112: note = TfR9.1B scFv VL
DVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 317 - protein, 119 aa, synthetic construct
  REGION 1..119: note = TfR9.2A scFv VH
QVQLVQSGAEVKKPGASVKVSCKASGYTFTEYAMHWVRQAPGQRLEWMGGINPNDGGTSYNQKFKGRVTITRDTSASTAYMELSSLRSEDTAVYYCASQRWLLRGGDYWGQGTLVTVSS

SEQ ID NO: 318 - protein, 119 aa, synthetic construct
  REGION 1..119: note = TfR9.2B scFv VH
QVQLVQSGAEVKKPGASVKVSCKASGYTFTEYAMHWVRQAPGQRLEWMGGINPNDGGTSYNQKFKGRVTITVDTSASTAYMELRSLRSEDTAVYYCASQRWLLRGGDYWGQGTLVTVSS

SEQ ID NO: 319 - protein, 119 aa, synthetic construct; identical to SEQ ID NO: 318
  REGION 1..119: note = TfR9.3A scFv VH

SEQ ID NO: 320 - protein, 119 aa, synthetic construct
  REGION 1..119: note = TfR9.3B scFv VH
EVQLVQSGAEVKKPGATVKISCKVSGYTFTEYAMHWVQQAPGKGLEWMGGINPNDGGTSYNQKFKGRVTITADKSTDTAYMELRSLRSEDTAVYYCATQRWLLRGGDYWGQGTLVTVSS

SEQ ID NO: 321 - protein, 119 aa, synthetic construct
  REGION 1..119: note = TfR9.4A scFv VH
QVQLVQSGAEVKKPGASVKVSCKVSGYTLTEYAMHWVRQAPGKGLEWMGGINPNDGGTSYNQKFKGRVTMTEDTSTDTAYMELSSLRSEDTAVYYCATQRWLLRGGDYWGQGTLVTVSS

SEQ ID NO: 322 - protein, 119 aa, synthetic construct
  REGION 1..119: note = TfR9.4B scFv VH
QVQLVQSGAEVKKPGASVKVSCKVSGYTLTEYAMHWVRQAPGKGLEWMGGINPNDGGTSYNQKFKGRVTMTEDKSTDTAYMELRSLRSEDTAVYYCATQRWLLRGGDYWGQGTLVTVSS

SEQ ID NO: 323 - protein, 119 aa, synthetic construct
  REGION 1..119: note = TfR9.5A scFv VH
QVQLVQSGAEVKKPGASVKVSCKASGYTFTEYAMHWVRQAPGQGLEWMGGINPNDGGTSYNQKFKGRVTSTRDTSISTAYMELSRLRSDDTVVYYCASQRWLLRGGDYWGQGTLVTVSS

SEQ ID NO: 324 - protein, 119 aa, synthetic construct
  REGION 1..119: note = TfR9.5B scFv VH
QVQLVQSGAEVKKPGASVKVSCKASGYTFTEYAMHWVRQAPGQGLEWMGGINPNDGGTSYNQKFKGRVTLTVDTSISTAYMELSRLRSDDTVVYYCASQRWLLRGGDYWGQGTLVTVSS

SEQ ID NO: 325 - protein, 121 aa, synthetic construct
  REGION 1..121: note = TfR12.1A scFv VH
QVQLVQSGAEVKKPGASVKVSCKASGYTFTSYWMHWVRQAPGQGLEWMGNIYPGSGSSNYDEKFKNRVTMTRDTSTSTVYMELSSLRSEDTAVYYCARSSYYGNFDTMDNWGQGTTVTVSS

SEQ ID NO: 326 - protein, 106 aa, synthetic construct
  REGION 1..106: note = TfR12.1A scFv VL
DIQLTQSPSFLSASVGDRVTITCSASSSIRYMNWYQQKPGKAPKLLIYDTSTLASGVPSRFSGSGSGTEFTLTISSLQPEDFATYYCHQRSSYPWTFGGGTKVEIK

SEQ ID NO: 327 - protein, 121 aa, synthetic construct
  REGION 1..121: note = TfR12.1B scFv VH
QVQLVQSGAEVKKPGASVKVSCKASGYTFTSYWMHWVRQAPGQGLEWMGNIYPGSGSSNYDEKFKNRVTLTVDTSTSTAYMELSSLRSEDTAVYYCARSSYYGNFDTMDNWGQGTTVTVSS

SEQ ID NO: 328 - protein, 106 aa, synthetic construct
  REGION 1..106: note = TfR12.1B scFv VL
QIQLTQSPSFLSASVGDRVTITCSASSSIRYMNWYQQKPGKAPKRLIYDTSTLASGVPSRFSGSGSGTSYTLTISSLQPEDFATYYCHQRSSYPWTFGGGTKVEIK

SEQ ID NO: 329 - protein, 106 aa, synthetic construct
  REGION 1..106: note = TfR12.2 scFv VL
EIVLTQSPATLSLSPGERATLSCSASSSIRYMNWYQQKPGQAPRLLIYDTSTLASGIPARFSGSGSGTDFTLTISSLEPEDFAVYYCHQRSSYPWTFGGGTKVEIK

SEQ ID NO: 330 - protein, 121 aa, synthetic construct
  REGION 1..121: note = TfR12.2B scFv VH
QVQLVQSGAEVKKPGASVKVSCKASGYTFTSYWMHWVRQAPGQGLEWMGNIYPGSGSSNYDEKFKNRVTLTVDTSTSTVYMELSSLRSEDTAVYYCARSSYYGNFDTMDNWGQGTTVTVSS

SEQ ID NO: 331 - protein, 106 aa, synthetic construct
  REGION 1..106: note = TfR12.2B scFv VL
QIVLTQSPATLSLSPGERATLSCSASSSIRYMNWYQQKPGQAPRRLIYDTSTLASGIPARFSGSGSGTSFTLTISSLEPEDFAVYYCHQRSSYPWTFGGGTKVEIK

SEQ ID NO: 332 - protein, 106 aa, synthetic construct
  REGION 1..106: note = TfR12.3A scFv VL
DIQMTQSPSSLSASVGDRVTITCSASSSIRYMNWYQQKPGKAPKLLIYDTSTLASGVPSRFSGSGSGTDFTLTISSLQPEDFATYYCHQRSSYPWTFGGGTKVEIK

SEQ ID NO: 333 - protein, 106 aa, synthetic construct
  REGION 1..106: note = TfR12.3B scFv VL
QIQMTQSPSSLSASVGDRVTITCSASSSIRYMNWYQQKPGKAPKRLIYDTSTLASGVPSRFSGSGSGTDYTLTISSLQPEDFATYYCHQRSSYPWTFGGGTKVEIK

SEQ ID NO: 334 - protein, 121 aa, synthetic construct
  REGION 1..121: note = TfR12.4a scFv VL
QVQLVQSGAEVKKPGASVKVSCKASGYTFTSYWMHWVRQAPGQGLEWMGNIYPGSGSSNYDEKFKNRVTSTRDTSISTAYMELSRLRSDDTVVYYCARSSYYGNFDTMDNWGQGTTVTVSS

SEQ ID NO: 335 - protein, 121 aa, synthetic construct; identical to SEQ ID NO: 334
  REGION 1..121: note = TfR scFv VH

SEQ ID NO: 336 - protein, 106 aa, synthetic construct
  REGION 1..106: note = TfR scFv VH
QIQMTQSPSSLSASVGDRVTITCSASSSIRYMNWYQQKPGKAPKRLIYDTSTLASGVPSRFSGSGSGTSYTLTISSLQPEDFATYYCHQRSSYPWTFGGGTKVEIK

SEQ ID NO: 337 - protein, 121 aa, synthetic construct
  REGION 1..121: note = TfR scFv VH
QVQLVQSGAEVKKPGASVKVSCKASGYTFTSYWMHWVRQAPGQGLEWMGNIYPGSGSSSYAQKFQGRVTMTVDTSTNTVYMELSSLRSEDTAVYYCTRSSYYGNFDTMDNWGQGTMVTVSS

SEQ ID NO: 338 - protein, 106 aa, synthetic construct
  REGION 1..106: note = TfR scFv VL
DIQLTQSPSFLSASVGDRVTITCRASSSIRYMNWYQQKPGKAPKRLIYDTSTLQSGVPSRFSGSGSGTEYTLTISSLQPEDFATYYCHQRSSYPWTFGGGTKVEIK

SEQ ID NO: 339 - protein, 121 aa, synthetic construct
  REGION 1..121: note = TfR scFv VH
QVQLVQSGAEVKKPGASVKVSCKASGYTFTSYWMHWVRQAPGQGLEWMGIIYPGSGSSNYAQKFQGRVTMTRDTSTSTVYMELSSLRSEDTAVYYCTRSSYYGNFDTMDNWGQGTTVTVSS

SEQ ID NO: 340 - protein, 106 aa, synthetic construct
  REGION 1..106: note = TfR scFv VL
DIQLTQSPSFLSASVGDRVTITCRASSSIRYMNWYQQKPGKAPKRLIYDTSTLQSGVPSRFSGSGSGTEFTLTISSLQPEDFATYYCHQRSSYPWTFGGGTKVEIK

SEQ ID NO: 341 - protein, 106 aa, synthetic construct
  REGION 1..106: note = TfR scFv VL
EIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 342 - protein, 106 aa, synthetic construct
  REGION 1..106: note = TfR scFv VL
DIQMTQSPSSLSASVGDRVTITCRASSSIRYLNWYQQKPGKAPKRLIYDTSSLQSGVPSRFSGSGSGTDFTLTISSLQPEDFATYYCHQRSSYPWTFGGGTKVEIK

SEQ ID NO: 343 - protein, 121 aa, synthetic construct
  REGION 1..121: note = TfR scFv VH
QVQLVQSGAEVKKPGASVKVSCKASGYTFTSYWMHWVRQAPGQGLEWMGNIYPGSGSSSYAQKFQGRVTMTRDTSTSTVYMELSSLRSEDTAVYYCTRSSYYGNFDTMDNWGQGTTVTVSS

SEQ ID NO: 344 - protein, 121 aa, synthetic construct
  REGION 1..121: note = TfR scFv VH
QVQLVQSGAEVKKPGASVKVSCKASGYTFTSYWMHWVRQAPGQGLEWMGNIYPGSGSSNYAQKFQGRVTMTRDTSTSTVYMELSSLRSEDTAVYYCTRSSYYGNFDTMDNWGQGTTVTVSS

SEQ ID NO: 345 - protein, 121 aa, synthetic construct
  REGION 1..121: note = TfR scFv VH
QVQLVQSGAEVKKPGASVKVSCKASGYTFTSYWMHWVRQAPGQGLEWMGNIYPGSGSSNYAQKFQGRVTLTVDTSTSTVYMELSSLRSEDTAVYYCTRSSYYGNFDTMDNWGQGTTVTVSS

SEQ ID NO: 346 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLQRGGDYWGQGTLVTVSS

SEQ ID NO: 347 - protein, 112 aa, synthetic construct
  REGION 1..112: note = TfR scFv VL
DVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQSFGGGTKVEIK

SEQ ID NO: 348 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLLRGGDHWGQGTLVTVSS

SEQ ID NO: 349 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLERGGDYWGQGTLVTVSS

SEQ ID NO: 350 - protein, 112 aa, synthetic construct
  REGION 1..112: note = TfR scFv VL
DVVMTQSPLSLPVTLGQPASISCKSSQSLLDSDGKIYLNWLQQRPGQSPRRLIYLVSKLDSGVPDRFSGSGSGTDFTLKISRVEAEDVGVYYCWQGTHFPQTFGGGTKVEIK

SEQ ID NO: 351 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLLIGGDYWGQGTLVTVSS

SEQ ID NO: 352 - protein, 112 aa, synthetic construct
  REGION 1..112: note = TfR scFv VL
DVVMTQSPLSLPVTLGQPASISCKSSQSDLDSDGKTYLNWLQQRPGQSPRRLIYLVSKLDSGVPDRFSGSGSGTDFTLKISRVEAEDVGVYYCWQGTSFPQTFGGGTKVEIK

SEQ ID NO: 353 - protein, 112 aa, synthetic construct
  REGION 1..112: note = TfR scFv VL
DVVMTQSPLSLPVTLGQPASISCKTSQSLLDPDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 354 - protein, 112 aa, synthetic construct
  REGION 1..112: note = TfR scFv VL
DVVMTQSPLSLPVTLGQPASISCKSSQSLKDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 355 - protein, 112 aa, synthetic construct
  REGION 1..112: note = TfR scFv VL
DVVMTQSPLSLPVTLGQPASISCKSSKSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 356 - protein, 112 aa, synthetic construct
  REGION 1..112: note = TfR scFv VL
DVVMTQSPLSLPVTLGQPASISCKSSVSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 357 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLLRGGDYWGQGTLVTVSS

SEQ ID NO: 358 - protein, 112 aa, synthetic construct
  REGION 1..112: note = TfR scFv VL
DVVMTQSPLSLPVTLGQPASISCKSSQSLLDSDGVTYLNWLQQRPGQSPRRLIYLVSKLDSGVPDRFSGSGSGTDFTLKISRVEAEDVGVYYCWQGTHFPQTFGGGTKVEIK

SEQ ID NO: 359 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTHYNQKFKGRVTMTVDTSTSTAYMELSSLRSEDTAVYYCASQRWLLRGGDYWGQGTLVTVSS

SEQ ID NO: 360 - protein, 112 aa, synthetic construct
  REGION 1..112: note = TfR scFv VL
DVVMTQSPLSLPVTLGQPASISCKSSRSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 361 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYSQKFKGRVTMTVDTSTSTAYMELSSLRSEDTAVYYCASQRWLERGGDYWGQGTLVTVSS

SEQ ID NO: 362 - protein, 112 aa, synthetic construct
  REGION 1..112: note = TfR scFv VL
DVVMTQSPLSLPVTLGQPASISCKSSQSLLDKDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 363 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDSGTSYNQKFKGRVTMTVDTSTSTAYMELSSLRSEDTAVYYCASQRWLERGGDYWGQGTLVTVSS

SEQ ID NO: 364 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSNLRSEDTAVYYCASQRWLLKGGDYWGQGTLVTVSS

SEQ ID NO: 365 - protein, 112 aa, synthetic construct
  REGION 1..112: note = TfR scFv VL
DVVMTQSPLSLPVTLGQPASISCKSSQSLLDSDGITYLNWLQQRPGQSPRRLIYLVSKLDSGVPDRFSGSGSGTDFTLKISRVEAEDVGVYYCWQGTHFPQTFGGGTKVEIK

SEQ ID NO: 366 - protein, 112 aa, synthetic construct
  REGION 1..112: note = TfR scFv VL
DVVMTQSPLSLPVTLGQPASISCKSSTSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 367 - protein, 112 aa, synthetic construct
  REGION 1..112: note = TfR scFv VL
DVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTKFPQTFGGGTKVEIK

SEQ ID NO: 368 - protein, 112 aa, synthetic construct
  REGION 1..112: note = TfR scFv VL
DVVMTQSPLSLPVTLGQPASISCKSSQSLLDSDGRTYLNWLQQRPGQSPRRLIYLVSKLDSGVPDRFSGSGSGTDFTLKISRVEAEDVGVYYCWQGTHFPQTFGGGTKVEIK

SEQ ID NO: 369 - protein, 119 aa, synthetic construct
  REGION 1..119: note = TfR scFv VH
QVQLVQSGAEVKKPGASVKVSCKASGYTFTEYAMHWVRQAPGQGLEWMGGINPKDGGTSYNQKFKGRVTMTVDTSTSTAYMELSSLRSEDTAVYYCASQGWLLRGGDYWGQGTLVTVSS

SEQ ID NO: 370 - protein, 112 aa, synthetic construct
  REGION 1..112: note = TfR scFv VL
DVVMTQSPLSLPVTLGQPASISCKSSQSLIDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 371 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLMRGGDYWGQGTLVTVSS

SEQ ID NO: 372 - protein, 119 aa, synthetic construct
  REGION 1..119: note = TfR scFv VH
QVQLVQSGAEVKKPGASVKVSCKASGYTFTEHAMHWVRQAPGQGLEWMGGINPNDGGTSTNQKFKGRVTMTVDTSTSTAYMELSSLRSEDTAVYYCASQGWLLRGGDYWGQGTLVTVSS

SEQ ID NO: 373 - protein, 112 aa, synthetic construct
  REGION 1..112: note = TfR scFv VL
DVVMTQSPLSLPVTLGQPASISCKSSQSLLDEDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 374 - protein, 112 aa, synthetic construct
  REGION 1..112: note = TfR scFv VL
DVVMTQSPLSLPVTLGQPASISCKSSQSLEDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 375 - protein, 119 aa, synthetic construct
  REGION 1..119: note = TfR scFv VH
QVQLVQSGAEVKKPGASVKVSCKASGYAFTEYAMHWVRQAPGQGLEWMGGINPNDGGTSYNQKFKGRVTMTVDTSTSTAYMELSSLRSEDTAVYYCASQRWLERGGNYWGQGTLVTVSS

SEQ ID NO: 376 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLFRGGDYWGQGTLVTVSS

SEQ ID NO: 377 - protein, 112 aa, synthetic construct
  REGION 1..112: note = TfR scFv VL
DVVMTQSPLSLPVTLGQPASISCKSSQSLQDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 378 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRSLLRGGDYWGQGTLVTVSS

SEQ ID NO: 379 - protein, 112 aa, synthetic construct
  REGION 1..112: note = TfR scFv VL
DVVMTQSPLSLPVTLGQPASISCKPSQSLLDGDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 380 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLYRGGDYWGQGTLVTVSS

SEQ ID NO: 381 - protein, 112 aa, synthetic construct
  REGION 1..112: note = TfR scFv VL
DVVMTQSPLSLPVTLGQPASISCKSSQSLLDEDGKTYLNWLQQRPGQSPRRLIYQVSKLDSGVPDRFSGSGSGTDFTLKISRVEAEDVGVYYCWQGTHFPQTFGGGTKVEIK

SEQ ID NO: 382 - protein, 112 aa, synthetic construct
  REGION 1..112: note = TfR scFv VL
DVVMTQSPLSLPVTLGQPASISCKTSQSLLDPDGKTYLNWLQQRPGQSPRRLIYLVSKLDSGVPDRFSGSGSGTDFTLKISRVEAEDVGVYYCWQSTHFPQTFGGGTKVEIK

SEQ ID NO: 383 - protein, 112 aa, synthetic construct
  REGION 1..112: note = TfR scFv VL
DVVMTQSPLSLPVTLGQPASISCKSSQSLLDSDGKTYLNWLQQRPGQSPRRLIYLVSKLDSGVPDRFSGSGSGTDFTLKISRVEAEDVGVYHCWQGTHFPQSFGGGTKVEIK

SEQ ID NO: 384 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTFYNQKFKGRVTMTVDTSTSTAYMELSSLRSEDTAVYYCASQGWLLRGGDYWGQGTLVTVSS

SEQ ID NO: 385 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYAQKFKGRVTMTVDTSTSTAYMELSSLRSEDTAVYYCASQRWLNRGGDYWGQGTLVTVSS

SEQ ID NO: 386 - protein, 112 aa, synthetic construct
  REGION 1..112: note = TfR scFv VL
DVVMTQSPLSLPVTLGQPASISCKASQSLLDEDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 387 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLDRGGDYWGQGTLVTVSS

SEQ ID NO: 388 - protein, 112 aa, synthetic construct
  REGION 1..112: note = TfR scFv VL
DVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTQFPQTFGGGTKVEIK

SEQ ID NO: 389 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRELLRGGDYWGQGTLVTVSS

SEQ ID NO: 390 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYLQKFKGRVTMTVDTSTSTAYMELSSLRSEDTAVYYCASQRWLLRGGDYWGQGTLVTVSS

SEQ ID NO: 391 - protein, 127 aa, synthetic construct
  REGION 1..127: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQRWLLRGGDYWGQGTLVTVSSGGSEGKSS

SEQ ID NO: 392 - protein, 112 aa, synthetic construct
  REGION 1..112: note = TfR scFv VL
DVVMTQSPLSLPVTLGQPASISCKSSQSLLDSIGKTYLNWLQQRPGQSPRRLIYLVSKLDSGVPDRFSGSGSGTDFTLKISRVEAEDVGVYYCWQGTHFPQTFGGGTKVEIK

SEQ ID NO: 393 - protein, 119 aa, synthetic construct
  REGION 1..119: note = TfR scFv VH
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLQRGGDYWGQGTLVTVSS

SEQ ID NO: 394 - protein, 119 aa, synthetic construct
  REGION 1..119: note = TfR scFv VH
QVQLVQSGAEVKKPGASVKVSCKASGYTFTEYAMHWVRQAPGQGLEWMGGIDPRSGGTSYNQKFKGRVTMTVDTSTSTAYMELSSLRSEDTAVYYCASQGWLKRGGDYWGQGTLVTVSS

SEQ ID NO: 395 - protein, 112 aa, synthetic construct
  REGION 1..112: note = TfR scFv VL
DVVMTQSPLSLPVTLGQPASISCKASQSLLDEEGKTYLNWLQQRPGQSPRRLIYLVSKLDSGVPDRFSGSGSGTDFTLKISRVEAEDVGVYYCWQGTHFPQTFGGGTKVEIK

SEQ ID NO: 396 - protein, 119 aa, synthetic construct
  REGION 1..119: note = TfR scFv VH
QVQLVQSGAEVKKPGASVKVSCKASGYTFTEYAMHWVRQAPGQGLEWMGGIDPRSGGTSYNQKFKGRVTMTVDTSTSTAYMELSSLRSEDTAVYYCASQGWLQRGGDYWGQGTLVTVSS

SEQ ID NO: 397 - protein, 119 aa, synthetic construct
  REGION 1..119: note = TfR scFv VH
QVQLVQSGAEVKKPGASVKVSCKASGYTFTEYAMHWVRQAPGQGLEWMGGIDPNTGGTSYNQKFKGRVTMTVDTSTSTAYMELSSLRSEDTAVYYCASQGWLLRGGDYWGQGTLVTVSS

SEQ ID NO: 398 - protein, 119 aa, synthetic construct
  REGION 1..119: note = TfR scFv VH
QVQLVQSGAEVKKPGASVKVSCKASGYTFTEYAMHWVRQAPGQGLEWMGGIAPGTGGTSYNQKFKGRVTMTVDTSTSTAYMELSSLRSEDTAVYYCASQGWLLRGGDYWGQGTLVTVSS

SEQ ID NO: 399 - protein, 119 aa, synthetic construct
  REGION 1..119: note = TfR9.1B.39.38 scFv VH
QVQLVQSGAEVKKPGASVKVSCKASGYTFTEYAMHWVRQAPGQGLEWMGGIAPNTGGTSYNQKFKGRVTMTVDTSTSTAYMELSSLRSEDTAVYYCASQGWLLRGGDYWGQGTLVTVSS

SEQ ID NO: 400 - protein, 112 aa, synthetic construct
  REGION 1..112: note = TfR9.1B.39.38 scFv VL
DVVMTQSPLSLPVTLGQPASISCKASQSLLDEGGKTYLNWLQQRPGQSPRRLIYLVSKLDSGVPDRFSGSGSGTDFTLKISRVEAEDVGVYYCWQGTHFPQTFGGGTKVEIK

SEQ ID NO: 401 - protein, 119 aa, synthetic construct
  REGION 1..119: note = TfR9.1B.39.39 scFv VH
QVQLVQSGAEVKKPGASVKVSCKASGYTFTEYAMHWVRQAPGQGLEWMGGINPSFGGTSYNQKFKGRVTMTVDTSTSTAYMELSSLRSEDTAVYYCASQGWLLRGGDYWGQGTLVTVSS

SEQ ID NO: 402 - protein, 119 aa, synthetic construct
  REGION 1..119: note = TfR9.1B.39.40 scFv VH
QVQLVQSGAEVKKPGASVKVSCKASGYTFTEYAMHWVRQAPGQGLEWMGGINPKEGGTSYNQKFKGRVTMTVDTSTSTAYMELSSLRSEDTAVYYCASQGWLLRGGDYWGQGTLVTVSS

SEQ ID NO: 403 - protein, 112 aa, synthetic construct
  REGION 1..112: note = TfR9.1B.39.40 scFv VL
DVVMTQSPLSLPVTLGQPASISCKASQSLLDEDQKTYLNWLQQRPGQSPRRLIYLVSKLDSGVPDRFSGSGSGTDFTLKISRVEAEDVGVYYCWQGTHFPQTFGGGTKVEIK

SEQ ID NO: 404 - protein, 247 aa, synthetic construct
  REGION 1..247: note = TfR15.WH8.1.1
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRAMNWYQQKPGQAPRRLIYDTSNRATGIPARFSGSGSGTDFTLTISSLEPEDFAVYYCHQRSSYPWTFGGGTKVEIK

SEQ ID NO: 405 - protein, 444 aa, synthetic construct
  REGION 1..444: note = TD1 heavy chain hIgG1 NSLF (N325S, L328F) knob (T366W)
QVQLVQSGSELKKPGASVKVSCKASGYAFTSYGLSWVRQAPGQGLEWMGWINTYSGVPTYAQGFTGRFVFSLDTSVSTAYLQISSLKAEDTAVYYCARTMADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 406 - protein, 710 aa, synthetic construct
  REGION 1..710: note = TD1 heavy chain hIgG1 NSLF (N325S, L328F) hole scFv (T366S, L368A, Y407V)-(G4S)3 linker-VH-20AA linker-VL with mutations (H435R, Y436F)
QVQLVQSGSELKKPGASVKVSCKASGYAFTSYGLSWVRQAPGQGLEWMGWINTYSGVPTYAQGFTGRFVFSLDTSVSTAYLQISSLKAEDTAVYYCARTMADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSQVQLVQSGAEVKKPGASVKVSCKASGYTFTEYAMHWVRQAPGQGLEWMGGIAPNTGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEGGKTYLNWLQQRPGQSPRRLIYLVSKLDSGVPDRFSGSGSGTDFTLKISRVEAEDVGVYYCWQGTHFPQTFGGGTKVEIK

SEQ ID NO: 407 - protein, 219 aa, synthetic construct
  REGION 1..219: note = TD1 Light Chain
DVVMTQSPLSLPVTLGQPASISCKSSRSLLYSAGKTYLSWFQQRPGQSPRRLIYLVSKLDSGVPDRFSGSGSGTDFTLKISRVEAEDVGVYYCWQGIDFHQTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 408 - protein, 449 aa, synthetic construct; identical to SEQ ID NO: 194
  REGION 1..449: note = Isotype control heavy chain hIgG1 NSLF (N325S, L328F) knob (T366W)

SEQ ID NO: 409 - protein, 715 aa, synthetic construct
  REGION 1..715: note = Isotype control heavy chain hIgG1 NSLF (N325S, L328F) hole scFv (T366S, L368A, Y407V)-(G4S)3 linker-VH-20AA linker-VL with mutations (H435R, Y436F)
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSQVQLVQSGAEVKKPGASVKVSCKASGYTFTEYAMHWVRQAPGQGLEWMGGIAPNTGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEGGKTYLNWLQQRPGQSPRRLIYLVSKLDSGVPDRFSGSGSGTDFTLKISRVEAEDVGVYYCWQGTHFPQTFGGGTKVEIK

SEQ ID NO: 410 - protein, 214 aa, synthetic construct; identical to SEQ ID NO: 188
  REGION 1..214: note = Isotype control light chain

SEQ ID NO: 411 - protein, 444 aa, synthetic construct
  REGION 1..444: note = TD1 heavy chain hIgG1 NSLF (N325S, L328F)
QVQLVQSGSELKKPGASVKVSCKASGYAFTSYGLSWVRQAPGQGLEWMGWINTYSGVPTYAQGFTGRFVFSLDTSVSTAYLQISSLKAEDTAVYYCARTMADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 412 - protein, 17 aa, synthetic construct
  REGION 1..17: note = TfR9.1B.39 variant CDR H2
GIDPRSGGTSYNQKFKG

SEQ ID NO: 413 - protein, 17 aa, synthetic construct
  REGION 1..17: note = TfR9.1B.39 variant CDR H2
GIDPNTGGTSYNQKFKG

SEQ ID NO: 414 - protein, 17 aa, synthetic construct
  REGION 1..17: note = TfR9.1B.39 variant CDR H2
GIAPGTGGTSYNQKFKG

SEQ ID NO: 415 - protein, 17 aa, synthetic construct
  REGION 1..17: note = TfR9.1B.39 variant CDR H2
GIAPNTGGTSYNQKFKG

SEQ ID NO: 416 - protein, 17 aa, synthetic construct
  REGION 1..17: note = TfR9.1B.39 variant CDR H2
GINPSFGGTSYNQKFKG

SEQ ID NO: 417 - protein, 17 aa, synthetic construct
  REGION 1..17: note = TfR9.1B.39 variant CDR H2
GINPKEGGTSYNQKFKG

SEQ ID NO: 418 - protein, 10 aa, synthetic construct
  REGION 1..10: note = TfR9.1B.39 variant CDR H3
QGWLLRGGDY

SEQ ID NO: 419 - protein, 10 aa, synthetic construct
  REGION 1..10: note = TfR9.1B.39 variant CDR H3
QGWLKRGGDY

SEQ ID NO: 420 - protein, 10 aa, synthetic construct
  REGION 1..10: note = TfR9.1B.39 variant CDR H3
QGWLQRGGDY

SEQ ID NO: 421 - protein, 16 aa, synthetic construct
  REGION 1..16: note = TfR9.1B.39 variant CDR L1
KASQSLLDEDGKTYLN

SEQ ID NO: 422 - protein, 16 aa, synthetic construct
  REGION 1..16: note = TfR9.1B.39 variant CDR L1
KASQSLLDEEGKTYLN

SEQ ID NO: 423 - protein, 16 aa, synthetic construct
  REGION 1..16: note = TfR9.1B.39 variant CDR L1
KASQSLLDEGGKTYLN

SEQ ID NO: 424 - protein, 16 aa, synthetic construct
  REGION 1..16: note = TfR9.1B.39 variant CDR L1
KASQSLLDEDQKTYLN

SEQ ID NO: 425 - protein, 247 aa, synthetic construct
  REGION 1..247: note = TFR.15.WH8.1.24A
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSAYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 426 - protein, 247 aa, synthetic construct
  REGION 1..247: note = TFR.15.WH8.1.24K
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSKYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 427 - protein, 247 aa, synthetic construct
  REGION 1..247: note = TFR.15.WH8.1.24D
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSDYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 428 - protein, 247 aa, synthetic construct
  REGION 1..247: note = TFR.15.WH8.1.24N
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSNYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 429 - protein, 247 aa, synthetic construct
  REGION 1..247: note = TFR.15.WH8.1.24H
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSHYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 430 - protein, 247 aa, synthetic construct
  REGION 1..247: note = TFR.15.WH8.1.26K
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYKGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 431 - protein, 247 aa, synthetic construct
  REGION 1..247: note = TFR.15.WH8.1.26Q
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYQGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 432 - protein, 247 aa, synthetic construct
  REGION 1..247: note = TFR.15.WH8.1.26H
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYHGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 433 - protein, 247 aa, synthetic construct
  REGION 1..247: note = TFR.15.WH8.1.26E
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYEGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 434 - protein, 247 aa, synthetic construct
  REGION 1..247: note = TFR.15.WH8.1.26S
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYSGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 435 - protein, 247 aa, synthetic construct
  REGION 1..247: note = TFR.15.WH8.1.42Q
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMNWYQQKPGQAPRRLIYDTSNRATGIPARFSGSGSGTDFTLTISSLEPEDFAVYYCHQRSSYPWTFGGGTKVEIK

SEQ ID NO: 436 - protein, 247 aa, synthetic construct
  REGION 1..247: note = TFR.15.WH8.1.42H
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRHMNWYQQKPGQAPRRLIYDTSNRATGIPARFSGSGSGTDFTLTISSLEPEDFAVYYCHQRSSYPWTFGGGTKVEIK

SEQ ID NO: 437 - protein, 247 aa, synthetic construct
  REGION 1..247: note = TFR.15.WH8.1.42D
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRDMNWYQQKPGQAPRRLIYDTSNRATGIPARFSGSGSGTDFTLTISSLEPEDFAVYYCHQRSSYPWTFGGGTKVEIK

SEQ ID NO: 438 - protein, 247 aa, synthetic construct
  REGION 1..247: note = TFR.15.WH8.1.42S
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRSMNWYQQKPGQAPRRLIYDTSNRATGIPARFSGSGSGTDFTLTISSLEPEDFAVYYCHQRSSYPWTFGGGTKVEIK

SEQ ID NO: 439 - protein, 247 aa, synthetic construct
  REGION 1..247: note = TFR.15.WH8.1.42K
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRKMNWYQQKPGQAPRRLIYDTSNRATGIPARFSGSGSGTDFTLTISSLEPEDFAVYYCHQRSSYPWTFGGGTKVEIK

SEQ ID NO: 440 - protein, 247 aa, synthetic construct
  REGION 1..247: note = TFR.15.WH8.1.42A.LH
EIVLTQSPATLSLSPGERATLSCRASSSIRAMNWYQQKPGQAPRRLIYDTSNRATGIPARFSGSGSGTDFTLTISSLEPEDFAVYYCHQRSSYPWTFGGGTKVEIKGGSEGKSSGSGSESKSTGGSQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSS

SEQ ID NO: 441 - protein, 242 aa, synthetic construct
  REGION 1..242: note = TFR.15.WH8.1.42A.3x(G4S)
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGGGSGGGGSGGGGSEIVLTQSPATLSLSPGERATLSCRASSSIRAMNWYQQKPGQAPRRLIYDTSNRATGIPARFSGSGSGTDFTLTISSLEPEDFAVYYCHQRSSYPWTFGGGTKVEIK

SEQ ID NO: 442 - protein, 248 aa, synthetic construct
  REGION 1..248: note = TFR.15.WH8.1.42A.L1
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSSPNSASHSGSAPQTSSAPGSQEIVLTQSPATLSLSPGERATLSCRASSSIRAMNWYQQKPGQAPRRLIYDTSNRATGIPARFSGSGSGTDFTLTISSLEPEDFAVYYCHQRSSYPWTFGGGTKVEIK

SEQ ID NO: 443 - protein, 121 aa, synthetic construct
  REGION 1..121: note = TFR.15.WH8.1
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSS

SEQ ID NO: 444 - protein, 121 aa, synthetic construct
  REGION 1..121: note = TFR.15.WH8.1.24A
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSAYYGNFDTMDNWGQGTTVTVSS

SEQ ID NO: 445 - protein, 121 aa, synthetic construct
  REGION 1..121: note = TFR.15.WH8.1.24K
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSKYYGNFDTMDNWGQGTTVTVSS

SEQ ID NO: 446 - protein, 121 aa, synthetic construct
  REGION 1..121: note = TFR.15.WH8.1.24D
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSDYYGNFDTMDNWGQGTTVTVSS

SEQ ID NO: 447 - protein, 121 aa, synthetic construct
  REGION 1..121: note = TFR.15.WH8.1.24N
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSNYYGNFDTMDNWGQGTTVTVSS

SEQ ID NO: 448 - protein, 121 aa, synthetic construct
  REGION 1..121: note = TFR.15.WH8.1.24H
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSHYYGNFDTMDNWGQGTTVTVSS

SEQ ID NO: 449 - protein, 121 aa, synthetic construct
  REGION 1..121: note = TFR.15.WH8.1.26K
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYKGNFDTMDNWGQGTTVTVSS

SEQ ID NO: 450 - protein, 121 aa, synthetic construct
  REGION 1..121: note = TFR.15.WH8.1.26Q
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYQGNFDTMDNWGQGTTVTVSS

SEQ ID NO: 451 - protein, 121 aa, synthetic construct
  REGION 1..121: note = TFR.15.WH8.1.26H
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYHGNFDTMDNWGQGTTVTVSS

SEQ ID NO: 452 - protein, 121 aa, synthetic construct
  REGION 1..121: note = TFR.15.WH8.1.26E
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYEGNFDTMDNWGQGTTVTVSS

SEQ ID NO: 453 - protein, 121 aa, synthetic construct
  REGION 1..121: note = TFR.15.WH8.1.26S
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYSGNFDTMDNWGQGTTVTVSS

SEQ ID NO: 454 - protein, 106 aa, synthetic construct
  REGION 1..106: note = TFR.15.WH8.1.42A VL
EIVLTQSPATLSLSPGERATLSCRASSSIRAMNWYQQKPGQAPRRLIYDTSNRATGIPARFSGSGSGTDFTLTISSLEPEDFAVYYCHQRSSYPWTFGGGTKVEIK

SEQ ID NO: 455 - protein, 106 aa, synthetic construct
  REGION 1..106: note = TfR.15.WH8.1 VL
EIVLTQSPATLSLSPGERATLSCRASSSIRQMNWYQQKPGQAPRRLIYDTSNRATGIPARFSGSGSGTDFTLTISSLEPEDFAVYYCHQRSSYPWTFGGGTKVEIK

SEQ ID NO: 456 - protein, 106 aa, synthetic construct; identical to SEQ ID NO: 455
  REGION 1..106: note = TfR.15.WH8.1 VL

SEQ ID NO: 457 - protein, 106 aa, synthetic construct
  REGION 1..106: note = TfR.15.WH8.1 VL
EIVLTQSPATLSLSPGERATLSCRASSSIRDMNWYQQKPGQAPRRLIYDTSNRATGIPARFSGSGSGTDFTLTISSLEPEDFAVYYCHQRSSYPWTFGGGTKVEIK

SEQ ID NO: 458 - protein, 106 aa, synthetic construct
  REGION 1..106: note = TfR.15.WH8.1 VL
EIVLTQSPATLSLSPGERATLSCRASSSIRSMNWYQQKPGQAPRRLIYDTSNRATGIPARFSGSGSGTDFTLTISSLEPEDFAVYYCHQRSSYPWTFGGGTKVEIK

SEQ ID NO: 459 - protein, 106 aa, synthetic construct; identical to SEQ ID NO: 458
  REGION 1..106: note = TfR.15.WH8.1 VL

SEQ ID NO: 460 - protein, 5 aa, synthetic construct
  REGION 1..5: note = TfR.15.WH8.1 CDR h1
SYYMH

SEQ ID NO: 461 - protein, 12 aa, synthetic construct
  REGION 1..12: note = TfR.15.WH8.1 CDR H3
SAYYGNFDTMDN

SEQ ID NO: 462 - protein, 12 aa, synthetic construct
  REGION 1..12: note = TfR.15.WH8.1 CDR H3
SKYYGNFDTMDN

SEQ ID NO: 463 - protein, 12 aa, synthetic construct
  REGION 1..12: note = TfR.15.WH8.1 CDR H3
SDYYGNFDTMDN

SEQ ID NO: 464 - protein, 12 aa, synthetic construct
  REGION 1..12: note = TfR.15.WH8.1 CDR H3
SNYYGNFDTMDN

SEQ ID NO: 465 - protein, 12 aa, synthetic construct
  REGION 1..12: note = TfR.15.WH8.1 CDR H3
SHYYGNFDTMDN

SEQ ID NO: 466 - protein, 12 aa, synthetic construct
  REGION 1..12: note = TfR.15.WH8.1 CDR H3
SSYKGNFDTMDN

SEQ ID NO: 467 - protein, 12 aa, synthetic construct
  REGION 1..12: note = TfR.15.WH8.1 CDR H3
SSYQGNFDTMDN

SEQ ID NO: 468 - protein, 12 aa, synthetic construct
  REGION 1..12: note = TfR.15.WH8.1 CDR H3
SSYHGNFDTMDN

SEQ ID NO: 469 - protein, 12 aa, synthetic construct
  REGION 1..12: note = TfR.15.WH8.1 CDR H3
SSYEGNFDTMDN

SEQ ID NO: 470 - protein, 12 aa, synthetic construct
  REGION 1..12: note = TfR.15.WH8.1 CDR H3
SSYSGNFDTMDN

SEQ ID NO: 471 - protein, 10 aa, synthetic construct
  REGION 1..10: note = TFR.15.WH8.1 CDR-L1
RASSSIRAMN

SEQ ID NO: 472 - protein, 10 aa, synthetic construct
  REGION 1..10: note = TFR.15.WH8.1 CDR-L1
RASSSIRQMN

SEQ ID NO: 473 - protein, 10 aa, synthetic construct
  REGION 1..10: note = TFR.15.WH8.1 CDR-L1
RASSSIRHMN

SEQ ID NO: 474 - protein, 10 aa, synthetic construct
  REGION 1..10: note = TFR.15.WH8.1 CDR-L1
RASSSIRDMN

SEQ ID NO: 475 - protein, 10 aa, synthetic construct
  REGION 1..10: note = TFR.15.WH8.1 CDR-L1
RASSSIRSMN

SEQ ID NO: 476 - protein, 10 aa, synthetic construct
  REGION 1..10: note = TFR.15.WH8.1 CDR-L1
RASSSIRKMN

(19) United States
(12) Patent Application Publication
(10) Pub. No.: US 2025/0270339 A1
(43) Pub. Date: Aug. 28, 2025

(54) TRANSFERRIN RECEPTOR ANTIGEN-BINDING DOMAINS AND USES THEREFOR

(71) Applicant: ALECTOR LLC, South San Francisco, CA (US)

(72) Inventors: Eric BROWN, South San Francisco, CA (US); Hamid SALIMI, San Mateo, CA (US); Angie Grace YEE, San Francisco, CA (US); Margaret L. TANG, Mountain View, CA (US); Wei-Hsien HO, Belmont, CA (US); Tarangsri NIVITCHANYONG, Castro Valley, CA (US); Rajkumar GANESAN, Thousand Oaks, CA (US); Lu SHAN, Belmont, CA (US); Thunga BIENLY, Daly City, CA (US); Raymond Ka-Hang TONG, San Francisco, CA (US); Alexander Hyun-min YANG, San Jose, CA (US)

(21) Appl. No.: 19/039,352

(22) Filed: Jan. 28, 2025

Related U.S. Application Data
(63) Continuation of application No. PCT/US2023/071239, filed on Jul. 28, 2023.
(60) Provisional application No. 63/513,820, filed on Jul. 14, 2023.
(60) Provisional application No. 63/495,511, filed on Apr. 11, 2023.
(60) Provisional application No. 63/489,693, filed on Mar. 10, 2023.
(60) Provisional application No. 63/374,967, filed on Sep. 8, 2022.
(60) Provisional application No. 63/369,898, filed on Jul. 29, 2022.

(51) Int. Cl. C07K 16/28; A61K 39/00

(52) U.S. Cl. CPC C07K 16/2881; A61K 2039/505; C07K 2317/24; C07K 2317/31; C07K 2317/52; C07K 2317/622; C07K 2317/732; C07K 2317/734; C07K 2317/77; C07K 2317/92; C07K 2319/30

(57) ABSTRACT

The present disclosure is generally directed to antigen-binding domains that specifically bind to human transferrin receptor (TfR) and their use in transport across the blood brain barrier (BBB).

### CROSS-REFERENCE TO RELATED APPLICATIONS

This application claims the priority benefit of U.S. Provisional Appl. Nos. 63/369,898, filed Jul. 29, 2022; 63/374,967, filed Sep. 8, 2022; 63/489,693, filed Mar. 10, 2023; 63/495,511, filed Apr. 11, 2023; and 63/513,820, filed Jul. 14, 2023; each of which is herein incorporated by reference in its entirety.

### REFERENCE TO SEQUENCE LISTING SUBMITTED ELECTRONICALLY

The content of the electronically submitted sequence listing (Name: 4503_022PC05_SeqListing_ST26.xml; Size: 660,697 bytes; and Date of Creation: Jul. 13, 2023) is herein incorporated by reference in its entirety.

### FIELD OF THE PRESENT DISCLOSURE

The present disclosure relates to antigen-binding domains that specifically bind to human transferrin receptor (TfR). These antigen-binding domains can cross the blood brain barrier and can transport other agents (e.g., therapeutically active agents) associated with the antigen-binding domain across the blood brain barrier.

### BACKGROUND

Passive transfer of substances from blood to brain is restricted by the blood brain barrier (BBB). The BBB provides precise control of central nervous system (CNS) homeostasis allowing for proper neuronal function and also protecting neural tissue from toxins and pathogens. Alterations of the BBB are an important component of pathology and progression of different neurological diseases. However, the BBB poses a problem with regard to delivering therapeutics to the CNS. While recombinant proteins and antibody therapeutics have shown much success outside the CNS, such biologics do not cross the BBB efficiently. As a result, delivery of some therapeutics to the CNS has relied on injection of the therapeutic directly into the CNS. However, such injections are invasive procedures that have efficacy that is limited by the rapid export of cerebral spinal fluid (CSF) containing the therapeutic from the brain to the blood. Alternatively, a therapeutic intended for the CNS may be administered systemically at a high dose to allow for sufficient penetration of the BBB by the therapeutic. However, this approach may result in unintended effects due to the high dose in the periphery or increased manufacturing and formulation burdens to achieve the high dose. Accordingly, improved products and methods for delivering therapeutics across the BBB are needed.

### SUMMARY OF THE PRESENT DISCLOSURE

Provided herein are antigen-binding domains, fusion proteins, antibodies, and multi-specific proteins that specifically bind to human transferrin receptor (TfR), and methods of making and using the same.

In some aspects, provided herein is an antigen-binding domain that specifically binds to the apical domain of the human transferrin receptor (TfR), wherein the antigen binding domain localizes to brain parenchyma in a subject following peripheral injection, and wherein the antigen-binding domain is not within a modified CH3 domain.

In some aspects, provided herein is an antigen-binding domain that specifically binds to human transferrin receptor (TfR), wherein the antigen-binding domain comprises heavy chain variable region (VH) complementarity determining region (CDR) 1, VH CDR2, VH CDR3 and light chain variable region (VL) CDR1, CDR2, and CDR3 sequences comprising the amino acid sequences of: SEQ ID NOs:94, 415, 418, 423, 150, and 151, respectively; SEQ ID NOs:74-76 and 135-137, respectively; SEQ ID NOs:77-79 and 138-140, respectively; SEQ ID NOs:80-82 and 135-137, respectively; SEQ ID NOs:83-85 and 141-143, respectively; SEQ ID NOs:74, 86, 76 and 135-137, respectively; SEQ ID NOs:87-89, and 143-145, respectively; SEQ ID NOs:90-92 and 146-148, respectively; SEQ ID NOs:83, 93, 85 and 141-143, respectively; SEQ ID NOs:94-96 and 149-151, respectively; SEQ ID NOs:97, 86, 98, and 135-137, respectively; SEQ ID NOs:99, 100, 101, and 152-154, respectively; SEQ ID NOs:99, 102, 103, and 155-157, respectively; SEQ ID NOs:104-106, 138, 158, and 159, respectively; SEQ ID NOs:107-109 and 160-162, respectively; SEQ ID NOs:99, 110, 103, and 155-157, respectively; SEQ ID NOs:99, 111, 112, 152, 163, and 164, respectively; SEQ ID NOs:90, 113, 114, 165, 166, and 157, respectively; SEQ ID NOs:83, 115, 85, 141, 142, and 167, respectively; SEQ ID NOs:116, 110, 103, and 155-157, respectively; SEQ ID NOs:117-119 and 168-170, respectively; SEQ ID NOs:116, 110, 103, and 168-170, respectively; SEQ ID NOs:90, 120, 114, 165, 166, and 157, respectively; SEQ ID NOs:90, 113, 114, 171, 166, and 157, respectively; SEQ ID NOs:97, 121, 122, 135, 136, and 172, respectively; SEQ ID NOs:74, 123, 124, 173, 136, and 137, respectively; SEQ ID NOs:74, 125, 126, 174, 136, and 137, respectively; SEQ ID NOs:99, 127, 128, 155, 175, and 176, respectively; SEQ ID NOs:90, 129, 130, 165, 166, and 157, respectively; SEQ ID NOs:131, 132, 133, 177, 166, and 178, respectively; SEQ ID NOs:131, 132, 133, and 179-181, respectively; SEQ ID NOs:90, 134, 114, 165, 166, and 157, respectively; SEQ ID NOs:83, 302, 303, 141, 142, and 182, respectively; SEQ ID NOs:99, 305, 103, 307, 308, and 157, respectively; SEQ ID NOs:99, 306, 103, 307, 308, and 157, respectively; SEQ ID NOs:99, 306, 103, 307, 309, and 157, respectively; SEQ ID NOs:99, 306, 103, 310, 311, and 157, respectively; SEQ ID NOs:99, 305, 103, 310, 311, and 157, respectively; SEQ ID NOs:99, 312, 103, 307, 308, and 157, respectively; SEQ ID NOs:99, 312, 103, 307, 309, and 157, respectively; SEQ ID NOs:99, 312, 103, 310, 311, and 157, respectively; SEQ ID NOs:94, 95, 418, 421, 150, and 151, respectively; SEQ ID NOs:94, 412, 419, 422, 150, and 151, respectively; SEQ ID NOs:94, 412, 420, 422, 150, and 151, respectively; SEQ ID NOs:94, 413, 418, 422, 150, and 151, respectively; SEQ ID NOs:94, 414, 418, 422, 150, and 151, respectively; SEQ ID NOs:94, 416, 418, 422, 150, and 151, respectively; SEQ ID NOs:94, 417, 418, 424, 150, and 151, respectively; SEQ ID NOs:460, 312, 103, 307, 309, and 157, respectively; SEQ ID NOs:460, 312, 461, 307, 309, and 157, respectively; SEQ ID NOs:460, 312, 462, 307, 309, and 157, respectively; SEQ ID NOs:460, 312, 463, 307, 309, and 157, respectively; SEQ ID NOs:460, 312, 464, 307, 309, and 157, respectively; SEQ ID NOs:460, 312, 465, 307, 309, and 157, respectively; SEQ ID NOs:460, 312, 466, 307, 309, and 157, respectively; SEQ ID NOs:460, 312, 467, 307, 309, and 157, respectively; SEQ ID NOs:460, 312, 468, 307, 309, and 157, respectively; SEQ ID NOs:460, 312, 469, 307, 309, and 157, respectively; SEQ ID NOs:460, 312, 470, 307, 309, and 157, respectively; SEQ ID NOs:460, 312, 103, 471, 309, and 157, respectively; SEQ ID NOs:460, 312, 103, 472, 309, and 157, respectively; SEQ ID NOs:460, 312, 103, 473, 309, and 157, respectively; SEQ ID NOs:460, 312, 103, 474, 309, and 157, respectively; SEQ ID NOs:460, 312, 103, 475, 309, and 157, respectively; SEQ ID NOs:460, 312, 103, 476, 309, and 157, respectively; or SEQ ID NOs:460, 312, 103, 471, 309, and 157, respectively.

In some aspects, the antigen-binding domain comprises a VH and a VL, wherein the VH and VL comprise amino acid sequences at least 80%, at least 85%, at least 90%, at least 95%, or at least 99% identical to the amino acid sequences of: SEQ ID NOs:399 and 400, respectively; SEQ ID NOs:10 and 11, respectively; SEQ ID NOs:12 and 13, respectively; SEQ ID NOs:14 and 15, respectively; SEQ ID NOs:16 and 17, respectively; SEQ ID NOs:18 and 19, respectively; SEQ ID NOs:20 and 21, respectively; SEQ ID NOs:22 and 23, respectively; SEQ ID NOs:24 and 25, respectively; SEQ ID NOs:26 and 27, respectively; SEQ ID NOs:28 and 29, respectively; SEQ ID NOs:30 and 31, respectively; SEQ ID NOs:32 and 33, respectively; SEQ ID NOs:34 and 35, respectively; SEQ ID NOs:36 and 37, respectively; SEQ ID NOs:38 and 39, respectively; SEQ ID NOs:40 and 41, respectively; SEQ ID NOs:42 and 43, respectively; SEQ ID NOs:44 and 45, respectively; SEQ ID NOs:46 and 47, respectively; SEQ ID NOs:48 and 49, respectively; SEQ ID NOs:50 and 51, respectively; SEQ ID NOs:52 and 53, respectively; SEQ ID NOs:54 and 55, respectively; SEQ ID NOs:56 and 57, respectively; SEQ ID NOs:58 and 59, respectively; SEQ ID NOs:60 and 61, respectively; SEQ ID NOs:62 and 63, respectively; SEQ ID NOs:64 and 65, respectively; SEQ ID NOs:66 and 67, respectively; SEQ ID NOs:68 and 69, respectively; SEQ ID NOs:70 and 71, respectively; SEQ ID NOs:72 and 73, respectively; SEQ ID NOs:313 and 314, respectively; SEQ ID NOs:315 and 316, respectively; SEQ ID NOs:317 and 314, respectively; SEQ ID NOs:318 and 316, respectively; SEQ ID NOs:319 and 314, respectively; SEQ ID NOs:320 and 316, respectively; SEQ ID NOs:321 and 314, respectively; SEQ ID NOs:322 and 316, respectively; SEQ ID NOs:323 and 314, respectively; SEQ ID NOs:324 and 316, respectively; SEQ ID NOs:325 and 326, respectively; SEQ ID NOs:327 and 328, respectively; SEQ ID NOs:325 and 329, respectively; SEQ ID NOs:330 and 331, respectively; SEQ ID NOs:325 and 332, respectively; SEQ ID NOs:330 and 333, respectively; SEQ ID NOs:334 and 332, respectively; SEQ ID NOs:335 and 336, respectively; SEQ ID NOs:337 and 338, respectively; SEQ ID NOs:38 and 33, respectively; SEQ ID NOs:339 and 340, respectively; SEQ ID NOs:339 and 341, respectively; SEQ ID NOs:339 and 342, respectively; SEQ ID NOs:343 and 340, respectively; SEQ ID NOs:343 and 342, respectively; SEQ ID NOs:344 and 340, respectively; SEQ ID NOs:344 and 341, respectively; SEQ ID NOs:344 and 342, respectively; SEQ ID NOs:345 and 340, respectively; SEQ ID NOs:345 and 341, respectively; SEQ ID NOs:345 and 342, respectively; SEQ ID NOs:346 and 347, respectively; SEQ ID NOs:348 and 316, respectively; SEQ ID NOs:349 and 347, respectively; SEQ ID NOs:349 and 350, respectively; SEQ ID NOs:351 and 352, respectively; SEQ ID NOs:349 and 353, respectively; SEQ ID NOs:346 and 354, respectively; SEQ ID NOs:349 and 355, respectively; SEQ ID NOs:349 and 356, respectively; SEQ ID NOs:357 and 316, respectively; SEQ ID NOs:349 and 358, respectively; SEQ ID NOs:349 and 316, respectively; SEQ ID NOs:359 and 360, respectively; SEQ ID NOs:361 and 362, respectively; SEQ ID NOs:363 and 316, respectively; SEQ ID NOs:364 and 365, respectively; SEQ ID NOs:349 and 366, respectively; SEQ ID NOs:346 and 316, respectively; SEQ ID NOs:346 and 367, respectively; SEQ ID NOs:349 and 368, respectively; SEQ ID NOs:369 and 316, respectively; SEQ ID NOs:346 and 370, respectively; SEQ ID NOs:371 and 316, respectively; SEQ ID NOs:372 and 356, respectively; SEQ ID NOs:357 and 358, respectively; SEQ ID NOs:349 and 373, respectively; SEQ ID NOs:346 and 374, respectively; SEQ ID NOs:375 and 316, respectively; SEQ ID NOs:376 and 316, respectively; SEQ ID NOs:346 and 377, respectively; SEQ ID NOs:378 and 379, respectively; SEQ ID NOs:380 and 381, respectively; SEQ ID NOs:349 and 382, respectively; SEQ ID NOs:357 and 383, respectively; SEQ ID NOs:349 and 358, respectively; SEQ ID NOs:384 and 316, respectively; SEQ ID NOs:385 and 316, respectively; SEQ ID NOs:357 and 386, respectively; SEQ ID NOs:387 and 388, respectively; SEQ ID NOs:359 and 316, respectively; SEQ ID NOs:389 and 316, respectively; SEQ ID NOs:390 and 316, respectively; SEQ ID NOs:391 and 392, respectively; SEQ ID NOs:393 and 356, respectively; SEQ ID NOs:390 and 392, respectively; SEQ ID NOs:357 and 386, respectively; SEQ ID NOs:394 and 395, respectively; SEQ ID NOs:396 and 395, respectively; SEQ ID NOs:397 and 395, respectively; SEQ ID NOs:398 and 395, respectively; SEQ ID NOs:401 and 395, respectively; SEQ ID NOs:402 and 403, respectively; SEQ ID NOs:443 and 341, respectively; SEQ ID NOs:444 and 341, respectively; SEQ ID NOs:445 and 341, respectively; SEQ ID NOs:446 and 341, respectively; SEQ ID NOs:447 and 341, respectively; SEQ ID NOs:448 and 341, respectively; SEQ ID NOs:449 and 341, respectively; SEQ ID NOs:450 and 341, respectively; SEQ ID NOs:451 and 341, respectively; SEQ ID NOs:452 and 341, respectively; SEQ ID NOs:453 and 341, respectively; SEQ ID NOs:443 and 454, respectively; SEQ ID NOs:443 and 455, respectively; SEQ ID NOs:443 and 456, respectively; SEQ ID NOs:443 and 457, respectively; SEQ ID NOs:443 and 458, respectively; or SEQ ID NOs:443 and 459, respectively.

In some aspects, provided herein is an antigen-binding domain that specifically binds to human TfR, wherein the antigen-binding domain comprises a VH and a VL, wherein the VH comprises the amino acid sequence of SEQ ID NO: 399, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 313, 315, 317, 318, 319, 320, 321, 322, 323, 324, 325, 327, 330, 334, 335, 337, 38, 339, 343, 344, 345, 346, 348, 349, 351, 357, 359, 361, 363, 364, 369, 371, 372, 375, 376, 378, 380, 384, 385, 387, 389, 390, 391, 393, 394, 396, 397, 398, 401, 402, 443, 444, 445, 446, 447, 448, 449, 450, 451, 452, or 453.

In some aspects, provided herein is an antigen-binding domain that specifically binds to human TfR, wherein the antigen-binding domain comprises a VH and a VL, wherein the VL comprises the amino acid sequence of SEQ ID NO:400, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 314, 316, 326, 328, 329, 331, 332, 333, 336, 338, 340, 341, 342, 347, 350, 352, 353, 354, 355, 356, 358, 360, 362, 365, 366, 367, 368, 370, 373, 374, 377, 379, 381, 382, 383, 386, 388, 392, 395, 403, 454, 455, 456, 457, 458, or 459.

In some aspects, an antigen-binding domain provided herein comprises a VH and VL comprising the amino acid sequences of: SEQ ID NOs:399 and 400, respectively; SEQ ID NOs:10 and 11, respectively; SEQ ID NOs:12 and 13, respectively; SEQ ID NOs:14 and 15, respectively; SEQ ID NOs:16 and 17, respectively; SEQ ID NOs:18 and 19, respectively; SEQ ID NOs:20 and 21, respectively; SEQ ID NOs:22 and 23, respectively; SEQ ID NOs:24 and 25, respectively; SEQ ID NOs:26 and 27, respectively; SEQ ID NOs:28 and 29, respectively; SEQ ID NOs:30 and 31, respectively; SEQ ID NOs:32 and 33, respectively; SEQ ID NOs:34 and 35, respectively; SEQ ID NOs:36 and 37, respectively; SEQ ID NOs:38 and 39, respectively; SEQ ID NOs:40 and 41, respectively; SEQ ID NOs:42 and 43, respectively; SEQ ID NOs:44 and 45, respectively; SEQ ID NOs:46 and 47, respectively; SEQ ID NOs:48 and 49, respectively; SEQ ID NOs:50 and 51, respectively; SEQ ID NOs:52 and 53, respectively; SEQ ID NOs:54 and 55, respectively; SEQ ID NOs:56 and 57, respectively; SEQ ID NOs:58 and 59, respectively; SEQ ID NOs:60 and 61, respectively; SEQ ID NOs:62 and 63, respectively; SEQ ID NOs:64 and 65, respectively; SEQ ID NOs:66 and 67, respectively; SEQ ID NOs:68 and 69, respectively; SEQ ID NOs:70 and 71, respectively; SEQ ID NOs:72 and 73, respectively; SEQ ID NOs:313 and 314, respectively; SEQ ID NOs:315 and 316, respectively; SEQ ID NOs:317 and 314, respectively; SEQ ID NOs:318 and 316, respectively; SEQ ID NOs:319 and 314, respectively; SEQ ID NOs:320 and 316, respectively; SEQ ID NOs:321 and 314, respectively; SEQ ID NOs:322 and 316, respectively; SEQ ID NOs:323 and 314, respectively; SEQ ID NOs:324 and 316, respectively; SEQ ID NOs:325 and 326, respectively; SEQ ID NOs:327 and 328, respectively; SEQ ID NOs:325 and 329, respectively; SEQ ID NOs:330 and 331, respectively; SEQ ID NOs:325 and 332, respectively; SEQ ID NOs:330 and 333, respectively; SEQ ID NOs:334 and 332, respectively; SEQ ID NOs:335 and 336, respectively; SEQ ID NOs:337 and 338, respectively; SEQ ID NOs:38 and 33, respectively; SEQ ID NOs:339 and 340, respectively; SEQ ID NOs:339 and 341, respectively; SEQ ID NOs:339 and 342, respectively; SEQ ID NOs:343 and 340, respectively; SEQ ID NOs:343 and 342, respectively; SEQ ID NOs:344 and 340, respectively; SEQ ID NOs:344 and 341, respectively; SEQ ID NOs:344 and 342, respectively; SEQ ID NOs:345 and 340, respectively; SEQ ID NOs:345 and 341, respectively; SEQ ID NOs:345 and 342, respectively; SEQ ID NOs:346 and 347, respectively; SEQ ID NOs:348 and 316, respectively; SEQ ID NOs:349 and 347, respectively; SEQ ID NOs:349 and 350, respectively; SEQ ID NOs:351 and 352, respectively; SEQ ID NOs:349 and 353, respectively; SEQ ID NOs:346 and 354, respectively; SEQ ID NOs:349 and 355, respectively; SEQ ID NOs:349 and 356, respectively; SEQ ID NOs:357 and 316, respectively; SEQ ID NOs:349 and 358, respectively; SEQ ID NOs:349 and 316, respectively; SEQ ID NOs:359 and 360, respectively; SEQ ID NOs:361 and 362, respectively; SEQ ID NOs:363 and 316, respectively; SEQ ID NOs:364 and 365, respectively; SEQ ID NOs:349 and 366, respectively; SEQ ID NOs:346 and 316, respectively; SEQ ID NOs:346 and 367, respectively; SEQ ID NOs:349 and 368, respectively; SEQ ID NOs:369 and 316, respectively; SEQ ID NOs:346 and 370, respectively; SEQ ID NOs:371 and 316, respectively; SEQ ID NOs:372 and 356, respectively; SEQ ID NOs:357 and 358, respectively; SEQ ID NOs:349 and 373, respectively; SEQ ID NOs:346 and 374, respectively; SEQ ID NOs:375 and 316, respectively; SEQ ID NOs:376 and 316, respectively; SEQ ID NOs:346 and 377, respectively; SEQ ID NOs:378 and 379, respectively; SEQ ID NOs:380 and 381, respectively; SEQ ID NOs:349 and 382, respectively; SEQ ID NOs:357 and 383, respectively; SEQ ID NOs:349 and 358, respectively; SEQ ID NOs:384 and 316, respectively; SEQ ID NOs:385 and 316, respectively; SEQ ID NOs:357 and 386, respectively; SEQ ID NOs:387 and 388, respectively; SEQ ID NOs:359 and 316, respectively; SEQ ID NOs:389 and 316, respectively; SEQ ID NOs:390 and 316, respectively; SEQ ID NOs:391 and 392, respectively; SEQ ID NOs:393 and 356, respectively; SEQ ID NOs:390 and 392, respectively; SEQ ID NOs:357 and 386, respectively; SEQ ID NOs:394 and 395, respectively; SEQ ID NOs:396 and 395, respectively; SEQ ID NOs:397 and 395, respectively; SEQ ID NOs:398 and 395, respectively; SEQ ID NOs:401 and 395, respectively; SEQ ID NOs:402 and 403, respectively; SEQ ID NOs:443 and 341, respectively; SEQ ID NOs:444 and 341, respectively; SEQ ID NOs:445 and 341, respectively; SEQ ID NOs:446 and 341, respectively; SEQ ID NOs:447 and 341, respectively; SEQ ID NOs:448 and 341, respectively; SEQ ID NOs:449 and 341, respectively; SEQ ID NOs:450 and 341, respectively; SEQ ID NOs:451 and 341, respectively; SEQ ID NOs:452 and 341, respectively; SEQ ID NOs:453 and 341, respectively; SEQ ID NOs:443 and 454, respectively; SEQ ID NOs:443 and 455, respectively; SEQ ID NOs:443 and 456, respectively; SEQ ID NOs:443 and 457, respectively; SEQ ID NOs:443 and 458, respectively; or SEQ ID NOs:443 and 459, respectively.

In some aspects, the antigen-binding domain specifically binds to the apical domain of the human transferrin receptor (TfR), the antigen binding domain localizes to brain parenchyma in a subject following peripheral injection, and the antigen-binding domain is not within a modified CH3 domain.

In some aspects, the antigen-binding domain is capable of crossing the blood brain barrier (BBB). In some aspects, the antigen-binding domain binds to cynomolgus monkey TfR. In some aspects, the antigen-binding domain is internalized in blood-brain barrier epithelial cells. In some aspects, the blood-brain barrier epithelial cells are HCMEC/D3 cells.

In some aspects, the antigen-binding domain binds human TfR with an affinity between 500 nM and 10 μM. In some aspects, the antigen-binding domain binds human TfR with an affinity between 2 μM and 8 μM. In some aspects, the antigen-binding domain binds human TfR with an affinity between 2 μM and 5 μM. In some aspects, the antigen-binding domain binds human TfR with an affinity between 750 nM and 2 μM. In some aspects, the antigen-binding domain binds human TfR with an affinity between 50 nM and 500 nM. In some aspects, the antigen-binding domain binds human TfR with an affinity between 100 nM and 250 nM. In some aspects, the antigen-binding domain binds human TfR with an affinity between 1 nM and 50 nM. In some aspects, the antigen-binding domain binds human TfR with an affinity of 6.7 nM to 3.5 μM. In some aspects, the antigen-binding domain binds to cynomolgus TfR with an affinity of 38 nM to 2.3 μM. In some aspects, the antigen binding domain binds to human TfR with an affinity of about 3.5 μM. In some aspects, the antigen binding domain binds to cynomolgus TfR with an affinity of about 1.5 μM. In some aspects, the affinity is measured by high throughput surface plasmon resonance (SPR) detection.

In some aspects, the antigen-binding domain does not reduce cell-surface expression of TfR on HCMEC/D3 cells by more than 60% relative to cell-surface expression of TfR on HCMEC/D3 cells treated with an isotype control. In some aspects, the antigen-binding domain does not reduce cell-surface expression of TfR on HCMEC/D3 cells by more than 40% relative to cell-surface expression of TfR on HCMEC/D3 cells treated with an isotype control. In some aspects, the antigen-binding domain does not significantly increase cell-surface expression of TfR on HCMEC/D3 cells relative to cell-surface expression of TfR on HCMEC/D3 cells treated with an isotype control. In some aspects, the antigen-binding domain accumulates at least 4-fold or at least 5-fold more than an isotype control in vessel-depleted mouse brain.

In some aspects, the antigen-binding domain specifically binds to human TfR at least 5-fold more than binding to an irrelevant protein. In some aspects, the antigen-binding domain specifically binds to cynomolgus TfR at least 5-fold more than binding to an irrelevant protein. In some aspects, the antigen-binding domain binds to human TfR at least 5-fold more than binding to an irrelevant protein and/or specifically binds to cynomolgus TfR at least 5-fold more than binding to an irrelevant protein. In some aspects, the antigen-binding domain does not significantly reduce TfR expression levels in a primate brain following intravenous administration of the antigen-binding domain.

In some aspects, the antigen-binding domain comprises a VH and a VL on a single polypeptide chain. In some aspects, the antigen-binding domain comprises a single-chain fragment variable (scFv). In some aspects, the scFv is in the orientation VH-linker-VL. In some aspects, the scFv is in the orientation VL-linker-VH. In some aspects, the linker is about 5 to about 25 amino acids, is about 5 to about 20 amino acids, is about 10 to about 25 amino acids, or is about 10 to about 20 amino acids. In some aspects, the linker comprises the amino acid sequence of GGSEGKSSGSGSESKSTGGS (SEQ ID NO:183) or GGGGSGGGGSGGGGSGGGGS (SEQ ID NO:288). In some aspects, the antigen-binding domain comprises a VH on a first polypeptide and a VL on a second polypeptide.

In some aspects, the antigen-binding domains comprises the amino acid sequence of any one of SEQ ID NOs:404, 185, 186, 189, 190, 192, 193, 195-259, and 261-284.

In some aspects, the antigen-binding domain is a murine, chimeric, humanized, or human antigen-binding domain, optionally wherein the antigen-binding domain is a humanized antigen-binding domain.

In some aspects, provided herein is an antigen-binding domain that specifically binds to human TfR, wherein the antigen-binding domain is a VHH and comprises (i) the VH CDR1, VH CDR2, and VH CDR3 of an antigen-binding domain provided herein or (ii) the VH of an antigen-binding domain provided herein, optionally wherein the VHH is capable of crossing the blood brain barrier (BBB).

In some aspects, provided herein is a fusion protein comprising an antigen-binding domain provided herein and a heterologous protein or peptide. In some aspects, provided herein the heterologous protein or peptide comprises the amino acid sequence of beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (GCase or GBA), progranulin (PGRN), Prosaposin (PSAP), Glycoprotein nonmetastatic protein B (GPNMB), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33 or sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), Paired immunoglobin like type 2 receptor alpha (PILRA), Membrane Spanning 4-Domains A4A (MS4A4A), Membrane Spanning 4-Domains A 6A (MS4A6A), or Transmembrane Protein 106B (TMEM106b), clusterin (APOJ), Reelin, ubiquitin protein ligase E3A (UBE3A), Tripeptidyl Peptidase 1 (CLN2/TPP1), Alpha-L-Iduronidase (IDUA), Iduronate 2-Sulfatase (IDS), glucosamine (N-acetyl)-6-sulfatase (GNS), heparan-alpha-glucosaminide N-acetyltransferase (HGSNAT), N-acetyl-alpha-glucosaminidase (NAGLU), or N-sulfoglucosamine sulfohydrolase (SGSH), or a portion thereof.

In some aspects, provided herein is a fusion protein further comprising a Fc domain. In some aspects, the Fc domain is capable of binding FcRn.

In some aspects, provided herein is a fusion protein comprising (i) a single scFv or VHH or Fab antigen-binding domain that binds to human TfR and (ii) and two copies of a heterologous protein or peptide. In some aspects, the fusion protein comprising the single scFv, Fab or VHH antigen-binding domain that binds to human TfR is linked to the C-terminus of one of the two copies of the heterologous protein or peptide. In some aspects, the two copies of the heterologous protein or peptide are linked to the N-terminus of the Fc domain. In some aspects, the single scFv, Fab, or VHH antigen-binding domain that binds to human TfR is linked to the N-terminus of the Fc domain of the fusion protein provided herein. In some aspects, the two copies of the heterologous protein or peptide are linked to the C-terminus of the Fc domain of the fusion protein provided herein.

In some aspects, provided herein is a fusion protein comprising (i) an antibody that binds to human TfR, wherein the antibody comprises two heavy chains and two light chains; and (ii) two copies of a heterologous protein or peptide, wherein each copy of the heterologous protein or peptide is linked to the C-termini of one of the two antibody heavy chains. In some aspects, provided herein is a fusion protein comprising (i) two scFv, Fab, or VHH antigen-binding domains that bind to human TfR, (ii) a Fc domain; and (iii) two copies of a heterologous protein or peptide, wherein the two scFv, Fab, or VHH antigen-binding domains that bind to human TfR are linked to the C-terminus of the Fc domain, and the two copies of the heterologous protein or peptide are linked to the N-terminus of the Fc domain. In some aspects, provided herein is a fusion protein comprising (i) a single scFv, VHH, or Fab antigen-binding domain that binds to human TfR, (ii) a Fc domain, and (iii) a single copy of a heterologous protein or peptide, wherein the single scFv, VHH, or Fab antigen-binding domain that binds to human TfR is linked to the C-terminus of the Fc domain, and the heterologous protein or polypeptide linked to N-terminus of the Fc domain. In some aspects, provided herein is a fusion protein comprising (i) a single scFv, VHH, or Fab antigen-binding domain that binds to human TfR, (ii) a Fc domain, and (iii) a single copy of the heterologous protein or peptide, wherein the single scFv, VHH, or Fab antigen-binding domain that binds to human TfR is linked to the N-terminus of the Fc domain, and the heterologous protein or polypeptide linked to the C-terminus of the Fc domain. In some aspects, provided herein is a fusion protein comprising (i) a single scFv, VHH, or Fab antigen-binding domain that binds to human TfR, (ii) a Fc domain, and (iii) a single copy of the heterologous protein or peptide, wherein the single scFv, VHH, or Fab antigen-binding domain that binds to human TfR and the heterologous protein or polypeptide are both linked to the N-terminus of the Fc domain.

In some aspects, the Fc domain is a heterodimeric Fc, optionally comprising knob and hole mutations. In some aspects, the Fc is a single chain monovalent Fc. In some aspects, the Fc is a modified Fc with a modification or modifications listed in Table 1 or 2. In some aspects, the Fc comprises a mutation that reduces effector function, optionally wherein the mutation that reduces effector function comprises (i) L234A, L235A, and/or P331S, and/or (ii) N325S and/or L328F, and/or (iii) P329G or P329S.

In some aspects, provided herein is an antibody comprising an antigen-binding domain provided herein. In some aspects, provided herein is an antibody or antigen-binding fragment thereof that binds to the same human TfR epitope as an antigen-binding domain provided herein. In some aspects, provided herein is an antibody or antigen-binding fragment thereof that competitively inhibits binding to human TfR of an antigen-binding domain provided herein.

In some aspects, provided herein is a multi-specific protein comprising a first antigen-binding domain that is an antigen-binding domain provided herein linked to a second antigen-binding domain. In some aspects, the second antigen-binding domain specifically binds to a CNS antigen. In some aspects, provided herein is a multi-specific protein comprising an antigen-binding domain provided herein linked to an antibody or antigen-binding fragment thereof. In some aspects, the antibody or antigen-binding fragment thereof specifically binds to a CNS antigen. In some aspects, the antibody or antigen-binding fragment thereof comprises a heavy chain constant region. In some aspects, the antigen-binding domain provided herein is linked, optionally via an amino acid linker, to the C-terminus of the heavy chain constant region. In some aspects, the multi-specific protein is bispecific. In some aspects, the multi-specific protein is bivalent, trivalent, or tetravalent. In some aspects, the multi-specific protein is bivalent. In some aspects, the multi-specific protein is trivalent, optionally wherein the trivalent protein comprises one antigen-binding domain that binds to human TfR and two antigen-binding domains that bind to a CNS antigen. In some aspects, the multi-specific protein is tetravalent, optionally wherein the tetravalent protein comprises two antigen-binding domains that bind to human TfR and two antigen-binding domains that bind to a CNS antigen.

In some aspects, provided herein is a multi-specific protein that is trivalent and bi-specific and comprises an antigen-binding domain provided herein linked to an antibody that binds to a CNS antigen, wherein the antibody comprises two heavy chains and two light chains, and wherein the antigen-binding domain is an scFv linked, optionally via an amino acid linker, to the C-terminus of one of the two antibody heavy chains.

In some aspects, provided herein is a multi-specific protein that is trivalent and bi-specific and comprises an antigen-binding domain provided herein linked to an antibody that binds to a CNS antigen, wherein the antibody comprises two heavy chains and two light chains, and wherein the antigen-binding domain is an scFv linked, optionally via an amino acid linker, to the N-terminus of one of the two antibody heavy chains.

In some aspects, provided herein is a multi-specific protein that is tetravalent and bi-specific and comprises two antigen-binding domains provided herein, and an antibody that binds to a CNS antigen, wherein the antibody comprises two heavy chains and two light chains, wherein each of the two antigen-binding domains is an scFv, Fab, or VHH, wherein one of the two antigen-binding domain is linked, optionally via an amino acid linker, to the C-terminus of one of the antibody heavy chains, and wherein the other antigen-binding domain is linked, optionally via an amino acid linker, to the C-terminus of the other antibody heavy chain.

In some aspects, provided herein is a multi-specific protein that is tetravalent and bi-specific and comprises two antigen-binding domains provided herein, and an antibody that binds to a CNS antigen, wherein the antibody comprises two heavy chains and two light chains, wherein each of the two the antigen-binding domains is an scFv, Fab, or VHH, wherein one of the two antigen-binding domains is linked, optionally via an amino acid linker, to the N-terminus of one of the antibody heavy chains, and wherein the other antigen-binding domain is linked, optionally via an amino acid linker, to the N-terminus of the other antibody heavy chain.

In some aspects, provided herein is a multi-specific protein that is tetravalent and bi-specific and comprises two antigen-binding domains provided herein, and an antibody that binds to a CNS antigen, wherein the antibody comprises two heavy chains and two light chains, wherein each of the two the antigen-binding domains is an scFv, Fab, or VHH, wherein one of the two antigen-binding domains is linked, optionally via an amino acid linker, to the N-terminus of one of the antibody heavy chains, and wherein the other antigen-binding domain is linked, optionally via an amino acid linker, to the C-terminus of the other antibody heavy chain.

In some aspects, the two antigen-binding domains provided herein are two copies of the same antigen-binding domain.

In some aspects, provided herein is a multispecific protein that is bivalent and bi-specific, comprising: (i) a Fc domain, (ii) a single antigen-binding domain provided herein, wherein the antigen-binding domain is a single scFv, VHH, or Fab antigen-binding domain that binds to human TfR and is linked to the N-terminus of the Fc domain, and (iii) a second antigen binding domain that specifically binds a CNS antigen, wherein the second antigen-binding domain is a single scFv, VHH or Fab linked to the C-terminus of the Fc domain.

In some aspects, provided herein is a multispecific protein that is bivalent and bi-specific, comprising: (i) a Fc domain, (ii) a single antigen-binding domain provided herein, wherein the antigen-binding domain is a single scFv, VHH, or Fab antigen-binding domain that binds to human TfR and is linked to the C-terminus of the Fc domain, and (iii) a second antigen binding domain that specifically binds a CNS antigen, wherein the second antigen-binding domain is a single scFv, VHH or Fab linked to the N-terminus of the Fc domain.

In some aspects, the Fc domain is a heterodimeric Fc, optionally comprising knob and hole mutations. In some aspects, the Fc is a modified Fc with a modification, or modifications, listed in Table 1 or 2. In some aspects, the Fc comprises a mutation that reduces effector function, optionally wherein the mutation that reduces effector function comprises (i) L234A, L235A, and/or P331S, and/or (ii) N325S and/or L328F, and/or (iii) P329G or P329S. In some aspects, the antibody or antigen-binding fragment thereof comprises a mutation that reduces effector function, optionally wherein the mutation that reduces effector function comprises (i) L234A, L235A, and/or P331S, and/or (ii) N325S and/or L328F, and/or (iii) P329G or P329S. In some aspects, the antibody or antigen-binding fragment thereof comprises a constant region comprising a knob mutation and a mutation that reduces effector function, optionally wherein the mutation that reduces effector function comprises (i) L234A, L235A, and/or P331S, and/or (ii) N325S and/or L328F, and/or (iii) P329G or P329S. In some aspects, the antibody or antigen-binding fragment thereof comprises a constant region comprising a hole mutation and a mutation that reduces effector function, optionally wherein the mutation that reduces effector function comprises (i) L234A, L235A, and/or P331S, and/or (ii) N325S and/or L328F, and/or (iii) P329G or P329S.

In some aspects, the antibody or antigen-binding fragment thereof comprises a constant region comprising a knob mutation and a constant region comprising a hole mutation. In some aspects, the antigen-binding domain is linked, optionally via an amino acid linker, to the constant region comprising a hole mutation. In some aspects, the antigen-binding domain is linked, optionally via an amino acid linker, to the constant region comprising a knob mutation.

In some aspects, the amino acid linker is a glycine-serine linker. In some aspects, the glycine-serine linker comprises the amino acid sequence (GGGGS)x3 (SEQ ID NO:184) or the amino acid sequence (GGSGG)x3 (SEQ ID NO:289).

In some aspects, the CNS antigen is a brain antigen. In some aspects, the CNS antigen is not TfR.

In some aspects, the antibody or antigen-binding fragment thereof is an IgG antibody or antigen-binding fragment thereof. In some aspects, the IgG antibody or antigen-binding fragment thereof is an IgG1 antibody or antigen-binding fragment thereof or an IgG4 antibody or antigen-binding fragment thereof.

In some aspects, the multi-specific protein binds human TfR with an equilibrium dissociation constant (KD) of about 65 nM to about 4 μM and/or binds cynomolgus monkey TfR with a KD of about 37 nM to about 1.3 μM.

In some aspects, the multi-specific protein is internalized in blood-brain barrier epithelial cells greater than 10-fold or greater than 40-fold as compared to internalization by an isotype control, optionally wherein the blood-brain barrier epithelial cells are HCMEC/D3 cells. In some aspects, the multi-specific protein does not reduce cell-surface expression of TfR on HCMEC/D3 cells by more than 60% relative to cell-surface expression of TfR on HCMEC/D3 cells treated with an isotype control. In some aspects, the multi-specific protein does not reduce cell-surface expression of TfR on HCMEC/D3 cells by more than 40% relative to cell-surface expression of TfR on HCMEC/D3 cells treated with an isotype control. In some aspects, the multi-specific protein does not significantly increase cell-surface expression of TfR on HCMEC/D3 cells relative to cell-surface expression of TfR on HCMEC/D3 cells treated with an isotype control.

In some aspects, the multi-specific protein accumulates at least 4- or 5-fold more than an isotype control in vessel-depleted mouse brain. In some aspects, the second antigen-binding domain specifically binds to human TfR at least 5-fold more than binding to an irrelevant protein. In some aspects, the second antigen-binding domain specifically binds to cynomolgus TfR at least 5-fold more than binding to an irrelevant protein. In some aspects, the second antigen-binding domain binds to human TfR at least 5-fold more than binding to an irrelevant protein and/or specifically binds to cynomolgus TfR at least 5-fold more than binding to an irrelevant protein.

In some aspects, the CNS antigen is beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (GCase or GBA), progranulin (PGRN), Prosaposin (PSAP), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), sialic acid binding Ig-like lectin 11 (Siglec11), glycoprotein nonmetastatic melanoma protein B (GPNMB), Paired immunoglobin like type 2 receptor alpha (PILRA), Membrane Spanning 4-Domains A4A (MS4A4A), Membrane Spanning 4-Domains A 6A (MS4A6A), MS4A4E, Transmembrane Protein 106B (TMEM106b), ubiquitin protein ligase E3A (UBE3A), CR1, ABCA1, ABCA7, HLA-DR1, HLA-DR5, IL1RAP, TREML2, IL-34, SORL1, or ADAM1. In some aspects, the CNS antigen is MS4A4A, optionally wherein (i) the antigen-binding domain, antibody, or antigen-binding domain that binds to MS4A4A comprises a VH comprising the MS4A4A-binding VH sequence of SEQ ID NO:406 and/or a VL comprising the VL sequence of SEQ ID NO:407; and/or (ii) the antigen-binding domain that binds to human TfR comprises the scFv sequence of SEQ ID NO: 406.

In some aspects, the multi-specific protein comprises the amino acid sequences of SEQ ID NOs:405-407.

In some aspects, the multi-specific protein is capable of binding FcRn.

In some aspects, a fusion protein, antibody or antigen-binding fragment thereof, or multi-specific protein provided herein is linked to an imaging agent.

In some aspects, a fusion protein, antibody or antigen-binding fragment thereof, or multi-specific protein provided herein is capable of crossing the BBB.

In some aspects, provided herein is a composition comprising a first polynucleotide, a second polynucleotide, and a third polynucleotide, wherein the first, second, and third polynucleotides encode a multi-specific protein provided herein, wherein the first polynucleotide encodes a first heavy chain, the second polynucleotide encodes a second heavy chain and the antigen-binding domain that specifically binds to human TfR, and the third polynucleotide encodes a light chain.

In some aspects, provided herein is a composition comprising a first polynucleotide, a second polynucleotide, and a third polynucleotide, wherein the first, second, and third polynucleotides encode a multi-specific protein provided herein, wherein the first polynucleotide encodes a first heavy chain and a first antigen-binding domain that specifically binds to human TfR, the second polynucleotide encodes a second heavy chain and a second antigen-binding domain that specifically binds to human TfR, and the third polynucleotide encodes a light chain, optionally wherein the first and second antigen-binding domains that bind to human TfR comprise the same amino acid sequence.

In some aspects, the first heavy chain comprises a knob mutation and the second heavy chain comprises a hole mutation.

In some aspects, the first heavy chain comprises a hole mutation and the second heavy chain comprises a knob mutation. In some aspects, the first heavy chain comprises a hole mutation and the second heavy chain comprises a knob mutation.

In some aspects, the ratio of the first, second, and third polynucleotides is about 1:3:6.

In some aspects, provided herein is a composition comprising a first polynucleotide and a second polynucleotide, wherein the first and second polynucleotides encode a multi-specific protein provided herein, wherein the first polynucleotide encodes a heavy chain and an antigen-binding domain that bind to human TfR, and wherein the second polynucleotide encodes a light chain.

In some aspects, provided herein is a host cell comprising a composition provided herein.

In some aspects, provided herein is an isolated polynucleotide comprising a nucleic acid molecule encoding the heavy chain of an antigen-binding domain provided herein. In some aspects, provided herein is an isolated polynucleotide comprising a nucleic acid molecule encoding a light chain variable region of an antigen-binding domain provided herein.

In some aspects, provided herein is an isolated vector comprising a polynucleotide provided herein.

In some aspects, provided herein is an isolated vector comprising a nucleic acid molecule encoding a heavy chain variable region of an antigen-binding domain provided herein and a nucleic acid molecule encoding a light chain variable region of an antigen-binding domain.

In some aspects, provided herein is a host cell comprising a polynucleotide provided herein or a vector provided herein. In some aspects, the host cell is selected from the group consisting of E. coli, Pseudomonas, Bacillus, Streptomyces, yeast, CHO, YB/20, NS0, PER-C6, HEK-293T, NIH-3T3, HeLa, BHK, Hep G2, SP2/0, R1.1, B-W, L-M, COS 1, COS 7, BSC1, BSC40, BMT10 cell, plant cell, insect cell, and human cell in tissue culture.

In some aspects, provided herein is a method of producing an antigen-binding domain or multi-specific protein comprising culturing a host cell provided herein so that the antigen-binding domain or multi-specific protein is produced, optionally wherein the method further comprises isolating the antigen-binding domain or multi-specific protein from the culture.

In some aspects, provided herein is an isolated antigen-binding domain or multi-specific protein thereof produced by a method provided herein.

In some aspects, provided herein is a pharmaceutical composition comprising (i) a antigen-binding domain, fusion protein, antibody or antigen-binding fragment thereof, or multi-specific protein provided herein and (ii) a pharmaceutically acceptable carrier. In some aspects, the concentration of the fusion protein, antibody or an antigen-binding fragment thereof, or multi-specific protein is increased in the brain following administration to a subject as compared to an isotype control. In some aspects, the administration increases delivery of fusion protein, antibody or antigen-binding fragment thereof, multi-specific protein, or pharmaceutical composition into the brain by at least 50%, at least 100%, at least 200%, at least 500% or at least 1000% as compared to an isotype control.

In some aspects, provided herein is a method of treating a neurological disease or disorder in a subject comprising administering a fusion protein, antibody or antigen-binding fragment thereof, multi-specific protein, or pharmaceutical composition provided herein to the subject. In some aspects, the administration increases delivery of the fusion protein, antibody or antigen-binding fragment thereof, multi-specific protein, or pharmaceutical composition into the brain by at least 50%, at least 100%, at least 200%, at least 500% or at least 1000% as compared to an isotype control. In some aspects, the administration increases delivery of fusion protein, antibody or antigen-binding fragment thereof, multi-specific protein, or pharmaceutical composition into the frontal cortex, the entorhinal cortex and/or the hippocampus.

In some aspects, the neurological disease or disorder is selected from a neuropathy disorder, a neurodegenerative disease, cancer, an ocular disease disorder, a seizure disorder, a lysosomal storage disease, amyloidosis, a viral or microbial disease, ischemia, a behavioral disorder, and CNS inflammation. In some aspects, the neurological disease or disorder is selected from Alzheimer's disease (AD), Huntington's disease, dystonia, ataxia, Bell's palsy, stroke, dementia, Lewy body dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, encephalitis, traumatic brain injury, and limbic-predominant age-related TDP-43 encephalopathy (LATE). In some aspects, the dementia is frontotemporal dementia (FTD). In some aspects, the neurological disease or disorder is Alzheimer's disease. In some aspects, the Alzheimer's disease is early onset Alzheimer's disease, prodromal Alzheimer's disease, mild Alzheimer's disease, or late onset Alzheimer's disease. In some aspects, the neurological disease or disorder is Parkinson's disease. In some aspects, the neurological disease or disorder is frontal temporal epilepsy. In some aspects, the neurological disease or disorder is autism. In some aspects, the neurological disease or disorder is lissencephaly.

In some aspects, provided herein is a method of treating a lysosomal storage disease in a subject comprising administering a fusion protein, antibody or antigen-binding fragment thereof, multi-specific protein, or pharmaceutical composition provided herein to the subject. In some aspects, the lysosomal storage disease is selected from Gaucher disease, Ceroid lipofuscinosis (Batten disease), Mucopolysaccharidosis (MPS) Type I, MPS Type II, and MPS Type III.

In some aspects, provided herein is a method of transporting a fusion protein, antibody or an antigen-binding fragment thereof, or multi-specific protein across the BBB of a subject, comprising administering to the subject a fusion protein, antibody or antigen-binding fragment thereof, multi-specific protein, or pharmaceutical composition provided herein.

In some aspects, the concentration of the fusion protein, antibody or an antigen-binding fragment thereof, or multi-specific protein is increased in the brain following administration as compared to an isotype control. In some aspects, the concentration of the fusion protein, antibody or antigen-binding fragment thereof, multi-specific protein, or pharmaceutical composition in the brain is increased by at least 50%, at least 100%, at least 200%, at least 500% or at least 1000% as compared to an isotype control.

In some aspects, the administration of the fusion protein, antibody or an antigen-binding fragment thereof, or multi-specific protein does not result in reticulocyte count reduced in the subject by more than 10%, as compared to administration of an isotype control. In some aspects, the administration of the fusion protein, antibody or an antigen-binding fragment thereof, or multi-specific protein does not result in reticulocyte count reduction in the subject, as compared to an isotype control.

In some aspects, provided herein is a method of increasing the concentration of a CNS binding antigen in the CSF of a subject, comprising administering the multi-specific protein provided herein to the subject, wherein the concentration of the CNS binding antigen is increased as compared to administering the CNS binding antigen alone to the subject.

In some aspects, provided herein is a method of imaging a CNS antigen within a subject, comprising administering to the subject a fusion protein, antibody or antigen-binding fragment thereof, or multi-specific protein provided herein and locating the imaging agent within the subject.

In some aspects, provided herein is a method of detecting a CNS antigen in vitro, comprising contacting an in vitro sample with a fusion protein, antibody or antigen-binding fragment thereof, or multi-specific protein provided herein and locating the imaging agent within the sample.

In some aspects, provided herein is a use of a fusion protein, antibody or antigen-binding fragment thereof, multi-specific protein, or pharmaceutical composition provided herein in a method provided herein.

In some aspects, provided herein is a fusion protein, antibody or antigen-binding fragment thereof, multi-specific protein, or pharmaceutical composition provided herein for use in a method provided herein.

It is to be understood that one, some, or all of the properties of the various aspects described herein can be combined to form other aspects of the present disclosure. These and other aspects of the disclosure will be immediately apparent to one of skill in the art. These and other aspects of the disclosure are further described by the detailed description that follows.

### BRIEF DESCRIPTION OF THE DRAWINGS

FIG. 1A shows an example of a 2+1 bispecific antibody.

FIG. 1B shows an example of a 2+2 bispecific antibody.

FIG. 1C shows an example of a 2+2 bispecific antibody with two different scFvs.

FIG. 1D shows exemplary formats comprising a TfR antigen-binding domain. (i) shows an exemplary 2+1 bispecific antibody format; (ii) shows an exemplary 1+1 antibody format; and (iii) shows another exemplary 1+1 antibody format, with two different VHH domains.

FIG. 1E shows exemplary formats of fusion proteins comprising a TfR antigen-binding domain and a heterologous protein or polypeptide. The circular “pacman” shape represents a heterologous protein or polypeptide. (i), (ii), (iii) and (iv) show exemplary 1+1 fusion protein formats. (v) and (vi) show exemplary 2+1 fusion protein formats. (vii) shows an exemplary 2+2 fusion protein format.

FIG. 2 shows TfR surface levels on hCMEC/D3 cells after treatment with 2+1 anti-TfR bispecific antibodies, evaluated via FACS. (See Example 13.)

FIG. 3 shows TfR surface levels on hCMEC/D3 cells after treatment with humanized 2+1 anti-TfR bispecific antibodies, evaluated via FACS. (See Example 13.)

FIG. 4 shows total TfR protein levels in hCMEC/D3 cells after treatment with 2+1 anti-TfR bispecific antibodies, evaluated via Western blot. (See Example 13.)

FIG. 5 shows the brain PK of 2+1 anti-TfR bispecific antibodies as fold change over isotype control. (See Example 17.)

FIG. 6 shows the serum PK of 2+1 anti-TfR bispecific antibodies in wild-type and hTfR ECD^{+/-} knock-in mice. (See Example 19.)

FIG. 7 shows total vessel TfR levels after hu-TfR^{-/-} KI mice were injected with anti-TfR antibodies (i.e., TfR-9.1B.39 and TfR-15.WH8) compared to those from isotype treated animals. (See Example 20.)

FIG. 8 shows blood reticulocyte levels after 2+1 anti-TfR bispecific antibody treatment. (See Example 21.)

FIG. 9 shows a modest antibody-dependent cellular cytotoxicity (ADCC) response of 2+1 anti-TfR bispecific antibodies against a BBB cell line. (See Example 22.)

FIG. 10 shows that NSLF and LALAPS strongly ameliorate ADCC signal from 2+1 anti-TfR bispecific antibodies. (See Example 22.)

FIG. 11 shows a 2+1 anti-TfR bispecific antibody activity as measured by sTREM2 levels in an in vitro assay. (See Example 24.)

FIG. 12 shows absolute reticulocyte counts in non-human primates (NIP) following administration of a 2+1 anti-TfR bispecific antibody. (See Example 26.)

FIG. 13 shows serum and CSF levels of a 2+1 anti-TfR bispecific antibody after a first and second dosing. (See Example 27.)

FIG. 14 shows antibody concentration in NHP brain fractions following administration of a 2+1 anti-TfR bispecific antibody. (See Example 28.)

FIG. 15 shows soluble TREM2 (sTREM2) levels in serum and CSF of NHPs following administration of a 2+1 anti-TfR bispecific antibody. (See Example 29.)

FIG. 16 shows levels of CSF-1 in the CSF of NHPs following administration of a 2+1 anti-TfR bispecific antibody. (See Example 29.)

FIG. 17 shows relative TfR protein levels (determined by a sandwich Meso Scale Discovery method) in cynomolgus brain sections 48 hours after a 2nd dose of 2+1 anti-TfR antibody. Tfr protein concentrations for each animal were normalized and are shown as a percentage of the TfR level in the average of the control-treated animals (hIgG1 Iso). (See Example 30.)

### DETAILED DESCRIPTION OF THE PRESENT DISCLOSURE

The present disclosure relates to antigen-binding domains that specifically binds to human transferrin receptor (TfR), antibodies an antigen-binding fragments thereof comprising such antigen-binding domains, methods of making and using such antigen-binding domains, antibodies, and antigen-binding fragments thereof, pharmaceutical compositions comprising such antigen-binding domains, antibodies, and antigen-binding fragments thereof, nucleic acids encoding such antigen-binding domains, antibodies, and antigen-binding fragments thereof, and host cells comprising nucleic acids encoding such antigen-binding domains, antibodies, and antigen-binding fragments thereof.

The techniques and procedures described or referenced herein are generally well understood and commonly employed using conventional methodology by those skilled in the art, such as, for example, the widely utilized methodologies such as those described in Sambrook et al. Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003); Monoclonal Antibodies: A Practical Approach (P. Shepherd and C. Dean, eds., Oxford University Press, 2000).

### Definitions

The terms “central nervous system” and “CNS” refer to the complex of nerve tissues that control bodily function and includes the brain, spinal cord.

The terms “blood brain barrier” and “BBB” refer to a network of brain capillary endothelial cells that are closely sealed by tight junctions.

A “central nervous system antigen” or “CNS antigen” is an antigen expressed in the CNS, including the brain, which can be targeted with an antibody or small molecule. Examples of such antigens include, without limitation: beta-secretase 1 (BACE1), amyloid beta (Abeta), epidermal growth factor receptor (EGFR), human epidermal growth factor receptor 2 (HER2), tau, apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (Gcase or GBA), progranulin (PGRN), Prosaposin (PSAP), gamma secretase, death receptor 6 (DR6), amyloid precursor protein (APP), p75 neurotrophin receptor (p75NTR), interleukin 6 receptor (IL6R), TNF receptor 1 (TNFR1), interleukin 1 beta (IL1(3)), caspase 6, sortilin (SORT), triggering receptor expressed on myeloid cells 2 (TREM2), CD33 or sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), glycoprotein nonmetastatic melanoma protein B (GPNMB), Paired immunoglobin like type 2 receptor alpha (PILRA), Membrane Spanning 4-Domains A4A (MS4A4A), Membrane Spanning 4-Domains A 6A (MS4A6A), ubiquitin protein ligase E3A (UBE3A), or Transmembrane Protein 106B (TMEM106b).

A “brain antigen” is a CNS antigen expressed in the brain.

The terms “Transferrin receptor,” “TfR,” “TfR polypeptide,” and “TfR protein” are used interchangeably herein to refer to any native TfR from any vertebrate source, including mammals such as primates (e.g., humans and cynomolgus monkeys (cynos)) and rodents (e.g., mice and rats), unless otherwise indicated. TfR is also referred to as transferrin receptor protein 1, TR, tfR1, Trfr, T9, and p90. In some aspects, the term encompasses both wild-type sequences and naturally occurring variant sequences, e.g., splice variants or allelic variants. In some aspects, the term encompasses “full-length,” unprocessed TfR, as well as any form of TfR that results from processing in the cell. In some aspects, the TfR is human TfR. As used herein, the term “human TfR” refers to a polypeptide with the amino acid sequence of SEQ ID NO:8.

(SEQ ID NO: 8)
MMDQARSAFSNLFGGEPLSYTRFSLARQVDGDNSHVEMKLAVDEEENADN
NTKANVTKPKRCSGSICYGTIAVIVFFLIGFMIGYLGYCKGVEPKTECER
LAGTESPVREEPGEDFPAARRLYWDDLKRKLSEKLDSTDFTGTIKLLNEN
SYVPREAGSQKDENLALYVENQFREFKLSKVWRDQHFVKIQVKDSAQNSV
IIVDKNGRLVYLVENPGGYVAYSKAATVTGKLVHANFGTKKDFEDLYTPV
NGSIVIVRAGKITFAEKVANAESLNAIGVLIYMDQTKFPIVNAELSFFGH
AHLGTGDPYTPGFPSFNHTQFPPSRSSGLPNIPVQTISRAAAEKLFGNME
GDCPSDWKTDSTCRMVTSESKNVKLTVSNVLKEIKILNIFGVIKGFVEPD
HYVVVGAQRDAWGPGAAKSGVGTALLLKLAQMFSDMVLKDGFQPSRSIIF
ASWSAGDFGSVGATEWLEGYLSSLHLKAFTYINLDKAVLGTSNFKVSASP
LLYTLIEKTMQNVKHPVTGQFLYQDSNWASKVEKLTLDNAAFPFLAYSGI
PAVSFCFCEDTDYPYLGTTMDTYKELIERIPELNKVARAAAEVAGQFVIK
LTHDVELNLDYERYNSQLLSFVRDLNQYRADIKEMGLSLOWLYSARGDFF
RATSRLTTDFGNAEKTDRFVMKKLNDRVMRVEYHFLSPYVSPKESPFRHV
FWGSGSHTLPALLENLKLRKQNNGAFNETLFRNQLALATWTIQGAANALS
GDVWDIDNEF

As used herein, the terms “antibody” and “immunoglobulin” are used interchangeably and refer to an antibody molecule that recognizes and specifically binds to a target, such as a protein, polypeptide, peptide, carbohydrate, polynucleotide, lipid, or combinations of the foregoing (e.g., a glycoprotein), through at least one antigen recognition site within the variable region of the immunoglobulin molecule. The term “antibody” encompasses monoclonal antibodies, chimeric antibodies, humanized antibodies, human antibodies, multi-specific (e.g. bi-specific) antibodies, and any other immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1, and IgA2), based on the identity of their heavy-chain constant regions referred to as alpha, delta, epsilon, gamma, and mu, respectively. The different classes of antibodies have different and well-known subunit structures and three-dimensional configurations. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6.

The terms “anti-TfR antibody,” “antibody that binds to TfR,” and “antibody that specifically binds TfR” refer to an antibody that is capable of binding TfR with sufficient affinity such that the antibody is useful as a diagnostic and/or therapeutic agent in targeting TfR. In one aspect, the extent of binding of an anti-TfR antibody to an unrelated, non-TfR polypeptide is less than about 10% of the binding of the antibody to TfR as measured, e.g., by a radioimmunoassay (RIA). In certain aspects, an antibody that binds to TfR has a dissociation constant (KD) of <20 μM, <15 μM, <12 μM, <10 μM, <7.5 μM, <5 μM, <, 2.5 μM, <1 M, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M). In certain aspects, an anti-TfR antibody binds to an epitope of TfR that is conserved among TfR from different species.

The term “antibody fragment” refers to a portion of an antibody. An “antigen-binding fragment” of an antibody refers to a portion of an antibody that binds to an antigen. An antigen-binding fragment of an antibody can comprise the antigenic determining regions of an antibody (e.g., the complementarity determining regions (CDRs)). Examples of antigen-binding fragments of antibodies include, but are not limited to Fab, Fab′, F(ab′)2, and Fv fragments, linear antibodies, and single chain antibodies. An antigen-binding fragment of an antibody can be monovalent or multi-valent (e.g., bi-valent). An antigen-binding fragment of an antibody can be monospecific or multi-specific (e.g., bi-specific.) An antigen-binding fragment of an antibody can be derived from any animal species, such as rodents (e.g., mouse, rat, or hamster) and humans or can be artificially produced.

An “antigen-binding domain” or “antigen-binding region” refers to a monovalent portion of an antibody that binds to an antigen. An “antigen-binding domain” can comprise the antigenic determining regions of an antibody (e.g., the complementarity determining regions (CDRs)). An antibody or antigen-binding fragment thereof (including mono-specific and multi-specific (e.g., bi-specific) antibodies or antigen-binding fragments thereof can comprise an antigen-binding domain. In some aspects, an antigen-binding domain is not present in the context of an antibody.

The terms “anti-TfR antigen-binding domain,” “antigen-binding domain that binds to TfR,” “anti-TfR antigen-binding region,” “antigen-binding region that binds to TfR,” and “TfR binding domain” refer to an antigen-binding domain that binds to TfR with sufficient affinity such that the antigen-binding domain is useful as a diagnostic and/or therapeutic agent in targeting TfR. In one aspect, the extent of binding of an anti-TfR antigen-binding domain to an unrelated, non-TfR polypeptide is less than about 10% of the binding of the antigen-binding domain to TfR as measured, e.g., by a radioimmunoassay (RIA). In certain aspects, an antibody that binds to TfR has a dissociation constant (KD) of <20 μM, <15 μM, <12 μM, <10 μM, <7.5 μM, <5 μM, <2.5 μM, <1 M, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹M to 10⁻¹³ M). In certain aspects, an anti-TfR antigen-binding domain binds to an epitope of TfR that is conserved among TfR from different species.

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant regions can be native sequence constant regions (e.g., human native sequence constant regions) or amino acid sequence variants thereof. In some cases, the intact antibody can have one or more effector functions.

“Native IgG antibodies” are usually heterotetrameric glycoproteins of about 150,000 Daltons, composed of two identical light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

Papain digestion of antibodies produces two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

“Fv” is the minimum antibody fragment which comprises a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) can recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. In some aspects, the scFv polypeptide further comprises a polypeptide linker between the V_{H} and V_{L} domains, which enables the scFv to form the desired structure for antigen binding.

The term “diabodies” refers to small antibody fragments prepared by constructing scFv fragments (see preceding paragraph) with short linkers (about 5-10) residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” scFv fragments in which the V_{H} and V_{L} domains of the two antibodies are present on different polypeptide chains.

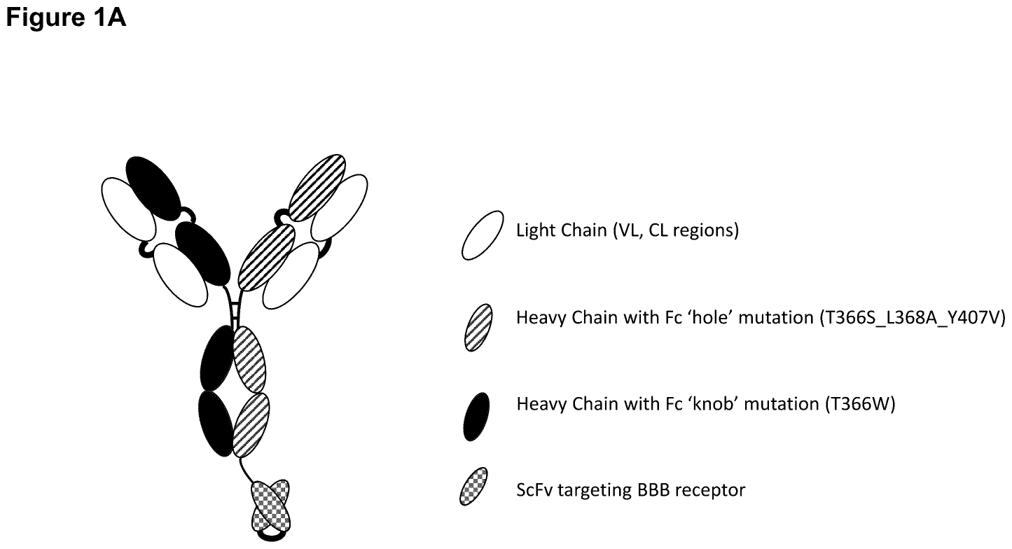

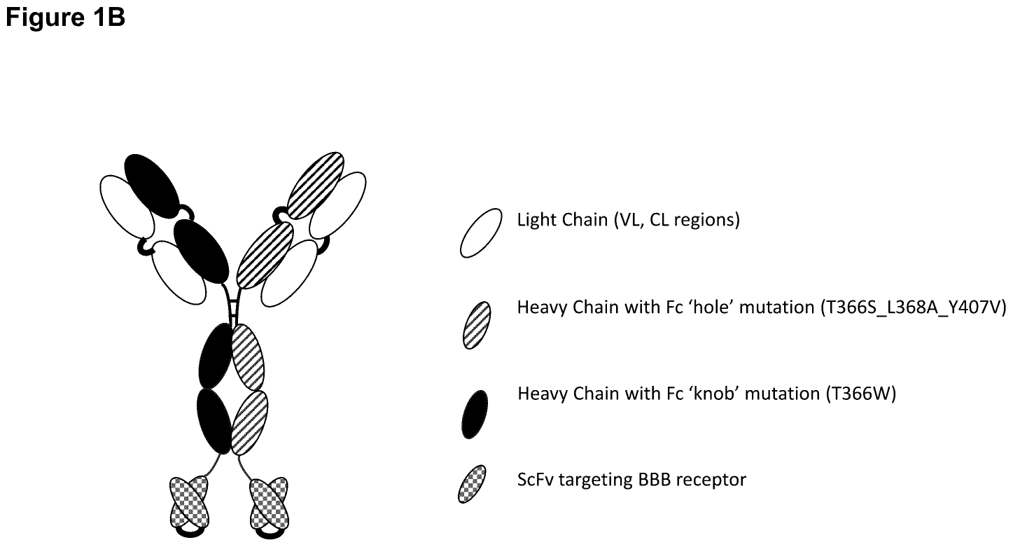

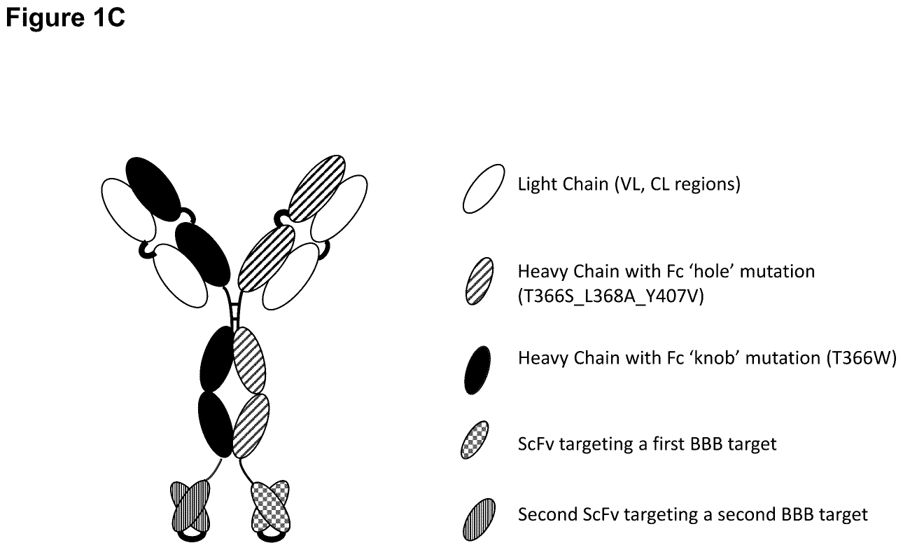

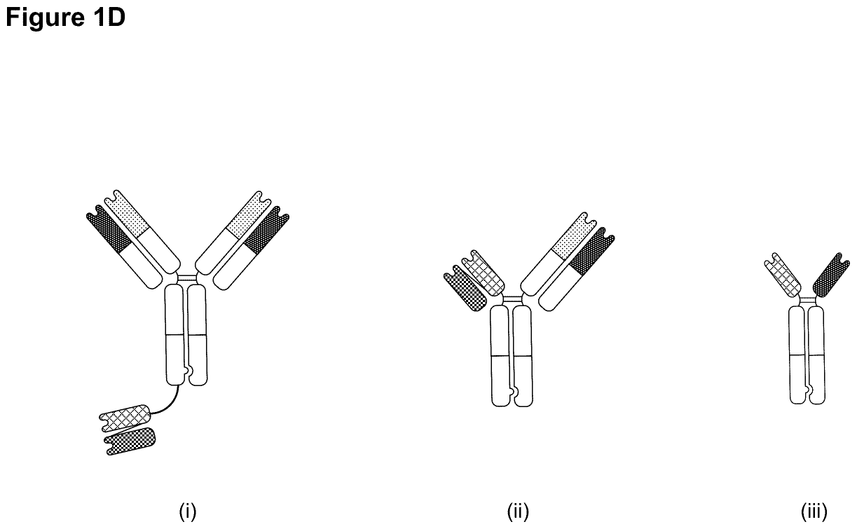

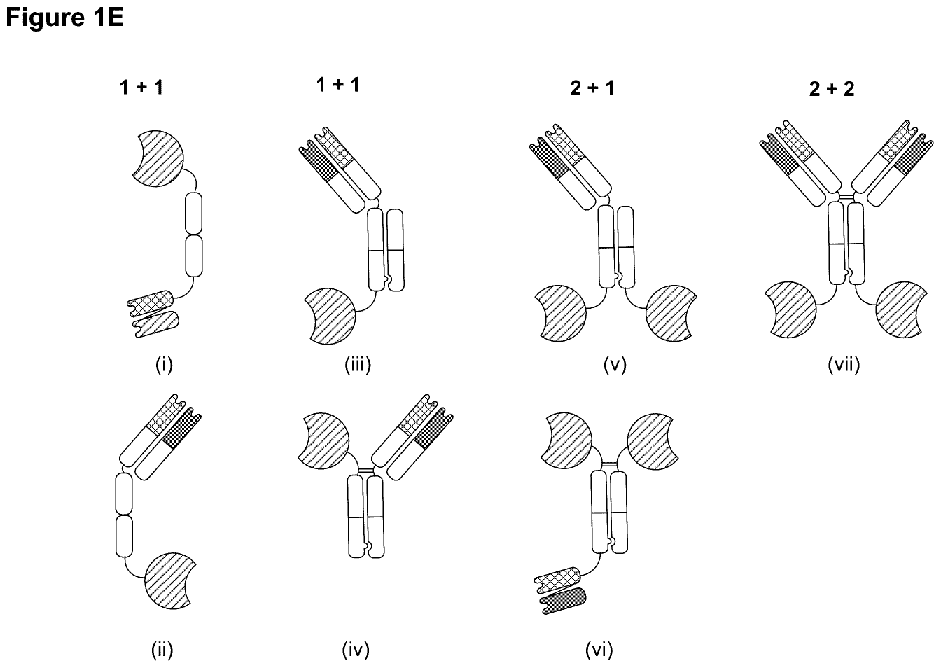

As used herein, a “2+1 antibody format” of a TfR antibody refers to a trivalent, bi-specific antibody format comprising (i) a single antigen-binding domain that binds to human TfR and (ii) an antibody, wherein the antibody comprises two heavy chains and two light chains; wherein the single antigen-binding domain that binds to human TfR is linked to the C-terminus of one of the two antibody heavy chains. This format is exemplified in FIGS. 1A, and (i) in ID.

As used herein, a “2+2 antibody format” of a TfR antibody refers to a tetravalent, bi-specific antibody format comprising (i) two antigen-binding domains that bind to human TfR and (ii) an antibody, wherein the antibody comprises two heavy chains and two light chains; wherein one antigen-binding domain that binds to human TfR is linked to the C-terminus of one of the two antibody heavy chains, and the other antigen-binding domain that binds to human TfR is linked to the C-terminus of the other of the two antibody heavy chains. The two antigen-binding domains that bind to human TfR can comprise the same amino acid sequence. The two antigen-binding domains that bind to human TfR can comprise different amino acid sequences. This format is exemplified in FIGS. 1B and 1C.

As used herein, a “1+1 antibody format” of a TfR antibody refers to a bivalent, bi-specific antibody format comprising (i) one antigen-binding domain that binds to human TfR; and (ii) an antigen binding domain that binds an antigen other than human TfR, optionally comprising an Fc domain. This format is exemplified in FIG. 1D. Exemplary 1+1 fusion protein formats are exemplified in FIG. 1E.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in a sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs). The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDRH1), amino acid positions 50 to 65 (CDRH2), and amino acid positions 95 to 102 (CDRH3). Using the Kabat numbering system, CDRs within an antibody light chain molecule are typically present at amino acid positions 24 to 34 (CDRL1), amino acid positions 50 to 56 (CDRL2), and amino acid positions 89 to 97 (CDRL3). In some aspects, the CDRs of the antibodies described herein have been determined according to the Kabat numbering scheme. Chothia refers instead to the location of the structural loops (Chothia and Lesk, J. Mol. Biol. 196:901-917 (1987)). The end of the Chothia CDRH1 loop, when numbered using the Kabat numbering convention, varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops and are used by Oxford Molecular's AbM antibody modeling software. In some aspects, the CDRs can be “contact” CDRs. The “contact” CDRs are based on an analysis of the available complex crystal structures. The residues from each of these CDRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| H1 | H31-H35 | H26-H35 | H26-H32 | H30-H35 (Chothia |
| H2 | H50-H65 | H50-H58 | H53-H55 | H47-H58 |
| H3 | H95-H102 | H95-H102 | H96-H101 | H93-H101 |

CDRs can comprise “extended CDRs” as follows: 24-36 or 24-34 (L1), 46-56 or 50-56 (L2), and 89-97 or 89-96 (L3) in the VL, and 26-35 (H1), 50-65 or 49-65 (H2), and 93-102, 94-102, or 95-102 (H3) in the VH. The variable-domain residues are numbered according to Kabat et al., supra, for each of these extended-CDR definitions.

The terms “VH” and “VH domain” are used interchangeably to refer to the heavy chain variable region of an antibody.

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄. Heavy chain amino acid sequences are well known in the art. In some aspects, the heavy chain is a human heavy chain.

The terms “VL” and “VL domain” are used interchangeably to refer to the light chain variable region of an antibody.

As used herein, the term “light chain” when used in reference to an antibody can refer to any distinct type, e.g., kappa (κ) or lambda (λ) based on the amino acid sequence of the constant regions. Light chain amino acid sequences are well known in the art. In some aspects, the light chain is a human light chain.

As used herein, the term “constant region” is a region of an antibody that is not the variable region of the antibody, e.g., a carboxyl terminal portion of a light and/or heavy chain which is not directly involved in binding of an antibody to antigen, but which can exhibit various effector functions, such as interaction with the Fc receptor. The constant region of an immunoglobulin molecule generally has a more conserved amino acid sequence relative to an immunoglobulin variable domain. In certain aspects, an antibody or antigen-binding fragment comprises a constant region or portion thereof that is sufficient for antibody-dependent cell-mediated cytotoxicity (ADCC).

A “constant domain” means a domain within a constant region that is capable of forming an immunoglobulin fold. Constant domains include the CH1, CH2, CH3, and CL domains.

The term “monoclonal” when referring to an antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody or antibody portion, and any other modified immunoglobulin molecule comprising an antigen recognition site. Furthermore, a “monoclonal” antibody or antigen-binding fragment thereof refers to such antibodies and antigen-binding fragments thereof made in any number of manners including but not limited to by hybridoma, phage selection, recombinant expression, and transgenic animals.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g. murine) sequences. Typically, humanized antibodies or antigen-binding fragments thereof are human immunoglobulins in which residues from the complementarity determining regions (CDRs) are replaced by residues from the CDRs of a molecule originating from a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). The humanized antibody or antigen-binding fragment thereof can be further modified by the substitution of additional residues either in the Fv framework region and/or within the replaced non-human residues to refine and optimize the specificity, affinity, and/or capability of the antibody or antigen-binding fragment thereof. In general, the humanized antibody or antigen-binding fragment thereof will comprise VH and VL that comprise substantially all of at least one, and typically two or three, of the CDR regions that correspond to the non-human immunoglobulin, whereas all or substantially all of the FR regions are those of a human immunoglobulin consensus sequence. The humanized antibody or antigen-binding fragment thereof can also comprise at least a portion of an immunoglobulin constant region or Fc region, typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Framework” or “FR” residues are those variable-domain residues other than the CDR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework can comprise the same amino acid sequence thereof, or it can comprise pre-existing amino acid sequence changes. In some aspects, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, in some aspects those changes occur at only three, two, or one of positions 71H, 73H and 78H; for instance, the amino acid residues at those positions can by 71A, 73T and/or 78A. In some aspects, the V_{L} acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the V_{L}, the subgroup can be subgroup kappa I, kappa II, kappa III or kappa IV as in Kabat et al., supra. Additionally, for the V_{H}, the subgroup can be subgroup I, subgroup II, or subgroup III as in Kabat et al., supra.

An “amino-acid modification” at a specified position, e.g., of an antibody of the present disclosure, refers to the substitution or deletion of the specified residue, or the insertion of at least one amino acid residue adjacent the specified residue. Insertion “adjacent” to a specified residue means insertion within one to two residues thereof. The insertion can be N-terminal or C-terminal to the specified residue. In some aspects, an amino acid modification is a substitution.

Antibody “effector functions” refer to those biological activities attributable to the Fc region (a native sequence Fc region or amino acid sequence variant Fc region) of an antibody and vary with the antibody isotype.

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region can be removed, for example, during production or purification of the antibody, or by recombinantly engineering the nucleic acid encoding a heavy chain of the antibody. Accordingly, a composition of intact antibodies can comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, in some aspects one or more amino acid substitution(s). In some aspects, the variant Fc region has at least one amino acid substitution compared to a native sequence Fc region or to the Fc region of a parent polypeptide, e.g. from about one to about ten amino acid substitutions, and in some aspects from about one to about five amino acid substitutions in a native sequence Fc region or in the Fc region of the parent polypeptide. In some aspects, the variant Fc region possesses at least 80% homology with a native sequence Fc region and/or with an Fc region of a parent polypeptide, at least 90% homology therewith, or at least 95% homology therewith.

“Fc receptor” or “FcR” describes a receptor that binds to the Fc region of an antibody. In some aspects, an FcR is a native sequence human FcR. In some aspects, a FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the equilibrium dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas KA is calculated from the quotient of kₒₙ/k_{off}. kₒₙ refers to the association rate constant of, e.g., an antibody or antigen-binding fragment thereof to an antigen, and k_{off} refers to the dissociation rate constant of, e.g., an antibody or antigen-binding fragment thereof from an antigen. The kₒₙ and k_{off} can be determined by techniques known to one of ordinary skill in the art, such as BIAcore® or KinExA.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of about any of 10⁻⁴ M or lower, 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In some aspects, the term “specific binding” refers to binding where a molecule binds to a particular polypeptide or epitope on a particular polypeptide without substantially binding to any other polypeptide or polypeptide epitope.

The term “linker” or “linked” refers to the covalent linkage between two polypeptides or two heterologous molecules. In some aspects, a linker is a chemical linker. In some aspects, the linker comprises a peptide bond, and the two polypeptides or two heterologous molecules are linked to each other either directly to or via one or more additional amino acids. A glycine linker is one that comprises one or more glycines but no other amino acids, e.g., GGGG (SEQ ID NO:285). A glycine-rich linker is one that comprises one or more glycines and can contain other amino acids as long as glycine is the predominant species in the linker e.g., GGGNGG, wherein N is any amino acid (SEQ ID NO:286). A glycine-serine linker is one which contains both glycine and serine in any proportion, e.g., GGGS (SEQ ID NO:287). Similarly, a proline linker is one that comprises one or more prolines but no other amino acids. A proline-rich linker is one that comprises one or more prolines and can contain other amino acids so long as proline is the predominant species in the linker.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

The term “epitope” includes any determinant capable of being bound by an antibody. An epitope is a region of an antigen that is bound by an antibody that targets that antigen, and when the antigen is a polypeptide, includes specific amino acids that directly contact the antibody. Most often, epitopes reside on polypeptides, but in some instances, can reside on other kinds of molecules, such as nucleic acids. Epitope determinants can include chemically active surface groupings of molecules such as amino acids, sugar side chains, phosphoryl or sulfonyl groups, and can have specific three-dimensional structural characteristics, and/or specific charge characteristics. Generally, antibodies specific for a particular target antigen will preferentially recognize an epitope on the target antigen in a complex mixture of polypeptides and/or macromolecules.

An antibody that “binds to the same epitope” as a reference antibody refers to an antibody that contacts the same amino acid residues on the antigen as the reference antibody. The ability of an antibody to bind to the same epitope as a reference antibody can be determined using peptide scanning mutagenesis or high throughput alanine scanning mutagenesis. In the latter methodology, a comprehensive mutation library of antigen, or a portion thereof (e.g., the extracellular domain), can be generated by mutating each individual amino acid residue to alanine (or if the amino acid residue is alanine, then to another residue such as serine) and testing each mutant for binding to a target antibody or antigen-binding fragment thereof.

An antibody is said to “competitively inhibit” binding of a reference antibody to a given epitope if it preferentially binds to that epitope or an overlapping epitope such that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some aspects, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

The term “expression system” refers to one or more nucleic acid molecules comprising coding sequence and control sequence(s) in operable linkage, along with a host cell and/or other in vitro transcription and translation machinery, such that one or more proteins encoded by the nucleic acid molecule(s) are capable of being produced.

The term “vector,” as used herein, is intended to refer to a nucleic acid molecule capable of transporting another nucleic acid to which it has been linked. One type of vector is a “plasmid,” which refers to a circular double stranded DNA into which additional DNA segments can be ligated. Another type of vector is a phage vector. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” can be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

The terms “administer,” “administering,” “administration,” and the like, as used herein, refer to methods that can be used to deliver a drug, e.g., an anti-human antibody or antigen-binding fragment thereof, to the desired site of biological action.

An “effective amount” refers to at least an amount effective, at dosages and for periods of time necessary, to achieve the desired therapeutic result. An effective amount can be provided in one or more administrations. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” can be considered in the context of administering one or more therapeutic agents, and a single agent can be considered to be given in an effective amount if, in conjunction with one or more other agents, a desirable result can be or is achieved.

As used herein, the terms “subject” and “patient” are used interchangeably. The subject can be a mammal such as a non-human animal (e.g., cow, pig, horse, cat, dog, rat, mouse, monkey or other primate, etc.). In some aspects, the subject is a cynomolgus monkey. In some aspects, the subject is a human.

As used herein, administration “in conjunction” or “in combination” with another compound or composition includes simultaneous administration and/or administration at different times. Administration in conjunction also encompasses administration as a co-formulation or administration as separate compositions, including at different dosing frequencies or intervals, and using the same route of administration or different routes of administration. In some aspects, administration in conjunction is administration as a part of the same treatment regimen.

A “neurological disorder” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease.

A “Lysosomal storage disorder” or (LSD) as used herein refers to an inherited metabolic disease characterized by the accumulation of substrates, such as undigested or partially digested macromolecules, in excess in various cells of organs, which ultimately results in cellular dysfunction and clinical abnormalities. LSDs have been defined as deficiencies in lysosomal function generally classified by the accumulated substrate and include sphingolipidoses, oligosaccharidoses, mucolipidoses, mucopolysaccharidoses, lipoprotein storage disorders, neuronal ceroid lipofuscinoses, and others. LSDs may also include other deficiencies or defects in proteins that result in accumulation of macromolecules, such as proteins necessary for normal post-translational modification of lysosomal enzymes, or proteins important for proper lysosomal trafficking. LSDs are diseases caused by defects in single genes. Enzyme defects cause nearly seventy percent of the LSDs, and the rest are defects in enzyme activator or associated proteins.

“Protein replacement therapy” or “PRT” refers to a medical treatment that supplements or replaces a protein in a patient in whom that particular protein is deficient or absent.

An “enzyme replacement therapy enzyme” or “ERT enzyme” refers to an enzyme that is deficient in a lysosomal storage disorder. An “ERT enzyme variant” refers to a functional variant, including allelic and splice variants, of a wild-type ERT enzyme or a fragment thereof, where the ERT enzyme variant has at least 50%, at least 55%, at least 60%, at least 65%, at least 70%, at least 75%, at least 80%, at least 85%, at least 90%, or at least 95% of the activity of the corresponding wild-type ERT enzyme or fragment thereof, e.g., when assayed under identical conditions. A “catalytically active fragment” of an ERT enzyme refers to a portion of a full-length ERT enzyme or a variant thereof, where the catalytically active fragment has at least 50%, at least 55%, at least 60%, at least 65%, at least 70%, at least 75%, at least 80%, at least 85%, at least 90%, or at least 95% of the activity of the corresponding full-length ERT enzyme or variant thereof, e.g., when assayed under identical conditions.

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range are also provided.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially of” are open-ended, allowing for the presence of more than that which is recited so long as basic or novel characteristics of that which is recited is not changed by the presence of more than that which is recited, but excludes prior art aspects.

### Anti-TfR Antigen-Binding Domains

Provided herein are antigen-binding domains that specifically bind to human TfR.

Such antigen-binding domains can be capable of crossing the blood brain barrier (BBB) and capable of transporting other agents (e.g., therapeutically active agents) associated with the antigen-binding domain across the BBB. Accordingly, in some aspects, provided herein are antigen-binding domains that specifically bind to human TfR that are capable of being internalized in BBB epithelial cells such as HCMEC/D3 cells.

In some aspects, an antigen-binding domain that specifically binds to human TfR comprises the six CDRs of an antibody listed in Tables 12 and 13 (i.e., the three VH CDRs of the antibody listed in Table 12 and the three VL CDRs of the same antibody listed in Table 13).

In some aspects, an antigen-binding domain that specifically binds to human TfR comprises the six CDRs of an antibody listed in Tables 12 and 13 and 19, 21, 24 and 25. In some aspects, the CDRs of such an antigen-binding domain can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In some aspects, an antigen-binding domain that specifically binds to human TfR comprises the six Chothia CDRs of an antibody listed in Tables 12, 13, 19, 21, 24 and 25. In some aspects, such as an antigen-binding domain that specifically binds to human TfR comprises one or more CDRs, in which the Chothia and Kabat CDRs have the same amino acid sequence. In some aspects, provided herein are antigen-binding domains that specifically binds to human TfR and comprise combinations of Kabat CDRs and Chothia CDRs.

In some aspects, the CDRs of an antigen-binding domain that specifically binds to human TfR can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dubel, eds., Chapter 31, pp. 422-439, Springer-Verlag, Berlin (2001). In some aspects, provided herein are antigen-binding domains that specifically bind to human TfR and comprise VH and VL CDRs of an antibody listed in Tables 12, 13, 19, 21, 24 and 25 as determined by the method in MacCallum R M et al.

In some aspects, the CDRs of an antigen-binding domain that specifically binds to human TfR can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.). In some aspects, provided herein are antigen-binding domains that specifically bind to human TfR and comprise VH and VL CDRs of an antibody listed in Tables 12, 13, 19, 21, 24 and 25 as determined by the AbM numbering scheme.

In some aspects, an antigen-binding domain that specifically binds to human TfR comprises the six IMGT CDRs of an antibody listed in Tables 12, 13, 19, 21, 24 and 25 according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

In some aspects, an antigen-binding domain that specifically binds to human TfR provided herein is described by its VL domain alone, or its VH domain alone, or by its 3 VL CDRs alone, or its 3 VH CDRs alone. See, for example, Rader C et al., (1998) PNAS 95: 8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352: 624-628, which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also Kim S J & Hong H J, (2007) J Microbiol 45: 572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In some aspects, an antigen-binding domain that specifically binds to human TfR comprises the VH of an antibody listed in Tables 19, 21, 24 and 25.

In some aspects, an antigen-binding domain that specifically binds to human TfR comprises the VL of antibody listed in Tables 19, 21, 24 and 25.

In some aspects, an antigen-binding domain that specifically binds to human TfR comprises the VH and the VL of an antibody listed in Table 11 (i.e., the VH of the antibody listed in Table 11 and the VL of the same antibody listed in Table 11) or Tables 19, 21, 24 and 25 (i.e., the VH of the antibody listed in Tables 19, 21, 24 and 25 and the VL of the same antibody listed in Tables 19, 21, 24 and 25).

In some aspects, an antigen-binding domain that specifically binds to human TfR comprises (i) a VH comprising an amino acid sequence that is at least 80% identical to a VH amino acid sequence of an antibody in Tables 11, 19, 21, 24 and 25 and (ii) a VL comprising an amino acid sequence that is at least 80% identical to the VL amino acid sequence of the same antibody in Tables 11, 19, 21, 24 and 25. In some aspects, the antigen-binding domain that specifically binds to human TfR also comprises the CDRs of the antibody in Tables 12, 13, 19, 21, 24 and 25 (e.g., the non-identical amino acids in the VH and/or VL are outside of the CDRs).

In some aspects, an antigen-binding domain that specifically binds to human TfR comprises (i) a VH comprising an amino acid sequence that is at least 85% identical to a VH amino acid sequence of an antibody in Tables 11, 19, 21, 24 and 25 and (ii) a VL comprising an amino acid sequence that is at least 85% identical to the VL amino acid sequence of the same antibody in Tables 11, 19, 21, 24 and 25. In some aspects, the antigen-binding domain that specifically binds to human TfR also comprises the CDRs of the antibody in Tables 12, 13, 19, 21, 24 and 25 (e.g., the non-identical amino acids in the VH and/or VL are outside of the CDRs).

In some aspects, an antigen-binding domain that specifically binds to human TfR comprises (i) a VH comprising an amino acid sequence that is at least 90% identical to a VH amino acid sequence of an antibody in 11, 19, 21, 24 and 25 and (ii) a VL comprising an amino acid sequence that is at least 90% identical to the VL amino acid sequence of the same antibody in Tables 11, 19, 21, 24 and 25. In some aspects, the antigen-binding domain that specifically binds to human TfR also comprises the CDRs of the antibody in Tables 12, 13, 19, 21, 24 and 25 (e.g., the non-identical amino acids in the VH and/or VL are outside of the CDRs).

In some aspects, an antigen-binding domain that specifically binds to human TfR comprises (i) a VH comprising an amino acid sequence that is at least 95% identical to a VH amino acid sequence of an antibody in Tables 11, 19, 21, 24 and 25 and (ii) a VL comprising an amino acid sequence that is at least 95% identical to the VL amino acid sequence of the same antibody in Tables 11, 19, 21, 24 and 25. In some aspects, the antigen-binding domain that specifically binds to human TfR also comprises the CDRs of the antibody in Tables 12, 13, 19, 21, 24 and 25 (e.g., the non-identical amino acids in the VH and/or VL are outside of the CDRs).

In some aspects, an antigen-binding domain that specifically binds to human TfR comprises (i) a VH comprising an amino acid sequence that is at least 96% identical to a VH amino acid sequence of an antibody in Tables 11, 19, 21, 24 and 25 and (ii) a VL comprising an amino acid sequence that is at least 96% identical to the VL amino acid sequence of the same antibody in Tables 11, 19, 21, 24 and 25. In some aspects, the antigen-binding domain that specifically binds to human TfR also comprises the CDRs of the antibody in Tables 12, 13, 19, 21, 24 and 25 (e.g., the non-identical amino acids in the VH and/or VL are outside of the CDRs).

In some aspects, an antigen-binding domain that specifically binds to human TfR comprises (i) a VH comprising an amino acid sequence that is at least 97% identical to a VH amino acid sequence of an antibody in Tables 11, 19, 21, 24 and 25 and (ii) a VL comprising an amino acid sequence that is at least 97% identical to the VL amino acid sequence of the same antibody in Tables 11, 19, 21, 24 and 25. In some aspects, the antigen-binding domain that specifically binds to human TfR also comprises the CDRs of the antibody in Tables 12, 13, 19, 21, 24 and 25 (e.g., the non-identical amino acids in the VH and/or VL are outside of the CDRs).

In some aspects, an antigen-binding domain that specifically binds to human TfR comprises (i) a VH comprising an amino acid sequence that is at least 98% identical to a VH amino acid sequence of an antibody in Tables 11, 19, 21, 24 and 25 and (ii) a VL comprising an amino acid sequence that is at least 98% identical to the VL amino acid sequence of the same antibody in Tables 11, 19, 21, 24 and 25. In some aspects, the antigen-binding domain that specifically binds to human TfR also comprises the CDRs of the antibody in Tables 12, 13, 19, 21, 24 and 25 (e.g., the non-identical amino acids in the VH and/or VL are outside of the CDRs).

In some aspects, an antigen-binding domain that specifically binds to human TfR comprises (i) a VH comprising an amino acid sequence that is at least 99% identical to a VH amino acid sequence of an antibody in Tables 11, 19, 21, 24 and 25 and (ii) a VL comprising an amino acid sequence that is at least 99% identical to the VL amino acid sequence of the same antibody in Tables 11, 19, 21, 24 and 25. In some aspects, the antigen-binding domain that specifically binds to human TfR also comprises the CDRs of the antibody in Tables 12, 13, 19, 21, 24 and 25 (e.g., the non-identical amino acids in the VH and/or VL are outside of the CDRs).

In some aspects, provided herein is an antigen-binding domain that binds to the same TfR epitope as an antibody comprising a VH amino acid sequence of an antibody in Tables 11, 19, 21, 24 and 25 and a VL amino acid sequence of the same antibody in Tables 11, 19, 21, 24 and 25.

In some aspects, provided herein is an antigen-binding domain that competitively inhibits binding to TfR of as an antibody comprising a VH amino acid sequence of an antibody in Tables 11, 19, 21, 24 and 25 and a VL amino acid sequence of the same antibody in Tables 11, 19, 21, 24 and 25.

In some aspects, an antigen-binding domain that specifically binds to human TfR comprises a VH and a VL on a single polypeptide chain (e.g., a VH and VL in Tables 11, 19, 21, 24 and 25). In some aspects, the antigen-binding domain comprises an scFv. The scFv can comprise a VH that is N-terminal to a VL or a VL that is N-terminal to a VH. The scFv can comprise a linker, e.g., between a VH and a VL. Accordingly, the scFv can be in the orientation VH-linker-VL or VL-linker-VH. Such a linker can be about 5 to about 25 amino acids in length. Such a linker can be about 5 to about 20 amino acids in length. Such a linker can be about 10 to about 25 amino acids in length. Such a linker can be about 10 to about 20 amino acids in length. Such a linker can be, e.g., a glycine linker, a glycine-rich linker, or a glycine-serine linker. Such a linker can comprise the amino acid sequence of GGSEGKSSGSGSESKSTGGS (SEQ ID NO:183). Such a linker can comprise the amino acid sequence of

| (SEQ ID NO: 288) | |
|---|---|
| | GGGGSGGGGSGGGGSGGGGS. |

In some aspects, an antigen-binding domain that specifically binds to human TfR comprises a VH on a first polypeptide and a VL on a second polypeptide (e.g., a Fab).

In some aspects, an antigen-binding domain that specifically binds to human TfR comprises the antigen-binding fragment of a heavy chain only antibody (e.g., a VHH or nanobody).

In some aspects, an antigen-binding domain that specifically binds to human TfR is a murine antigen-binding domain. In some aspects, an antigen-binding domain that specifically binds to human TfR is a chimeric antigen-binding domain. In some aspects, an antigen-binding domain that specifically binds to human TfR is a humanized antigen-binding domain. In some aspects, an antigen-binding domain that specifically binds to human TfR is a human antigen-binding domain

In some aspects, an antigen-binding domain provided herein that specifically binds to human TfR also binds to cynomolgus monkey TfR.

In some embodiments, an antigen-binding domain provided herein bind to human TfR with “low” affinity. In some aspects, the antigen-binding domain binds human TfR with an affinity between 500 nM and 10 uM. In some aspects, the antigen-binding domain binds human TfR with an affinity between 2 μM and 5 μM. In some aspects, the antigen-binding domain binds human TfR with an affinity between 1 μM and 5 μM. In some aspects, the antigen-binding domain binds human TfR with an affinity between 2 μM and 8 μM. In some aspects, the antigen-binding domain binds human TfR with an affinity between 750 nM and 2 μM.

In some embodiments, an antigen-binding domain provided herein bind to human TfR with “medium” affinity. In some embodiments, an antigen-binding domain provided herein bind to human TfR with “medium” affinity. In some aspects, the antigen-binding domain binds human TfR with an affinity between 10 nM and 500 nM. In some aspects, the antigen-binding domain binds human TfR with an affinity between 50 nM and 500 nM. In some aspects, the antigen-binding domain binds human TfR with an affinity between 100 nM and 250 nM. In some aspects, the antigen-binding domain binds human TfR with an affinity between 10 nM and 100 nM. In some aspects, the antigen-binding domain binds human TfR with an affinity between 250 nM and 500 nM.

In some embodiments, an antigen-binding domain provided herein bind to human TfR with “high” affinity. In some aspects, the antigen-binding domain binds human TfR with an affinity less than 10 nM. In some aspects, the antigen-binding domain binds human TfR with an affinity between 0.01 nM and 10 nM. In some aspects, the antigen-binding domain binds human TfR with an affinity between 0.1 nM and 10 nM. In some aspects, the antigen-binding domain binds human TfR with an affinity between 0.01 nM and 1 nM.

In some aspects, an antigen-binding domain provided herein specifically binds to human TfR with an affinity of no more than 250 nM (e.g., 10 μM to 250 nM, 5 μM to 250 nM, 1 nM to 250 nM or 3 nM to 250 nM), an affinity of no more than 200 nM (e.g., 10 μM to 200 nM, 5 μM to 200 nM, 1 nM to 200 nM or 3 nM to 200 nM), or an affinity of no more than 150 nM (e.g., 10 μM to 150 nM, 5 μM to 150 nM, 1 nM to 150 nM or 3 nM to 150 nM), optionally wherein the affinity is measured using surface plasmon resonance. Surface plasmon resonance can be measured, e.g., using the Carterra LSA platform. In some aspects, an antigen-binding domain provided herein that specifically binds to human TfR binds to human TfR as measured by ELISA OD₄₅₀.

In some aspects, an antigen-binding domain provided herein that specifically binds to human TfR binds to cynomolgus TfR with an affinity of no more than 250 nM (e.g., 10 μM to 250 nM, 5 μM to 250 nM, 1 μM to 250 nM, 1 nM to 250 nM or 3 nM to 250 nM), an affinity of no more than 200 nM (e.g., 10 μM to 200 nM, 5 μM to 200 nM, 1 μM to 200 nM, 1 nM to 200 nM or 3 nM to 200 nM), or an affinity of no more than 150 nM (e.g., 10 μM to 150 nM, 5 μM to 150 nM, 1 μM to 150 nM, 1 nM to 150 nM or 3 nM to 150 nM), optionally wherein the affinity is measured using surface plasmon resonance. Surface plasmon resonance can be measured, e.g., using the Carterra LSA platform. In some aspects, an antigen-binding domain provided herein that specifically binds to human TfR with an affinity of 0.1 μM to 10 μM, 0.1 μM to 100 μM, 0.1 μM to 100 μM, 0.1 μM to 1 nM, 1 μM to 10 μM, 1 μM to 100 μM, 1 μM to 1 nM, 1 μM to 10 nM, 1 μM to 100 nM, 1 μM to 150 nM, or 1 μM to 250 nM.

In some aspects, an antigen-binding domain provided herein that specifically binds to human TfR binds to each of human TfR and cynomolgus TfR with an affinity of no more than 250 nM (e.g., 1 μM to 250 nM, 10 μM to 250 nM, 1 nM to 250 nM or 3 nM to 250 nM), an affinity of no more than 200 nM (e.g., 1 μM to 200 nM, 10 μM to 250 nM, 1 nM to 200 nM or 3 nM to 200 nM), or an affinity of no more than 150 nM (e.g., 1 μM to 150 nM, 10 μM to 250 nM, 1 nM to 150 nM or 3 nM to 150 nM), optionally wherein the affinity is measured using surface plasmon resonance. Surface plasmon resonance can be measured, e.g., using the Carterra LSA platform. In some aspects, an antigen-binding domain provided herein that specifically binds to human TfR binds to each of human TfR and cynomolgus TfR with an affinity of 1 μM to 100 μM, 1 μM to 1 nM, 1 μM to 10 nM, 1 μM to 100 nM, 1 μM to 150 nM, or 1 μM to 250 nM.

In some aspects, an antigen-binding domain provided herein that specifically binds to human TfR binds to human TfR with an affinity of 6.7 nM to 3.5 μM and binds to cynomolgus TfR with an affinity of 38 nM to 2.3 μM. In some aspects, an antigen-binding domain provided herein that specifically binds to human TfR binds to human TfR with an affinity of 6.7 nM to 340 nM and binds to cynomolgus TfR with an affinity of 18 nM to 870 nM, optionally wherein the affinity is measured using surface plasmon resonance. Surface plasmon resonance can be measured, e.g., using the Carterra LSA platform.

In some aspects, an antigen-binding domain provided herein that specifically binds to human TfR reduces cell surface expression of TfR on HCMED/D3 cells by 40-80% relative to cell surface expression of TfR on HCMEC/D3 cells treated with an isotype control. Cell surface expression can be measured, e.g., using Western blot or FACS.

In some aspects, an antigen-binding domain provided herein that specifically binds to human TfR does not significantly increase cell surface expression of TfR on HCMED/D3 cells relative to cell surface expression of TfR on HCMEC/D3 cells treated with an isotype control. Cell surface expression can be measured, e.g., using Western blot or FACS.

In some aspects, an antigen-binding domain provided herein that specifically binds to human TfR does not reduce cell surface expression of TfR on HCMED/D3 cells by more than 40%, 60%, or 80% relative to cell surface expression of TfR on HCMEC/D3 cells treated with an isotype control and does not significantly increase cell surface expression of TfR on HCMED/D3 cells relative to cell surface expression of TfR on HCMEC/D3 cells treated with an isotype control. Cell surface expression can be measured, e.g., using Western blot or FACS.

In some aspects, an antigen-binding domain provided herein that specifically binds to human TfR accumulates at least 4- or 5-fold more than an isotype control in vessel-depleted human TfR knock-in mouse brain after peripheral injection.

In some aspects, an antigen-binding domain provided herein that specifically binds to human TfR accumulates at least 2 fold more than an isotype control in vessel-depleted non-human primate brain after peripheral injection. In some aspects, an antigen-binding domain provided herein that specifically binds to human TfR accumulates at least 4- or 5-fold more than an isotype control in vessel-depleted non-human primate brain after peripheral injection. In some aspects, an antigen-binding domain provided herein that specifically binds to human TfR accumulates at least 8 fold more than an isotype control in vessel-depleted non-human primate brain after peripheral injection. In some aspects, an antigen-binding domain provided herein that specifically binds to human TfR accumulates 8 to 64 fold more than an isotype control in vessel-depleted non-human primate brain after peripheral injection. In some aspects, an antigen-binding domain provided herein that specifically binds to human TfR accumulates 8 to 64 fold more than an isotype control in vessel-depleted non-human primate brain after peripheral injection. In some aspects, an antigen-binding domain provided herein that specifically binds to human TfR accumulates 2 to 11 fold more than an isotype control in vessel-depleted non-human primate brain after peripheral injection.

In some aspects, an antigen-binding domain provided herein that specifically binds to human TfR at least 5-fold more than binding to an irrelevant protein. In some aspects, an antigen-binding domain provided herein that specifically binds to cynomolgus TfR at least 5-fold more than binding to an irrelevant protein. In some aspects, an antigen-binding domain provided herein that specifically binds to human TfR at least 5-fold more than binding to an irrelevant protein and/or specifically binds to cynomolgus TfR at least 5-fold more than binding to an irrelevant protein.

Also provided herein are antigen-binding domains that bind to the same epitope of TfR as a TfR antigen-binding domain provided herein. Also provided herein are antigen-binding domains that competitively inhibit binding to TfR of TfR antigen-binding domain provided herein.

### Agents Comprising Anti-TfR Antigen-Binding Domains

Provided herein are agents (e.g., fusion proteins, multi-specific (e.g., bispecific) proteins, antibodies, antigen-binding fragments thereof, etc.) comprising an antigen-binding domain that specifically binds to human TfR.

### Fusion Proteins

In some aspects, a fusion protein provided herein comprises: an antigen-binding domain that specifically binds to human TfR and a heterologous protein. In some aspects, a fusion protein provided herein comprises: (i) an antigen-binding domain that specifically binds to human TfR and (ii) a heterologous protein or polypeptide or fragment thereof. In some aspects, the heterologous protein is a protein or polypeptide or fragment thereof useful in protein replacement therapy (PRT). In some aspects, the heterologous polypeptide is an enzyme (e.g., an enzyme for use in enzyme replacement therapy (ERT)) or a catalytically active fragment thereof. In some aspects, the heterologous polypeptide or protein is an ERT enzyme or an ERT enzyme variant, or a catalytically active fragment thereof.

In some aspects, the heterologous polypeptide in a fusion protein provided herein is a growth factor. In some aspects, the heterologous polypeptide in a fusion protein provided herein is a decoy receptor. In some aspects, the heterologous polypeptide in a fusion protein provided herein is progranulin (PGRN), prosaposin (PSAP), or survival motor neuron protein (SMN). In some aspects, the heterologous protein is an enzyme selected from ubiquitin protein ligase E3A (UBE3A), α-L Iduronidase (IDUA), Iduronate-2-sulphatase (IDS), N-acetylgalactoslamine-6-sulphatase (GALNS), N-sulfoglucosamine sulfohydrolase (SGSH), N-acetylgalactosamine-4-sulphatase (arylsulphatase B; ARSB), acid sphingomyelinase (ASM), β-glucocerebrosidase (GCase or GBA), galactosylceramide beta-galactosidase, glucosylceramidase, beta-hexosaminidase A, beta-hexosaminidase B, arylsulphatase A, beta-galactosidase, acid ceramidase, alpha-glucosidase, lysosomal acid lipase, lysosomal protease, a synthetic enzyme replacement thereof, such as larodinase, idursulphase, elosulphase alpha or galsuphase, or a variant thereof, or a catalytically active fragment thereof. In some aspects, the heterologous protein is a protein or an enzyme selected from clusterin (APOJ), Reelin, Tripeptidyl Peptidase 1 (CLN2/TPP1), glucosamine (N-acetyl)-6-sulfatase (GNS), heparan-alpha-glucosaminide N-acetyltransferase (HGSNAT), and N-acetyl-alpha-glucosaminidase (NAGLU), α-L Iduronidase (IDUA), Iduronate-2-sulphatase (IDS), N-acetylgalactoslamine-6-sulphatase (GALNS), N-sulfoglucosamine sulfohydrolase (SGSH), N-acetylgalactosamine-4-sulphatase (arylsulphatase B; ARSB), acid sphingomyelinase (ASM), β-glucocerebrosidase (GCase or GBA), galactosylceramide beta-galactosidase, glucosylceramidase, beta-hexosaminidase A, beta-hexosaminidase B, arylsulphatase A, beta-galactosidase, acid ceramidase, alpha-glucosidase, lysosomal acid lipase, lysosomal protease, a synthetic enzyme replacement thereof, such as larodinase, idursulphase, elosulphase alpha or galsuphase, or a variant thereof, or a catalytically active fragment thereof.

Exemplary formats for a fusion protein disclosed herein are depicted in FIG. 1E.

For example, in some aspects, the heterologous protein or polypeptide in the fusion protein is N-terminal to the antigen-binding domain that specifically binds to human TfR. In some aspects, the heterologous protein or polypeptide in the fusion protein is C-terminal to the antigen-binding domain that specifically binds to human TfR. In some aspects, the heterologous fusion protein or polypeptide and the antigen-binding domain that specifically binds to human TfR are directly connected via a peptide bond. In some aspects, the heterologous fusion protein or polypeptide and the antigen-binding domain that specifically binds to human TfR are connected via a linker, e.g., a peptide linker. In some aspects, the fusion protein comprises an antigen-binding domain and a heterologous protein or polypeptide and an Fc portion. In some aspects, the antigen-binding domain and the heterologous protein or polypeptide are linked to the N-terminus of the Fc portion of the fusion protein. In some aspects, the antigen-binding domain and the heterologous protein are linked to the C-terminus of the Fc portion of the fusion protein. In other aspects, the antigen-binding domain is linked to the N-terminus of the Fc portion and the heterologous protein or polypeptide is linked to the C-terminus of the Fc portion of the fusion protein. In other aspects, the antigen-binding domain is linked to the C-terminus of the Fc portion and the heterologous protein or polypeptide is linked to the N-terminus of the Fc portion of the fusion protein.

In some aspects, disclosed herein is a fusion protein comprising (i) a single scFv, or VHH, or Fab antigen-binding domain that binds to human TfR and (ii) a heterologous protein or polypeptide, wherein the fusion protein comprises two copies of the heterologous protein or polypeptide. In some aspects, the fusion protein comprises an Fc domain. The Fc may be a heterodimeric Fc, comprising a first Fc polypeptide with a knob mutation and a second Fc polypeptide with a hole mutation (a “knob-hole Fc”, described herein). In some aspects, the single scFv, Fab or VHH antigen-binding domain that binds to human TfR is linked to the C-terminus of the Fc domain (i.e., to one of the two heavy chains of the Fc domain) while the two copies of the heterologous protein or polypeptide are linked to the N-terminus of the Fc. In some aspects, the single scFv, Fab or VHH antigen-binding domain that binds to human TfR is linked to the N-terminus of the Fc domain (i.e., to one of the two heavy chains of the Fc domain) while the two copies of the heterologous protein or polypeptide are linked to the C-terminus of the Fc domain. Examples of this 2+1 format are shown as (v) and (vi) in FIG. 1E.

In some aspects, disclosed herein is a fusion protein comprising (i) an antibody that binds to human TfR, wherein the antibody comprises two heavy chains and two light chains; and (ii) two copies of a heterologous protein or polypeptide linked to the C-terminus of the two antibody heavy chains.

In some aspects, the fusion protein comprises (i) two scFv, Fab, or VHH antigen-binding domains that bind to human TfR linked to the C-terminus of the heavy chain and (ii) two copies of the fusion proteins or fusion protein variants linked to the N-terminus of the Fc domain.

In some aspects, disclosed herein is a fusion protein comprising (i) a single scFv, VHH, or Fab antigen-binding domain that binds to human TfR and (ii) a Fc domain, and (iii) a single copy of the heterologous protein or peptide, wherein the single scFv, VHH, or Fab antigen-binding domain that binds to human TfR is linked to the C-terminus of the Fc domain and the heterologous protein or polypeptide linked to N-terminus of the Fc domain. In some embodiments, the Fc is a single chain, engineered monovalent Fc domain. An example of this single chain 1+1 format of a fusion protein is shown as (i) in FIG. 1E.

In some aspects, disclosed herein is a fusion protein comprising (i) a single scFv, VHH, or Fab antigen-binding domain that binds to human TfR,(ii) a Fc domain, and (iii) a single copy of the heterologous protein or polypeptide, wherein the single scFv, VHH, or Fab antigen-binding domain that binds to human TfR is linked to the N-terminus of the Fc domain and the heterologous protein or polypeptide linked to C-terminus of the Fc domain. In some embodiments, the Fc is a single chain, engineered monovalent Fc. An example of this 1+1 format of a fusion protein is shown as (ii) in FIG. 1E.

In some aspects, disclosed herein is a fusion protein comprising (i) a single scFv, VHH, or Fab antigen-binding domain that binds to human TfR, and (ii) a Fc domain, and (iii) a single copy of the heterologous protein or polypeptide, wherein the single scFv, VHH, or Fab antigen-binding domain that binds to human TfR and the heterologous protein or polypeptide are both linked to the N-terminus or C-terminus of the Fc domain. An example of this 1+1 format of a fusion protein is shown as (iv) in FIG. 1E.

### Bispecific and Multispecific Proteins

In some aspects, an antibody or antigen-binding fragment thereof provided herein comprises an antigen-binding domain that specifically binds to human TfR. In some aspects, an antibody or antigen-binding fragment thereof comprises an antigen-binding domain that specifically binds to human TfR and an antigen-binding domain that specifically binds to a CNS antigen or a brain antigen. In some aspects, the CNS antigen or brain antigen is not TfR. Also provided herein are antibodies or antigen-binding fragments thereof that bind to the same epitope of TfR as a TfR antigen-binding domain provided herein. Also provided herein are antibodies or antigen-binding fragments thereof that competitively inhibit binding to TfR of a TfR antigen-binding domain provided herein.

In some aspects, a multi-specific protein provided herein comprises a first antigen-binding domain that binds to human TfR and a second antigen-binding domain. The first antigen-binding domain that binds to human TfR can be any antigen-binding domain that binds to human TfR provided herein. The second antigen-binding domain can be an antigen-binding domain that specifically binds to a CNS antigen or a brain antigen. In some aspects, the CNS antigen or brain antigen is not TfR.

In some aspects, a multi-specific protein provided herein comprises an antigen-binding domain that binds to human TfR linked to an antibody or antigen-binding fragment thereof. The antibody or antigen-binding fragment thereof can bind a CNS antigen or brain antigen. In some aspects, the CNS antigen or brain antigen is not TfR. In some aspects, such a multi-specific protein can be in a 2+1 antibody format (as shown in FIG. 1A) or a 2+2 antibody format (as shown in FIGS. 1B and 1C).

In some aspects, a multi-specific protein provided herein comprises a TfR antigen-binding domain that is an scFv linked to an antibody that binds to a CNS antigen, wherein the antibody comprises two heavy chains and two light chains. In some aspects, the scFv is linked to the C-terminus of one of the two antibody heavy chains, e.g., via a protein linker.

In some aspects, the multi-specific protein comprises 1) an antigen-binding domain that binds TfR, 2) a second antigen-binding domain that binds a different CNS or brain antigen, and 3) an Fc region, wherein the TfR antigen-binding domain and the second antigen-binding domain are connected or linked to the Fc region of the multi-specific protein. In other aspects, the multi-specific protein comprises 1) an antigen-binding domain that comprises a heavy chain variable region and binds TfR, 2) a second antigen binding domain that comprises a heavy-chain variable region and binds a different CNS or brain antigen, and 3) an Fc region, wherein the TfR antigen-binding domain and the second antigen-binding domain are connected or linked to the Fc region of the multi-specific protein. In some aspects, the multi-specific protein comprises an antigen-binding domain that binds TfR, a second antigen-binding domain that binds a different CNS or brain antigen, and an Fc region. In some aspects, the TfR antigen-binding domain and the second antigen-binding domain are connected or linked to the N-terminus of the Fc portion of the multi-specific protein. In other aspects, the TfR antigen-binding domain is connected or linked to the N-terminus of an Fc portion of the multi-specific protein and the second antigen-binding domain is linked to the C-terminus of the Fc portion of the multi-specific protein. In other aspects, the TfR antigen-binding domain is connected or linked to the C-terminus of an Fc portion of the multi-specific protein and the second antigen-binding domain is linked to the N-terminus of the Fc portion of the multi-specific protein.

In some aspects, a multi-specific protein provided herein comprises two copies of a TfR antigen-binding domain that is an scFv and an antibody that binds to a CNS antigen, wherein the antibody comprises two heavy chains and two light chains, wherein one of the two copies of the antigen-binding domain is linked to the C-terminus of one of the antibody heavy chains, and wherein the other copy of the antigen-binding domain is linked to the C-terminus of the other antibody heavy chain. In some aspects, the scFvs are linked to the heavy chains via a protein linker.

In some aspects, disclosed herein is a trivalent, bi-specific protein comprising (i) a single scFv, Fab or VHH antigen-binding domain that binds to human TfR and (ii) an antibody, wherein the antibody comprises two heavy chains and two light chains; and wherein the single scFv, Fab or VHH antigen-binding domain that binds to human TfR is linked to the C-terminus or N-terminus of one of the two antibody heavy chains. The Fc may be a heterodimeric Fc such as knob and hole Fc. An example of this 2+1 format with a knob-hole Fc is shown as (i) in FIG. 1D.

In some aspects, disclosed herein is a tetravalent, bi-specific protein comprising (i) two scFv, Fab or VHH antigen-binding domains that bind to human TfR and (ii) an antibody, wherein the antibody comprises two heavy chains and two light chains; and wherein one scFv, Fab, or VHH antigen-binding domain that binds to human TfR is linked to the C-terminus of one of the two antibody heavy chains, and the other scFv, Fab, or VHH antigen-binding domain that binds to human TfR is linked to the C-terminus or N-terminus of the other of the two antibody heavy chains. In some aspects, the two scFv, Fab or VHH antigen-binding domains that bind to human TfR can comprise the same amino acid sequence. In some aspects, the Fc is a heterodimeric Fc, such as an Fc domain with knob and hole mutations.

In some aspects, disclosed herein is a bivalent, bispecific protein comprising (i) a single scFv, VHH, or Fab antigen-binding domain that binds to human TfR linked to the N-terminus of an Fc domain of an Fc dimer, and (ii) a second antigen binding domain, wherein the second antigen binding domain does not specifically bind human TfR and is linked to the N-terminus of the second Fc domain of an Fc dimer. In some aspects, the second antigen binding domain binds a CNS or brain antigen other than TfR. An example of this 1+1 format is shown as (iii) in FIG. 1D.

In some aspects, disclosed herein is a bivalent, bi-specific protein comprising (i) a single scFv, VHH, or Fab antigen-binding domain that binds to human TfR linked to the N-terminus or C-terminus of an Fc domain of an Fc dimer and (ii) a second antigen binding domain, wherein the second antigen binding domain does not specifically bind human TfR and comprises a single scFv, VHH or Fab linked to N-terminus of the Fc or C-terminus of a second Fc domain of the Fc dimer. In some aspects, the Fc is a heterodimeric Fc, such as an Fc domain with knob and hole mutations. An example of this 1+1 format is shown as (ii) in FIG. 1D, where a single scFv is linked to the N terminus of an Fc domain of an Fc region and a second antigen binding domain is linked to the N terminus of a second Fc domain of the Fc region.

As provided herein, a fusion protein, antibody or antigen-binding fragment thereof, or multi-specific protein provided herein can be multi-specific, e.g., bi-specific. Many different formats and uses of bi-specific binding molecules are known in the art (reviewed in, e.g., Kontermann; Drug Discov Today, 2015 July; 20(7):838-47; MAbs, 2012 March-April; 4(2):182-97). A bispecific protein according to the present invention is not limited to any particular bispecific format or method of producing it. Accordingly, bispecific protein of the present disclosure can include various configurations having a first antigen-binding domain that binds to human TfR and a second antigen-binding domain, e.g., that binds to a CNS antigen or a brain antigen.

Bi-specific molecules include, e.g., a kappa-lambda body, a dual-affinity re-targeting molecule (DART), a knob-in-hole antibody, a strand-exchange engineered domain body (SEEDbody), and a DuoBody. In some aspects, a bispecific fusion protein, antibody or antigen-binding fragment thereof, or multi-specific protein provided herein comprises a knob mutation and a hole mutation. In some aspects, the knob mutation comprises the amino acid substitution T366W according to EU numbering. In some aspects, the hole mutation comprises the amino acids substitutions T366S, L368A, and Y407V according to EU numbering. Examples of bispecific molecules that can be used in the present disclosure include (i) a single antibody that has two arms comprising different antigen-binding domains; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody. Other examples of antibody structures are described in WO2019/246288, which is incorporated by reference.

In some aspects, a bispecific protein provided herein is selected from one of the following formats: CrossMab, DAF (two-in-one), DAF (four-in-one), DutaMab, DT-IgG, Charge pair, Fab-arm exchange, Triomab, LUZ-Y, Fcab, kappalambda-body, Orthogonal Fab, DVD-IgG, IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG (four-in-one), Nanobody, Nanbody-HAS, BiTE, TandAb, scDiabody-CH3, Diabody-CH3, Triple Body, miniantibody, minibody, TriBi minibody, scFv-CH3 KIH, Fab-scFv, scFV-CH-CL-scFV, F(ab′)2, F(ab′)2-scFv2, scFV-KIH, Fab-scFv-Fc, Tetravalent HCAb, scDiabody-Fc, Diabody-Fc, Tandem scFv-Fc, Intrabody, ImmTAC, HSAbody, scDiabody-HAS, Tandem scFv-toxin, IgG-IgG, Cov-X-body, and scFv1-PEG-scFv2, as described in Spiess, C., et al., Alternative molecular formats and therapeutic applications for bispecific antibodies Mol. Immunol. (2015), which is incorporated by reference herein.

In some aspects, a fusion protein, antibody or antigen-binding fragment thereof or multi-specific protein provided herein is bivalent. In some aspects, a multi-specific protein provided herein is multivalent (e.g., bivalent). In some aspects, a fusion protein, antibody or antigen-binding fragment thereof, or multi-specific protein provided herein is trivalent (e.g., in the 2+1 antibody format). In some aspects, a trivalent format comprises a single TfR antigen-binding domain provided herein and two antigen-binding domains that bind to a CNS antigen or a brain antigen. The two antigen-binding domains that bind to a CNS antigen or a brain antigen can comprise the same amino acid sequence or can comprise different amino acid sequences. In some aspects, the TfR antigen-binding domain is an scFv. In some aspects, the TfR antigen-binding domain is a VHH.

In some aspects, a fusion protein, antibody or antigen-binding fragment thereof, or multi-specific protein provided herein is tetravalent (e.g., in the 2+2 antibody format). In some aspects, a tetravalent format comprises two TfR antigen-binding domains provided herein and two antigen-binding domains that bind to a CNS antigen or a brain antigen. The two TfR antigen-binding domains can comprise the same amino acid sequence or can comprise different amino acid sequences. In some aspects, the two TfR antigen-binding domains comprise the same amino acid sequence. In some aspects, one or both of the TfR antigen-binding domains is an scFv. The two antigen-binding domains that bind to a CNS antigen or a brain antigen can comprise the same amino acid sequence or can comprise different amino acid sequences.

A fusion protein, antibody or antigen-binding fragment thereof, or multi-specific protein provided herein can comprise a linker, e.g., linking a TfR antigen-binding domain to a heterologous protein, antibody or antigen-binding fragment thereof, or other antigen-binding domain. The linker can be e.g., a glycine linker, a glycine-rich linker, or a glycine-serine linker. The linker can comprise the amino acid sequence (GGGGS)x3 (SEQ ID NO:184). The linker can comprise the amino acid sequence (GGSGG)x3 (SEQ ID NO:289).

A fusion protein, antibody or antigen-binding fragment thereof, or multi-specific protein provided herein can comprise a constant region. In some aspects, a TfR antigen-binding domain provided herein is linked to the constant region, e.g., the C-terminus of the constant region. In some aspects, a constant domain is a human constant domain. In some aspects, a constant domain is a murine, rat, rabbit, or monkey (e.g., cynomolgus) constant domain. The constant region can be a heavy chain constant region. The constant region can be a human constant region. The constant region can be a human heavy chain constant region. The constant region can be an IgG constant region. The constant region can be an IgG1 constant region. The constant region can be an IgG2 constant region. The constant region can be an IgG4 constant region. The constant region can be a human IgG constant region. The constant region can be a human IgG1 constant region. The constant region can be a human IgG2 constant region. The constant region can be a human IgG4 constant region. The linker can comprise the amino acid sequence GGSGG (no repeats) (SEQ ID NO:304). The linker may be 1 to 20 amino acids in length.

In some aspects a fusion protein, antibody or antigen-binding fragment thereof, or multi-specific protein provided herein comprises a heavy chain and a light chain. With respect to the heavy chain, in some aspects, the heavy chain of an antigen-binding protein described herein can be an alpha (α), delta (δ), epsilon (ε), gamma (γ) or mu (μ) heavy chain. In some aspects, the heavy chain can comprise a human alpha (α), delta (δ), epsilon (ε), gamma (γ) or mu (μ) heavy chain. In some aspects, the heavy chain comprises a human gamma (γ) heavy chain constant region. In some aspects, the heavy chain of comprises the amino acid sequence of an IgG1 heavy chain constant region. In some aspects, the heavy chain comprises the amino acid sequence of an IgG2 (e.g., IgG2a or IgG2b) heavy chain constant region. In some aspects, the heavy chain comprises the amino acid sequence of an IgG4 heavy chain constant region. With respect to the light chain, in some aspects, the light chain is a kappa light chain. In some aspects, the light chain is a lambda light chain. In some aspects, the light chain is a human kappa light chain or a human lambda light chain.

In some aspects, a fusion protein, antibody or antigen-binding fragment thereof, or multi-specific protein provided herein comprises constant regions comprising the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In some aspects, a fusion protein, antibody or antigen-binding fragment thereof, or multi-specific protein provided herein comprises constant regions comprising the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In some aspects, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

Non-limiting examples of human constant region sequences have been described in e.g., U.S. Pat. No. 5,693,780 and Kabat E A et al., (1991) Sequences of Proteins of Immunological Interest, Fifth Edition, U.S. Department of Health and Human Services, NIH Publication No. 91-3242).

In some aspects, a constant region provided herein comprises a knob mutation. In some aspects, a constant region provided herein comprises a hole mutation. Accordingly, in some aspects, a fusion protein, antibody or antigen-binding fragment thereof, or multi-specific protein provided herein can comprise a constant region comprising a knob mutation and a constant region comprising a hole mutation.

### FC Domains

A fusion protein, antibody or antigen-binding fragment thereof, or multi-specific protein provided herein can comprise an Fc domain or fragment thereof. In some aspects, an Fc domain is of IgG class, the IgM class, or the IgA class. In some aspects, an Fc domain or fragment thereof is an IgG Fc domain or fragment thereof. In some aspects, an Fc domain or fragment thereof is a human IgG Fc domain or fragment thereof. In some aspects, an Fc domain or fragment thereof is a human IgG1 Fc domain or fragment thereof. In some aspects, an Fc domain or fragment thereof is a human IgG2 Fc domain or fragment thereof. In some aspects, an Fc domain or fragment thereof is a human IgG4 Fc domain or fragment thereof. In some aspects, an Fc domain or fragment thereof is a monovalent Fc.

In some aspects provided herein, a fusion protein, antibody or antigen-binding fragment thereof, or multi-specific protein provided comprises a modified Fc domain or fragment thereof. In some aspects, the modified Fc domain or fragment thereof is a modified IgG1 Fc comprising one or more modifications. For example, in some aspects, the IgG1 modified Fc comprises one or more amino acid substitutions (e.g., relative to a wild-type Fc domain of the same isotype). In some aspects, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), K322A (Hezareh et al. (2001) J Virol. 75(24) 12161-12168), S267E, L328F, A330L, M252Y, S254T, E430G, and/or T256E, where the amino acid position is according to the EU numbering convention. In some aspects, the Fc comprises the amino acid substitutions L234A, L235A, and P331S (LALAPS) according to EU numbering. In some aspects, the Fc comprises N325S and L328F mutations according to EU numbering. In some aspects, the Fc comprises P329G or P329S according to EU numbering. In some aspects, the Fc comprises K322A according to EU numbering.

TABLE 1
Exemplary Fc Domains

| Fc variants (mutations | |
|---|---|
| according to Eu | |
| numbering) | Description |
| huIgG1 WT | Human IgG1 Fc |
| huIgG1 NSLF (N325S, | Both polypeptides of huIgG1 Fc contain N325S and L328F |
| L328F) | mutations, which decreases binding to FcGR3a and increase |
| | binding to FcGR2a |
| huIgG1 LALAPS (L234A, | Both polypeptides of huIgG1 Fc contain L234A, L235A, and |
| L235A, P329S) | P329S mutations, which minimize effector function (FcGR2a, |
| | FcgR3a and complement binding) |
| huIgG1 LALAPSv2 (L234A, | Both polypeptides of huIgG1 Fc contain L234A, L235A, and |
| L235A, P331S) | P331S mutations, which minimize effector function (FcGR2a, |
| | FcgR3a and complement binding) |
| huIgG1 LALA (L234A, | Both polypeptides of huIgG1 Fc contain L234A, L235A |
| L235A) | mutations, which minimize effector function (FcGR2a, FcgR3a |
| | binding) |
| huIgG1 K322A | Both polypeptides of huIgG1 Fc contain K332A mutations, which |
| | minimizes complement activated effector function. |
| huIgG1 YTE (M252Y, | Both polypeptides of huIgG1 Fc contain M252Y, S254T, T256E |
| S254T, T256E) | mutations, which enhance affinity to FcRn to extend antibody half- |
| | life |
| huIgG1 LS (M428L, | Both huIgG1 Fc contain M428L, N434S mutations. |
| N434S) | This enhances affinity to FcRn to extend antibody half-life |
| huIgG1 LALAPS (L234A, | One huIgG1 Fc polypeptide contains L234A, L235A, and P329S |
| L235A, P329S) / huIgG1 | mutations. The other huIgG1 Fc polypeptide is wild-type. |
| WT | This tunes effector function activity (FcgR2, FcgR3a and |
| | complement binding) |
| huIgG1 LALAPSv2 (L234A, | One huIgG1 Fc polypeptide contains L234A, L235A, and P331S |
| L235A, P331S) / huIgG1 | mutations. The other huIgG1 Fc polypeptide is wild-type. |
| WT | This tunes effector function activity (FcgR2, FcgR3a and |
| | complement binding) |
| huIgG1 LALA (L234A, | One huIgG1 Fc polypeptide contains L234A and L235A |
| L235A)/huIgG1 WT | mutations. |
| | The other huIgG1 Fc polypeptide is wild-type. |
| | This tunes effector function activity (FcgR2, and FcgR3a binding) |
| HuIgG1 LALA (L234A, | One huIgG1 Fc polypeptide contains L234A and L235A |
| L235A)/hu IgG1 PS | mutations. |
| (P329S) | The other huIgG1 Fc polypeptide contains a P329S mutation. |
| | This tunes effector function activity (FcgR2, FcgR3a and |
| | complement binding). |
| HuIgG1 LALA (L234A, | One huIgG1 Fc polypeptide contains L234A and L235A |
| L235A)/huIgG1 PSv2 | mutations. |
| (P331S) | The other huIgG1 Fc polypeptide contains a P329S mutation. |
| | This tunes effector function activity (FcgR2, FcgR3a and |
| | complement binding). |
| hulgG4 WT | Human IgG4 Fc |
| hulgG4 S228P | Both human IgG4 Fc polypeptides contain S228P mutations |
| | This minimizes Fab arm exchange present in wt IgG4 Fc |
| Heterodimeric Fc (knob and | Fc “knob” polypeptide contains T366W mutation |
| hole) | Fc “hole” polypeptide contains T366S, L368A, Y407V mutations |
| | This promotes Fc heterodimerization and can be combined with |
| | any of the above Fc mutations. |

In some aspects provided herein, a fusion protein, antibody or antigen-binding fragment thereof, or multi-specific protein provided herein is a bi-specific fusion protein, antibody or antigen-binding fragment thereof, or multi-specific protein. Bi-specific molecules include, e.g., a kappa-lambda body, a dual-affinity re-targeting molecule (DART), a knob-in-hole antibody, a strand-exchange engineered domain body (SEEDbody), and a DuoBody. In some aspects, a bispecific fusion protein, antibody or antigen-binding fragment thereof, or multi-specific protein provided herein comprises a knob mutation and a hole mutation. In some aspects, the knob mutation comprises the amino acid substitution T366W according to EU numbering. In some aspects, the hole mutation comprises the amino acids substitutions T366S, L368A, and Y407V according to EU numbering.

In some aspects provided herein, a fusion protein, antibody or antigen-binding fragment thereof, or multi-specific protein provided herein comprises one or more mutations to promote heterodimerization of Fc domains. In some aspects, a dimerized Fc domain of a bispecific provided herein is formed by Fc domains that contain amino acid mutations, substitutions, additions, or deletions to promote heterodimerization in which different polypeptides comprising different Fc domains can dimerize to yield a heterodimer configuration. In some aspects, a bispecific of the present disclosure comprises a first Fc sequence comprising a first CH3 region, and a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions

Methods to promote heterodimerization of Fc domains include amino acid deletions, additions, or substitutions of the amino acid sequence of the Fc domain, such as by including a set of “knob-into-hole” deletions, additions, or substitutions or including amino acid deletions, additions, or substitutions to effect electrostatic steering of the Fc to favor attractive interactions among different polypeptide chains. Methods for promoting heterodimerization of complementary Fc polypeptides have been previously described in, for example, Ridgway et al, 1996, Protein Eng, 9:617-621; Merchant et al, 1998, Nature Biotechnol, 16:677-681; Moore et al, 2011, MAbs, 3:546-557; Von Kreudenstein et al, 2013, 5:646-654; Gunasekaran et al, 2010, J Biol Chem, 285:19637-19464; Leaver-Fay et al, 2016, Structure, 24:641-651; Ha et al, 2016, Frontiers in Immunology, 7:1; Davis et al, 2010, Protein Eng Des Sel, 23:195-202; WO1996/027011; WO1998/050431; WO2006/028936; WO2009/089004; WO2011/143545; WO2014/067011; WO2012/058768; WO2018/027025; US2014/0363426; US2015/0307628; US2018/0016354; US2015/0239991; US2017/0058054; U.S. Pat. Nos. 5,731,168; 7,183,076; 9,701,759; 9,605,084; 9,650,446; 8,216,805; 8,765,412; and 8,258,268.

In some aspects, complementary Fc polypeptides of an Fe heterodimer include a mutation to alter charge polarity across the Fc dimer interface such that co-expression of electrostatically matched Fc domains support favorable attractive interactions, thereby promoting desired Fc heterodimer formation; whereas unfavorable repulsive charge interactions suppress unwanted Fc homodimer formation (Guneskaran et al, 2010, J Biol Chem, 285:19637-19646). When co-expressed in a cell, association between the polypeptide chains is possible but the chains do not substantially self-associate due to charge repulsion.

Additionally, complementary Fc polypeptides of an Fc heterodimer include “knob-into-hole” configurations to promote heterodimerization of two Fc polypeptides. “Knob-into-hole” technology is described in e.g. U.S. Pat. Nos. 5,731,168; 7,695,936; 8,216,805; 8,765,412; Ridgway et al., Prot Eng 9, 617-621 (1996); and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In some aspects, a knob modification comprises the amino acid substitution T366W in one of the two subunits of the Fc domain, and the hole modification comprises the amino acid substitutions T366S, L368A and Y407V in the other one of the two subunits of the Fc domain. In some aspects, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)). Thus, in such configurations, a first Fc polypeptide comprises amino acid modifications to form the “knob” and a second Fc polypeptide comprises amino acid modifications to form the “hole” thus forming an Fc heterodimer comprising complementary Fc polypeptides.

Exemplary paired amino acid modifications of complementary Fc polypeptides of an Fc heterodimeric configuration are set forth below in the table below (EU numbering).

TABLE 2
Exemplary paired Fc modifications for heterodimeric Fc domains

| First Fc polypeptide | Second Fc polypeptide |
|---|---|
| T366Y | Y407T |
| T366W | T366S/L368W/Y407V |
| T366W | T366S/L368A/Y407V |
| T366W/S354C | T366S/L368A/Y407V/Y349C |
| T350V/L351Y/F405A/Y407V | T350V/T366L/K392L/T394W |
| K360D/D399M/Y407A | E345R/Q347R/T366V/K409V |
| K409D/K392D | D399K/E356K |
| K360E/K409W | Q347R/D399V/F405T |
| L360E/K409W/Y349C | Q347R/D399V/F405T/S354C |
| K370E/K409W | E357N/D399V/F405T |

Some agents provided herein comprise antigen-binding fragments of antibodies. Antigen-binding fragments of antibodies include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that can be bivalent and/or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. Some aspects, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

As provided herein, a fusion protein, antibody or antigen-binding fragment thereof, or multi-specific protein provided herein can be chimeric. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody.

As provided herein, a fusion protein, antibody or antigen-binding fragment thereof, or multi-specific protein provided herein can be humanized. Typically, a non-human antibody is humanized to reduce immunogenicity to humans, while retaining the specificity and affinity of the parental non-human antibody. In some aspects, a humanized antibody is substantially non-immunogenic in humans. In some aspects, a humanized antibody has substantially the same affinity for a target as an antibody from another species from which the humanized antibody is derived. See, e.g., U.S. Pat. Nos. 5,530,101, 5,693,761; 5,693,762; and 5,585,089. In some aspects, amino acids of an antibody variable domain that can be modified without diminishing the native affinity of the antigen-binding domain while reducing its immunogenicity are identified. See, e.g., U.S. Pat. Nos. 5,766,886 and 5,869,619. Generally, a humanized antibody comprises one or more variable domains in which CDRs (or portions thereof) are derived from a non-human antibody, and framework regions (FRs) (or portions thereof) are derived from human antibody sequences. A humanized antibody can comprise at least a portion of a human constant region. In some aspects, some FR residues in a humanized antibody are substituted with corresponding residues from a non-human antibody (e.g., the antibody from which the CDR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that can be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

As provided herein, a fusion protein, antibody or antigen-binding fragment thereof, or multi-specific protein provided herein can be human. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies can be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology.

Human variable regions from intact antibodies generated by such animals can be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al. Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3):927-937 (2005) and Vollmers et al. Methods and Findings in Experimental and Clinical Pharmacology 27(3):185-91 (2005). Human antibodies can also be generated by isolating Fv clone variable domain sequences selected from human-derived phage display libraries. Such variable domain sequences can then be combined with a desired human constant domain. Techniques for selecting human antibodies from antibody libraries are described below.

In some aspects provided herein, an antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J. Mol. Biol. 338(2): 299-310, 2004; Lee et al. J. Mol. Biol. 340(5): 1073-1093, 2004; Fellouse Proc. Natl. Acad. Sci. USA 101(34):12467-12472 (2004); and Lee et al. J. Immunol. Methods 284(-2):1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12: 725-734 (1993). Finally, naive libraries can also be made synthetically by cloning unrearranged V-gene segments from stem cells, and using PCR primers comprising random sequence to encode the highly variable CDR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

As provided herein, a fusion protein, antibody or antigen-binding fragment thereof, or multi-specific protein provided herein can comprise an antigen-binding domain that binds to a CNS antigen or a brain antigen. As provided herein, an antibody or antigen-binding fragment thereof, or multi-specific protein provided herein can comprise an antigen-binding domain that binds to a CNS antigen or a brain antigen. In some aspects, the CNS antigen or brain antigen can be beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33 or sialic acid binding Ig-like lectin 3 (Siglec3), Membrane Spanning 4-Domains A4A (MS4A4A), Membrane Spanning 4-Domains A 6A (MS4A6A), or Transmembrane Protein 106B (TMEM106b). In some aspects, the CNS antigen or brain antigen can be beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (GCase or GBA), progranulin (PGRN), Prosaposin (PSAP), ubiquitin protein ligase E3A (UBE3A), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33, sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), sialic acid binding Ig-like lectin 11 (Siglec11), glycoprotein nonmetastatic melanoma protein B (GPNMB), Paired immunoglobin like type 2 receptor alpha (PILRA), Membrane Spanning 4-Domains A4A (MS4A4A), Membrane Spanning 4-Domains A 6A (MS4A6A), MSA4A4E, Transmembrane Protein 106B (TMEM106b), CR1, ABCA1, ABCA7, HLA-DR1, HLA-DR5, IL1RAP, TREML2, IL-34, SORL1, and ADAM1.

In some aspects, the CNS or brain antigen is on a cancer cell within the central nervous system. In some aspects, the CNS or brain antigen is a cell surface target on a hematological cancer cell selected from B7H3, BCMA, CD125, CD166, CD19, CD20, CD205, CD22, CD25, CD30, CD37, CD39, CD73, and CD79b. In some aspects, the CNS or brain antigen is a tumor cell target selected from siglec-3 or CD33, siglec-5, siglec-7, siglec-9, siglec 14, PILRA, IL18-BP, MerTK, ACKR1, ALK, AXL, CD25, CD44v6, CD46, CD56 (NCAM), CDH6 (cadherin 6), CEACAM 5 (CD66E), EGFR, EGFR viii, ETBR, FGFR (1-4), Folate Receptor alpha, GAL-3BP (galectin binding protein), GD2, GD3, GloboH (globohexasylceramide), gp100, gpNMB, HER2, HER3, HER4, IGFR1, KIT, LIV1A, LRRC15 (leucine rich repeat containing 15), MET, NaPi2B, PDL1, PMEL17, PRAME, PSMA, PTK7 (CCK4; colon carcinoma kinase), RON, ROR1, TF (tissue factor), and TROP2.

As provided herein, a fusion protein, antibody or antigen-binding fragment thereof, or multi-specific protein provided herein can comprise an antigen-binding domain that binds to a CNS antigen or a brain antigen. As provided herein, a multi-specific protein provided herein can comprise an antigen-binding domain that binds to a CNS antigen or a brain antigen. The antigen-binding domain that binds to a CNS antigen or a brain antigen can comprise a VH and a VL. Exemplary CNS antigen-binding VH and VL sequences are provided below. Additional VH and VL and antigen-binding domain sequences are found in US2017/0224702, US 2018/0002433, US 2021/0236634, and US 2021/0238265, each of which is herein incorporated by reference in its entirety.

TABLE 3
Exemplary CNS Antigens

| CNS Antigen | VH | VL |
|---|---|---|
| BACE1 | EVQLVESGGGLVQPGGSLRL | DIQMTQSPSSLSASVGDRVTITC |
| | SCAASGFTFSGYAIHWVRQA | RASQDVSTAVAWYQQKPGKAPKL |
| | PGKGLEWVGWISPAGGSTDY | LIYSASFLYSGVPSRFSGSGSGT |
| | ADSVKGRFTISADTSKNTAY | DFTLTISSLQPEDFATYYCQQSY |
| | LQMNSLRAEDTAVYYCARGP | TTPPTFGQGTKVEIKR (SEQ |
| | FSPWVMDYWGQGTLVTVSS | ID NO: 291) |
| | (SEQ ID NO: 290) | |
| BACE1 | EVQLVESGGGLVQPGGSLRL | DIQMTQSPSSLSASVGDRVTITC |
| | SCAASGFNFYYSSIHWVRQA | RASQSVSSAVAWYQQKPGKAPKL |
| | PGKGLEWVASISPYSGYTSY | LIYSASSLYSGVPSRFSGSGSGT |
| | ADSVKGRFTISADTSKNTAY | DFTLTISSLQPEDFATYYCQQYS |
| | LQMNSLRAEDTAVYYCARQP | YSPFTFGQGTKVEIKR (SEQ |
| | THYYYYAKGYKAMDYWGQGT | ID NO: 293) |
| | LVTVSS (SEQ ID NO: | |
| | 292) | |
| Alpha-synuclein | EVQLLESGGGLVQPGGSLRL | EIVLTQSPGTLSLSPGERATLSC |
| | SCAASGFTFSRFTMTWVRQA | RASQSVSRSYLAWYQQKPGQAPR |
| | PGKGLEWVSAISGSGGGTSY | LLIYGASSRATGIPDRFSGSGSG |
| | ADSVKGRLTVSRDNSKNTLY | TDFTLTVSRLEPEDFAVYYCQQY |
| | LQMNSLRAEDTAVYYCAKNW | GSSPWTFGQGTKVEIK (SEQ |
| | APFDYWGQGTLVTVSS | ID NO: 295) |
| | (SEQ ID NO: 294) | |
| Alpha-synuclein | EVQLLESGGGLVQTGGSLRL | EIVLTQSPGTLSLSPGERATLSC |
| | SCAASGFTFSSYAMTWVRQA | RASQSVSSSYLAWYQQKPGQAPR |
| | PGKGLEWVSAIRSNGDRTDY | LLIYGASSRATGIPDRFSGSGSG |
| | ADSVKGRFTISRDNSQNTLY | TDFTLTISRLEPEDFAVYYCQQY |
| | LQMNSLRAEDTAVYYCAKNW | GSSPWTFGQGTKVEIK (SEQ |
| | APFDSWGQGTLVTVSS | ID NO: 297) |
| | (SEQ ID NO: 296) | |
| Abeta | QVQLVQSGAEVKKPGSSVKV | DIVMTQTPLSLSVTPGQPASISC |
| | SCKASGYDFTRYYINWVRQA | KSSQSLLYSRGKTYLNWLLQKPG |
| | PGQGLEWMGWINPGSGNTKY | QSPQLLIYAVSKLDSGVPDRFSG |
| | NEKFKGRVTITADESTSTAY | SGSGTDFTLKISRVEAEDVGVYY |
| | MELSSLRSEDTAVYYCAREG | CVQGTHYPFTFGQGTKLEIK |
| | ITVYWGQGTTVTVSS (SEQ | (SEQ ID NO: 299) |
| | ID NO: 298) | |
| Tau | EVQLVESGGGLVQPGGSLKL | SYELTQPPSVSVSPGQTARISCF |
| | SCAASGFNFNISAIHWVRQA | GDTLPKQYTYWYQQKPGQAPVLV |
| | SGKGLEWVGRIRSKSHNYAT | IYKDTERPSGIPERFSGSSSGTT |
| | LYAASLKGRFTLSRDDSRNT | VTLTISGVQAEDEADYYCLSADN |
| | AYLQMSSLQTEDMAVYYCTV | SATWVFGGGTKVTVL (SEQ ID |
| | LSANYDTFDYWGQGTLVTVS | NO: 301) |
| | S (SEQ ID NO: 300) | |

As provided herein, a fusion protein, antibody or antigen-binding fragment thereof, or multi-specific protein provided herein can be capable of crossing the BBB as a result of the fact that the anti-TfR antigen-binding domain in the fusion protein, antibody or antigen-binding fragment thereof, or multi-specific protein is capable of crossing the BBB.

In some aspects, a fusion protein, antibody or antigen-binding fragment thereof, or multi-specific protein provided herein is internalized in blood-brain barrier epithelial cells greater than 10-fold as compared to internalization by an isotype control. The blood-brain barrier endothelial cells can be, e.g., HCMEC/D3 cells.

In some aspects, a fusion protein, antibody or antigen-binding fragment thereof, or multi-specific protein provided herein does not reduce cell-surface expression of TfR on HCMEC/D3 cells by more than 60% relative to cell-surface expression of TfR on HCMEC/D3 cells treated with an isotype control. In some aspects, a fusion protein, antibody or antigen-binding fragment thereof, or multi-specific protein provided herein does not reduce cell-surface expression of TfR on HCMEC/D3 cells by more than 40% relative to cell-surface expression of TfR on HCMEC/D3 cells treated with an isotype control. Cell surface expression can be measured, e.g., using Western blot or FACS.

In some aspects, a fusion protein, antibody or antigen-binding fragment thereof, or multi-specific protein provided herein does not significantly increase cell-surface expression of TfR on HCMEC/D3 cells relative to cell-surface expression of TfR on HCMEC/D3 cells treated with an isotype control. Cell surface expression can be measured, e.g., using Western blot or FACS.

In some aspects, a fusion protein, antibody or antigen-binding fragment thereof, or multi-specific protein provided herein does not reduce cell-surface expression of TfR on HCMEC/D3 cells by more than 60% relative to cell-surface expression of TfR on HCMEC/D3 cells treated with an isotype control and does not significantly increase cell-surface expression of TfR on HCMEC/D3 cells relative to cell-surface expression of TfR on HCMEC/D3 cells treated with an isotype control. Cell surface expression can be measured, e.g., using Western blot or FACS. In some aspects, a fusion protein, antibody or antigen-binding fragment thereof, or multi-specific protein provided herein does not reduce cell-surface expression of TfR on HCMEC/D3 cells by more than 40% relative to cell-surface expression of TfR on HCMEC/D3 cells treated with an isotype control and does not significantly increase cell-surface expression of TfR on HCMEC/D3 cells relative to cell-surface expression of TfR on HCMEC/D3 cells treated with an isotype control. Cell surface expression can be measured, e.g., using Western blot or FACS.

In some aspects, a fusion protein, antibody or antigen-binding fragment thereof, or multi-specific protein provided herein accumulates at least 4-fold more than an isotype control in vessel-depleted mouse brain. In some aspects, a fusion protein, antibody or antigen-binding fragment thereof, or multi-specific protein provided herein accumulates at least 5-fold more than an isotype control in vessel-depleted mouse brain.

In some aspects, a fusion protein, antibody or antigen-binding fragment thereof, or multi-specific protein provided herein binds human TfR with an equilibrium dissociation constant (KD) of about 6.7 nM to about 3.5 μM. In some aspects, a fusion protein, antibody or antigen-binding fragment thereof, or multi-specific protein provided herein binds human TfR with an equilibrium dissociation constant (KD) of about 6.7 nM to about 340 nM. In some aspects, a fusion protein, antibody or antigen-binding fragment thereof, or multi-specific protein provided herein binds cynomolgus monkey TfR with a KD of about 38 nM to about 2.3 μM. In some aspects, a fusion protein, antibody or antigen-binding fragment thereof, or multi-specific protein provided herein binds cynomolgus monkey TfR with a KD of about 18 nM to about 870 nM. In some aspects, a fusion protein, antibody or antigen-binding fragment thereof, or multi-specific protein provided herein binds human TfR with an equilibrium dissociation constant (KD) of about 6.7 nM to about 3.5 μM and herein binds cynomolgus monkey TfR with a KD of about 38 nM to about 2.3 μM. In some aspects, a fusion protein, antibody or antigen-binding fragment thereof, or multi-specific protein provided herein binds human TfR with an equilibrium dissociation constant (KD) of about 6.7 nM to about 340 nM and binds cynomolgus monkey TfR with a KD of about 18 nM to about 870 nM.

### Polynucleotides and Methods of Making Anti-TfR Antigen-Binding Domains and Agents Comprising the Same

In some aspects, provided herein are polynucleotides comprising a nucleotide sequence encoding an antigen-binding domain that specifically bind to human TfR, fusion protein, antibody, antigen-binding fragment thereof, or multi-specific protein described herein or a domain thereof described herein, and vectors, e.g., vectors comprising such polynucleotides for recombinant expression in host cells (e.g., E. coli and mammalian cells).

In some aspects, a polynucleotide provided herein comprises a nucleic acid molecule encoding the heavy chain of an antigen-binding domain that specifically binds to human TfR provided herein. In some aspects, a polynucleotide provided herein comprises a nucleic acid molecule encoding the light chain of an antigen-binding domain that specifically binds to human TfR provided herein. In some aspects, a polynucleotide provided herein comprises a nucleic acid molecule encoding the heavy chain of an antigen-binding domain that specifically bind to human TfR provided herein and a nucleic acid molecule encoding the light chain of an antigen-binding domain that specifically bind to human TfR provided herein.

In some aspects, combinations or compositions of polynucleotides are provided herein. In some aspects, a combination or composition comprises a first polynucleotide, a second polynucleotide, and a third polynucleotide, wherein the first, second, and third polynucleotides encode a multi-specific protein provided herein, e.g., wherein the first polynucleotide encodes a first heavy chain, the second polynucleotide encodes a second heavy chain and an antigen-binding domain that specifically binds to human TfR provided herein, and the third polynucleotide encodes a light chain. In some aspects, the antigen-binding domains that bind to human TfR is an scFv. In some aspects, the first heavy chain comprises a knob mutation and the second heavy chain comprises a hole mutation. In some aspects, the first heavy chain comprises a hole mutation and the second heavy chain comprises a knob mutation.

In some aspects, a combination or composition comprises a first polynucleotide, a second polynucleotide, and a third polynucleotide, wherein the first, second, and third polynucleotides encode a multi-specific protein provided herein, wherein the first polynucleotide encodes a first heavy chain and a first antigen-binding domain that specifically binds to human TfR, the second polynucleotide encodes a second heavy chain and a second antigen-binding domain that specifically binds to human TfR, and the third polynucleotide encodes a light chain. In some aspects, the first and second antigen-binding domains that bind to human TfR comprise the same amino acid sequence. In some aspects, the first and second antigen-binding domains that bind to human TfR comprise different amino acid sequences. In some aspects, the first and/or second antigen-binding domains that bind to human TfR are scFvs. In some aspects, the first heavy chain comprises a knob mutation and the second heavy chain comprises a hole mutation. In some aspects, the first heavy chain comprises a hole mutation and the second heavy chain comprises a knob mutation.

In some aspects, a combination or composition comprises a first polynucleotide and a second polynucleotide, wherein the first and second polynucleotides encode a multi-specific protein provided herein, wherein the first polynucleotide encodes a heavy chain and an antigen-binding domain that bind to human TfR provided herein, and wherein the second polynucleotide encodes a light chain.

Also provided herein are polynucleotides comprising a nucleotide sequence encoding an antigen-binding domain that specifically bind to human TfR, fusion protein, antibody, antigen-binding fragment thereof, or multi-specific protein described herein, or a domain thereof described herein, that are optimized, e.g., by codon/RNA optimization, replacement with heterologous signal sequences, and/or elimination of mRNA instability elements. Methods to generate optimized nucleic acids for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly.

A polynucleotide comprising a nucleotide sequence encoding an antigen-binding domain that specifically bind to human TfR, fusion protein, antibody, antigen-binding fragment thereof, or multi-specific protein described herein, or a domain thereof described herein, can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising, e.g., the sequence encoding the light chain and/or heavy chain of an antigen-binding domain, antibody, or antigen-binding fragment thereof. The amplified nucleic acids can be cloned into vectors for expression in host cells and for further cloning, for example, to generate an antigen-binding domain that specifically bind to human TfR, fusion protein, antibody, antigen-binding fragment thereof, or multi-specific protein described herein, or a domain thereof described herein.

Polynucleotides provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure.

In some embodiments, polynucleotides provided herein are in the form of RNA. In some embodiments, polynucleotides provided herein are in the form of RNA encoding a fusion protein provided herein. In some embodiments, a polynucleotide provided herein is a synthetic messenger RNA (mRNA). In some embodiments, the synthetic mRNA has at least one nucleoside modification. In some embodiments, the at least one nucleoside modification is selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some aspects, provided herein are polynucleotides encoding a fusion protein comprising an antigen-binding protein provided herein and a heterologous polypeptide. In some aspects, the heterologous polypeptide comprises an antigen binding domain that binds to beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (GCase or GBA), progranulin (PGRN), Prosaposin (PSAP), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33 or sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), glycoprotein nonmetastatic melanoma protein B (GPNMB), Paired immunoglobin like type 2 receptor alpha (PILRA), Membrane Spanning 4-Domains A4A (MS4A4A), Membrane Spanning 4-Domains A 6A (MS4A6A), or Transmembrane Protein 106B (TMEM106b), or a portion thereof. In some aspects, the heterologous polypeptide comprises an antigen binding domain that binds to ubiquitin protein ligase E3A (UBE3A). In some aspects, provided herein are polynucleotides encoding a multispecific protein provided herein. In some aspects, the multispecific protein comprises an antigen binding domain that binds to beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (GCase or GBA), progranulin (PGRN), Prosaposin (PSAP), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33 or sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), glycoprotein nonmetastatic melanoma protein B (GPNMB), Paired immunoglobin like type 2 receptor alpha (PILRA), Membrane Spanning 4-Domains A4A (MS4A4A), Membrane Spanning 4-Domains A 6A (MS4A6A), or Transmembrane Protein 106B (TMEM106b), or a portion thereof. In some aspects, the multispecific protein comprises an antigen binding domain that binds to ubiquitin protein ligase E3A (UBE3A). In some embodiments, the polynucleotide is mRNA (e.g., synthetic mRNA).

In some embodiments, disclosed herein are polynucleotides encoding a fusion protein disclosed herein comprising a heterologous polypeptide. In some aspects, the heterologous polypeptide is an ERT enzyme or an ERT enzyme variant, or a catalytically active fragment thereof. In some aspects, the heterologous polypeptide comprises β-glucocerebrosidase (GCase or GBA), progranulin (PGRN), Prosaposin (PSAP), or a catalytically active fragment thereof. In some aspects, the heterologous polypeptide in a fusion protein provided herein is a growth factor. In some aspects, the heterologous polypeptide in a fusion protein provided herein is a decoy receptor. In some aspects, the heterologous polypeptide in a fusion protein provided herein is progranulin (PGRN), prosaposin (PSAP), or survival motor neuron protein (SMN). In some aspects, the heterologous protein is an enzyme selected from α-L Iduronidase (IDUA), Iduronate-2-sulphatase (IDS), N-acetylgalactoslamine-6-sulphatase (GALNS), N-sulfoglucosamine sulfohydrolase (SGSH), N-acetylgalactosamine-4-sulphatase (arylsulphatase B; ARSB), acid sphingomyelinase (ASM), β-glucocerebrosidase (GCase or GBA), galactosylceramide beta-galactosidase, glucosylceramidase, beta-hexosaminidase A, beta-hexosaminidase B, arylsulphatase A, beta-galactosidase, acid ceramidase, alpha-glucosidase, lysosomal acid lipase, lysosomal protease, a synthetic enzyme replacement thereof, such as larodinase, idursulphase, elosulphase alpha or galsuphase, or a variant thereof, or a catalytically active fragment thereof. In some aspects, the heterologous protein is an enzyme selected from clusterin (APOJ), Reelin, ubiquitin protein ligase E3A (UBE3A), Tripeptidyl Peptidase 1 (CLN2/TPP1), glucosamine (N-acetyl)-6-sulfatase (GNS), heparan-alpha-glucosaminide N-acetyltransferase (HGSNAT), and N-acetyl-alpha-glucosaminidase (NAGLU), α-L Iduronidase (IDUA), Iduronate-2-sulphatase (IDS), N-acetylgalactoslamine-6-sulphatase (GALNS), N-sulfoglucosamine sulfohydrolase (SGSH), N-acetylgalactosamine-4-sulphatase (arylsulphatase B; ARSB), acid sphingomyelinase (ASM), β-glucocerebrosidase (GCase or GBA), galactosylceramide beta-galactosidase, glucosylceramidase, beta-hexosaminidase A, beta-hexosaminidase B, arylsulphatase A, beta-galactosidase, acid ceramidase, alpha-glucosidase, lysosomal acid lipase, lysosomal protease, a synthetic enzyme replacement thereof, such as larodinase, idursulphase, elosulphase alpha or galsuphase, or a variant thereof, or a catalytically active fragment thereof α-L Iduronidase (IDUA), Iduronate-2-sulphatase (IDS), N-acetylgalactoslamine-6-sulphatase (GALNS), N-sulfoglucosamine sulfohydrolase (SGSH), N-acetylgalactosamine-4-sulphatase (arylsulphatase B; ARSB), acid sphingomyelinase (ASM), β-glucocerebrosidase (GCase or GBA), galactosylceramide beta-galactosidase, glucosylceramidase, beta-hexosaminidase A, beta-hexosaminidase B, arylsulphatase A, beta-galactosidase, acid ceramidase, alpha-glucosidase, lysosomal acid lipase, lysosomal protease, a synthetic enzyme replacement thereof, such as larodinase, idursulphase, elosulphase alpha or galsuphase, or a variant thereof, or a catalytically active fragment thereof. In some embodiments, the polynucleotide is mRNA (e.g., synthetic mRNA).

In certain aspects, provided herein are vectors (e.g., expression vectors) comprising polynucleotides comprising nucleotide sequences encoding an antigen-binding domain that specifically bind to human TfR, fusion protein, antibody, antigen-binding fragment thereof, or multi-specific protein described herein, or a domain thereof described herein, for recombinant expression in a host cell, e.g., in a mammalian host cell. A vector for the production of the antigen-binding domain that specifically bind to human TfR, fusion protein, antibody, antigen-binding fragment thereof, or multi-specific protein described herein, or a domain thereof described herein, can be produced, e.g., by recombinant DNA technology using techniques well known in the art. These methods include, for example, in vitro recombinant DNA techniques, synthetic techniques, and in vivo genetic recombination. Also provided are replicable vectors comprising a nucleotide sequence encoding an antigen-binding domain that specifically bind to human TfR, fusion protein, antibody, antigen-binding fragment thereof, or multi-specific protein described herein, or a domain thereof described herein, operably linked to a promoter. Such vectors can, for example, include the nucleotide sequence encoding the constant region of an antigen-binding domain, antibody or antigen-binding fragment thereof (see, e.g., International Publication Nos. WO 86/05807 and WO 89/01036; and U.S. Pat. No. 5,122,464), and variable domains of the antigen-binding domain, antibody or antigen-binding fragment thereof can be cloned into such a vector for expression of the entire heavy, the entire light chain, or both the entire heavy and light chains. In some embodiments, the vector is gene therapy vector (e.g., an AAV or lentiviral vector).

In certain aspects, provided herein are expression systems comprising polynucleotides comprising nucleotide sequences encoding an antigen-binding domain that specifically bind to human TfR, fusion protein, antibody, antigen-binding fragment thereof, or multi-specific protein described herein, or a domain thereof described herein. An expression system can be included on a vector. An expression system can also be integrated into a host cell chromosome. In some aspects, an expression system is a cell free expression system. In some aspects, an expressions system comprises a host cell comprising a polynucleotide and/or vector provided herein.

Accordingly, also provided herein are cells, e.g. host cells, comprising polynucleotides and/or vectors for recombinantly expressing an antigen-binding domain that specifically bind to human TfR, fusion protein, antibody, antigen-binding fragment thereof, or multi-specific protein described herein, or a domain thereof described herein. In some aspects, for the expression of double-chained antigen-binding proteins, vectors encoding both the heavy and light chains, individually, can be co-expressed in the host cell for expression of the entire immunoglobulin. In some aspects, a host cell contains two different vectors, a first vector comprising a polynucleotide encoding a heavy chain of an antigen-binding protein described herein, and a second vector comprising a polynucleotide encoding a light chain of the an antigen-binding protein. In some aspects, a first host cell comprises a first vector comprising a polynucleotide encoding a heavy chain, and a second host cell comprises a second vector comprising a polynucleotide encoding a light chain. In some aspects, provided herein is a population of host cells comprising such first host cell and such second host cell.

In some aspects, provided herein are methods for producing an antigen-binding domain that specifically binds to human TfR, fusion protein, antibody, antigen-binding fragment thereof, or multi-specific protein described herein, or a domain thereof described herein in a host cell. In some aspects, provided herein are methods for producing a single chain antigen-binding domain that specifically binds to human TfR, an Fc domain, and a heterologous protein or polypeptide. In some aspects, provided herein are methods for producing a single chain antigen-binding domain that specifically binds to human TfR, an Fc domain, and a second antigen-binding domain, as described herein. An expression vector can be transferred to a cell (e.g., host cell) by conventional techniques, and the resulting cells can then be cultured by conventional techniques to produce an antigen-binding domain that specifically binds to human TfR, fusion protein, antibody, antigen-binding fragment thereof, or multi-specific protein described herein, or a domain thereof described herein.

A variety of host-expression vector systems can be utilized to express an antigen-binding domain that specifically bind to human TfR, fusion protein, antibody, antigen-binding fragment thereof, or multi-specific protein described herein, or a domain thereof described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antigen-binding domain that specifically bind to human TfR, fusion protein, antibody, antigen-binding fragment thereof, or multi-specific protein described herein, or a domain thereof described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing an antigen-binding domain that specifically bind to human TfR, fusion protein, antibody, antigen-binding fragment thereof, or multi-specific protein described herein, or a domain thereof described herein are CHO cells, for example CHO cells from the CHO GS System™ (Lonza). In some aspects, cells for expressing an antigen-binding domain that specifically bind to human TfR, fusion protein, antibody, antigen-binding fragment thereof, or multi-specific protein described herein, or a domain thereof described herein as described herein are human cells, e.g., human cell lines. In some aspects, a mammalian expression vector is pOptiVEC™ or pcDNA3.3. In some aspects, bacterial cells, such as Escherichia coli, or eukaryotic cells (e.g., mammalian cells) are used for the expression of an antigen-binding domain that specifically bind to human TfR, fusion protein, antibody, antigen-binding fragment thereof, or multi-specific protein described herein, or a domain thereof described herein. For example, mammalian cells such as Chinese hamster ovary (CHO) cells in conjunction with a vector such as the major intermediate early gene promoter element from human cytomegalovirus is an effective expression system for antibodies (Foecking MK & Hofstetter H (1986) Gene 45: 101-105; and Cockett M I et al., (1990) Biotechnology 8: 662-667). In some aspects, an antigen-binding domain that specifically bind to human TfR, fusion protein, antibody, antigen-binding fragment thereof, or multi-specific protein described herein, or a domain thereof described herein is produced by CHO cells or NS0 cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Once an antigen-binding domain that specifically bind to human TfR, fusion protein, antibody, antigen-binding fragment thereof, or multi-specific protein described herein, or a domain thereof described herein has been produced by recombinant expression, it can be purified by any method known in the art for purification, for example, by chromatography (e.g., ion exchange, affinity, particularly by affinity for the specific antigen after Protein A, and sizing column chromatography), centrifugation, differential solubility, or by any other standard technique for the purification of proteins. Further, the antigen-binding domain that specifically bind to human TfR, fusion protein, antibody, antigen-binding fragment thereof, or multi-specific protein described herein, or a domain thereof described herein can be fused to heterologous polypeptide sequences to facilitate purification.

In some aspects, once an antigen-binding domain that specifically bind to human TfR, fusion protein, antibody, antigen-binding fragment thereof, or multi-specific protein described herein is isolated or purified. Generally, an isolated or purified antigen-binding domain that specifically bind to human TfR, fusion protein, antibody, antigen-binding fragment thereof, or multi-specific protein described herein is one that is substantially free of other proteins. For example, in some aspects, a preparation of an antigen-binding domain that specifically bind to human TfR, fusion protein, antibody, antigen-binding fragment thereof, or multi-specific protein described herein is substantially free of cellular material and/or chemical precursors.

### Pharmaceutical Compositions

Provided herein are compositions comprising an antigen-binding domain that specifically bind to human TfR, fusion protein, antibody, antigen-binding fragment thereof, or multi-specific protein described herein. In some aspects, the antigen-binding domain that specifically bind to human TfR, fusion protein, antibody, antigen-binding fragment thereof, or multi-specific protein having the desired degree of purity is present in a formulation comprising, e.g., a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed. Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some aspects, a pharmaceutical composition comprises an antigen-binding domain that specifically bind to human TfR, fusion protein, antibody, antigen-binding fragment thereof, or multi-specific protein described herein, and a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Pharmaceutical compositions described herein are, in some aspects, for use as a medicament. The compositions to be used for in vivo administration can be sterile. This is readily accomplished by filtration through, e.g., sterile filtration membranes.

Also provided herein are pharmaceutical compositions comprising a polynucleotide encoding an antigen-binding domain that specifically binds to human TfR, a fusion protein, an antibody or antigen-binding fragment thereof, or multi-specific protein described herein. In some embodiments, the polynucleotide is RNA. In some embodiments, the polynucleotide is synthetic mRNA. In some embodiments, the polynucleotide is a modified mRNA. In some embodiments, the pharmaceutical composition comprising a polynucleotide further comprises a lipid-based transfection reagent.

A pharmaceutical composition described herein can be used to exert a biological effect(s) in vivo or in vitro. For example, a pharmaceutical composition described herein can be used to cross a blood brain barrier, e.g., in a subject.

In some aspects, a pharmaceutical composition provided herein is used to treat diseases or conditions such as a neuropathy disorder, a neurodegenerative disease, cancer, an ocular disease disorder, a seizure disorder, a lysosomal storage disease, amyloidosis, a viral or microbial disease, ischemia, a behavioral disorder, and CNS inflammation. In some aspects, a pharmaceutical composition provided herein is used to treat diseases or conditions such as Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury. In some aspects, a pharmaceutical composition provided herein is used to treat frontotemporal dementia.

In some aspects, a pharmaceutical composition provided herein is formulated for intravenous administration. In some aspects, a pharmaceutical composition provided herein is formulated for subcutaneous administration.

### Methods of Using Anti-TfR Antigen-Binding Domains and Agents Comprising the Same

Antigen-binding domains that specifically bind to human TfR, fusion proteins, antibodies, antigen-binding fragments thereof, and multi-specific proteins comprising such antigen-binding domains as provided herein can advantageously be transported across a blood brain barrier. Accordingly, provided herein are methods of administering or transporting an antigen-binding protein that specifically binds to human TfR or a fusion protein, antibody, antigen-binding fragment thereof, or multi-specific protein comprising an antigen-binding protein that specifically binds to human TfR, across the blood brain barrier of a subject comprising administering to the subject an antigen-binding protein that specifically binds to human TfR or a fusion protein, antibody, antigen-binding fragment thereof, or multi-specific protein comprising an antigen-binding protein that specifically binds to human TfR.

In view of the ability of antigen-binding domains that specifically bind to human TfR and fusion proteins, antibodies, antigen-binding fragments thereof, and multi-specific proteins comprising such antigen-binding domains as provided herein to be transported across a blood brain barrier, they can be used to treat a neurological disease or disorder. In some aspects, a method of treating a neurological disease or disorder in a subject comprises administering to the subject an antigen-binding protein that specifically binds to human TfR or a fusion protein, antibody, antigen-binding fragment thereof, or multi-specific protein comprising an antigen-binding protein that specifically binds to human TfR. The neurological disease or disorder can be, for example, a neuropathy disorder, a neurodegenerative disease, cancer, an ocular disease disorder, a seizure disorder, a lysosomal storage disease, amyloidosis, a viral or microbial disease, ischemia, a behavioral disorder, or CNS inflammation. The neurological disease or disorder can be, for example, a neurodegenerative disease (such as Lewy body disease, postpoliomyelitis syndrome, Shy-Draeger syndrome, olivopontocerebellar atrophy, Parkinson's disease, Gaucher disease, multiple system atrophy, striatonigral degeneration, spinocerebellar ataxia, spinal muscular atrophy), a tauopathy (such as Alzheimer disease and supranuclear palsy), a prion disease (such as bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, a nervous system heterodegenerative disorders (such as Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (such as Pick's disease, and spinocerebellar ataxia), cancer of the CNS and/or brain (such as glioblastoma or brain metastases resulting from cancer elsewhere in the body), Alzheimer's disease (AD), stroke, dementia, muscular dystrophy (MD), multiple sclerosis (MS), amyotrophic lateral sclerosis (ALS), limbic-predominant age-related TDP-43 encephalopathy (LATE), cystic fibrosis, Angelman's syndrome, Liddle syndrome, Parkinson's disease, Pick's disease, Paget's disease, cancer, or traumatic brain injury. In some aspects, the neurological disease or disorder is dementia. In some aspects, the neurological disease or disorder is frontotemporal dementia. In some aspects, the neurological disease or disorder is Alzheimer's disease. In some aspects, the neurological disease or disorder is Parkinson's disease. In some aspects, the neurological disease or disorder is frontal temporal epilepsy. In some embodiments, the neurological disease or disorder is autism. In some aspects, the neurological disease or disorder is lissencephaly.

In some aspects, provided herein is a method of treating a lysosomal storage disease with a fusion protein disclosed herein. In some aspects, the lysosomal storage disease is selected from Gaucher disease, Ceroid lipofuscinosis (Batten disease), Mucopolysaccharidosis (MPS) Type I, MPS Type II and MPS Type III.),

Antigen-binding domains that specifically bind to human TfR and fusion proteins, antibodies, antigen-binding fragments thereof, and multi-specific proteins comprising such antigen-binding domains as provided herein can be used to detect an antigen (e.g., a CNS antigen or a brain antigen). Antigen-binding domains that specifically bind to human TfR and fusion proteins, antibodies, antigen-binding fragments thereof, and multi-specific proteins comprising such antigen-binding domains for such purposes can be labeled. Exemplary labels include, for example, radioisotopes (e.g., ⁶⁴CU) and fluorescent labels. Accordingly, methods of detecting an antigen using an antigen-binding protein that specifically binds to human TfR or a fusion protein, antibody, antigen-binding fragment thereof, or multi-specific protein comprising an antigen-binding protein that specifically binds to human TfR described herein are provided. In some aspects, a method of detecting an antigen in the CNS (e.g., brain) of a subject comprises administering an antigen-binding protein that specifically binds to human TfR or a fusion protein, antibody, antigen-binding fragment thereof, or multi-specific protein comprising an antigen-binding protein that specifically binds to human TfR to the antigen in the CNS (e.g., brain). Such methods can further comprise, e.g., performing Positron emission tomography (PET) imaging on the subject. In some aspects, disclosed herein is a method of detecting a CNS antigen in vitro, comprising contacting an in vitro sample with a fusion protein, antibody or antigen-binding fragment thereof, or multi-specific protein disclosed herein and locating the imaging agent within the sample.

Antigen-binding domains that specifically bind to human TfR and fusion proteins, antibodies, antigen-binding fragments thereof, and multi-specific proteins comprising such antigen-binding domains as provided herein can be used for prognostic, diagnostic, monitoring, and/or screening applications, including in vivo applications well known and standard to the skilled artisan and based on the present description. In some aspects, provided herein is an antigen-binding protein that specifically binds to human TfR or a fusion protein, antibody, antigen-binding fragment thereof, or multi-specific protein comprising an antigen-binding protein that specifically binds to human TfR for use as a diagnostic. In some aspects, the antigen-binding protein that specifically binds to human TfR or a fusion protein, antibody, antigen-binding fragment thereof, or multi-specific protein comprising an antigen-binding protein that specifically binds to human TfR comprises a detectable label.

All references cited herein, including patent applications and publications, are hereby incorporated by reference in their entirety.

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Production of Avi-His Tagged Variants of Transferrin Receptor (TfR)

Mammalian expression of human, cynomolgus macaque (cyno) and mouse variants of Transferrin Receptor antigens (SEQ ID NOs:1-7) was performed by cloning synthetic genes based on cDNA into mammalian expression vectors, followed by transient transfection and expression in Expi293 cells. Constructs included a heterologous signal peptide and C-terminal Avi-His tag to allow for purification and biotinylation. Briefly, plasmids encoding the antigens were transfected using the Expifectamine 293 Transfection kit (ThermoFisher A14524) according to the manufacturer's specifications. Five days after transfection, the culture supernatants were harvested, clarified by centrifugation, and purified using HisPur Ni-NTA resin (Thermo Scientific 88223) in a drip-column format. 200 mL of culture supernatant was filtered using 0.2 um filtration unit and 3 mL of resin slurry in PBS was added to the filtered supernatant. The sample was incubated overnight with shaking at 4° C. and the following day the beads were loaded onto 20 mL drip columns, washed with 10 mL of His-Select wash buffer (Millipore Sigma H5288), and eluted with 5 mL of His-Pur elution buffer (Millipore Sigma H5413). Eluate was buffer exchanged into PBS using Amicon Ultra-15 centrifugal filter units (Millipore UFC9010). Quantification of the antibody concentration was determined by measuring the absorbance at 280 nm using the Nanodrop 8000 (ThermoFisher). Purity of the antigens was determined by SDS-PAGE. The antigens were analyzed with size exclusion chromatography (SEC) for aggregation. Some antigens were biotinylated using a BirA biotin-protein ligase kit (AVIDITY), according to the manufacturer's instructions.

The apical domain of TfR presents a means for viruses (Helguera, G., et al., Virology 86(7): 4024-4028 (April 2012)) and antibodies (Kariolis M., et al., Sci. Transl. Med. 12(545): eaay1359 (May 2020)) to enter endothelial cells without perturbing the native function of the receptor. Tagged variants of the apical domain of Tfr were generated. These constructs included amino acids 69-263 of the full length human TfR sequence, with either a C-terminal Avi-His tag (SEQ ID NO:4) or a C-terminal myc-V5-His tag (SEQ ID NO:5) (sequences in Table 4 below). The resulting clones were expressed in Expi293 cells and purified by HisPur Ni-NTA resin as described above.

DNA fragments that encoded a permuted form of the human TfR (huTfR) and cynomologous TfR (cynoTfR) apical domain and N-term His/Avi tags were ordered from GeneArt and cloned into pcDNA 3.4 vector (SEQ ID NOs:6 and 7). The resulting clones were expressed in Expi293 cells and purified by Ni-NTA agarose (QIAGEN 30230) using the manufacturer's protocol.

TABLE 4
Avi-His tagged variants of TfR

| SEQ | | |
|---|---|---|
| ID | Descrip- | |
| NO | tion | Sequence |
| 1 | huTfR | CKGVEPKTECERLAGTESPVREEPGEDFPAARRLYWDDLKRKLSEKLDSTDFTGTIK |
| | Extra- | LLNENSYVPREAGSQKDENLALYVENQFREFKLSKVWRDQHFVKIQVKDSAQNSVII |
| | cellular | VDKNGRLVYLVENPGGYVAYSKAATVTGKLVHANFGTKKDFEDLYTPVNGSIVIVRA |
| | Domain | GKITFAEKVANAESLNAIGVLIYMDQTKFPIVNAELSFFGHAHLGTGDPYTPGFPSF |
| | (ECD) | NHTQFPPSRSSGLPNIPVQTISRAAAEKLFGNMEGDCPSDWKTDSTCRMVTSESKNV |
| | Avi-His | KLTVSNVLKEIKILNIFGVIKGFVEPDHYVVVGAQRDAWGPGAAKSGVGTALLLKLA |
| | | QMFSDMVLKDGFQPSRSIIFASWSAGDFGSVGATEWLEGYLSSLHLKAFTYINLDKA |
| | | VLGTSNFKVSASPLLYTLIEKTMQNVKHPVTGQFLYQDSNWASKDTDYPYLGTTMDT |
| | | YKELIERIPELNKVARAAAEVAGQFVIKLTHDVELNLDYERYNSQLLSFVRDLNQYR |
| | | ADIKEMGLSLQWLYSARGDFFRATSRLTTDFGNAEKTDRFVMKKLNDRVMRVEYHFL |
| | | SPYVSPKESPFRHVFWGSGSHTLPALLENLKLRKQNNGAFNETLFRNQLALATWTIQ |
| | | GAANALSGDVWDIDNEFGGGLNDIFEAQKIEWHEHHHHHHHHH |
| 2 | Cyno | CKGVEPKTECERLAGTESPAREEPEEDFPAAPRLYWDDLKRKLSEKLDTTDFTSTIK |
| | TfR | LLNENLYVPREAGSQKDENLALYIENQFREFKLSKVWRDQHFVKIQVKDSAQNSVII |
| | ECD | VDKNGGLVYLVENPGGYVAYSKAATVTGKLVHANFGTKKDFEDLDSPVNGSIVIVRA |
| | Avi-His | GKITFAEKVANAESLNAIGVLIYMDQTKFPIVKADLSFFGHAHLGTGDPYTPGFPSF |
| | | NHTQFPPSQSSGLPNIPVQTISRAAAEKLFGNMEGDCPSDWKTDSTCKMVTSENKSV |
| | | KLTVSNVLKETKILNIFGVIKGFVEPDHYVVVGAQRDAWGPGAAKSSVGTALLLKLA |
| | | QMFSDMVLKDGFQPSRSIIFASWSAGDFGSVGATEWLEGYLSSLHLKAFTYINLDKA |
| | | VLGTSNFKVSASPLLYTLIEKTMQDVKHPVTGRSLYQDSNWASKVEKLTLDNAAFPF |
| | | LAYSGIPAVSFCFCEDTDYPYLGTTMDTYKELVERIPELNKVARAAAEVAGQFVIKL |
| | | THDTELNLDYERYNSQLLLFLRDLNQYRADVKEMGLSLQWLYSARGDFFRATSRLTT |
| | | DFRNAEKRDKFVMKKLNDRVMRVEYYFLSPYVSPKESPFRHVFWGSGSHTLSALLES |
| | | LKLRRQNNSAFNETLFRNQLALATWTIQGAANALSGDVWDIDNEFGGGLNDIFEAQK |
| | | IEWHEHHHHHHHHH |
| 3 | Mouse | CKRVEQKEECVKLAETEETDKSETMETEDVPTSSRLYWADLKTLLSEKLNSIEFADT |
| | TfR | IKQLSQNTYTPREAGSQKDESLAYYIENQFHEFKFSKVWRDEHYVKIQVKSSIGQNM |
| | (muTfR) | VTIVQSNGNLDPVESPEGYVAFSKPTEVSGKLVHANFGTKKDFEELSYSVNGSLVIV |
| | ECD | RAGEITFAEKVANAQSFNAIGVLIYMDKNKFPVVEADLALFGHAHLGTGDPYTPGFP |
| | Avi-His | SFNHTQFPPSQSSGLPNIPVQTISRAAAEKLFGKMEGSCPARWNIDSSCKLELSQNQ |
| | | NVKLIVKNVLKERRILNIFGVIKGYEEPDRYVVVGAQRDALGAGVAAKSSVGTGLLL |
| | | KLAQVFSDMISKDGFRPSRSIIFASWTAGDFGAVGATEWLEGYLSSLHLKAFTYINL |
| | | DKVVLGTSNFKVSASPLLYTLMGKIMQDVKHPVDGKSLYRDSNWISKVEKLSFDNAA |
| | | YPFLAYSGIPAVSFCFCEDADYPYLGTRLDTYEALTQKVPQLNQMVRTAAEVAGQLI |
| | | IKLTHDVELNLDYEMYNSKLLSFMKDLNQFKTDIRDMGLSLQWLYSARGDYFRATSR |
| | | LTTDFHNAEKTNRFVMREINDRIMKVEYHFLSPYVSPRESPFRHIFWGSGSHTLSAL |
| | | VENLKLRQKNITAFNETLFRNQLALATWTIQGVANALSGDIWNIDNEFGGGLNDIFE |
| | | AQKIEWHEHHHHHHHHH |
| 4 | huTfR | KIQVKDSAQNSVIIVDKNGRLVYLVENPGGYVAYSKAATVTGKLVHANFGTKKDFED |
| | Apical | LYTPVNGSIVIVRAGKITFAEKVANAESLNAIGVLIYMDQTKFPIVNAELSFFGHAH |
| | Domain- | LGTGDPYTPGFPSFNHTQFPPSRSSGLPNIPVQTISRAAAEKLFGNMEGDCPSDWKT |
| | Avi-His | DSTCRMVTSESKNVKLTVSNVLKEGGGLNDIFEAQKIEWHEHHHHHHHHH |
| 5 | huTfR | KIQVKDSAQNSVIIVDKNGRLVYLVENPGGYVAYSKAATVTGKLVHANFGTKKDFED |
| | Apical | LYTPVNGSIVIVRAGKITFAEKVANAESLNAIGVLIYMDQTKFPIVNAELSFFGHAH |
| | Domain- | LGTGDPYTPGFPSFNHTQFPPSRSSGLPNIPVQTISRAAAEKLFGNMEGDCPSDWKT |
| | myc-V5- | DSTCRMVTSESKNVKLTVSNVLKERAAAGSGSEQKLISEEDLGKPIPNPLLGLDSTH |
| | His | HHHHHHHHH |
| 6 | HuTfR | HHHHHHHHGLNDIFEAQKIEWHESSGLPNIPVQTISRAAAEKLFGNMEGDCPSDWKT |
| | apical | DSTCRMVTSESKNVKLTVSNDSAQNSVIIVDKNGRLVYLVENPGGYVAYSKAATVTG |
| | permuted | KLVHANFGTKKDFEDLYTPVNGSIVIVRAGKITFAEKVANAESLNAIGVLIYMDQTK |
| | N-term | FPIVNAELS |
| | Avi-His | |
| 7 | Cyno | HHHHHHHHHHGLNDIFEAQKIEWHESSGLPNIPVQTISRAAAEKLFGNMEGDCPSDW |
| | TfR | KTDSTCKMVTSENKSVKLTVSNDSAQNSVIIVDKNGGLVYLVENPGGYVAYSKAATV |
| | apical | TGKLVHANFGTKKDFEDLDSPVNGSIVIVRAGKITFAEKVANAESLNAIGVLIYMDQ |
| | permuted | TKFPIVKADLS |
| | N-term | |
| | Avi-His | |

### Example 2: Production of CHO Cell Lines Over-Expressing TfR

Several cell lines were generated for screening the binding of anti-TfR antibodies obtained as described herein. Briefly, pLenti-EF1a constructs each expressing full length human TfR and mouse TfR (SEQ ID NOs: 8 and 9, respectively) were used to generate CHO cells stably expressing human TfR and mouse TfR. Lentiviral constructs (Genecopoeia) were used to express human TfR with hygromycin selection and mouse TfR with hygromycin selection. Lentivirus was generated from transfection of 293T cells using the ViraSafe™ Lentiviral Packaging System (Cellfliolabs VPK-206). Subsequently, the supernatant containing lentivirus was used to transduce Chinese Hamster Ovary (CHO) cells. At 2 days post transduction, puromycin or hygromycin was added to the medium as a selection pressure for the human TfR and mouse TfR. The resulting CHO cells stably expressing human TfR and mouse TfR were analyzed for cell surface expression by flow cytometry.

TABLE 5
Full-length TfR sequences

| SEQ | | |
|---|---|---|
| ID | Descrip- | |
| NO | tion | Sequence |
| 8 | Full | MMDQARSAFSNLFGGEPLSYTRFSLARQVDGDNSHVEMKLAVDEEENADNNTKANVT |
| | length | KPKRCSGSICYGTIAVIVFFLIGFMIGYLGYCKGVEPKTECERLAGTESPVREEPGE |
| | human | DFPAARRLYWDDLKRKLSEKLDSTDFTGTIKLLNENSYVPREAGSQKDENLALYVEN |
| | TfR | QFREFKLSKVWRDQHFVKIQVKDSAQNSVIIVDKNGRLVYLVENPGGYVAYSKAATV |
| | | TGKLVHANFGTKKDFEDLYTPVNGSIVIVRAGKITFAEKVANAESLNAIGVLIYMDQ |
| | | TKFPIVNAELSFFGHAHLGTGDPYTPGFPSFNHTQFPPSRSSGLPNIPVQTISRAAA |
| | | EKLFGNMEGDCPSDWKTDSTCRMVTSESKNVKLTVSNVLKEIKILNIFGVIKGFVEP |
| | | DHYVVVGAQRDAWGPGAAKSGVGTALLLKLAQMFSDMVLKDGFQPSRSIIFASWSAG |
| | | DFGSVGATEWLEGYLSSLHLKAFTYINLDKAVLGTSNFKVSASPLLYTLIEKTMQNV |
| | | KHPVTGQFLYQDSNWASKVEKLTLDNAAFPFLAYSGIPAVSFCFCEDTDYPYLGTTM |
| | | DTYKELIERIPELNKVARAAAEVAGQFVIKLTHDVELNLDYERYNSQLLSFVRDLNQ |
| | | YRADIKEMGLSLQWLYSARGDFFRATSRLTTDFGNAEKTDRFVMKKLNDRVMRVEYH |
| | | FLSPYVSPKESPFRHVFWGSGSHTLPALLENLKLRKQNNGAFNETLFRNQLALATWT |
| | | IQGAANALSGDVWDIDNEF |
| 9 | Full | MMDQARSAFSNLFGGEPLSYTRFSLARQVDGDNSHVEMKLAADEEENADNNMKASVR |
| | length | KPKRFNGRLCFAAIALVIFFLIGFMSGYLGYCKRVEQKEECVKLAETEETDKSETME |
| | Mouse | TEDVPTSSRLYWADLKTLLSEKLNSIEFADTIKQLSQNTYTPREAGSQKDESLAYYI |
| | TfR | ENQFHEFKFSKVWRDEHYVKIQVKSSIGQNMVTIVQSNGNLDPVESPEGYVAFSKPT |
| | | EVSGKLVHANFGTKKDFEELSYSVNGSLVIVRAGEITFAEKVANAQSFNAIGVLIYM |
| | | DKNKFPVVEADLALFGHAHLGTGDPYTPGFPSFNHTQFPPSQSSGLPNIPVQTISRA |
| | | AAEKLFGKMEGSCPARWNIDSSCKLELSQNQNVKLIVKNVLKERRILNIFGVIKGYE |
| | | EPDRYVVVGAQRDALGAGVAAKSSVGTGLLLKLAQVFSDMISKDGFRPSRSIIFASW |
| | | TAGDFGAVGATEWLEGYLSSLHLKAFTYINLDKVVLGTSNFKVSASPLLYTLMGKIM |
| | | QDVKHPVDGKSLYRDSNWISKVEKLSFDNAAYPFLAYSGIPAVSFCFCEDADYPYLG |
| | | TRLDTYEALTQKVPQLNQMVRTAAEVAGQLIIKLTHDVELNLDYEMYNSKLLSFMKD |
| | | LNQFKTDIRDMGLSLQWLYSARGDYFRATSRLTTDFHNAEKTNRFVMREINDRIMKV |
| | | EYHFLSPYVSPRESPFRHIFWGSGSHTLSALVENLKLRQKNITAFNETLFRNQLALA |
| | | TWTIQGVANALSGDIWNIDNEF |

### Example 3: Generation of TfR Humanized Mice

Mouse lines were generated at Taconic Biosciences GmbH (Germany) to humanize the extracellular domains of TfR. CRISPR was used to replace the mouse ECD with the human version, while retaining the mouse intracellular and transmembrane portions under the control of the mouse promotor. Brain slices from these mice were evaluated by JUC and/or Western Blot to confirm expression and localization of the human ECD in vivo (data not shown).

### Example 4: Generation of Anti-TfR Hybridoma Antibodies

In order to obtain antibodies against TfR, the following procedures were used to generate hybridomas. BALB/c mice or Sprague Dawley rats (Charles River Laboratories, Wilmington, MA) were immunized twice a week by subcutaneous or intraperitoneal injections of purified extracellular domain polypeptides of human, cyno, and/or mouse TfR (obtained as described above in Example 1) with or without adjuvant. After a total of 7-8 injections and three days following the final boost, the lymph nodes were harvested from the mice or rats for hybridoma cell line generation.

Sera from the animals were analyzed for reactivity to TfR by FACS on CHO cells overexpressing human or mouse TfR and by ELISA against human, cyno and mouse TfR Avi-His proteins (Example 1). Lymphocytes from animals whose sera demonstrated strong binding to CHO cells overexpressing human or mouse TfR were isolated and fused with SP2/mIL-6 (CRL-2016, American Type Culture Collection, Rockville, MD) or Sp2ab (ENZ-70008, Enzo Life Sciences, Farmingdale, NY) mouse myeloma cells via electrofusion (Hybrimune, BTX, Holliston, MA) and incubated at 37° C., 5% CO2, overnight in Clonacell-HY Medium C (Stemcell Technologies, Vancouver, BC, Canada, Cat #03803).

The following day, the fused cells were centrifuged and resuspended in Clonacell-HY Medium E (Stemcell Technologies, Cat #03805) with HAT (Sigma Aldrich, Cat #H0262). The cells were seeded into T225 flasks and grown for 7 days at 37° C., 5% CO2, and then the hybridoma libraries were cryopreserved. The libraries were later thawed and recovered overnight in Clonacell-HY Medium E. The following day, IgG positive hybridomas were single cell sorted into Falcon 96-well U-bottom plates using a FACS Aria II cell sorter (BD Biosciences, San Jose, CA). 768 hybridomas were sorted from the mouse fusions and 768 hybridomas were sorted from the rat fusions. After the cells were grown in 200 ul of Clonacell-HY Medium E at 37° C. with 5% CO2 for 11 days, tissue culture supernatants from each well were screened by FACS on CHO cells overexpressing human or mouse TfR (as described below).

### Example 5: Screening of Anti-TfR Antibody Hybridoma Supernatants by FACS

A total of 1536 anti-TfR hybridoma supernatants were initially screened for their ability to differentially bind CHO cells overexpressing human or mouse TfR cells compared to CHO parental cells by FACS. Overexpressing cells were harvested, washed and labeled with Pacific Blue, FITC and Pacific Orange dyes (ThermoFisher) to create uniquely barcoded cell populations. Barcoded cells (5×10⁴ of each cell population) were aliquoted into 96-well U-bottom plates and incubated with 50 μl of hybridoma cell culture supernatant or 1 g/ml of commercially available purified mouse anti-human TfR monoclonal antibody (Sigma Aldrich, Cat #SAB4700515) and rat anti-mouse TfR antibody (Invitrogen, Waltham, MA, Cat #R17217) on ice for 30 minutes. After this primary incubation, the supernatants were removed via centrifugation, the cells were washed twice with 175 μl of ice-cold FACS buffer (PBS+1% FBS+2 mM EDTA), and the cells were then further incubated on ice for 20 minutes with anti-mouse IgG Fc-allophycocyanin (APC) or anti-rat IgG Fc-APC (Jackson Labs, Cat #115-136-071 and Cat #112-136-071, respectively) (diluted 1:1000). Following this secondary antibody incubation, the cells were again washed twice with ice-cold FACS buffer and resuspended in a final volume of 50 μl of FACS buffer. Binding intensity on cells was analyzed using the FACS Canto system (BD Biosciences), and the ratio of APC Mean Fluorescence Intensity (MFI) on each barcoded cell population was determined for each anti-TfR hybridoma supernatant tested.

A total of 189 mouse hybridoma and 115 rat hybridoma clones displayed greater than 3-fold difference in binding (as determined by MFI) to CHO cells stably overexpressing human TfR compared to the isotype control. A total of 15 rat hybridomas displayed greater than 2-fold differential binding to CHO cells stably overexpressing mouse TfR compared to the isotype control (data not shown). None of the hybridoma clones were cross-reactive to human and mouse TfR by FACS.

TABLE 6
FACS Results for selected mouse hybridoma supernatants
(shown as fold over CHO parental)

Fold over parental (CHO WT)

| Antibody | CHO + huTfR | CHO + muTfR |
|---|---|---|
| TfR1 | 228 | 1 |
| TfR2 | 458 | 1 |
| TfR3 | 38 | 1 |
| TfR4 | 61 | 1 |
| TfR5 | 561 | 1 |
| TfR6 | 451 | 1 |
| TfR7 | 87 | 1 |
| TfR8 | 215 | 1 |
| TfR9 | 203 | 1 |
| TfR10 | 389 | 1 |
| TfR11 | 290 | 1 |
| TfR13 | 392 | 1 |
| TfR14 | 6 | 1 |
| TfR15 | 119 | 1 |
| TfR16 | 534 | 1 |
| TfR17 | 73 | 1 |
| TfR18 | 75 | 1 |
| TfR22 | 290 | 1 |
| TfR23 | 279 | 1 |
| TfR24 | 370 | 1 |
| TfR25 | 135 | 1 |
| TfR26 | 285 | 1 |
| TfR27 | 279 | 1 |
| TfR28 | 157 | 1 |
| TfR30 | 169 | 1 |
| TfR31 | 90 | 1 |
| TfR32 | 137 | 1 |

TABLE 7
FACS Results for selected rat hybridoma supernatants
(shown as fold over CHO parental)

Fold over parental (CHO WT)

| Antibody | CHO + huTfR | CHO + muTfR |
|---|---|---|
| TfR33 | 123 | 1 |
| TfR34 | 84 | 1 |
| TfR35 | 306 | 1 |
| TfR36 | 433 | 1 |
| TfR37 | 290 | 1 |
| TfR38 | 439 | 1 |
| TfR39 | 245 | 1 |
| TfR40 | 65 | 1 |
| TfR41 | 92 | 1 |
| TfR42 | 369 | 1 |
| TfR43 | 231 | 1 |
| TfR44 | 84 | 1 |
| TfR45 | 550 | 1 |
| TfR46 | 370 | 1 |
| TfR47 | 442 | 1 |
| TfR48 | 96 | 1 |
| TfR49 | 297 | 1 |
| TfR50 | 322 | 1 |
| TfR51 | 86 | 1 |
| TfR52 | 466 | 1 |
| TfR53 | 167 | 1 |
| TfR54 | 646 | 1 |

### Example 6: Screening of Anti-TfR Antibody Hybridoma Supernatants by Recombinant TfR Protein Binding Assay

Hybridoma culture supernatants from 319 hybridomas obtained as described above were screened for their ability to bind Avi-His-tagged huTfR ECD, huTfR apical domain, and muTfR ECD (as described in Example 1) as compared to binding to an irrelevant Avi-His-tagged control protein. Briefly, 96-well polystyrene plates were coated with 5 μg/ml of streptavidin (Thermo Fisher, Cat #PI21125) in coating buffer (0.05M carbonate buffer, pH 9.6, Sigma, Cat #C3041) overnight at 4° C. Coated plates were then blocked with ELISA diluent (PBS+0.5% BSA+0.05% Tween20) for one hour. Blocking buffer was removed and Avi-His-tagged human TfR, human TfR apical domain, mouse TfR and an irrelevant Avi-His-tagged protein were added at 1 μg/ml in ELISA diluent and captured for 1 hour at room temperature. After washing three times with 300 μl of PBST (PBS+0.05% Tween20, Thermo 28352), the hybridoma cell culture supernatants were added (50 μl/well) to each well. After 30 minutes of incubation at room temperature, the plates were washed three times with 300 μl of PBST. Anti-mouse IgG Fc-HRP or anti-rat IgG Fc-HRP (Jackson Immunoresearch, Cat #115-035-071 and 112-036-071, respectively) secondary antibodies were diluted 1:5000 in ELISA diluent, added to each well at 50 μl/well, and incubated for 30 minutes at room temperature with shaking. After a final set of washes (3×300 μl in PBST), 50 μl/well of BioFx TMB substrate (Surmodics, Cat #TMBW-1000-01) was added to the wells. The reaction was then quenched after 5-10 mins with 50 μl/well of 2N sulfuric acid. The plates were read for absorbance at 450 nm on a SpectraMax M5 (Molecular Devices, Sunnyvale, CA) using SoftMax Pro software.

From this hybridoma supernatant screen, a total of 112 anti-TfR hybridoma clones were identified that displayed greater than 5-fold difference in binding to recombinant huTfR Avi-His over background, and 86 of these anti-TfR hybridoma clones also bound to the huTfR apical domain. Binding data (shown as a ratio of binding to recombinant huTfR Avi-His over irrelevant protein) for selected mouse anti-TfR hybridoma clones and rat anti-TfR hybridoma clones are shown in Tables 8 and 9, respectively.

TABLE 8
ELISA (OD450) results for selected mouse hybridoma supernatants
(shown as fold over irrelevant protein)

Ratio over irrelevant

| Antibody | huTfR apical Avi-His | huTfR Avi-His |
|---|---|---|
| TfR1 | 22 | 9 |
| TfR2 | 5 | 1 |
| TfR3 | 37 | 14 |
| TfR4 | 42 | 20 |
| TfR5 | 15 | 12 |
| TfR6 | 37 | 17 |
| TfR7 | 48 | 25 |
| TfR8 | 37 | 16 |
| TfR9 | 10 | 5 |
| TfR10 | 35 | 14 |
| TfR11 | 39 | 25 |
| TfR13 | 22 | 7 |
| TfR14 | 29 | 19 |
| TfR15 | 34 | 21 |
| TfR16 | 18 | 16 |
| TfR17 | 26 | 22 |
| TfR18 | 32 | 21 |
| TfR22 | 33 | 24 |
| TfR23 | 31 | 24 |
| TfR24 | 31 | 17 |
| TfR25 | 33 | 21 |
| TfR26 | 35 | 22 |
| TfR27 | 34 | 22 |
| TfR28 | 30 | 22 |
| TfR30 | 29 | 8 |
| TfR31 | 20 | 17 |
| TfR32 | 25 | 13 |

TABLE 9
ELISA (OD450) results for selected rat hybridoma supernatants
(shown as fold over irrelevant protein)

Fold over irrelevant

| Antibody | huTfR apical Avi-His | huTfR Avi-His |
|---|---|---|
| TfR33 | 23 | 9 |
| TfR34 | 6 | 9 |
| TfR35 | 23 | 29 |
| TfR36 | 22 | 30 |
| TfR37 | 8 | 4 |
| TfR38 | 32 | 31 |
| TfR39 | 22 | 17 |
| TfR40 | 19 | 11 |
| TfR41 | 17 | 8 |
| TfR42 | 25 | 19 |
| TfR43 | 31 | 24 |
| TfR44 | 16 | 10 |
| TfR45 | 34 | 29 |
| TfR46 | 17 | 5 |
| TfR47 | 5 | 7 |
| TfR48 | 9 | 17 |
| TfR49 | 20 | 22 |
| TfR50 | 13 | 7 |
| TfR51 | 23 | 19 |
| TfR52 | 26 | 8 |
| TfR53 | 26 | 12 |
| TfR54 | 23 | 11 |

### Example 7: In Vitro Internalization of Anti-TfR Antibodies into a Blood-Brain Barrier Endothelial Cell Line

Anti-TfR hybridoma antibodies were purified and screened for their ability to internalize into an hCMEC/D3 cell line.

Supernatants from the anti-TfR hybridoma clones were purified using ProPlus Phytip columns (Biotage, Uppsala, Sweden, Cat #PTH 91-20-07) on a Hamilton STAR platform (Hamilton Company, Reno, NV). Briefly, antibodies from the supernatants were captured by protein A coupled on resin-packed tips, washed twice with PBS, eluted with Pierce IgG elution buffer (ThermoFisher, Cat #21004), and neutralized with 1 M Tris-HCl pH 8 to a final pH of 6.0. The concentrations of the purified antibodies were determined by measuring the absorbance at 280 nm using the Nanodrop 8000 (ThermoFisher). Hybridoma purified antibodies were then tested for their ability to internalize.

Internalization into endothelial cells at the blood-brain barrier is the first stage of transcytosis across the BBB and into the brain. To identify anti-TfR antibodies with internalization ability, HCMEC/D3 cells were seeded at 2.5*10∝cells/well in a black wall clear bottom 96-well plate (#3904, Corning). Next day, cells were treated with 6 μg/ml of anti-TfR antibodies pre-conjugated with an equal concentration of pHrodo-Red labeling reagent (Z25612, Invitrogen) in 100 μl of culture medium (EBM2, Lonza). A human IgG isotype and an anti-TfR antibody with known internalization ability were included in the assay as negative and positive controls, respectively.

Plates were then placed into an IncuCyte machine (live-cell analysis system), and images were captured every 2 hrs over a 24 hr time course. Images were then processed and analyzed using IncuCyte software. Internalization data at the 24 hr timepoint (pHrodo Red positive area μm²/image) is summarized below in Table 10 as relative fold-change to isotype hIgG1.

Data was reported as total red area per well and is shown in Table 10 as a fold change over an irrelevant mouse IgG antibody used as a negative control. Also included in this assay was an anti-TfR antibody used as a positive control.

TABLE 10
Internalization of purified anti-TfR hybridoma antibodies

| | Fold over isotype |
|---|---|
| Antibody | control |
| TfR1 | 25.0 |
| TfR2 | 18.0 |
| TfR3 | 7.2 |
| TfR4 | 47.1 |
| TfR5 | 72.3 |
| TfR6 | 76.8 |
| TfR7 | 6.2 |
| TfR8 | 7.9 |
| TfR9 | 26.2 |
| TfR10 | 11.9 |
| TfR11 | 2.0 |
| TfR13 | 22.5 |
| TfR14 | 14.2 |
| TfR15 | 1.2 |
| TfR16 | 2.2 |
| TfR17 | 7.6 |
| TfR18 | 1.2 |
| TfR22 | 1.9 |
| TfR23 | 1.6 |
| TfR24 | 9.9 |
| TfR25 | 6.7 |
| TfR26 | 8.6 |
| TfR27 | 1.4 |
| TfR28 | 3.2 |
| TfR30 | 714.7 |
| TfR31 | 2.5 |
| TfR32 | 10.2 |
| anti-TfR positive | 16.7 |
| control | |
| Isotype control | 1.0 |

### Example 8: Molecular Cloning of Anti-TfR Antibodies

Anti-TfR antibodies from the hybridomas described above were cloned as follows. 1-2×10⁵ hybridoma cells were harvested, washed with PBS, and resuspended in 200 μl of RNAlater (Invitrogen, Cat #. AM7021). Samples were stored at −80° C. and sent to Abterra Biosciences (San Diego, CA) for sequencing. Briefly, RNA was extracted and cDNA synthesis was performed. The variable regions of IgG/IgM, IgK, and IgL were amplified using proprietary primers in a 5′ RACE strategy. Hybridoma variable region amplicons were sequenced on the Illumina MiSeq platform (Illumina, San Diego, CA). Reads from the hybridomas were processed through Abterra's Reptor analysis pipeline.

Out of the 72 apical domain positive anti-TfR hybridoma clones, a total of 32 unique sequences were identified. Amino acid sequences of the variable heavy chains and variable light chains of unique mouse-derived apical domain binding anti-TfR antibodies are provided below in Table 11. Out of the 24 apical domain positive anti-TfR hybridoma clones from rats, an additional 21 unique sequences were identified.

TABLE 11
Anti-huTfR apical domain antibody sequences
from mouse hybridomas

| Anti- | | |
|---|---|---|
| body | VH sequence | VL sequence |
| TfR1 | QVQLQQSGPELVKPGASVKMSCKASGYTF | DIVMTQSPSSLTVTAGEKVTMRCKSSQSLL |
| | TNYYIHWVKQRPGQGLEWIGWIYPGDGST | NSGNQKNYLTWYQQKPGQPPKLLIYWASTR |
| | KYNEKFKGKTTLTADKSSSTAYMLLSSLT | ESGVPDRFTGSGSGTDFTLTISSVQAEDLA |
| | SEDSAIYFCARDGSSAPKFTYWGQGTLVT | VYYCQNDYSYPLTFGAGTKLELK (SEQ |
| | VSA (SEQ ID NO: 10) | ID NO: 11) |
| TfR2 | EVQLQQSGPELVKPGASVKISCKASGYTF | DILLTQSPAILSVSPGERVSFSCRASQSIG |
| | TDYNMHWVKQSHGKSLEWIGYIYPYNGGT | TSIHWYQQRTNGSPRLLIKYASESISGIPS |
| | GYNQKFKSKATLTVDNSSSTAYMELRSLT | RFSGSGSGTDFTLSINNVESEDIADYYCQQ |
| | SEDSAVYYCATLYDGYYFDYWGQGTTLTV | SNSWPFTFGSGTKLEIK (SEQ ID NO: |
| | SS (SEQ ID NO: 12) | 13) |
| TfR3 | QVQLRQSGPELVKPGASVRISCKASGYTF | DIVMTQSPSSLTVTAGEKVTMSCKSSQSLL |
| | TSFYIHWVKQRPGQGLEWIGWIYPGDVNT | NSGNQKNYLTWYQQKPGQPPKLLIYWASTR |
| | HYSDKFKGKATLTADKSSSTAYMQLSSLT | ESGVPDRFTGVGSGTDFTLTISSVQAEDLA |
| | SEDSAVYFCAREGYGYFAYWGQGTLVTVS | VYYCQNDYSYPLTFGSGTKLELK (SEQ |
| | A (SEQ ID NO: 14) | ID NO: 15) |
| TfR4 | QVQLQQSGAELVRPGTSVKVSCKASGYAF | SIVMTQTPKFLLVSAGDRVTITCKASQSVS |
| | TNYLIEWVKQRPGQGLEWIGVINPGSGGT | NDVAWYQQKPGQSPKLLIYYASNRYTGVPD |
| | TYNEKFKGKATLTADKSSSTAYMQLSGLT | RFTGSGYGTDFTFTISTVQAEDLAVYFCHQ |
| | SDDSAVYFCARRSPYFDYWGQGTTLTVSS | DYISPTFGGGTKLEIK (SEQ ID NO: |
| | (SEQ ID NO: 16) | 17) |
| TfR5 | QVQLQQSGPELVKPGASVKMSCKASGYTF | DIVMTQSPSSLTVTAGEKVTMRCKSSQSLL |
| | TNYYIHWVKQRPGQGLEWIGWIYPGDGNT | NSGNQKNYLTWYQQKPGQPPKLLFYWASTR |
| | NYNEKFKGKTTLTADKSSSTAYMLLSSLT | ESGVPDRFTGSGSGTDFTLTISSVQAEDLA |
| | SEDSAIYFCARDGSSAPKFTYWGQGTLVT | VYYCQNDYSYPLTFGAGTKLELK (SEQ |
| | VSA (SEQ ID NO: 18) | ID NO: 19) |
| TfR6 | DVQLVESGGGLVQPGGSRKLSCAASGFTF | DIVMTQSHKFMSTSVGDRVSITCKASQDVS |
| | SSFGMHWVRQAPEKGLEWVAYISSGSSAI | TAVAWYQQKPGQSPKLLIYWASTRPTGVPD |
| | YYADTVKGRFTVSRDNPKNTLFLQMTSLR | RFTGSGSGTDYTLTISSVQAEDLALYSCQQ |
| | SEDTAIYYCATSYVYDPPWFAYWGQGTLV | HFSTPWTFGGGTKLEIK (SEQ ID NO: |
| | TVSA (SEQ ID NO: 20) | 21) |
| TfR7 | DVQLQESGPGLVKPSQSLSLTCTVTGYSI | DIVMTQSQKFMSTSVGDRVSVTCKASQNVL |
| | TSDYAWNWLRQFPGNKLEWMGYISYSGYS | TNVAWYQQKPGHSPKALIYSSSSRNSGVPD |
| | RHNPSLKSRISITRDTSKNQFFLQLNSVT | RFTGSGSGTDFTLTISNVQSEDLAEYFCQQ |
| | SEDTATYYCARGDWDFDYWGQGTTLTVSS | YNTYPYTFGGGTKLEIK (SEQ ID NO: |
| | (SEQ ID NO: 22) | 23) |
| TfR8 | QVQLQQSGAELVRPGTSVKVSCKASGYAF | SIVMTQTPKFLLVSAGDRVTITCKASQSVS |
| | TNYLIEWVKQRPGQGLEWIGVINPGSGGT | NDVAWYQQKPGQSPKLLIYYASNRYTGVPD |
| | NYNEKFKGKATLTADKSSSTAYMQLSSLT | RFTGSGYGTDFTFTISTVQAEDLAVYFCHQ |
| | SDDSAVYFCARRSPYFDYWGQGTTLTVSS | DYISPTFGGGTKLEIK (SEQ ID NO: |
| | (SEQ ID NO: 24) | 25) |
| TfR9 | EVQLQQSGPELVKPGASVKISCKTSGYTF | DVVMTQTPLTLSVTIGQPASISCKSSQSLL |
| | TEYAMHWVKQSHGKSLEWIGGINPNDGGT | DSDGKTYLNWLLQRPGQSPKRLIYLVSKLD |
| | SYNQKFKGKATLTVDKSSSTAYMELRSLT | SGVPDRFTGSGSGTDFTLKISRVEAEDLGV |
| | SEDSAVYYCSSQRWLLRGGDYWGQGTTLT | YYCWQGTHFPQTFGGGTKLEIK (SEQ ID |
| | VSS (SEQ ID NO: 26) | NO: 27) |
| TfR10 | QVQLQQSGPELVKPGASVKMSCKASGYTF | DIVMTQSPSSLTVTAGEKVTMSCKSSQSLL |
| | TSYYIHWVKQRPGQGLEWIGWIYPGDGNT | NSGNQKNYLTWYQQKPGQPPKLLIYWASTR |
| | NYNEKFKGKTTLTADKSSSTAYMLLSSLT | ESGVPDRFTGSGSGTDFTLTISSVQAEDLA |
| | SEDSAIYFCARDGSIPFAYWGQGTLVTVS | VYYCQNDYSYPLTFGAGTKLELK (SEQ |
| | A (SEQ ID NO: 28) | ID NO: 29) |
| TfR11 | EVQLQQSGTVLARPGASVKMSCKASGYSF | DIKMTQSPSSMYASLGERVILTCKASQDIN |
| | TSYWMHWVKQRPGQGLEWIGAIFPGNIDT | SYLSWFQQKPGKSPKTLIYHVNRLVDGVPS |
| | IYNQKFKDKAKLTAVTSASTAYMELSSLT | RFSGSGSGQDYSLTISSLEYEDMGIYYCLQ |
| | NEDSAVYFCTRLYYDYVWGAGTTVTVSS | YDEFPYTFGGGTKLEIK (SEQ ID NO: |
| | (SEQ ID NO: 30) | 31) |
| TfR12 | QVQLQQPGSELVRPGASVKLSCKASGYTF | QIVLTQSPAIMSASPGEKVTMTCSASSSIR |
| | TSYWMHWVKQRPGQGLEWIGNIYPGSGSS | YMNWYQQKPVTSPKRWIYDTSTLASGVPAR |
| | NYDEKFKNKATLTVDTSSNTAYMQLSSLT | FSGSGSGTSYSLTISSMEAEDAATYYCHQR |
| | SEDSAVYYCTRSSYYGNFDTMDNWGQGTS | SSYPWTFGGGTKLEIK (SEQ ID NO: |
| | VTVSS (SEQ ID NO: 32) | 33) |
| TfR13 | DVKLVESGGGLVKPGGSLKLSCAASGFTF | DILLTQSPAILSVSPGERVSFSCRASQSIG |
| | SSYTMSWVRQTPEKRLEWVATISSGGSYT | TSIHWYQQRTNGSPRLLIKYASGSISGIPS |
| | YFPDSVKGRFTISRDNAKNTLYLQMSSLK | RFSGSGSGTDFTLSINSVESEDIADYYCQQ |
| | SEDTAIYYCTRAHYYAYDYWGQGTTLTVS | SNSWPTSTFGAGTKLELK (SEQ ID NO: |
| | S (SEQ ID NO: 34) | 35) |
| TfR14 | QVQLQQSGPELVKPGASVKISCKASGYAF | DIVLTQSPASLAVSLGQRATISCRASESVD |
| | SSSWMNWVKQRPGQGLEWIGRIYPGDGDT | NYGISFMNWFQQKPGQPPKLLIYAASNQGS |
| | NYNGKFKGKATLTADKSSSTAYMQLSSLT | GVPARFSGSGSGTDFSLNIHPMEEDDTAMY |
| | SVDSAVYFCARDPIIDYYYYAMDYWGQGT | FCQQSKEVPWTFGGGTKLEIK (SEQ ID |
| | SVTVSS (SEQ ID NO: 36) | NO: 37) |
| TfR15 | QVQLQQPGSELVRPGASVKLSCKASGYTF | QIVLTQSPAIMSASPGEKVTMTCSASSSIR |
| | TSYWMHWVKQRPGQGLEWIGNIYPGSGSS | YMNWYQQKPVTSPKRWIYDTSTLASGVPAR |
| | NYDEKFKSKATLTVDTSSSTAYMQLNSLT | FSGSGSGTSYSLTISSMEAEDAATYYCHQR |
| | SEDSAVYYCTRSSYYGNFDTMDNWGQGTS | SSYPWTFGGGTKLEIK (SEQ ID NO: |
| | VTVSS (SEQ ID NO: 38) | 33) |
| TfR16 | EVQLQQSGTVLARPGASVKMSCKTSGYSF | DIKMTQSPSPMFASLGERVTITCKASQDIN |
| | SSYWMHWVKQRPGQGLEWIGTIYPGNSDT | SYLSWFQQKPGKSPKTLIYRANRLLDGVPS |
| | IYNQNFKGKAKLTAVTSASTAYMELSSLT | RFSGSGSGQDYSLTISSLDYEDVGIYYCLQ |
| | NEDSAVYYCSTIYYFSNWGQGTTLTVSS | YDVFPYTFGGGTKLEIK (SEQ ID NO: |
| | (SEQ ID NO: 40) | 41) |
| TfR17 | DVQLQESGPGLVKPSQSLSLTCTVTGYSI | QIVLTQSPAIMSASPGEKVTMTCSASSSIS |
| | TSDYAWNWIRQFPGNKLEWMGYISYSGST | SMHWYQQKPGTSPKRWISDTSKLASGIPAR |
| | SYNPSLKSRISITRDTSKNQFFLQLNSVT | FSGSGSGTSYSLTISSMAAEDAAAYYCHQR |
| | TEDTATYYCASWGYRYLEDYWGQGTSVTV | SSYPWTFGGGTKLEIK (SEQ ID NO: |
| | SS (SEQ ID NO: 42) | 43) |
| TfR18 | QVQLQQSGAELVRPGTSVKVSCKASGYAF | SIVMTQTPKFLLVSAGDRVTITCKASQSVS |
| | TNYLIEWIKQRPGQGLEWIGVINPGNNDT | NDVAWYQQKPGQSPKLLIYYASNRYTGVPD |
| | TYNEMFKAKTTLTADKSSNTAYMQLSSLT | RFTGSGYGTDFTFTISTVQAEDLAVYFCHQ |
| | SDDSAVYFCARRSPYFDYWGQGTTLTVSS | DYSSPTFGGGTKLEIK (SEQ ID NO: |
| | (SEQ ID NO: 44) | 45) |
| TfR19 | QVQLQQPGSELVRPGASVKLSCKASGYTF | QIVLTQSPAIMSASPGEKVTMTCSASSSIR |
| | TSYWTHWVKQRPGQGLEWIGNIYPGSGSS | YMNWYQQKPVTSPKRWIYDTSTLASGVPAR |
| | NYDEKFKSKATLTVDTSSSTAYMQLSSLT | FSGSGSGTSYSLTISSMEAEDAATYYCHQR |
| | SEDSAVYFCTRSSYYGNFDTMDNWGQGTS | SSYPWTFGGGTKLEIK (SEQ ID NO: |
| | VTVSS (SEQ ID NO: 46) | 33) |
| TfR20 | DVQLQESGPGLVKPSQSLSLTCTVTGYSI | DIQMTQSPASLSASVGETVTITCRASEYIN |
| | TSDHAWNWIRQFPGNKLEWMGYISYSGSS | SYLAWYQQKQGKSPQLLVFKEKTLAEGVPS |
| | SYNPSLKSRISITRDTSKNQFFLQLNSVT | RFSGSGSGTQFSLKINSLQPEDFGSYYCQH |
| | TEDTATYYCARSGWGMDYWGQGTSVTVSS | HYGNPWTFGGGTKLEIK (SEQ ID NO: |
| | (SEQ ID NO: 48) | 49) |
| TfR21 | QVQLQQPGSELVRPGASVKLSCKASGYTF | DIQMTQSPASLSASVGETVTITCRASEYIN |
| | TSYWTHWVKQRPGQGLEWIGNIYPGSGSS | SYLAWYQQKQGKSPQLLVFKEKTLAEGVPS |
| | NYDEKFKSKATLTVDTSSSTAYMQLSSLT | RFSGSGSGTQFSLKINSLQPEDFGSYYCQH |
| | SEDSAVYFCTRSSYYGNFDTMDNWGQGTS | HYGNPWTFGGGTKLEIK (SEQ ID NO: |
| | VTVSS (SEQ ID NO: 50) | 51) |
| TfR22 | DVQLQESGPGLVKPSQSLSLTCTVTGYSI | QIVLTQSPAIMSASPGERVTMTCSASSSIS |
| | TSDYAWNWIRQFPGNKLEWMGYISYSGST | SMHWYQQKPGTSPKKWIYDTSKLASGVPAR |
| | RYNPSLKSRISITRDTSKNQFFLQLNSVT | FSGSGSGTSYSLTISSMEAEDAATYYCHQR |
| | TEDTATYYCTSWGYRYLEDYWGQGTSVTV | SSYPWTFGGGTKLEIK (SEQ ID NO: |
| | SA (SEQ ID NO: 52) | 53) |
| TfR23 | DVQLQESGPGLVKPSQSLSLTCTVTGYSI | QIVLTQSPAIMSASPGERVTMTCSASSSIS |
| | TSDYAWNWIRQFPGNKLEWMGYISYSGST | SLHWYQQKPGTSPKKWIYDTSKLASGVPAR |
| | SYNPSLKSRISITRDTSKNQFFLQLNSVT | FSGSGSGTSYSLAISSMEAEDAATYYCHQR |
| | TEDTATYYCTSWGYRYLEDYWGQGTSVTV | SSYPWTFGGGTKLEIK (SEQ ID NO: |
| | SS (SEQ ID NO: 54) | 55) |
| TfR24 | QVQLQQSGPELVKPGTSVKMSCKASGYTF | DIVMTQSPSSLTVTAGERVTMSCKSSQSLL |
| | TSYYIHWVKQRPGQGLEWIGWIYPGDDNT | NSGNQKNYLTWFQQKPGQPPKLLIYWASTR |
| | HYNENFKGKTTLTADKSSSTAYMLLSSLT | ESGVPDRFTGSGSGTDFTVTISSVQAEDLA |
| | SEDSAIYFCARDYYGRFAYWGQGTLVTVS | VYYCQNDYGYPLTFGAGTKLELK (SEQ |
| | A (SEQ ID NO: 56) | ID NO: 57) |
| TfR25 | QVQLQQSGPELVKPGTSVKMSCKASGYTF | DMVMTQSPSSLTVTAGEKVTMSCKSSQSLF |
| | TNYYIHWMKQRPGQGLEWIGWIYPGDGNT | NSENQKNYLTWYQQKPGQPPKLLISWASTR |
| | HYNERFKGKTTLTADKSSSTVYMFLTSLT | ESGVPDRFTGSGSGTDFTLTINSVQAEDLA |
| | SEDSAIYFCAKDKDMPGMDYWGQGTSVTV | VYYCQNDYSYPLTFGAGTKLELK (SEQ |
| | SS (SEQ ID NO: 58) | ID NO: 59) |
| TfR26 | QVQLHQSGPELVKPGASVKMSCKASGYTF | DIVMTQSPSSLTVTSGEKVTMSCKSSQSLL |
| | TNYYIHWVKQRPGQGLEWIGWIYPGDGVT | NSENQKNYLTWYQQKPGQPPKLLIYWASTR |
| | HYNEKFKGKTTLTADKSSSTAYMLLSSLT | ESGVPDRFTGSGSGPDFTLTISSVQAEDLA |
| | SEDSAIYFCARDKDVPGMDYWGQGTSVTV | VYYCQNDYSYPLTFGAGTKLELK (SEQ |
| | SS (SEQ ID NO: 60) | ID NO: 61) |
| TfR27 | QVQLQQPGSELVRPGASVKLSCKASGYTF | QIFLTQSPAIMSASPGERVTMTCSASSSIR |
| | TSYWMHWVKQRPGQGLEWIGNIYPGTGST | YMNWYQQKPGTSPKRWIYDTSELASGVPAR |
| | NYDEKFKSKATLTVDTSSSTAYMQLSSLT | FTGSGSGTSYSLTISSMEAEDAATYYCHQR |
| | SEDSAVYYCTRSSYYGNFDTMDYWGQGTS | SGYPWTFGGGTKLEIK (SEQ ID NO: |
| | VTVSS (SEQ ID NO: 62) | 63) |
| TfR28 | DVQLQESGPGLVKPSQSLSLTCTVTGYSI | QIVLTQSPAIMSASPREKVTMTCSASSSIS |
| | TSDYAWNWIRQFPGNKLEWMGYISYSGST | SMHWYQQKPGTSPKRWIYDTSKLASGVPAR |
| | SYNPSLRSRISITRDTSKNQFFLQLNSVT | FSGSGSGTSYSLTISSMEAEDAATYYCHQR |
| | TEDTATYYCAIRGYRYEEDYWGQGTTLTV | SSYPWTFGGGTKLEIK (SEQ ID NO: |
| | SS (SEQ ID NO: 64) | 65) |
| TfR29 | QVQLQQSGPQLVRPGASVKISCKASGYSF | QIVLTQSPAIMSASPGEKVTMTCSASSSIS |
| | TNYWLHWVKQRPGQGLEWIGMIDPSDSET | YMHWYQQKPGTSPKRWIYDTSKLASGVPAR |
| | RLNQKFKDKATLTVDKSSSTAYMQLSSPT | FSGSGSGTSYSLTISSMEAEDAATYYCHQR |
| | SEDSAVYYCARWGGGYYGFAYWGQGTLVT | SSYTFGGGTKLEIK (SEQ ID NO: |
| | VSA (SEQ ID NO: 66) | 67) |
| TfR30 | QVQLQQSGPQLVRPGASVKISCKASGYSF | DIVMTQSQKFMSTSVGDRVSITCKASQNVR |
| | TNYWLHWVKQRPGQGLEWIGMIDPSDSET | TAVAWYQQKPGQSPKALIYLASNRHTGVPD |
| | RLNQKFKDKATLTVDKSSSTAYMQLSSPT | RFTGSGSGTDFTLTISNVQSEDLADYFCLQ |
| | SEDSAVYYCARWGGGYYGFAYWGQGTLVT | HWNYPWTFGGGTKLEIK (SEQ ID NO: |
| | VSA (SEQ ID NO: 68) | 69) |
| TfR31 | DVQLQESGPGLVKPSQSLSLTCTVTGYSI | QIVLTQSPAIMSASPGEKVTMTCSASSSIS |
| | TSDYAWNWIRQFPGNKLEWMGYISYSGRT | SMHWYQLKPGTSPKKWIYDTSKLASGVPAR |
| | SYNPSLKSRISFTRDTSKNQFFLQLNSVT | FSGSGSGTSYSLTISSMEAEDAATYYCHQR |
| | TEDTATYYCAGWGYRYLEDYWGQGTSVTV | SSYPWTFGGGTKLEIK (SEQ ID NO: |
| | SS (SEQ ID NO: 70) | 71) |
| TfR32 | QVQLQQSGADLVRPGTSVKVSCKASGNAF | SIVMTQTPKFLLVSAGDRVTITCKASQSVS |
| | SNYLIEWVKQRPGQGLEWIGVINPGSGET | NDVAWFQQKPGQSPKLLIYYASNRYTGVPD |
| | NYNEKFKGKATLTADKSSSTAYMQLSSLT | RFTGSGYGTDFTFTIRNVQAEDLAVYFCQQ |
| | SDDSAVYFCAIRYDGPLLDHWGQGTSVTV | DYISPTFGAGTKLELK (SEQ ID NO: |
| | SS (SEQ ID NO: 72) | 73) |

TABLE 12
Anti-huTfR apical domain antibody sequences
from mouse hybridomas (Kabat)

| Antibody | HVR*-H1 | HVR-H2 | HVR-H3 |
|---|---|---|---|
| TfR1 | NYYIH (SEQ | WIYPGDGSTKYNEKFKG | DGSSAPKFTY (SEQ |
| | ID NO: 74) | (SEQ ID NO: 75) | ID NO: 76) |
| TfR2 | DYNMH (SEQ | YIYPYNGGTGYNQKFKS | LYDGYYFDY (SEQ |
| | ID NO: 77) | (SEQ ID NO: 78) | ID NO: 79) |
| TfR3 | SFYIH (SEQ | WIYPGDVNTHYSDKFKG | EGYGYFAY (SEQ |
| | ID NO: 80) | (SEQ ID NO: 81) | ID NO: 82) |
| TfR4 | NYLIE (SEQ | VINPGSGGTTYNEKFKG | RSPYFDY (SEQ ID |
| | ID NO: 83) | (SEQ ID NO: 84) | NO: 85) |
| TfR5 | NYYIH (SEQ | WIYPGDGNTNYNEKFKG | DGSSAPKFTY (SEQ |
| | ID NO: 74) | (SEQ ID NO: 86) | ID NO: 76) |
| TfR6 | SFGMH (SEQ | YISSGSSAIYYADTVKG | SYVYDPPWFAY |
| | ID NO: 87) | (SEQ ID NO: 88) | (SEQ ID NO: 89) |
| TfR7 | SDYAWN (SEQ | YISYSGYSRHNPSLKS | GDWDFDY (SEQ ID |
| | ID NO: 90) | (SEQ ID NO: 91) | NO: 92) |
| TfR8 | NYLIE (SEQ | VINPGSGGTNYNEKFKG | RSPYFDY (SEQ ID |
| | ID NO: 83) | (SEQ ID NO: 93) | NO: 85) |
| TfR9 | EYAMH (SEQ | GINPNDGGTSYNQKFKG | QRWLLRGGDY( SEQ |
| | ID NO: 94) | (SEQ ID NO: 95) | ID NO: 96) |
| TfR10 | SYYIH (SEQ | WIYPGDGNTNYNEKFKG | DGSIPFAY (SEQ ID |
| | ID NO: 97) | (SEQ ID NO: 86) | NO: 98) |
| TfR11 | SYWMH (SEQ | AIFPGNIDTIYNQKFKD | LYYDYV (SEQ ID |
| | ID NO: 99) | (SEQ ID NO: 100) | NO: 101) |
| TfR12 | SYWMH (SEQ | NIYPGSGSSNYDEKFKN | SSYYGNFDTMDN |
| | ID NO: 99) | (SEQ ID NO: 102) | (SEQ ID NO: 103) |
| TfR13 | SYTMS (SEQ | TISSGGSYTYFPDSVKG | AHYYAYDY (SEQ |
| | ID NO: 104) | (SEQ ID NO: 105) | ID NO: 106) |
| TfR14 | SSWMN (SEQ | RIYPGDGDTNYNGKFKG | DPIIDYYYYAMDY |
| | ID NO: 107) | (SEQ ID NO: 108) | (SEQ ID NO: 109) |
| TfR15 | SYWMH (SEQ | NIYPGSGSSNYDEKFKS | SSYYGNFDTMDN |
| | ID NO: 99) | (SEQ ID NO: 110) | (SEQ ID NO: 103) |
| TfR16 | SYWMH (SEQ | TIYPGNSDTIYNQNFKG | IYYFCN (SEQ ID |
| | ID NO: 99) | (SEQ ID NO: 111) | NO: 112) |
| TfR17 | SDYAWN (SEQ | YISYSGSTSYNPSLKS | WGYRYLEDY (SEQ |
| | ID NO: 90) | (SEQ ID NO: 113) | ID NO: 114) |
| TfR18 | NYLIE (SEQ | VINPGNNDTTYNEMFKA | RSPYFDY (SEQ ID |
| | ID NO: 83) | (SEQ ID NO: 115) | NO: 85) |
| TfR19 | SYWTH (SEQ | NIYPGSGSSNYDEKFKS | SSYYGNFDTMDN |
| | ID NO: 116) | (SEQ ID NO: 110) | (SEQ ID NO: 103) |
| TfR20 | SDHAWN (SEQ | YISYSGSSSYNPSLKS | SGWGMDY (SEQ ID |
| | ID NO: 117) | (SEQ ID NO: 118) | NO: 119) |
| TfR21 | SYWTH (SEQ | NIYPGSGSSNYDEKFKS | SSYYGNFDTMDN |
| | ID NO: 116) | (SEQ ID NO: 110) | (SEQ ID NO: 103) |
| TfR22 | SDYAWN (SEQ | YISYSGSTRYNPSLKS | WGYRYLEDY (SEQ |
| | ID NO: 90) | (SEQ ID NO: 120) | ID NO: 114) |
| TfR23 | SDYAWN (SEQ | YISYSGSTSYNPSLKS | WGYRYLEDY (SEQ |
| | ID NO: 90) | (SEQ ID NO: 113) | ID NO: 114) |
| TfR24 | SYYIH (SEQ | WIYPGDDNTHYNENFKG | DYYGRFAY (SEQ |
| | ID NO: 97) | (SEQ ID NO: 121) | ID NO: 122) |
| TfR25 | NYYIH (SEQ | WIYPGDGNTHYNERFKG | DKDMPGMDY (SEQ |
| | ID NO: 74) | (SEQ ID NO: 123) | ID NO: 124) |
| TfR26 | NYYIH (SEQ | WIYPGDGVTHYNEKFKG | DKDVPGMDY (SEQ |
| | ID NO: 74) | (SEQ ID NO: 125) | ID NO: 126) |
| TfR27 | SYWMH (SEQ | NIYPGTGSTNYDEKFKS | SSYYGNFDTMDY |
| | ID NO: 99) | (SEQ ID NO: 127) | (SEQ ID NO: 128) |
| TfR28 | SDYAWN (SEQ | YISYSGSTSYNPSLRS | RGYRYEEDY (SEQ |
| | ID NO: 90) | (SEQ ID NO: 129) | ID NO: 130) |
| TfR29 | NYWLH (SEQ | MIDPSDSETRLNQKFKD | WGGGYYGFAY |
| | ID NO: 131) | (SEQ ID NO: 132) | (SEQ ID NO: 133) |
| TfR30 | NYWLH (SEQ | MIDPSDSETRLNQKFKD | WGGGYYGFAY |
| | ID NO: 131) | (SEQ ID NO: 132) | (SEQ ID NO: 133) |
| TfR31 | SDYAWN (SEQ | YISYSGRTSYNPSLKS | WGYRYLEDY (SEQ |
| | ID NO: 90) | (SEQ ID NO: 134) | ID NO: 114) |
| TfR32 | NYLIE (SEQ | VINPGSGETNYNEKFKG | RYDGPLLDH (SEQ |
| | ID NO: 83) | (SEQ ID NO: 302) | ID NO: 303) |
| TfR12.EB | SYWMH (SEQ | NIYPGSGSSSYAQKFQG | SSYYGNFDTMDN |
| | ID NO: 99) | (SEQ ID NO: 305) | (SEQ ID NO: 103) |
| TfR15.WH1 | SYWMH (SEQ | IIYPGSGSSNYAQKFQG | SSYYGNFDTMDN |
| | ID NO: 99) | (SEQ ID NO: 306) | (SEQ ID NO: 103) |
| TfR15.WH2 | SYWMH (SEQ | IIYPGSGSSNYAQKFQG | SSYYGNFDTMDN |
| | ID NO: 99) | (SEQ ID NO: 306) | (SEQ ID NO: 103) |
| TfR15.WH3 | SYWMH (SEQ | IIYPGSGSSNYAQKFQG | SSYYGNFDTMDN |
| | ID NO: 99) | (SEQ ID NO: 306) | (SEQ ID NO: 103) |
| TfR15.WH4 | SYWMH (SEQ | NIYPGSGSSSYAQKFQG | SSYYGNFDTMDN |
| | ID NO: 99) | (SEQ ID NO: 305) | (SEQ ID NO: 103) |
| TfR15.WH6 | SYWMH (SEQ | NIYPGSGSSSYAQKFQG | SSYYGNFDTMDN |
| | ID NO: 99) | (SEQ ID NO: 305) | (SEQ ID NO: 103) |
| TfR15.WH7 | SYWMH (SEQ | NIYPGSGSSNYAQKFQG | SSYYGNFDTMDN |
| | ID NO: 99) | (SEQ ID NO: 312) | (SEQ ID NO: 103) |
| TfR15.WH8 | SYWMH (SEQ | NIYPGSGSSNYAQKFQG | SSYYGNFDTMDN |
| | ID NO: 99) | (SEQ ID NO: 312) | (SEQ ID NO: 103) |
| TfR15.WH9 | SYWMH (SEQ | NIYPGSGSSNYAQKFQG | SSYYGNFDTMDN |
| | ID NO: 99) | (SEQ ID NO: 312) | (SEQ ID NO: 103) |
| TfR15.WH10 | SYWMH (SEQ | NIYPGSGSSNYAQKFQG | SSYYGNFDTMDN |
| | ID NO: 99) | (SEQ ID NO: 312) | (SEQ ID NO: 103) |
| TfR15.WH11 | SYWMH (SEQ | NIYPGSGSSNYAQKFQG | SSYYGNFDTMDN |
| | ID NO: 99) | (SEQ ID NO: 312) | (SEQ ID NO: 103) |
| TfR15.WH12 | SYWMH (SEQ | NIYPGSGSSNYAQKFQG | SSYYGNFDTMDN |
| | ID NO: 99) | (SEQ ID NO: 312) | (SEQ ID NO: 103) |
| TfR9.1A scFv | EYAMH (SEQ | GINPNDGGTSYNQKFKG | QRWLLRGGDY |
| | ID NO: 94) | (SEQ ID NO: 95) | (SEQ ID NO: 96) |
| TfR9.1B scFv | EYAMH (SEQ | GINPNDGGTSYNQKFKG | QRWLLRGGDY |
| | ID NO: 94) | (SEQ ID NO: 95) | (SEQ ID NO: 96) |
| TfR9.2A scFv | EYAMH (SEQ | GINPNDGGTSYNQKFKG | QRWLLRGGDY |
| | ID NO: 94) | (SEQ ID NO: 95) | (SEQ ID NO: 96) |
| TfR9.2B scFv | EYAMH (SEQ | GINPNDGGTSYNQKFKG | QRWLLRGGDY |
| | ID NO: 94) | (SEQ ID NO: 95) | (SEQ ID NO: 96) |
| TfR9.3A scFv | EYAMH (SEQ | GINPNDGGTSYNQKFKG | QRWLLRGGDY |
| | ID NO: 94) | (SEQ ID NO: 95) | (SEQ ID NO: 96) |
| TfR9.3B scFv | EYAMH (SEQ | GINPNDGGTSYNQKFKG | QRWLLRGGDY |
| | ID NO: 94) | (SEQ ID NO: 95) | (SEQ ID NO: 96) |
| TfR9.4A scFv | EYAMH (SEQ | GINPNDGGTSYNQKFKG | QRWLLRGGDY |
| | ID NO: 94) | (SEQ ID NO: 95) | (SEQ ID NO: 96) |
| TfR9.4B scFv | EYAMH (SEQ | GINPNDGGTSYNQKFKG | QRWLLRGGDY |
| | ID NO: 94) | (SEQ ID NO: 95) | (SEQ ID NO: 96) |
| TfR9.5A scFv | EYAMH (SEQ | GINPNDGGTSYNQKFKG | QRWLLRGGDY |
| | ID NO: 94) | (SEQ ID NO: 95) | (SEQ ID NO: 96) |
| TfR9.5B scFv | EYAMH (SEQ | GINPNDGGTSYNQKFKG | QRWLLRGGDY |
| | ID NO: 94) | (SEQ ID NO: 95) | (SEQ ID NO: 96) |
| TfR12.1A scFv | SYWMH (SEQ | NIYPGSGSSNYDEKFKN | SSYYGNFDTMDN |
| | ID NO: 99) | (SEQ ID NO: 102) | (SEQ ID NO: 103) |
| TfR12.1B scFv | SYWMH (SEQ | NIYPGSGSSNYDEKFKN | SSYYGNFDTMDN |
| | ID NO: 99) | (SEQ ID NO: 102) | (SEQ ID NO: 103) |
| TfR12.2A scFv | SYWMH (SEQ | NIYPGSGSSNYDEKFKN | SSYYGNFDTMDN |
| | ID NO: 99) | (SEQ ID NO: 102) | (SEQ ID NO: 103) |
| TfR12.2B scFv | SYWMH (SEQ | NIYPGSGSSNYDEKFKN | SSYYGNFDTMDN |
| | ID NO: 99) | (SEQ ID NO: 102) | (SEQ ID NO: 103) |
| TfR12.3A scFv | SYWMH (SEQ | NIYPGSGSSNYDEKFKN | SSYYGNFDTMDN |
| | ID NO: 99) | (SEQ ID NO: 102) | (SEQ ID NO: 103) |
| TfR12.3B scFv | SYWMH (SEQ | NIYPGSGSSNYDEKFKN | SSYYGNFDTMDN |
| | ID NO: 99) | (SEQ ID NO: 102) | (SEQ ID NO: 103) |
| TfR12.4A scFv | SYWMH (SEQ | NIYPGSGSSNYDEKFKN | SSYYGNFDTMDN |
| | ID NO: 99) | (SEQ ID NO: 102) | (SEQ ID NO: 103) |
| TfR12.4B scFv | SYWMH (SEQ | NIYPGSGSSNYDEKFKN | SSYYGNFDTMDN |
| | ID NO: 99) | (SEQ ID NO: 102) | (SEQ ID NO: 103) |
| TfR15.WH8.1.1 | SYWMH (SEQ | NIYPGSGSSNYAQKFQG | SSYYGNFDTMDN |
| | ID NO: 99) | (SEQ ID NO: 312) | (SEQ ID NO: 103) |

*HVR = hypervariable region; also known as CDR

TABLE 13
Anti-huTfR apical domain antibody sequences from
mouse hybridomas (Kabat)

| Antibody | HVR-L1 | HVR-L2 | HVR-L3 |
|---|---|---|---|
| TfR1 | KSSQSLLNSGNQKNYLT | WASTRES (SEQ | QNDYSYPLT |
| | (SEQ ID NO: 135) | ID NO: 136) | (SEQ ID NO: 137) |
| TfR2 | RASQSIGTSIH (SEQ | YASESIS (SEQ | QQSNSWPFT |
| | ID NO: 138) | ID NO: 139) | (SEQ ID NO: 140) |
| TfR3 | KSSQSLLNSGNQKNYLT | WASTRES (SEQ | QNDYSYPLT |
| | (SEQ ID NO: 135) | ID NO: 136) | (SEQ ID NO: 137) |
| TfR4 | KASQSVSNDVA (SEQ | YASNRYT (SEQ | HQDYISPT |
| | ID NO: 141) | ID NO: 142) | (SEQ ID NO: 143) |
| TfR5 | KSSQSLLNSGNQKNYLT | WASTRES (SEQ | QNDYSYPLT |
| | (SEQ ID NO: 135) | ID NO: 136) | (SEQ ID NO: 137) |
| TfR6 | KASQDVSTAVA (SEQ | WASTRPT (SEQ | QQHFSTPWT |
| | ID NO: 143) | ID NO: 144) | (SEQ ID NO: 145) |
| TfR7 | KASQNVLTNVA (SEQ | SSSSRNS (SEQ | QQYNTYPYT |
| | ID NO: 146) | ID NO: 147) | (SEQ ID NO: 148) |
| TfR8 | KASQSVSNDVA (SEQ | YASNRYT (SEQ | HQDYISPT |
| | ID NO: 141) | ID NO: 142) | (SEQ ID NO: 143) |
| TfR9 | KSSQSLLDSDGKTYLN | LVSKLDS (SEQ | WQGTHFPQT |
| | (SEQ ID NO: 149) | ID NO: 150) | (SEQ ID NO: 151) |
| TfR10 | KSSQSLLNSGNQKNYLT | WASTRES (SEQ | QNDYSYPLT |
| | (SEQ ID NO: 135) | ID NO: 136) | (SEQ ID NO: 137) |
| TfR11 | KASQDINSYLS (SEQ | HVNRLVD (SEQ | LQYDEFPYT |
| | ID NO: 152) | ID NO: 153) | (SEQ ID NO: 154) |
| TfR12 | SASSSIRYMN (SEQ | DTSTLAS (SEQ | HQRSSYPWT |
| | ID NO: 155) | ID NO: 156) | (SEQ ID NO: 157) |
| TfR13 | RASQSIGTSIH (SEQ | YASGSIS (SEQ | QQSNSWPTST |
| | ID NO: 138) | ID NO: 158) | (SEQ ID NO: 159) |
| TfR14 | RASESVDNYGISFMN | AASNQGS (SEQ | QQSKEVPWT |
| | (SEQ ID NO: 160) | ID NO: 161) | (SEQ ID NO: 162) |
| TfR15 | SASSSIRYMN (SEQ | DTSTLAS (SEQ | HQRSSYPWT |
| | ID NO: 155) | ID NO: 156) | (SEQ ID NO: 157) |
| TfR16 | KASQDINSYLS (SEQ | RANRLLD (SEQ | LQYDVFPYT |
| | IDNO: 152) | ID NO: 163) | (SEQ ID NO: 164) |
| TfR17 | SASSSISSMH (SEQ | DTSKLAS (SEQ | HQRSSYPWT |
| | ID NO: 165) | ID NO: 166) | (SEQ ID NO: 157) |
| TfR18 | KASQSVSNDVA (SEQ | YASNRYT (SEQ | HQDYSSPT |
| | ID NO: 141) | ID NO: 142) | (SEQ ID NO: 167) |
| TfR19 | SASSSIRYMN (SEQ | DTSTLAS (SEQ | HQRSSYPWT |
| | ID NO: 155) | ID NO: 156) | (SEQ ID NO: 157) |
| TfR20 | RASEYINSYLA (SEQ | KEKTLAE (SEQ | QHHYGNPWT |
| | ID NO: 168) | ID NO: 169) | (SEQ ID NO: 170) |
| TfR21 | RASEYINSYLA (SEQ | KEKTLAE (SEQ | QHHYGNPWT |
| | ID NO: 168) | ID NO: 169) | (SEQ ID NO: 170) |
| TfR22 | SASSSISSMH (SEQ | DTSKLAS (SEQ | HQRSSYPWT |
| | ID NO: 165) | ID NO: 166) | (SEQ ID NO: 157) |
| TfR23 | SASSSISSLH (SEQ | DTSKLAS (SEQ | HQRSSYPWT |
| | ID NO: 171) | ID NO: 166) | (SEQ ID NO: 157) |
| TfR24 | KSSQSLLNSGNQKNYLT | WASTRES (SEQ | QNDYGYPLT |
| | (SEQ ID NO: 135) | ID NO: 136) | (SEQ ID NO: 172) |
| TfR25 | KSSQSLFNSENQKNYLT | WASTRES (SEQ | QNDYSYPLT |
| | (SEQ ID NO: 173) | ID NO: 136) | (SEQ ID NO: 137) |
| TfR26 | KSSQSLLNSENQKNYLT | WASTRES (SEQ | QNDYSYPLT |
| | (SEQ ID NO: 174) | ID NO: 136) | (SEQ ID NO: 137) |
| TfR27 | SASSSIRYMN (SEQ | DTSELAS (SEQ | HQRSGYPWT |
| | ID NO: 155) | ID NO: 175) | (SEQ ID NO: 176) |
| TfR28 | SASSSISSMH (SEQ | DTSKLAS (SEQ | HQRSSYPWT |
| | ID NO: 165) | ID NO: 166) | (SEQ ID NO: 157) |
| TfR29 | SASSSISYMH (SEQ | DTSKLAS (SEQ | HQRSSYT |
| | ID NO: 177) | ID NO: 166) | (SEQ ID NO: 178) |
| TfR30 | KASQNVRTAVA (SEQ | LASNRHT (SEQ | LQHWNYPWT |
| | ID NO: 179) | ID NO: 180) | (SEQ ID NO: 181) |
| TfR31 | SASSSISSMH (SEQ | DTSKLAS (SEQ | HQRSSYPWT |
| | ID NO: 165) | ID NO: 166) | (SEQ ID NO: 157) |
| TfR32 | KASQSVSNDVA (SEQ | YASNRYT (SEQ | QQDYISPT |
| | ID NO: 141) | ID NO: 142) | (SEQ ID NO: 182) |
| TfR12.EB | RASSSIRYMN (SEQ | DTSTLQS (SEQ | HQRSSYPWT |
| | ID NO: 307) | ID NO: 308) | (SEQ ID NO: 157) |
| TfR15.WH1 | RASSSIRYMN (SEQ | DTSTLQS (SEQ | HQRSSYPWT |
| | ID NO: 307) | ID NO: 308) | (SEQ ID NO: 157) |
| TfR15.WH2 | RASSSIRYMN (SEQ | DTSNRAT (SEQ | HQRSSYPWT |
| | ID NO: 307) | ID NO: 309) | (SEQ ID NO: 157) |
| TfR15.WH3 | RASSSIRYLN (SEQ | DTSSLQS (SEQ | HQRSSYPWT |
| | ID NO: 310) | ID NO: 311) | (SEQ ID NO: 157) |
| TfR15.WH4 | RASSSIR YMN (SEQ | DTSTLQS (SEQ | HQRSSYPWT |
| | ID NO: 307) | ID NO: 308) | (SEQ ID NO: 157) |
| TfR15.WH6 | RASSSIRYLN (SEQ | DTSSLQS (SEQ | HQRSSYPWT |
| | ID NO: 310) | ID NO: 311) | (SEQ ID NO: 157) |
| TfR15.WH7 | RASSSIRYMN (SEQ | DTSTLQS (SEQ | HQRSSYPWT |
| | ID NO: 307) | ID NO: 308) | (SEQ ID NO: 157) |
| TfR15.WH8 | RASSSIR YMN (SEQ | DTSNRAT (SEQ | HQRSSYPWT |
| | ID NO: 307) | ID NO: 309) | (SEQ ID NO: 157) |
| TfR15.WH9 | RASSSIRYLN (SEQ | DTSSLQS (SEQ | HQRSSYPWT |
| | ID NO: 310) | ID NO: 311) | (SEQ ID NO: 157) |
| TfR15.WH10 | RASSSIRYMN (SEQ | DTSTLQS (SEQ | HQRSSYPWT |
| | ID NO: 307) | ID NO: 308) | (SEQ ID NO: 157) |
| TfR15.WH11 | RASSSIR YMN (SEQ | DTSNRAT (SEQ | HQRSSYPWT |
| | ID NO: 307) | ID NO: 309) | (SEQ ID NO: 157) |
| TfR15.WH12 | RASSSIRYLN (SEQ | DTSSLQS (SEQ | HQRSSYPWT |
| | ID NO: 310) | ID NO: 311) | (SEQ ID NO: 157) |
| TfR9.1A scFv | KSSQSLLDSDGKTYLN | LVSKLDS (SEQ | WQGTHFPQT |
| | (SEQ ID NO: 149) | ID NO: 150) | (SEQ ID NO: 151) |
| TfR9.1B scFv | KSSQSLLDSDGKTYLN | LVSKLDS (SEQ | WQGTHFPQT |
| | (SEQ ID NO: 149) | ID NO: 150) | (SEQ ID NO: 151) |
| TfR9.2A scFv | KSSQSLLDSDGKTYLN | LVSKLDS (SEQ | WQGTHFPQT |
| | (SEQ ID NO: 149) | ID NO: 150) | (SEQ ID NO: 151) |
| TfR9.2B scFv | KSSQSLLDSDGKTYLN | LVSKLDS (SEQ | WQGTHFPQT |
| | (SEQ ID NO: 149) | ID NO: 150) | (SEQ ID NO: 151) |
| TfR9.3A scFv | KSSQSLLDSDGKTYLN | LVSKLDS (SEQ | WQGTHFPQT |
| | (SEQ ID NO: 149) | ID NO: 150) | (SEQ ID NO: 151) |
| TfR9.3B scFv | KSSQSLLDSDGKTYLN | LVSKLDS (SEQ | WQGTHFPQT |
| | (SEQ ID NO: 149) | ID NO: 150) | (SEQ ID NO: 151) |
| TfR9.4A scFv | KSSQSLLDSDGKTYLN | LVSKLDS (SEQ | WQGTHFPQT |
| | (SEQ ID NO: 149) | ID NO: 150) | (SEQ ID NO: 151) |
| TfR9.4B scFv | KSSQSLLDSDGKTYLN | LVSKLDS (SEQ | WQGTHFPQT |
| | (SEQ ID NO: 149) | ID NO: 150) | (SEQ ID NO: 151) |
| TfR9.5A scFv | KSSQSLLDSDGKTYLN | LVSKLDS (SEQ | WQGTHFPQT |
| | (SEQ ID NO: 149) | ID NO: 150) | (SEQ ID NO: 151) |
| TfR9.5B scFv | KSSQSLLDSDGKTYLN | LVSKLDS (SEQ | WQGTHFPQT |
| | (SEQ ID NO: 149) | ID NO: 150) | (SEQ ID NO: 151) |
| TfR12.1A scFv | SASSSIR YMN (SEQ | DTSTLAS (SEQ | HQRSSYPWT |
| | ID NO: 155) | ID NO: 156) | (SEQ ID NO: 157) |
| TfR12.1B scFv | SASSSIR YMN (SEQ | DTSTLAS (SEQ | HQRSSYPWT |
| | ID NO: 155) | ID NO: 156) | (SEQ ID NO: 157) |
| TfR12.2A scFv | SASSSIRYMN (SEQ | DTSTLAS (SEQ | HQRSSYPWT |
| | ID NO: 155) | ID NO: 156) | (SEQ ID NO: 157) |
| TfR12.2B scFv | SASSSIRYMN (SEQ | DTSTLAS (SEQ | HQRSSYPWT |
| | ID NO: 155) | ID NO: 156) | (SEQ ID NO: 157) |
| TfR12.3A scFv | SASSSIR YMN (SEQ | DTSTLAS (SEQ | HQRSSYPWT |
| | ID NO: 155) | ID NO: 156) | (SEQ ID NO: 157) |
| TfR12.3B scFv | SASSSIRYMN (SEQ | DTSTLAS (SEQ | HQRSSYPWT |
| | ID NO: 155) | ID NO: 156) | (SEQ ID NO: 157) |
| TfR12.4A scFv | SASSSIR YMN (SEQ | DTSTLAS (SEQ | HQRSSYPWT |
| | ID NO: 155) | ID NO: 156) | (SEQ ID NO: 157) |
| TfR12.4B scFv | SASSSIRYMN (SEQ | DTSTLAS (SEQ | HQRSSYPWT |
| | ID NO: 155) | ID NO: 156) | (SEQ ID NO: 157) |
| TfR15.WH8.1.1 | RASSSIRYMN (SEQ | DTSNRAT (SEQ | HQRSSYPWT |
| | ID NO: 307) | ID NO: 309) | (SEQ ID NO: 157) |

### Example 9: Reformatting Hybridoma Antibodies as 2+1 Bispecific Antibodies

Antibodies were selected for formatting into a 2+1 bispecific format based on various criteria: 1) the antibodies covered a broad range of affinities based on ELISA and FACS binding assays, 2) the antibodies covered a broad range of internalization activity, 3) no outstanding high-risk modification sites were identified in the CDRs, and 4) the antibodies were phylogenetically diverse from each other within the hybridoma sequences obtained.

An IgG isotype antibody with no target specificity (“inert isotype control antibody”) with knob-hole mutations in the constant domains of the heavy chains was used for formatting into a 2+1 bispecific antibody. An anti-TfR scFv (VH and VL) was appended to the IgG isotype antibody via a linker at the C-terminus of the constant domain with the “hole” mutation (“hole side” of the IgG isotype antibody). The IgG isotype antibody attached to a scFv is called a 2+1 bispecific antibody, as shown in FIG. 1A and (i) in FIG. 1D. The 20 amino acid linker connecting the VH and VL domains within the scFv was GGSEGKSSGSGSESKSTGGS (SEQ ID NO:183) (Bird et al, Science 1988. October 21; 242(4877):423-6), and the linker connecting the C-terminus of the Fc ‘hole’ domain to the scFv was (GGGGS)x3 (SEQ ID NO:184). DNA encoding the 2+1 bispecific antibodies were prepared by gene synthesis and cloned into the expression vector pcDNA3.4 (ThermoFisher).

The scFv (VH and VL) sequences were formatted into an expression construct of the heavy chain of the IgG with the “hole” mutation (“heavy chain-hole” construct) using the framework in SEQ ID NO:185. In some instances, the “heavy chain-hole” construct included a set of mutations (H435R, Y436F) to minimize binding to Protein A (Tustian et al., MAbs. May-June 2016; 8(4):828-38) (SEQ ID NO:186). The corresponding heavy chain-knob and the light chain expression constructs were also generated (SEQ ID NOs:187-188). In some instances, the heavy chain-hole and the heavy chain-knob also included mutations to reduce effector function (SEQ ID NOs:189-194).

An example structure of a 2+1 bispecific antibody comprised the following components: 1) Isotype control hIgG1 wildtype antibody with a knob ((T366W) mutation and a hole mutation (T366S_L368A_Y407V) in the constant regions, 2) a (G4S)x3 linker between the “hole side” of the hIgG1 antibody and a scFv, 3) a VH sequence of the scFv, 4) a 20 amino acid linker sequence between the VH and VL of the scFv, and 5) a VL sequence of the scFv.

TABLE 14
2 + 1 Bispecific antibody sequences

| SEQ | | |
|---|---|---|
| ID | | |
| NO. | Description | Sequence |
| 185 | Isotype control | EVRLLESGGGLVQPGGSLRLSCAASGFTFSNYAMGWVRQAPGKGLEWVSAISGSGGSTYYADSVKG |
| | heavy chain | RFTTSRDDSKNALYLQMNSLRAEDTAVYYCARGGPGWYAADVWGQGTTVTVSSASTKGPSVFPLAP |
| | hIgG1 wt hole scFv | SSKSTSGGTAALGCLVKDYFPEPVTVSWNSGALTSGVHTFPAVLQSSGLYSLSSVVTVPSSSLGTQ |
| | (T366S_L368A_Y407V)- | TYICNVNHKPSNTKVDKKVEPKSCDKTHTCPPCPAPELLGGPSVFLFPPKPKDTLMISRTPEVTCV |
| | (G4S)3 linker-VH- | VVDVSHEDPEVKFNWYVDGVEVHNAKTKPREEQYNSTYRVVSVLTVLHQDWLNGKEYKCKVSNKAL |
| | 20AA linker-VL | PAPIEKTISKAKGQPREPQVYTLPPSRDELTKNQVSLSCAVKGFYPSDIAVEWESNGQPENNYKTT |
| | | PPVLDSDGSFFLVSKLTVDKSRWQQGNVFSCSVMHEALHNHYTQKSLSLSPGK-GGGGSGGGGSGG |
| | | GGS-X1-GGSEGKSSGSGSESKSTGGS-X2, wherein X1 is the |
| | | amino acid sequence of any one of SEQ ID NOs: 10, 12, 14, 16, 18, |
| | | 20, 22, 24, 26, 28, 30, 32, 34, 36, 38, 40, 42, 44, 46, 48, 50, |
| | | 52, 54, 56, 58, 60, 62, 64, 66, 68, 70, 72, 313, 315, 317, 318, |
| | | 319, 320, 321, 322, 323, 324, 325, 327, 330, 334, 335, 337, 339, |
| | | 343, 344, 345, 346, 348, 349, 351, 357, 359, 361, 363, 364, 369, |
| | | 371, 372, 375, 376, 378, 380, 384, 385, 387, 389, 390, 391, 393, |
| | | 394, 396, 397, 398, 399, 401, 402, 443, 444, 445, 446, 447, 448, |
| | | 449, 450, 451, 452, and 453, and wherein X2 is the amino acid |
| | | sequence of any one of SEQ ID NOs: 11, 13, 15, 17, 19, 21, 23, 25, |
| | | 27, 29, 31, 33, 35, 37, 39, 41, 43, 45, 47, 49, 51, 53, 55, 57, |
| | | 59, 61, 63, 65, 67, 69, 71, 73, 314, 316, 326, 328, 329, 331, 332, |
| | | 333, 336, 338, 340, 341, 342, 347, 350, 352, 353, 354, 355, 356, |
| | | 358, 360, 362, 365, 366, 367, 368, 370, 373, 374, 377, 379, 381, |
| | | 382, 383, 386, 388, 392, 395, 400, 403, 454, 455, 456, 457, 458, |
| | | and 459. |
| 186 | Isotype control | EVRLLESGGGLVQPGGSLRLSCAASGFTFSNYAMGWVRQAPGKGLEWVSAISGSGGSTYYADSVKG |
| | heavy chain | RFTTSRDDSKNALYLQMNSLRAEDTAVYYCARGGPGWYAADVWGQGTTVTVSSASTKGPSVFPLAP |
| | hIgG1 wt hole scFv | SSKSTSGGTAALGCLVKDYFPEPVTVSWNSGALTSGVHTFPAVLQSSGLYSLSSVVTVPSSSLGTQ |
| | (T366S_L368A_Y407V)- | TYICNVNHKPSNTKVDKKVEPKSCDKTHTCPPCPAPELLGGPSVFLFPPKPKDTLMISRTPEVTCV |
| | (G4S)3 linker-VH- | VVDVSHEDPEVKFNWYVDGVEVHNAKTKPREEQYNSTYRVVSVLTVLHQDWLNGKEYKCKVSNKAL |
| | 20AA linker-VL with | PAPIEKTISKAKGQPREPQVYTLPPSRDELTKNQVSLSCAVKGFYPSDIAVEWESNGQPENNYKTT |
| | mutations (H435R, | PPVLDSDGSFFLVSKLTVDKSRWQQGNVFSCSVMHEALHNRFTQKSLSLSPGK-GGGGGGGGSGGG |
| | Y436F) | GS-X1-GGSEGKSSGSGSESKSTGGS-X2, wherein X1 is the |
| | | amino acid sequence of any one of SEQ ID NOs: 10, 12, 14, 16, 18, |
| | | 20, 22, 24, 26, 28, 30, 32, 34, 36, 38, 40, 42, 44, 46, 48, 50, |
| | | 52, 54, 56, 58, 60, 62, 64, 66, 68, 70, 72, 313, 315, 317, 318, |
| | | 319, 320, 321, 322, 323, 324, 325, 327, 330, 334, 335, 337, 339, |
| | | 343, 344, 345, 346, 348, 349, 351, 357, 359, 361, 363, 364, 369, |
| | | 371, 372, 375, 376, 378, 380, 384, 385, 387, 389, 390, 391, 393, |
| | | 394, 396, 397, 398, 399, 401, 402, 443, 444, 445, 446, 447, 448, |
| | | 449, 450, 451, 452, and 453, and wherein X2 is the amino acid |
| | | sequence of any one of SEQ ID NOs: 11, 13, 15, 17, 19, 21, 23, 25, |
| | | 27, 29, 31, 33, 35, 37, 39, 41, 43, 45, 47, 49, 51, 53, 55, 57, |
| | | 59, 61, 63, 65, 67, 69, 71, 73, 314, 316, 326, 328, 329, 331, 332, |
| | | 333, 336, 338, 340, 341, 342, 347, 350, 352, 353, 354, 355, 356, |
| | | 358, 360, 362, 365, 366, 367, 368, 370, 373, 374, 377, 379, 381, |
| | | 382, 383, 386, 388, 392, 395, 400, 403, 454, 455, 456, 457, 458, |
| | | and 459. |
| 187 | Isotype control | EVRLLESGGGLVQPGGSLRLSCAASGFTFSNYAMGWVRQAPGKGLEWVSAISGSGGSTYYADSVKG |
| | heavy chain | RFTTSRDDSKNALYLQMNSLRAEDTAVYYCARGGPGWYAADVWGQGTTVTVSSASTKGPSVFPLAP |
| | hIgG1 wt knob | SSKSTSGGTAALGCLVKDYFPEPVTVSWNSGALTSGVHTFPAVLQSSGLYSLSSVVTVPSSSLGTQ |
| | (T366W) | TYICNVNHKPSNTKVDKKVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLTVLHQDWLNGKEYKCKVSNKAL |
| | | PAPIEKTISKAKGQPREPQVYTLPPSRDELTKNQVSLWCLVKGFYPSDIAVEWESNGQPENNYKTT |
| | | PPVLDSDGSFFLYSKLTVDKSRWQQGNVFSCSVMHEALHNHYTQKSLSLSPGK |
| 188 | Isotype control | DIQMTQSPSSLSASVGDRVTITCRASQSISSYLNWYQQKPGKAPKLLIYAASSLQSGVPSRFSGSG |
| | light chain | SGTDFTLTISSLQPEDFATYYCQQADLPAFAFGGGTKVEIKRTVAAPSVFIFPPSDEQLKSGTASV |
| | | VCLLNNFYPREAKVQWKVDNALQSGNSQESVTEQDSKDSTYSLSSTLTLSKADYEKHKVYACEVTH |
| | | QGLSSPVTKSFNRGEC |
| 189 | Isotype control | EVRLLESGGGLVQPGGSLRLSCAASGFTFSNYAMGWVRQAPGKGLEWVSAISGSGGSTYYADSVKG |
| | heavy chain | RFTTSRDDSKNALYLQMNSLRAEDTAVYYCARGGPGWYAADVWGQGTTVTVSSASTKGPSVFPLAP |
| | hIgG1 LALAPS | SSKSTSGGTAALGCLVKDYFPEPVTVSWNSGALTSGVHTFPAVLQSSGLYSLSSVVTVPSSSLGTQ |
| | hole scFv | TYICNVNHKPSNTKVDKKVEPKSCDKTHTCPPCPAPEAAGGPSVFLFPPKPKDTLMISRTPEVTCV |
| | (T366S_L368A_Y407V) | VVDVSHEDPEVKFNWYVDGVEVHNAKTKPREEQYNSTYRVVSVLTVLHQDWLNGKEYKCKVSNKAL |
| | (L234A_L235A_P331S)- | PASIEKTISKAKGQPREPQVYTLPPSRDELTKNQVSLSCAVKGFYPSDIAVEWESNGQPENNYKTT |
| | (G4S)3 linker-VH- | PPVLDSDGSFFLVSKLTVDKSRWQQGNVFSCSVMHEALHNHYTQKSLSLSPGK-GGGGSGGGGSGG |
| | 20AA linker-VL | GGS-X1-GGSEGKSSGSGSESKSTGGS-X2, wherein X1 is the |
| | | amino acid sequence of any one of SEQ ID NOs: 10, 12, 14, 16, 18, |
| | | 20, 22, 24, 26, 28, 30, 32, 34, 36, 38, 40, 42, 44, 46, 48, 50, |
| | | 52, 54, 56, 58, 60, 62, 64, 66, 68, 70, 72, 313, 315, 317, 318, |
| | | 319, 320, 321, 322, 323, 324, 325, 327, 330, 334, 335, 337, 339, |
| | | 343, 344, 345, 346, 348, 349, 351, 357, 359, 361, 363, 364, 369, |
| | | 371, 372, 375, 376, 378, 380, 384, 385, 387, 389, 390, 391, 393, |
| | | 394, 396, 397, 398, 399, 401, 402, 443, 444, 445, 446, 447, 448, |
| | | 449, 450, 451, 452, and 453, and wherein X2 is the amino acid |
| | | sequence of any one of SEQ ID NOs: 11, 13, 15, 17, 19, 21, 23, 25, |
| | | 27, 29, 31, 33, 35, 37, 39, 41, 43, 45, 47, 49, 51, 53, 55, 57, |
| | | 59, 61, 63, 65, 67, 69, 71, 73, 314, 316, 326, 328, 329, 331, 332, |
| | | 333, 336, 338, 340, 341, 342, 347, 350, 352, 353, 354, 355, 356, |
| | | 358, 360, 362, 365, 366, 367, 368, 370, 373, 374, 377, 379, 381, |
| | | 382, 383, 386, 388, 392, 395, 400, 403, 454, 455, 456, 457, 458, |
| | | and 459. |
| 190 | Isotype control | EVRLLESGGGLVQPGGSLRLSCAASGFTFSNYAMGWVRQAPGKGLEWVSAISGSGGSTYYADSVKG |
| | heavy chain | RFTTSRDDSKNALYLQMNSLRAEDTAVYYCARGGPGWYAADVWGQGTTVTVSSASTKGPSVFPLAP |
| | hIgG1 LALAPS | SSKSTSGGTAALGCLVKDYFPEPVTVSWNSGALTSGVHTFPAVLQSSGLYSLSSVVTVPSSSLGTQ |
| | hole scFv | TYICNVNHKPSNTKVDKKVEPKSCDKTHTCPPCPAPEAAGGPSVFLFPPKPKDTLMISRTPEVTCV |
| | (T366S_L368A_Y407V) | VVDVSHEDPEVKFNWYVDGVEVHNAKTKPREEQYNSTYRVVSVLTVLHQDWLNGKEYKCKVSNKAL |
| | (L234A L235A_P331S)- | PASIEKTISKAKGQPREPQVYTLPPSRDELTKNQVSLSCAVKGFYPSDIAVEWESNGQPENNYKTT |
| | (G4S)3 linker-VH- | PPVLDSDGSFFLVSKLTVDKSRWQQGNVFSCSVMHEALHNRFTQKSLSLSPGK-GGGGSGGGGSGG |
| | 20AA linker-VL with | GGS-X1-GGSEGKSSGSGSESKSTGGS-X2, wherein X1 is the |
| | mutations (H435R, | amino acid sequence of any one of SEQ ID NOs: 10, 12, 14, 16, 18, |
| | Y436F) | 20, 22, 24, 26, 28, 30, 32, 34, 36, 38, 40, 42, 44, 46, 48, 50, |
| | | 52, 54, 56, 58, 60, 62, 64, 66, 68, 70, 72, 313, 315, 317, 318, |
| | | 319, 320, 321, 322, 323, 324, 325, 327, 330, 334, 335, 337, 339, |
| | | 343, 344, 345, 346, 348, 349, 351, 357, 359, 361, 363, 364, 369, |
| | | 371, 372, 375, 376, 378, 380, 384, 385, 387, 389, 390, 391, 393, |
| | | 394, 396, 397, 398, 399, 401, 402, 443, 444, 445, 446, 447, 448, |
| | | 449, 450, 451, 452, and 453, and wherein X2 is the amino acid |
| | | sequence of any one of SEQ ID NOs:11, 13, 15, 17, 19, 21, 23, 25, |
| | | 27, 29, 31, 33, 35, 37, 39, 41, 43, 45, 47, 49, 51, 53, 55, 57, |
| | | 59, 61, 63, 65, 67, 69, 71, 73, 314, 316, 326, 328, 329, 331, 332, |
| | | 333, 336, 338, 340, 341, 342, 347, 350, 352, 353, 354, 355, 356, |
| | | 358, 360, 362, 365, 366, 367, 368, 370, 373, 374, 377, 379, 381, |
| | | 382, 383, 386, 388, 392, 395, 400, 403, 454, 455, 456, 457, 458, |
| | | and 459. |
| 191 | Isotype control | EVRLLESGGGLVQPGGSLRLSCAASGFTFSNYAMGWVRQAPGKGLEWVSAISGSGGSTYYADSVKG |
| | heavy chain | RFTTSRDDSKNALYLQMNSLRAEDTAVYYCARGGPGWYAADVWGQGTTVTVSSASTKGPSVFPLAP |
| | hIgG1 LALAPS knob | SSKSTSGGTAALGCLVKDYFPEPVTVSWNSGALTSGVHTFPAVLQSSGLYSLSSVVTVPSSSLGTQ |
| | (T366W) | TYICNVNHKPSNTKVDKKVEPKSCDKTHTCPPCPAPEAAGGPSVFLFPPKPKDTLMISRTPEVTCV |
| | (L234A_L235A_P331S) | VVDVSHEDPEVKFNWYVDGVEVHNAKTKPREEQYNSTYRVVSVLTVLHQDWLNGKEYKCKVSNKAL |
| | | PASIEKTISKAKGQPREPQVYTLPPSRDELTKNQVSLWCLVKGFYPSDIAVEWESNGQPENNYKTT |
| | | PPVLDSDGSFFLYSKLTVDKSRWQQGNVFSCSVMHEALHNHYTQKSLSLSPGK |
| 192 | Isotype control | EVRLLESGGGLVQPGGSLRLSCAASGFTFSNYAMGWVRQAPGKGLEWVSAISGSGGSTYYADSVKG |
| | heavy chain | RFTTSRDDSKNALYLQMNSLRAEDTAVYYCARGGPGWYAADVWGQGTTVTVSSASTKGPSVFPLAP |
| | hIgG1 NSLF | SSKSTSGGTAALGCLVKDYFPEPVTVSWNSGALTSGVHTFPAVLQSSGLYSLSSVVTVPSSSLGTQ |
| | hole scFv | TYICNVNHKPSNTKVDKKVEPKSCDKTHTCPPCPAPELLGGPSVFLFPPKPKDTLMISRTPEVTCV |
| | (T366S_L368A_Y407V) | VVDVSHEDPEVKFNWYVDGVEVHNAKTKPREEQYNSTYRVVSVLTVLHQDWLNGKEYKCKVSSKAF |
| | (N325S_L328F)- | PAPIEKTISKAKGQPREPQVYTLPPSRDELTKNQVSLSCAVKGFYPSDIAVEWESNGQPENNYKTT |
| | (G4S)3 linker-VH- | PPVLDSDGSFFLVSKLTVDKSRWQQGNVFSCSVMHEALHNHYTQKSLSLSPGK-GGGGSGGGGSGG |
| | 20AA linker-VL | GGS-X1-GGSEGKSSGSGSESKSTGGS-X2, wherein X1 is the |
| | | amino acid sequence of any one of SEQ ID NOs: 10, 12, 14, 16, 18, |
| | | 20, 22, 24, 26, 28, 30, 32, 34, 36, 38, 40, 42, 44, 46, 48, 50, |
| | | 52, 54, 56, 58, 60, 62, 64, 66, 68, 70, 72, 313, 315, 317, 318, |
| | | 319, 320, 321, 322, 323, 324, 325, 327, 330, 334, 335, 337, 339, |
| | | 343, 344, 345, 346, 348, 349, 351, 357, 359, 361, 363, 364, 369, |
| | | 371, 372, 375, 376, 378, 380, 384, 385, 387, 389, 390, 391, 393, |
| | | 394, 396, 397, 398, 399, 401, 402, 443, 444, 445, 446, 447, 448, |
| | | 449, 450, 451, 452, and 453, and wherein X2 is the amino acid |
| | | sequence of any one of SEQ ID NOs:11, 13, 15, 17, 19, 21, 23, 25, |
| | | 27, 29, 31, 33, 35, 37, 39, 41, 43, 45, 47, 49, 51, 53, 55, 57, |
| | | 59, 61, 63, 65, 67, 69, 71, 73, 314, 316, 326, 328, 329, 331, 332, |
| | | 333, 336, 338, 340, 341, 342, 347, 350, 352, 353, 354, 355, 356, |
| | | 358, 360, 362, 365, 366, 367, 368, 370, 373, 374, 377, 379, 381, |
| | | 382, 383, 386, 388, 392, 395, 400, 403, 454, 455, 456, 457, 458, |
| | | and 459. |
| 193 | Isotype control | EVRLLESGGGLVQPGGSLRLSCAASGFTFSNYAMGWVRQAPGKGLEWVSAISGSGGSTYYADSVKG |
| | heavy chain | RFTTSRDDSKNALYLQMNSLRAEDTAVYYCARGGPGWYAADVWGQGTTVTVSSASTKGPSVFPLAP |
| | hIgG1 NSLF | SSKSTSGGTAALGCLVKDYFPEPVTVSWNSGALTSGVHTFPAVLQSSGLYSLSSVVTVPSSSLGTQ |
| | hole scFv | TYICNVNHKPSNTKVDKKVEPKSCDKTHTCPPCPAPELLGGPSVFLFPPKPKDTLMISRTPEVTCV |
| | (T366S_L368A_Y407V) | VVDVSHEDPEVKFNWYVDGVEVHNAKTKPREEQYNSTYRVVSVLTVLHQDWLNGKEYKCKVSSKAF |
| | (N325S_L328F)-(G4S)3 | PAPIEKTISKAKGQPREPQVYTLPPSRDELTKNQVSLSCAVKGFYPSDIAVEWESNGQPENNYKTT |
| | linker-VH-20AA | PPVLDSDGSFFLVSKLTVDKSRWQQGNVFSCSVMHEALHNRFTQKSLSLSPGK-GGGGSGGGGSGG |
| | linker- VL mutations | GGS-X1-GGSEGKSSGSGSESKSTGGS-X2, wherein X1 is the |
| | (H435R, Y436F) | amino acid sequence of any one of SEQ ID NOs: 10, 12, 14, 16, 18, |
| | | 20, 22, 24, 26, 28, 30, 32, 34, 36, 38, 40, 42, 44, 46, 48, 50, |
| | | 52, 54, 56, 58, 60, 62, 64, 66, 68, 70, 72, 313, 315, 317, 318, |
| | | 319, 320, 321, 322, 323, 324, 325, 327, 330, 334, 335, 337, 339, |
| | | 343, 344, 345, 346, 348, 349, 351, 357, 359, 361, 363, 364, 369, |
| | | 371, 372, 375, 376, 378, 380, 384, 385, 387, 389, 390, 391, 393, |
| | | 394, 396, 397, 398, 399, 401, 402, 443, 444, 445, 446, 447, 448, |
| | | 449, 450, 451, 452, and 453, and wherein X2 is the amino acid |
| | | sequence of any one of SEQ ID NOs: 11, 13, 15, 17, 19, 21, 23, 25, |
| | | 27, 29, 31, 33, 35, 37, 39, 41, 43, 45, 47, 49, 51, 53, 55, 57, |
| | | 59, 61, 63, 65, 67, 69, 71, 73, 314, 316, 326, 328, 329, 331, 332, |
| | | 333, 336, 338, 340, 341, 342, 347, 350, 352, 353, 354, 355, 356, |
| | | 358, 360, 362, 365, 366, 367, 368, 370, 373, 374, 377, 379, 381, |
| | | 382, 383, 386, 388, 392, 395, 400, 403, 454, 455, 456, 457, 458, |
| | | and 459. |
| 194 | Isotype control | EVRLLESGGGLVQPGGSLRLSCAASGFTFSNYAMGWVRQAPGKGLEWVSAISGSGGSTYYADSVKG |
| | heavy chain | RFTTSRDDSKNALYLQMNSLRAEDTAVYYCARGGPGWYAADVWGQGTTVTVSSASTKGPSVFPLAP |
| | hIgG1 NSLF | SSKSTSGGTAALGCLVKDYFPEPVTVSWNSGALTSGVHTFPAVLQSSGLYSLSSVVTVPSSSLGTQ |
| | knob (T366W) | TYICNVNHKPSNTKVDKKVEPKSCDKTHTCPPCPAPELLGGPSVFLFPPKPKDTLMISRTPEVTCV |
| | (N325S_L328F) | VVDVSHEDPEVKFNWYVDGVEVHNAKTKPREEQYNSTYRVVSVLTVLHQDWLNGKEYKCKVSSKAF |
| | | PAPIEKTISKAKGQPREPQVYTLPPSRDELTKNQVSLWCLVKGFYPSDIAVEWESNGQPENNYKTT |
| | | PPVLDSDGSFFLYSKLTVDKSRWQQGNVFSCSVMHEALHNHYTQKSLSLSPGK |

To generate the 2+1 bispecific antibodies, transient transfection of Expi293 cells (Invitrogen) was performed according to manufacturer's instructions. For 20 mL culture, 20 g of total DNA consisting of 3 expression plasmids (heavy chain-hole-scFv sequence, heavy chain-knob sequence, and light chain sequence) were used. The molar ratio of the heavy chain-knob, the heavy chain-hole-scFv, and the light chain plasmids were optimized to 1:3:6 to achieve high purity of the 2+1 bispecific antibodies. The cell culture supernatant was harvested at 5 days post transfection. Clarified supernatants were purified using drip column with MabSelect Sure resin (Cytiva), washed with PBS, and eluted with pH 3.5 elution buffer and neutralized with Tris-HCl to a final pH of 5.5-6.0. Neutralized eluates containing the antibodies were dialyzed into PBS. Quantification of the antibody concentration was determined by measuring the absorbance at 280 nm using the Nanodrop 8000 (ThermoFisher) or Lunatic (Unchained Labs). Purity of the 2+1 bispecific antibodies was determined by SDS-PAGE. The 2+1 bispecific antibodies were analyzed with size exclusion chromatography (SEC) for aggregation. Next, antibodies were produced and purified as 2+1 bispecific antibodies and analyzed via ELISA, FACS and internalization to confirm retention of binding in the 2+1 format.

### Example 10: Production and Characterization of 2+1 Anti-TfR Bispecific Antibodies

Anti-TfR hybridoma antibodies were converted into a 2+1 format and expressed and purified as described in Example 9. Retention of binding was confirmed via ELISA and FACS.

To determine binding retention of reformatted scFv 2+1 anti-TfR antibodies by ELISA, high binding ELISA plates (Thermo Fisher) were coated with streptavidin (1 μg/ml, Thermo Fisher) overnight at 4° C. Plates were then washed three times with phosphate buffered saline with 0.05% tween-20 (PBST) and incubated with block buffer (PBS+1% BSA, 1 h, room temperature). Plates were washed three times again with PBST and incubated with biotinylated human, cyno, and mouse TfR and an irrelevant antigen, human transferrin receptor (1 μg/ml, 1 h, room temperature). Plates were washed three times again with PBST and incubated with anti-TfR antibodies (1 μg/ml, 1 h, room temperature). Plates were washed three times again with PBST and incubated with a secondary detection antibody, anti-human horseradish peroxidase conjugated antibody (1:5000 dilution, 30 min, room temperature). Plates were washed three times one last time and incubated with TMB-ELISA Substrate Solution (Thermo Fisher) and then quenched with H₂SO₄. Anti-TfR binding was then analyzed by quantifying optical density at 450 nm (OD₄₅₀) using the SpectraMax M5 (Molecular Devices). Binding characteristics to TfR as scFv 2+1 anti-TfR antibodies are summarized below in Table 15 as fold-change relative to isotype hIgG.

To determine binding retention of reformatted 2+1 scFv anti-TfR antibodies by FACS, antibodies were evaluated for binding to HCMEC/D3 cells (a cell line derived from human brain endothelial cells), overexpressing human and mouse TfR CHO cell lines, and CHO cells for non-specific binding. Cells were seeded in tissue culture plates (50,000 cells/well) and washed and resuspended in FACS buffer (PBS+2% BSA+1 mM EDTA). Anti-TfR antibodies were then incubated with cells (5 μg/ml, 1 hr, on ice) and washed twice with FACS buffer. Cells were then incubated with allophycocyanin conjugated anti-mouse secondary antibody (Jackson Immunoresearch) (1:1000, 30 min, on ice) and then washed twice with FACS buffer. Cells were then resuspended in FACS buffer supplemented with 0.5% propidium iodide (Thermo Fisher). Anti-TfR binding was then analyzed on the iQue flow cytometer (IntelliCyt) by measuring the Mean Fluorescent Intensity (MFI). FACS data are summarized below in Table 15 as MFI fold-change relative to isotype hIgG1.

In total, 28 out of 32 reformatted antibodies retained binding affinity against TfR in the 2+1 format (Table 15). Importantly, only 6 of the 32 antibodies (TfR6, TfR9, TfR12, TfR15, TfR19, TfR27) showed strong cyno cross-reactivity, so only those were considered for further maturation.

TABLE 15
Binding characteristics of anti-TfR antibodies
reformatted into 2 + 1 bispecific format.

| | ELISA (Fold over isotype control) | | | | FACS (CHO + hTfR) |
|---|---|---|---|---|---|
| Anti- | huTfR | Cyno TfR | MuTfR | hTfR | Fold over |
| body | Avi-His | Avi-His | Avi-His | Apical | CHO parental |
| TfR1 | 11 | 1 | 1 | 13 | 28 |
| TfR2 | 1 | 1 | 1 | 1 | 1 |
| TfR3 | 9 | 1 | 1 | 9 | 7 |
| TfR4 | 30 | 1 | 1 | 40 | 305 |
| TfR5 | 24 | 1 | 1 | 42 | 298 |
| TfR6 | 5 | 21 | 1 | 1 | 19 |
| TfR7 | 27 | 1 | 1 | 45 | 314 |
| TfR8 | 26 | 1 | 1 | 32 | 293 |
| TfR9 | 23 | 10 | 1 | 26 | 62 |
| TfR10 | 25 | 1 | 1 | 35 | 93 |
| TfR11 | 28 | 4 | 1 | 44 | 107 |
| TfR12 | 28 | 36 | 1 | 40 | 152 |
| TfR13 | 6 | 1 | 1 | 8 | 17 |
| TfR14 | 5 | 4 | 1 | 37 | 2 |
| TfR15 | 29 | 28 | 1 | 40 | 875 |
| TfR16 | 29 | 1 | 1 | 39 | 1005 |
| TfR17 | 24 | 1 | 1 | 38 | 305 |
| TfR18 | 28 | 1 | 1 | 39 | 777 |
| TfR19 | 26 | 30 | 1 | 41 | 277 |
| TfR20 | 14 | 1 | 2 | 1 | 65 |
| TfR21 | 1 | 1 | 1 | 1 | 2 |
| TfR22 | 27 | 1 | 2 | 40 | 442 |
| TfR23 | 29 | 1 | 1 | 40 | 278 |
| TfR24 | 4 | 1 | 1 | 3 | 11 |
| TfR25 | 3 | 1 | 1 | 12 | 23 |
| TfR26 | 27 | 1 | 1 | 39 | 590 |
| TfR27 | 27 | 32 | 1 | 42 | 309 |
| TfR28 | 27 | 1 | 1 | 39 | 438 |
| TfR29 | 1 | 1 | 1 | 1 | 1 |
| TfR30 | 32 | 5 | 1 | 40 | 261 |
| TfR31 | 25 | 5 | 1 | 41 | 383 |
| TfR32 | 21 | 1 | 1 | 30 | 242 |
| isotype | 1 | 1 | 1 | 1 | 2 |
| control | | | | | |

### Example 11: In Vitro Internalization of 2+1 Anti-TfR Bispecific Antibodies in a BBB Endothelial Cell Line

Internalization of 2+1 anti-TfR bispecific antibodies into the hCMEC/D3 cell line was carried out as described previously (Example 7). 2+1 anti-TfR bispecific antibodies showed a wide range of internalization capability with many showing significantly higher internalization than an anti-TfR control antibody.

TABLE 16
Internalization characteristics of anti-TfR antibodies
reformatted into 2 + 1 bispecific antibody format
(Fold change of Integrated Intensity/Phase area per well)

| 2 + 1 bispecific | Fold change over isotype |
|---|---|
| antibody | control IgG |
| TfR1 | 483 |
| TfR2 | 1 |
| TfR3 | 0 |
| TfR4 | 110 |
| TfR5 | 91 |
| TfR6 | 1 |
| TfR7 | 32 |
| TfR8 | 52 |
| TfR9 | 529 |
| TfR10 | 561 |
| TfR11 | 35 |
| TfR12 | 239 |
| TfR13 | 218 |
| TfR14 | 2 |
| TfR15 | 56 |
| TfR16 | 28 |
| TfR17 | 5 |
| TfR18 | 16 |
| TfR19 | 16 |
| TfR20 | 1 |
| TfR21 | 0 |
| TfR22 | 146 |
| TfR23 | 35 |
| TfR24 | 2 |
| TfR25 | 1 |
| TfR26 | 223 |
| TfR27 | 43 |
| TfR28 | 18 |
| TfR29 | 10 |
| TfR30 | 41 |
| TfR31 | 40 |
| TfR32 | 304 |
| Anti-TfR IgG | 26 |
| Isotype control | 1 |

### Example 12: Humanization of Anti-TfR Mouse Antibodies

The scFv portion of TfR9, TfR12 and TfR15 were chosen for humanization. These antibodies were chosen for their cyno cross-reactivity, but also function (internalization), functional stability (ability to internalize after heat stress) and sequence diversity.

Humanization was carried out using a CDR transplantation method. One of the most common methods of humanizing a non-human antibody is to transplant the CDRs from a non-human antibody onto a human antibody acceptor framework. Frequently, such CDR transplantation results in attenuation or complete loss of affinity of the humanized antibody due to perturbation in its framework. As a result, certain residues from the mouse framework may need to be retained to replace the human residues at the corresponding positions (back mutations) in order to restore attenuated or lost affinity. Therefore, it is crucial to precisely predict the residues to be retained in the context of the selected human antibody germline acceptor framework that can retain functions and paratopes of the humanized antibody. In addition, retained or improved thermal stability and solubility are desired for good manufacturability and downstream development.

Structure-based antibody modeling was applied in the process of humanizing anti-TfR mouse monoclonal antibodies (mAbs) utilizing the BioMOE module of MOE (Molecular Operating Environment, Chemical Computing Group, Montreal, Canada). Briefly, VH and VL sequences of the mouse mAb sequence to be humanized were compared to human VL, VH, LJ, HJ functional germline sequences taken from IMGT (http://www.imgt.org/). Pseudo-genes and ORFs were excluded. Per one mouse mAb (query), five most similar VL and five most similar VH germline sequences were selected and combined with the most similar VJ and HJ genes, producing 25 humanized sequences. The CDRs to be transplanted onto the human framework were defined according to the AbM definition (http://www.bioinf.org.uk/abs/ #cdrdef).

Two humanized sequences were selected based on the frequency of their VH and VL frameworks in human repertoire. The query and the humanized sequences were used to create Fv homology models. The BioMOE module or the Antibody Modeler module of MOE (Molecular Operating Environment, Chemical Computing Group, Montreal, Canada) was utilized to create Fv homology models. AMBER10:EHT force field was used for energy minimization through the entire antibody homology modeling process. Based on the Fv homology models, molecular descriptors such as interaction energy between VL and VH, coordinate-based isoelectric point (3D pI), hydrophobic patch, and charged surface area were calculated, analyzed, and sorted by scoring metrics provided by MOE. These molecular descriptors were utilized to prioritize the humanized mAbs for downstream experimental procedures, including protein expression, purification, and binding affinity test, and functional assays.

The BioMOE module of MOE provides a tool, Mutation Site Properties, to visualize and classify potential residues for back-mutation. Back-mutation is defined as amino acid substitution which is reverted to the original query sequence replacing the humanized sequence. Using this tool, the original query (reference) was compared individually to the selected humanized variants for both the primary amino acid sequence and the 3D structure of the 3D Fv homology model.

The changes between the reference and the humanized variant were classified based on amino acid type difference, interaction potential with CDR residues, impact potential for VL/VH pairing, and potential change in hydrophobic and charged surface area in and near the CDRs.

Mutations that were near the CDRs or the VL/VH interface, have a significant charge difference or contain strong H-bond interactions were individually evaluated and the significantly disrupting mutations were reverted back to the original query residues. As a result, humanized sequences may contain up to five back mutations.

Humanized sequences are described for TfR9, TfR12, and TfR15 in Table 17 below.

TABLE 17
Humanized variants of TfR9, TfR12, and TfR15
Heavy chain and light chain sequences in scFv
are underlined.

| Antibody | Antibody Sequence |
|---|---|
| parental | EVQLQQSGPELVKPGASVKISCKTSGYTFTEYAMHWVKQSHGKSLE |
| TfR9 scFv | WIGGINPNDGGTSYNQKFKGKATLTVDKSSSTAYMELRSLTSEDSA |
| | VYYCSSQRWLLRGGDYWGQGTTLTVSSGGSEGKSSGSGSESKSTGG |
| | SDVVMTQTPLTLSVTIGQPASISCKSSQSLLDSDGKTYLNWLLQRP |
| | GQSPKRLIYLVSKLDSGVPDRFTGSGSGTDFTLKISRVEAEDLGVY |
| | YCWQGTHFPQTFGGGTKLEIK (SEQ ID NO: 195) |
| TfR9.1A | QVQLVQSGAEVKKPGASVKVSCKASGYTFTEYAMHWVRQAPGQGLE |
| scFv | WMGGINPNDGGTSYNQKFKGRVTMTRDTSTSTVYMELSSLRSEDTA |
| | VYYCASQRWLLRGGDYWGQGTLVTVSSGGSEGKSSGSGSESKSTGG |
| | SDVVMTQSPLSLPVTLGQPASISCKSSQSLLDSDGKTYLNWFQQRP |
| | GQSPRRLIYLVSKLDSGVPDRFSGSGSGTDFTLKISRVEAEDVGVY |
| | YCWQGTHFPQTFGGGTKVEIK (SEQ ID NO: 196) |
| TfR9.1B | QVQLVQSGAEVKKPGASVKVSCKASGYTFTEYAMHWVRQAPGQGLE |
| scFv | WMGGINPNDGGTSYNQKFKGRVTMTVDTSTSTAYMELSSLRSEDTA |
| | VYYCASQRWLLRGGDYWGQGTLVTVSSGGSEGKSSGSGSESKSTGG |
| | SDVVMTQSPLSLPVTLGQPASISCKSSQSLLDSDGKTYLNWLQQRP |
| | GQSPRRLIYLVSKLDSGVPDRFSGSGSGTDFTLKISRVEAEDVGVY |
| | YCWQGTHFPQTFGGGTKVEIK (SEQ ID NO: 197) |
| TfR9.2A | QVQLVQSGAEVKKPGASVKVSCKASGYTFTEYAMHWVRQAPGQRLE |
| scFv | WMGGINPNDGGTSYNQKFKGRVTITRDTSASTAYMELSSLRSEDTA |
| | VYYCASQRWLLRGGDYWGQGTLVTVSSGGSEGKSSGSGSESKSTGG |
| | SDVVMTQSPLSLPVTLGQPASISCKSSQSLLDSDGKTYLNWFQQRP |
| | GQSPRRLIYLVSKLDSGVPDRFSGSGSGTDFTLKISRVEAEDVGVY |
| | YCWQGTHFPQTFGGGTKVEIK (SEQ ID NO: 198) |
| TfR9.2B | QVQLVQSGAEVKKPGASVKVSCKASGYTFTEYAMHWVRQAPGQRLE |
| scFv | WMGGINPNDGGTSYNQKFKGRVTITVDTSASTAYMELRSLRSEDTA |
| | VYYCASQRWLLRGGDYWGQGTLVTVSSGGSEGKSSGSGSESKSTGG |
| | SDVVMTQSPLSLPVTLGQPASISCKSSQSLLDSDGKTYLNWLQQRP |
| | GQSPRRLIYLVSKLDSGVPDRFSGSGSGTDFTLKISRVEAEDVGVY |
| | YCWQGTHFPQTFGGGTKVEIK (SEQ ID NO: 199) |
| TfR9.3A | EVQLVQSGAEVKKPGATVKISCKVSGYTFTEYAMHWVQQAPGKGLE |
| scFv | WMGGINPNDGGTSYNQKFKGRVTITADTSTDTAYMELSSLRSEDTA |
| | VYYCATQRWLLRGGDYWGQGTLVTVSSGGSEGKSSGSGSESKSTGG |
| | SDVVMTQSPLSLPVTLGQPASISCKSSQSLLDSDGKTYLNWFQQRP |
| | GQSPRRLIYLVSKLDSGVPDRFSGSGSGTDFTLKISRVEAEDVGVY |
| | YCWQGTHFPQTFGGGTKVEIK (SEQ ID NO: 200) |
| TfR9.3B | EVQLVQSGAEVKKPGATVKISCKVSGYTFTEYAMHWVQQAPGKGLE |
| scFv | WMGGINPNDGGTSYNQKFKGRVTITADKSTDTAYMELRSLRSEDTA |
| | VYYCATQRWLLRGGDYWGQGTLVTVSSGGSEGKSSGSGSESKSTGG |
| | SDVVMTQSPLSLPVTLGQPASISCKSSQSLLDSDGKTYLNWLQQRP |
| | GQSPRRLIYLVSKLDSGVPDRFSGSGSGTDFTLKISRVEAEDVGVY |
| | YCWQGTHFPQTFGGGTKVEIK (SEQ ID NO: 201) |
| TfR9.4A | QVQLVQSGAEVKKPGASVKVSCKVSGYTLTEYAMHWVRQAPGKGLE |
| scFv | WMGGINPNDGGTSYNQKFKGRVTMTEDTSTDTAYMELSSLRSEDTA |
| | VYYCATQRWLLRGGDYWGQGTLVTVSSGGSEGKSSGSGSESKSTGG |
| | SDVVMTQSPLSLPVTLGQPASISCKSSQSLLDSDGKTYLNWFQQRP |
| | GQSPRRLIYLVSKLDSGVPDRFSGSGSGTDFTLKISRVEAEDVGVY |
| | YCWQGTHFPQTFGGGTKVEIK (SEQ ID NO: 202) |
| TfR9.4B | QVQLVQSGAEVKKPGASVKVSCKVSGYTLTEYAMHWVRQAPGKGLE |
| scFv | WMGGINPNDGGTSYNQKFKGRVTMTEDKSTDTAYMELRSLRSEDTA |
| | VYYCATQRWLLRGGDYWGQGTLVTVSSGGSEGKSSGSGSESKSTGG |
| | SDVVMTQSPLSLPVTLGQPASISCKSSQSLLDSDGKTYLNWLQQRP |
| | GQSPRRLIYLVSKLDSGVPDRFSGSGSGTDFTLKISRVEAEDVGVY |
| | YCWQGTHFPQTFGGGTKVEIK (SEQ ID NO: 203) |
| TfR9.5A | QVQLVQSGAEVKKPGASVKVSCKASGYTFTEYAMHWVRQAPGQGLE |
| scFv | WMGGINPNDGGTSYNQKFKGRVTSTRDTSISTAYMELSRLRSDDTV |
| | VYYCASQRWLLRGGDYWGQGTLVTVSSGGSEGKSSGSGSESKSTGG |
| | SDVVMTQSPLSLPVTLGQPASISCKSSQSLLDSDGKTYLNWFQQRP |
| | GQSPRRLIYLVSKLDSGVPDRFSGSGSGTDFTLKISRVEAEDVGVY |
| | YCWQGTHFPQTFGGGTKVEIK (SEQ ID NO: 204) |
| TfR9.5B | QVQLVQSGAEVKKPGASVKVSCKASGYTFTEYAMHWVRQAPGQGLE |
| scFv | WMGGINPNDGGTSYNQKFKGRVTLTVDTSISTAYMELSRLRSDDTV |
| | VYYCASQRWLLRGGDYWGQGTLVTVSSGGSEGKSSGSGSESKSTGG |
| | SDVVMTQSPLSLPVTLGQPASISCKSSQSLLDSDGKTYLNWLQQRP |
| | GQSPRRLIYLVSKLDSGVPDRFSGSGSGTDFTLKISRVEAEDVGVY |
| | YCWQGTHFPQTFGGGTKVEIK (SEQ ID NO: 205) |
| parental | QVQLQQPGSELVRPGASVKLSCKASGYTFTSYWMHWVKQRPGQGLE |
| TfR12 scFv | WIGNIYPGSGSSNYDEKFKNKATLTVDTSSNTAYMQLSSLTSEDSA |
| | VYYCTRSSYYGNFDTMDNWGQGTSVTVSSGGSEGKSSGSGSESKST |
| | GGSQIVLTQSPAIMSASPGEKVTMTCSASSSIRYMNWYQQKPVTSP |
| | KRWIYDTSTLASGVPARFSGSGSGTSYSLTISSMEAEDAATYYCHQ |
| | RSSYPWTFGGGTKLEIK (SEQ ID NO: 206) |
| TfR12.1A | QVQLVQSGAEVKKPGASVKVSCKASGYTFTSYWMHWVRQAPGQGLE |
| scFv | WMGNIYPGSGSSNYDEKFKNRVTMTRDTSTSTVYMELSSLRSEDTA |
| | VYYCARSSYYGNFDTMDNWGQGTTVTVSSGGSEGKSSGSGSESKST |
| | GGSDIQLTQSPSFLSASVGDRVTITCSASSSIRYMNWYQQKPGKAP |
| | KLLIYDTSTLASGVPSRFSGSGSGTEFTLTISSLQPEDFATYYCHQ |
| | RSSYPWTFGGGTKVEIK (SEQ ID NO: 207) |
| TfR12.1B | QVQLVQSGAEVKKPGASVKVSCKASGYTFTSYWMHWVRQAPGQGLE |
| scFv | WMGNIYPGSGSSNYDEKFKNRVTLTVDTSTSTAYMELSSLRSEDTA |
| | VYYCARSSYYGNFDTMDNWGQGTTVTVSSGGSEGKSSGSGSESKST |
| | GGSQIQLTQSPSFLSASVGDRVTITCSASSSIRYMNWYQQKPGKAP |
| | KRLIYDTSTLASGVPSRFSGSGSGTSYTLTISSLQPEDFATYYCHQ |
| | RSSYPWTFGGGTKVEIK (SEQ ID NO: 208) |
| TfR12.2A | QVQLVQSGAEVKKPGASVKVSCKASGYTFTSYWMHWVRQAPGQGLE |
| scFv | WMGNIYPGSGSSNYDEKFKNRVTMTRDTSTSTVYMELSSLRSEDTA |
| | VYYCARSSYYGNFDTMDNWGQGTTVTVSSGGSEGKSSGSGSESKST |
| | GGSEIVLTQSPATLSLSPGERATLSCSASSSIRYMNWYQQKPGQAP |
| | RLLIYDTSTLASGIPARFSGSGSGTDFTLTISSLEPEDFAVYYCHQ |
| | RSSYPWTFGGGTKVEIK (SEQ ID NO: 209) |
| TfR12.2B | QVQLVQSGAEVKKPGASVKVSCKASGYTFTSYWMHWVRQAPGQGLE |
| scFv | WMGNIYPGSGSSNYDEKFKNRVTLTVDTSTSTVYMELSSLRSEDTA |
| | VYYCARSSYYGNFDTMDNWGQGTTVTVSSGGSEGKSSGSGSESKST |
| | GGSQIVLTQSPATLSLSPGERATLSCSASSSIRYMNWYQQKPGQAP |
| | RRLIYDTSTLASGIPARFSGSGSGTSFTLTISSLEPEDFAVYYCHQ |
| | RSSYPWTFGGGTKVEIK (SEQ ID NO: 210) |
| TfR12.3A | QVQLVQSGAEVKKPGASVKVSCKASGYTFTSYWMHWVRQAPGQGLE |
| scFv | WMGNIYPGSGSSNYDEKFKNRVTMTRDTSTSTVYMELSSLRSEDTA |
| | VYYCARSSYYGNFDTMDNWGQGTTVTVSSGGSEGKSSGSGSESKST |
| | GGSDIQMTQSPSSLSASVGDRVTITCSASSSIRYMNWYQQKPGKAP |
| | KLLIYDTSTLASGVPSRFSGSGSGTDFTLTISSLQPEDFATYYCHQ |
| | RSSYPWTFGGGTKVEIK (SEQ ID NO: 211) |
| TfR12.3B | QVQLVQSGAEVKKPGASVKVSCKASGYTFTSYWMHWVRQAPGQGLE |
| scFv | WMGNIYPGSGSSNYDEKFKNRVTLTVDTSTSTVYMELSSLRSEDTA |
| | VYYCARSSYYGNFDTMDNWGQGTTVTVSSGGSEGKSSGSGSESKST |
| | GGSQIQMTQSPSSLSASVGDRVTITCSASSSIRYMNWYQQKPGKAP |
| | KRLIYDTSTLASGVPSRFSGSGSGTDYTLTISSLQPEDFATYYCHQ |
| | RSSYPWTFGGGTKVEIK (SEQ ID NO: 212) |
| TfR12.4A | QVQLVQSGAEVKKPGASVKVSCKASGYTFTSYWMHWVRQAPGQGLE |
| scFv | WMGNIYPGSGSSNYDEKFKNRVTSTRDTSISTAYMELSRLRSDDTV |
| | VYYCARSSYYGNFDTMDNWGQGTTVTVSSGGSEGKSSGSGSESKST |
| | GGSDIQMTQSPSSLSASVGDRVTITCSASSSIRYMNWYQQKPGKAP |
| | KLLIYDTSTLASGVPSRFSGSGSGTDFTLTISSLQPEDFATYYCHQ |
| | RSSYPWTFGGGTKVEIK (SEQ ID NO: 213) |
| TfR12.4B | QVQLVQSGAEVKKPGASVKVSCKASGYTFTSYWMHWVRQAPGQGLE |
| scFv | WMGNIYPGSGSSNYDEKFKNRVTLTVDTSISTAYMELSRLRSDDTV |
| | VYYCARSSYYGNFDTMDNWGQGTTVTVSSGGSEGKSSGSGSESKST |
| | GGSQIQMTQSPSSLSASVGDRVTITCSASSSIRYMNWYQQKPGKAP |
| | KRLIYDTSTLASGVPSRFSGSGSGTSYTLTISSLQPEDFATYYCHQ |
| | RSSYPWTFGGGTKVEIK (SEQ ID NO: 214) |
| TfR12.EB | QVQLVQSGAEVKKPGASVKVSCKASGYTFTSYWMHWVRQAPGQGLE |
| scFv | WMGNIYPGSGSSSYAQKFQGRVTMTVDTSTNTVYMELSSLRSEDTA |
| | VYYCTRSSYYGNFDTMDNWGQGTMVTVSSGGSEGKSSGSGSESKST |
| | GGSDIQLTQSPSFLSASVGDRVTITCRASSSIRYMNWYQQKPGKAP |
| | KRLIYDTSTLQSGVPSRFSGSGSGTEYTLTISSLQPEDFATYYCHQ |
| | RSSYPWTFGGGTKVEIK (SEQ ID NO: 215) |
| parental | QVQLQQPGSELVRPGASVKLSCKASGYTFTSYWMHWVKQRPGQGLE |
| TfR15 | WIGNIYPGSGSSNYDEKFKSKATLTVDTSSSTAYMQLNSLTSEDSA |
| | VYYCTRSSYYGNFDTMDNWGQGTSVTVSSGGSEGKSSGSGSESKST |
| | GGSQIVLTQSPAIMSASPGEKVTMTCSASSSIRYMNWYQQKPVTSP |
| | KRWIYDTSTLASGVPARFSGSGSGTSYSLTISSMEAEDAATYYCHQ |
| | RSSYPWTFGGGTKLEIK (SEQ ID NO: 216) |
| TfR15.WH1 | QVQLVQSGAEVKKPGASVKVSCKASGYTFTSYWMHWVRQAPGQGLE |
| | WMGIIYPGSGSSNYAQKFQGRVTMTRDTSTSTVYMELSSLRSEDTA |
| | VYYCTRSSYYGNFDTMDNWGQGTTVTVSSGGSEGKSSGSGSESKST |
| | GGSDIQLTQSPSFLSASVGDRVTITCRASSSIRYMNWYQQKPGKAP |
| | KRLIYDTSTLQSGVPSRFSGSGSGTEFTLTISSLQPEDFATYYCHQ |
| | RSSYPWTFGGGTKVEIK (SEQ ID NO: 217) |
| TfR15.WH2 | QVQLVQSGAEVKKPGASVKVSCKASGYTFTSYWMHWVRQAPGQGLE |
| | WMGIIYPGSGSSNYAQKFQGRVTMTRDTSTSTVYMELSSLRSEDTA |
| | VYYCTRSSYYGNFDTMDNWGQGTTVTVSSGGSEGKSSGSGSESKST |
| | GGSEIVLTQSPATLSLSPGERATLSCRASSSIRYMNWYQQKPGQAP |
| | RRLIYDTSNRATGIPARFSGSGSGTDFTLTISSLEPEDFAVYYCHQ |
| | RSSYPWTFGGGTKVEIK (SEQ ID NO: 218) |
| TfR15.WH3 | QVQLVQSGAEVKKPGASVKVSCKASGYTFTSYWMHWVRQAPGQGLE |
| | WMGIIYPGSGSSNYAQKFQGRVTMTRDTSTSTVYMELSSLRSEDTA |
| | VYYCTRSSYYGNFDTMDNWGQGTTVTVSSGGSEGKSSGSGSESKST |
| | GGSDIQMTQSPSSLSASVGDRVTITCRASSSIRYLNWYQQKPGKAP |
| | KRLIYDTSSLQSGVPSRFSGSGSGTDFTLTISSLQPEDFATYYCHQ |
| | RSSYPWTFGGGTKVEIK (SEQ ID NO: 219) |
| TfR15.WH4 | QVQLVQSGAEVKKPGASVKVSCKASGYTFTSYWMHWVRQAPGQGLE |
| | WMGNIYPGSGSSSYAQKFQGRVTMTRDTSTSTVYMELSSLRSEDTA |
| | VYYCTRSSYYGNFDTMDNWGQGTTVTVSSGGSEGKSSGSGSESKST |
| | GGSDIQLTQSPSFLSASVGDRVTITCRASSSIRYMNWYQQKPGKAP |
| | KRLIYDTSTLQSGVPSRFSGSGSGTEFTLTISSLQPEDFATYYCHQ |
| | RSSYPWTFGGGTKVEIK (SEQ ID NO: 220) |
| TfR15.WH6 | QVQLVQSGAEVKKPGASVKVSCKASGYTFTSYWMHWVRQAPGQGLE |
| | WMGNIYPGSGSSSYAQKFQGRVTMTRDTSTSTVYMELSSLRSEDTA |
| | VYYCTRSSYYGNFDTMDNWGQGTTVTVSSGGSEGKSSGSGSESKST |
| | GGSDIQMTQSPSSLSASVGDRVTITCRASSSIRYLNWYQQKPGKAP |
| | KRLIYDTSSLQSGVPSRFSGSGSGTDFTLTISSLQPEDFATYYCHQ |
| | RSSYPWTFGGGTKVEIK (SEQ ID NO: 221) |
| TfR15.WH7 | QVQLVQSGAEVKKPGASVKVSCKASGYTFTSYWMHWVRQAPGQGLE |
| | WMGNIYPGSGSSNYAQKFQGRVTMTRDTSTSTVYMELSSLRSEDTA |
| | VYYCTRSSYYGNFDTMDNWGQGTTVTVSSGGSEGKSSGSGSESKST |
| | GGSDIQLTQSPSFLSASVGDRVTITCRASSSIRYMNWYQQKPGKAP |
| | KRLIYDTSTLQSGVPSRFSGSGSGTEFTLTISSLQPEDFATYYCHQ |
| | RSSYPWTFGGGTKVEIK (SEQ ID NO: 222) |
| TfR15.WH8 | QVQLVQSGAEVKKPGASVKVSCKASGYTFTSYWMHWVRQAPGQGLE |
| | WMGNIYPGSGSSNYAQKFQGRVTMTRDTSTSTVYMELSSLRSEDTA |
| | VYYCTRSSYYGNFDTMDNWGQGTTVTVSSGGSEGKSSGSGSESKST |
| | GGSEIVLTQSPATLSLSPGERATLSCRASSSIRYMNWYQQKPGQAP |
| | RRLIYDTSNRATGIPARFSGSGSGTDFTLTISSLEPEDFAVYYCHQ |
| | RSSYPWTFGGGTKVEIK (SEQ ID NO: 223) |
| TfR15.WH9 | QVQLVQSGAEVKKPGASVKVSCKASGYTFTSYWMHWVRQAPGQGLE |
| | WMGNIYPGSGSSNYAQKFQGRVTMTRDTSTSTVYMELSSLRSEDTA |
| | VYYCTRSSYYGNFDTMDNWGQGTTVTVSSGGSEGKSSGSGSESKST |
| | GGSDIQMTQSPSSLSASVGDRVTITCRASSSIRYLNWYQQKPGKAP |
| | KRLIYDTSSLQSGVPSRFSGSGSGTDFTLTISSLQPEDFATYYCHQ |
| | RSSYPWTFGGGTKVEIK (SEQ ID NO: 224) |
| TfR15.WH10 | QVQLVQSGAEVKKPGASVKVSCKASGYTFTSYWMHWVRQAPGQGLE |
| | WMGNIYPGSGSSNYAQKFQGRVTLTVDTSTSTVYMELSSLRSEDTA |
| | VYYCTRSSYYGNFDTMDNWGQGTTVTVSSGGSEGKSSGSGSESKST |
| | GGSDIQLTQSPSFLSASVGDRVTITCRASSSIRYMNWYQQKPGKAP |
| | KRLIYDTSTLQSGVPSRFSGSGSGTEFTLTISSLQPEDFATYYCHQ |
| | RSSYPWTFGGGTKVEIK (SEQ ID NO: 225) |
| TfR15.WH11 | QVQLVQSGAEVKKPGASVKVSCKASGYTFTSYWMHWVRQAPGQGLE |
| | WMGNIYPGSGSSNYAQKFQGRVTLTVDTSTSTVYMELSSLRSEDTA |
| | VYYCTRSSYYGNFDTMDNWGQGTTVTVSSGGSEGKSSGSGSESKST |
| | GGSEIVLTQSPATLSLSPGERATLSCRASSSIRYMNWYQQKPGQAP |
| | RRLIYDTSNRATGIPARFSGSGSGTDFTLTISSLEPEDFAVYYCHQ |
| | RSSYPWTFGGGTKVEIK (SEQ ID NO: 226) |
| TfR15.WH12 | QVQLVQSGAEVKKPGASVKVSCKASGYTFTSYWMHWVRQAPGQGLE |
| | WMGNIYPGSGSSNYAQKFQGRVTLTVDTSTSTVYMELSSLRSEDTA |
| | VYYCTRSSYYGNFDTMDNWGQGTTVTVSSGGSEGKSSGSGSESKST |
| | GGSDIQMTQSPSSLSASVGDRVTITCRASSSIRYLNWYQQKPGKAP |
| | KRLIYDTSSLQSGVPSRFSGSGSGTDFTLTISSLQPEDFATYYCHQ |
| | RSSYPWTFGGGTKVEIK (SEQ ID NO: 227) |

TABLE 18
Variable heavy and variable light chain domains for
humanized variants of TfR9, TfR12, and TfR15

| Antibody | VH sequence | VL sequence |
|---|---|---|
| parental | EVQLQQSGPELVKPGASVKISCK | DVVMTQTPLTLSVTIGQPASISCKSS |
| TfR9 | TSGYTFTEYAMHWVKQSHGKSLE | QSLLDSDGKTYLNWLLQRPGQSPKRL |
| scFv | WIGGINPNDGGTSYNQKFKGKAT | IYLVSKLDSGVPDRFTGSGSGTDFTL |
| | LTVDKSSSTAYMELRSLTSEDSA | KISRVEAEDLGVYYCWQGTHFPQTFG |
| | VYYCSSQRWLLRGGDYWGQGTTL | GGTKLEIK (SEQ ID NO: 27) |
| | TVSS (SEQ ID NO: 26) | |
| TfR9.1A | QVQLVQSGAEVKKPGASVKVSCK | DVVMTQSPLSLPVTLGQPASISCKSS |
| scFv | ASGYTFTEYAMHWVRQAPGQGLE | QSLLDSDGKTYLNWFQQRPGQSPRRL |
| | WMGGINPNDGGTSYNQKFKGRVT | IYLVSKLDSGVPDRFSGSGSGTDFTL |
| | MTRDTSTSTVYMELSSLRSEDTA | KISRVEAEDVGVYYCWQGTHFPQTFG |
| | VYYCASQRWLLRGGDYWGQGTLV | GGTKVEIK (SEQ ID NO: 314) |
| | TVSS (SEQ ID NO: 313) | |
| TfR9.1B | QVQLVQSGAEVKKPGASVKVSCK | DVVMTQSPLSLPVTLGQPASISCKSS |
| scFv | ASGYTFTEYAMHWVRQAPGQGLE | QSLLDSDGKTYLNWLQQRPGQSPRRL |
| | WMGGINPNDGGTSYNQKFKGRVT | IYLVSKLDSGVPDRFSGSGSGTDFTL |
| | MTVDTSTSTAYMELSSLRSEDTA | KISRVEAEDVGVYYCWQGTHFPQTFG |
| | VYYCASQRWLLRGGDYWGQGTLV | GGTKVEIK (SEQ ID NO: 316) |
| | TVSS (SEQ ID NO: 315) | |
| TfR9.2A | QVQLVQSGAEVKKPGASVKVSCK | DVVMTQSPLSLPVTLGQPASISCKSS |
| scFv | ASGYTFTEYAMHWVRQAPGQRLE | QSLLDSDGKTYLNWFQQRPGQSPRRL |
| | WMGGINPNDGGTSYNQKFKGRVT | IYLVSKLDSGVPDRFSGSGSGTDFTL |
| | ITRDTSASTAYMELSSLRSEDTA | KISRVEAEDVGVYYCWQGTHFPQTFG |
| | VYYCASQRWLLRGGDYWGQGTLV | GGTKVEIK (SEQ ID NO: 314) |
| | TVSS (SEQ ID NO: 317) | |
| TfR9.2B | QVQLVQSGAEVKKPGASVKVSCK | DVVMTQSPLSLPVTLGQPASISCKSS |
| scFv | ASGYTFTEYAMHWVRQAPGQRLE | QSLLDSDGKTYLNWLQQRPGQSPRRL |
| | WMGGINPNDGGTSYNQKFKGRVT | IYLVSKLDSGVPDRFSGSGSGTDFTL |
| | ITVDTSASTAYMELRSLRSEDTA | KISRVEAEDVGVYYCWQGTHFPQTFG |
| | VYYCASQRWLLRGGDYWGQGTLV | GGTKVEIK (SEQ ID NO: 316) |
| | TVSS (SEQ ID NO: 318) | |
| TfR9.3A | EVQLVQSGAEVKKPGATVKISCK | DVVMTQSPLSLPVTLGQPASISCKSS |
| scFv | VSGYTFTEYAMHWVQQAPGKGLE | QSLLDSDGKTYLNWFQQRPGQSPRRL |
| | WMGGINPNDGGTSYNQKFKGRVT | IYLVSKLDSGVPDRFSGSGSGTDFTL |
| | ITADTSTDTAYMELSSLRSEDTA | KISRVEAEDVGVYYCWQGTHFPQTFG |
| | VYYCATQRWLLRGGDYWGQGTLV | GGTKVEIK (SEQ ID NO: 314) |
| | TVSS (SEQ ID NO: 319) | |
| TfR9.3B | EVQLVQSGAEVKKPGATVKISCK | DVVMTQSPLSLPVTLGQPASISCKSS |
| scFv | VSGYTFTEYAMHWVQQAPGKGLE | QSLLDSDGKTYLNWLQQRPGQSPRRL |
| | WMGGINPNDGGTSYNQKFKGRVT | IYLVSKLDSGVPDRFSGSGSGTDFTL |
| | ITADKSTDTAYMELRSLRSEDTA | KISRVEAEDVGVYYCWQGTHFPQTFG |
| | VYYCATQRWLLRGGDYWGQGTLV | GGTKVEIK (SEQ ID NO: 316) |
| | TVSS (SEQ ID NO: 320) | |
| TfR9.4A | QVQLVQSGAEVKKPGASVKVSCK | DVVMTQSPLSLPVTLGQPASISCKSS |
| scFv | VSGYTLTEYAMHWVRQAPGKGLE | QSLLDSDGKTYLNWFQQRPGQSPRRL |
| | WMGGINPNDGGTSYNQKFKGRVT | IYLVSKLDSGVPDRFSGSGSGTDFTL |
| | MTEDTSTDTAYMELSSLRSEDTA | KISRVEAEDVGVYYCWQGTHFPQTFG |
| | VYYCATQRWLLRGGDYWGQGTLV | GGTKVEIK (SEQ ID NO: 314) |
| | TVSS (SEQ ID NO: 321) | |
| TfR9.4B | QVQLVQSGAEVKKPGASVKVSCK | DVVMTQSPLSLPVTLGQPASISCKSS |
| scFv | VSGYTLTEYAMHWVRQAPGKGLE | QSLLDSDGKTYLNWLQQRPGQSPRRL |
| | WMGGINPNDGGTSYNQKFKGRVT | IYLVSKLDSGVPDRFSGSGSGTDFTL |
| | MTEDKSTDTAYMELRSLRSEDTA | KISRVEAEDVGVYYCWQGTHFPQTFG |
| | VYYCATQRWLLRGGDYWGQGTLV | GGTKVEIK (SEQ ID NO: 316) |
| | TVSS (SEQ ID NO: 322) | |
| TfR9.5A | QVQLVQSGAEVKKPGASVKVSCK | DVVMTQSPLSLPVTLGQPASISCKSS |
| scFv | ASGYTFTEYAMHWVRQAPGQGLE | QSLLDSDGKTYLNWFQQRPGQSPRRL |
| | WMGGINPNDGGTSYNQKFKGRVT | IYLVSKLDSGVPDRFSGSGSGTDFTL |
| | STRDTSISTAYMELSRLRSDDTV | KISRVEAEDVGVYYCWQGTHFPQTFG |
| | VYYCASQRWLLRGGDYWGQGTLV | GGTKVEIK (SEQ ID NO: 314) |
| | TVSS (SEQ ID NO: 323) | |
| TfR9.5B | QVQLVQSGAEVKKPGASVKVSCK | DVVMTQSPLSLPVTLGQPASISCKSS |
| scFv | ASGYTFTEYAMHWVRQAPGQGLE | QSLLDSDGKTYLNWLQQRPGQSPRRL |
| | WMGGINPNDGGTSYNQKFKGRVT | IYLVSKLDSGVPDRFSGSGSGTDFTL |
| | LTVDTSISTAYMELSRLRSDDTV | KISRVEAEDVGVYYCWQGTHFPQTFG |
| | VYYCASQRWLLRGGDYWGQGTLV | GGTKVEIK (SEQ ID NO: 316) |
| | TVSS (SEQ ID NO: 324) | |
| parental | QVQLQQPGSELVRPGASVKLSCK | QIVLTQSPAIMSASPGEKVTMTCSAS |
| TfR12 | ASGYTFTSYWMHWVKQRPGQGLE | SSIRYMNWYQQKPVTSPKRWIYDTST |
| scFv | WIGNIYPGSGSSNYDEKFKNKAT | LASGVPARFSGSGSGTSYSLTISSME |
| | LTVDTSSNTAYMQLSSLTSEDSA | AEDAATYYCHQRSSYPWTFGGGTKLE |
| | YVYCTRSSYYGNFDTMDNWGQGT | IK (SEQ ID NO: 33) |
| | SVTVSS (SEQ ID NO: 32) | |
| TfR12.1 | QVQLVQSGAEVKKPGASVKVSCK | DIQLTQSPSFLSASVGDRVTITCSAS |
| A scFv | ASGYTFTSYWMHWVRQAPGQGLE | SSIRYMNWYQQKPGKAPKLLIYDTST |
| | WMGNIYPGSGSSNYDEKFKNRVT | LASGVPSRFSGSGSGTEFTLTISSLQ |
| | MTRDTSTSTVYMELSSLRSEDTA | PEDFATYYCHQRSSYPWTFGGGTKVE |
| | VYYCARSSYYGNFDTMDNWGQGT | IK (SEQ ID NO: 326) |
| | TVTVSS (SEQ ID NO: 325) | |
| TfR12.1 | QVQLVQSGAEVKKPGASVKVSCK | QIQLTQSPSFLSASVGDRVTITCSAS |
| B scFv | ASGYTFTSYWMHWVRQAPGQGLE | SSIRYMNWYQQKPGKAPKRLIYDTST |
| | WMGNIYPGSGSSNYDEKFKNRVT | LASGVPSRFSGSGSGTSYTLTISSLQ |
| | LTVDTSTSTAYMELSSLRSEDTA | PEDFATYYCHQRSSYPWTFGGGTKVE |
| | VYYCARSSYYGNFDTMDNWGQGT | IK (SEQ ID NO: 328) |
| | TVTVSS (SEQ ID NO: 327) | |
| TfR12.2 | QVQLVQSGAEVKKPGASVKVSCK | EIVLTQSPATLSLSPGERATLSCSAS |
| A scFv | ASGYTFTSYWMHWVRQAPGQGLE | SSIRYMNWYQQKPGQAPRLLIYDTST |
| | WMGNIYPGSGSSNYDEKFKNRVT | LASGIPARFSGSGSGTDFTLTISSLE |
| | MTRDTSTSTVYMELSSLRSEDTA | PEDFAVYYCHQRSSYPWTFGGGTKVE |
| | VYYCARSSYYGNFDTMDNWGQGT | IK (SEQ ID NO: 329) |
| | TVTVSS (SEQ ID NO: 325) | |
| TfR12.2 | QVQLVQSGAEVKKPGASVKVSCK | QIVLTQSPATLSLSPGERATLSCSAS |
| B scFv | ASGYTFTSYWMHWVRQAPGQGLE | SSIRYMNWYQQKPGQAPRRLIYDTST |
| | WMGNIYPGSGSSNYDEKFKNRVT | LASGIPARFSGSGSGTSFTLTISSLE |
| | LTVDTSTSTVYMELSSLRSEDTA | PEDFAVYYCHQRSSYPWTFGGGTKVE |
| | VYYCARSSYYGNFDTMDNWGQGT | IK (SEQ ID NO: 331) |
| | TVTVSS (SEQ ID NO: 330) | |
| TfR12.3 | QVQLVQSGAEVKKPGASVKVSCK | DIQMTQSPSSLSASVGDRVTITCSAS |
| A scFv | ASGYTFTSYWMHWVRQAPGQGLE | SSIRYMNWYQQKPGKAPKLLIYDTST |
| | WMGNIYPGSGSSNYDEKFKNRVT | LASGVPSRFSGSGSGTDFTLTISSLQ |
| | MTRDTSTSTVYMELSSLRSEDTA | PEDFATYYCHQRSSYPWTFGGGTKVE |
| | VYYCARSSYYGNFDTMDNWGQGT | IK (SEQ ID NO: 332) |
| | TVTVSS (SEQ ID NO: 325) | |
| TfR12.3 | QVQLVQSGAEVKKPGASVKVSCK | QIQMTQSPSSLSASVGDRVTITCSAS |
| B scFv | ASGYTFTSYWMHWVRQAPGQGLE | SSIRYMNWYQQKPGKAPKRLIYDTST |
| | WMGNIYPGSGSSNYDEKFKNRVT | LASGVPSRFSGSGSGTDYTLTISSLQ |
| | LTVDTSTSTVYMELSSLRSEDTA | PEDFATYYCHQRSSYPWTFGGGTKVE |
| | VYYCARSSYYGNFDTMDNWGQGT | IK (SEQ ID NO: 333) |
| | TVTVSS (SEQ ID NO: 330) | |
| TfR12.4 | QVQLVQSGAEVKKPGASVKVSCK | DIQMTQSPSSLSASVGDRVTITCSAS |
| A scFv | ASGYTFTSYWMHWVRQAPGQGLE | SSIRYMNWYQQKPGKAPKLLIYDTST |
| | WMGNIYPGSGSSNYDEKFKNRVT | LASGVPSRFSGSGSGTDFTLTISSLQ |
| | STRDTSISTAYMELSRLRSDDTV | PEDFATYYCHQRSSYPWTFGGGTKVE |
| | VYYCARSSYYGNFDTMDNWGQGT | IK (SEQ ID NO: 332) |
| | TVTVSS (SEQ ID NO: 334) | |
| TfR12.4 | QVQLVQSGAEVKKPGASVKVSCK | QIQMTQSPSSLSASVGDRVTITCSAS |
| B scFv | ASGYTFTSYWMHWVRQAPGQGLE | SSIRYMNWYQQKPGKAPKRLIYDTST |
| | WMGNIYPGSGSSNYDEKFKNRVT | LASGVPSRFSGSGSGTSYTLTISSLQ |
| | LTVDTSISTAYMELSRLRSDDTV | PEDFATYYCHQRSSYPWTFGGGTKVE |
| | VYYCARSSYYGNFDTMDNWGQGT | IK (SEQ ID NO: 336) |
| | TVTVSS (SEQ ID NO: 335) | |
| TfR12.E | QVQLVQSGAEVKKPGASVKVSCK | DIQLTQSPSFLSASVGDRVTITCRAS |
| B scFv | ASGYTFTSYWMHWVRQAPGQGLE | SSIRYMNWYQQKPGKAPKRLIYDTST |
| | WMGNIYPGSGSSSYAQKFQGRVT | LQSGVPSRFSGSGSGTEYTLTISSLQ |
| | MTVDTSTNTVYMELSSLRSEDTA | PEDFATYYCHQRSSYPWTFGGGTKVE |
| | VYYCTRSSYYGNFDTMDNWGQGT | IK (SEQ ID NO: 338) |
| | MVTVSS (SEQ ID NO: 337) | |
| parental | QVQLQQPGSELVRPGASVKLSCK | QIVLTQSPAIMSASPGEKVTMTCSAS |
| TfR15 | ASGYTFTSYWMHWVKQRPGQGLE | SSIRYMNWYQQKPVTSPKRWIYDTST |
| | WIGNIYPGSGSSNYDEKFKSKAT | LASGVPARFSGSGSGTSYSLTISSME |
| | LTVDTSSSTAYMQLNSLTSEDSA | AEDAATYYCHQRSSYPWTFGGGTKLE |
| | VYYCTRSSYYGNFDTMDNWGQGT | IK (SEQ ID NO: 33) |
| | SVTVSS (SEQ ID NO: 38) | |
| TfR15. | QVQLVQSGAEVKKPGASVKVSCK | DIQLTQSPSFLSASVGDRVTITCRAS |
| WH1 | ASGYTFTSYWMHWVRQAPGQGLE | SSIRYMNWYQQKPGKAPKRLIYDTST |
| | WMGIIYPGSGSSNYAQKFQGRVT | LQSGVPSRFSGSGSGTEFTLTISSLQ |
| | MTRDTSTSTVYMELSSLRSEDTA | PEDFATYYCHQRSSYPWTFGGGTKVE |
| | VYYCTRSSYYGNFDTMDNWGQGT | IK (SEQ ID NO: 340) |
| | TVTVSS (SEQ ID NO: 339) | |
| TfR15. | QVQLVQSGAEVKKPGASVKVSCK | EIVLTQSPATLSLSPGERATLSCRAS |
| WH2 | ASGYTFTSYWMHWVRQAPGQGLE | SSIRYMNWYQQKPGQAPRRLIYDTSN |
| | WMGIIYPGSGSSNYAQKFQGRVT | RATGIPARFSGSGSGTDFTLTISSLE |
| | MTRDTSTSTVYMELSSLRSEDTA | PEDFAVYYCHQRSSYPWTFGGGTKVE |
| | VYYCTRSSYYGNFDTMDNWGQGT | IK (SEQ ID NO: 341) |
| | TVTVSS (SEQ ID NO: 339) | |
| TfR15. | QVQLVQSGAEVKKPGASVKVSCK | DIQMTQSPSSLSASVGDRVTITCRAS |
| WH3 | ASGYTFTSYWMHWVRQAPGQGLE | SSIRYLNWYQQKPGKAPKRLIYDTSS |
| | WMGIIYPGSGSSNYAQKFQGRVT | LQSGVPSRFSGSGSGTDFTLTISSLQ |
| | MTRDTSTSTVYMELSSLRSEDTA | PEDFATYYCHQRSSYPWTFGGGTKVE |
| | VYYCTRSSYYGNFDTMDNWGQGT | IK (SEQ ID NO: 342) |
| | TVTVSS (SEQ ID NO: 339) | |
| TfR15. | QVQLVQSGAEVKKPGASVKVSCK | DIQLTQSPSFLSASVGDRVTITCRAS |
| WH4 | ASGYTFTSYWMHWVRQAPGQGLE | SSIRYMNWYQQKPGKAPKRLIYDTST |
| | WMGNIYPGSGSSSYAQKFQGRVT | LQSGVPSRFSGSGSGTEFTLTISSLQ |
| | MTRDTSTSTVYMELSSLRSEDTA | PEDFATYYCHQRSSYPWTFGGGTKVE |
| | VYYCTRSSYYGNFDTMDNWGQGT | IK (SEQ ID NO: 340) |
| | TVTVSS (SEQ ID NO: 343) | |
| TfR15. | QVQLVQSGAEVKKPGASVKVSCK | DIQMTQSPSSLSASVGDRVTITCRAS |
| WH6 | ASGYTFTSYWMHWVRQAPGQGLE | SSIRYLNWYQQKPGKAPKRLIYDTSS |
| | WMGNIYPGSGSSSYAQKFQGRVT | LQSGVPSRFSGSGSGTDFTLTISSLQ |
| | MTRDTSTSTVYMELSSLRSEDTA | PEDFATYYCHQRSSYPWTFGGGTKVE |
| | VYYCTRSSYYGNFDTMDNWGQGT | IK (SEQ ID NO: 342) |
| | TVTVSS (SEQ ID NO: 343) | |
| TfR15. | QVQLVQSGAEVKKPGASVKVSCK | DIQLTQSPSFLSASVGDRVTITCRAS |
| WH7 | ASGYTFTSYWMHWVRQAPGQGLE | SSIRYMNWYQQKPGKAPKRLIYDTST |
| | WMGNIYPGSGSSNYAQKFQGRVT | LQSGVPSRFSGSGSGTEFTLTISSLQ |
| | MTRDTSTSTVYMELSSLRSEDTA | PEDFATYYCHQRSSYPWTFGGGTKVE |
| | VYYCTRSSYYGNFDTMDNWGQGT | IK (SEQ ID NO: 340) |
| | TVTVSS (SEQ ID NO: 344) | |
| TfR15. | QVQLVQSGAEVKKPGASVKVSCK | EIVLTQSPATLSLSPGERATLSCRAS |
| WH8 | ASGYTFTSYWMHWVRQAPGQGLE | SSIRYMNWYQQKPGQAPRRLIYDTSN |
| | WMGNIYPGSGSSNYAQKFQGRVT | RATGIPARFSGSGSGTDFTLTISSLE |
| | MTRDTSTSTVYMELSSLRSEDTA | PEDFAVYYCHQRSSYPWTFGGGTKVE |
| | VYYCTRSSYYGNFDTMDNWGQGT | IK (SEQ ID NO: 341) |
| | TVTVSS (SEQ ID NO: 344) | |
| TfR15. | QVQLVQSGAEVKKPGASVKVSCK | DIQMTQSPSSLSASVGDRVTITCRAS |
| WH9 | ASGYTFTSYWMHWVRQAPGQGLE | SSIRYLNWYQQKPGKAPKRLIYDTSS |
| | WMGNIYPGSGSSNYAQKFQGRVT | LQSGVPSRFSGSGSGTDFTLTISSLQ |
| | MTRDTSTSTVYMELSSLRSEDTA | PEDFATYYCHQRSSYPWTFGGGTKVE |
| | VYYCTRSSYYGNFDTMDNWGQGT | IK (SEQ ID NO: 342) |
| | TVTVSS (SEQ ID NO: 344) | |
| TfR15. | QVQLVQSGAEVKKPGASVKVSCK | DIQLTQSPSFLSASVGDRVTITCRAS |
| WH10 | ASGYTFTSYWMHWVRQAPGQGLE | SSIRYMNWYQQKPGKAPKRLIYDTST |
| | WMGNIYPGSGSSNYAQKFQGRVT | LQSGVPSRFSGSGSGTEFTLTISSLQ |
| | LTVDTSTSTVYMELSSLRSEDTA | PEDFATYYCHQRSSYPWTFGGGTKVE |
| | VYYCTRSSYYGNFDTMDNWGQGT | IK (SEQ ID NO: 340) |
| | TVTVSS (SEQ ID NO: 345) | |
| TfR15. | QVQLVQSGAEVKKPGASVKVSCK | EIVLTQSPATLSLSPGERATLSCRAS |
| WH11 | ASGYTFTSYWMHWVRQAPGQGLE | SSIRYMNWYQQKPGQAPRRLIYDTSN |
| | WMGNIYPGSGSSNYAQKFQGRVT | RATGIPARFSGSGSGTDFTLTISSLE |
| | LTVDTSTSTVYMELSSLRSEDTA | PEDFAVYYCHQRSSYPWTFGGGTKVE |
| | VYYCTRSSYYGNFDTMDNWGQGT | IK (SEQ ID NO: 341) |
| | TVTVSS (SEQ ID NO: 345) | |
| TfR15. | QVQLVQSGAEVKKPGASVKVSCK | DIQMTQSPSSLSASVGDRVTITCRAS |
| WH12 | ASGYTFTSYWMHWVRQAPGQGLE | SSIRYLNWYQQKPGKAPKRLIYDTSS |
| | WMGNIYPGSGSSNYAQKFQGRVT | LQSGVPSRFSGSGSGTDFTLTISSLQ |
| | LTVDTSTSTVYMELSSLRSEDTA | PEDFATYYCHQRSSYPWTFGGGTKVE |
| | VYYCTRSSYYGNFDTMDNWGQGT | IK (SEQ ID NO: 342) |
| | TVTVSS (SEQ ID NO: 345) | |

### Example 13: Effect of 2+1 Anti-TfR Bispecific Antibodies on TfR Recycling and Degradation in hCEMC/D3 Cells

An ideal BBB transport molecule will transport efficiently into the brain without affecting the expression level or localization of the target receptor, in this case TfR. To determine effects of 2+1 anti-TfR bispecific antibodies on recycling of the transferrin receptor, both FACS and Western blot methods were employed.

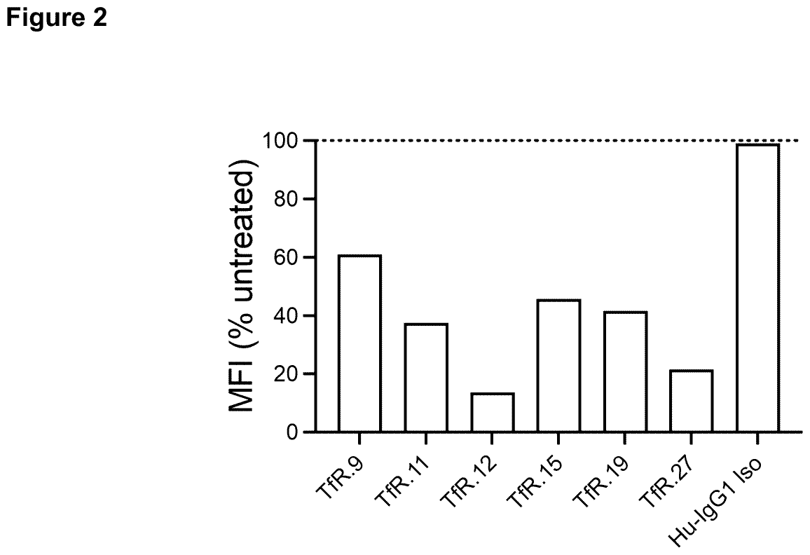

FACS method: HCMEC/D3 cells were seeded at 1.5*10≡cells/well in a 24-well plate in 100 μL of culture medium (EBM2, Lonza). Cells were then treated with 20 mg/ml of 2+1 anti-TfR bispecific antibodies for 24 hrs before being processed by FACS Canto system (BD Biosciences). A human IgG1 isotype was included in the assay as negative control. Since the 2+1 anti-TfR bispecific antibodies are specific for the TfR apical domain, the cells were stained with b3/25 Ab, which does not bind to this domain (Luria-Perez R., et al.; Bol Med Hosp Infant Mex. 2016; 73(6):372-379), thus avoiding antibody competition for receptor binding. To define effects of the 2+1 anti-TfR bispecific antibodies on TfR recycling, the Mean Fluorescence Intensity (MFI) for cells treated with each individual 2+1 anti-TfR bispecific antibody was normalized against that obtained for human IgG isotype. Treatment of HCMEC/D3 cells with the 2+1 anti-TfR bispecific antibodies resulted in 40-80% reduction in cell-surface expression of TfR (FIG. 2).

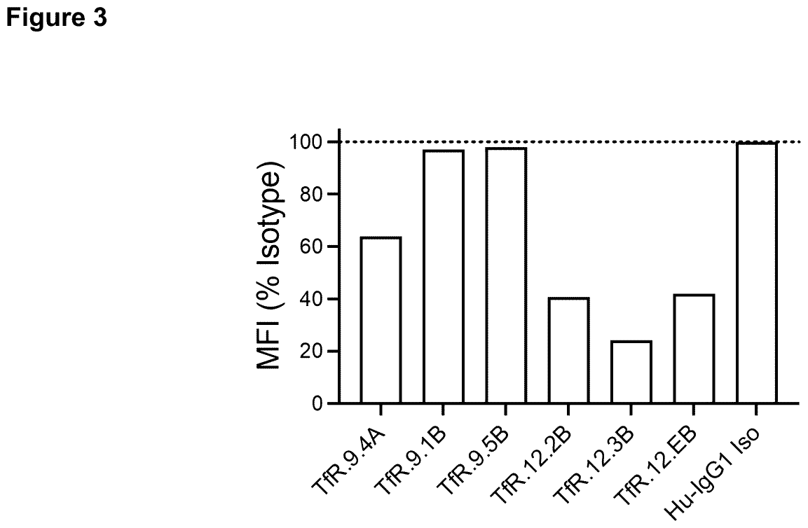

Two of these clones, namely TfR9 and TfR12, were selected for further engineering and a panel of humanized antibodies were generated (Examples 8 and 12). Interestingly, some antibody clones derived from TfR9 (i.e., TfR9.1B and TfR9.5B) displayed no adverse effect on receptor recycling. In contrast, all the clones generated from TfR12 strongly impaired receptor recycling (FIG. 3). Using the same strategy, a panel of humanized antibodies was generated based on TfR15 (Examples 8 and 12) and another antibody clone, TfR15.WH8, that showed no detrimental effects on TfR recycling (data not shown) was identified.

To validate the FACS data, the total amount of TfR protein in the cell lysates obtained from HCMEC/D3 following antibody treatment was quantified by Western blot. Western blot method: HCMEC/D3 cells were seeded at 3*10≡cells per well in a 12-well plate in 1 mL of culture medium (EBM2, Lonza). Cells were then exposed to 20 mg/ml of 2+1 anti-TfR bispecific antibodies for 24 hrs before being processed by Western Blot (WB). As controls, additional wells were treated with either human IgG1 isotype control (20 mg/ml) or PBS. Following two washes with ice-cold PBS, cells were collected into 1.5 ml microcentrifuge tubes using a scraper. After centrifugation (2000 g for 5 min), supernatants were removed from the tubes, and cells were lysed in RIPA buffer (R0278, Sigma-Aldrich) containing a cocktail of proteases inhibitors (cOmplete™, Mini Protease Inhibitor Cocktail, Roche, #11836153001). Cells were then incubated on a nutator at 4° C., followed by centrifugation at max speed in a benchtop centrifuge for 20 min at 4° C. Next, supernatants were transferred into new tubes, mixed with LDS Sample Buffer (Invitrogen, #NP0007), then incubated on a heat block (95° C.) for 5 min. Subsequently, samples were run on a protein gel electrophoresis, followed by protein transfer onto a nitrocellulose membrane. Membrane was treated with blocking buffer (TBST+5% milk powder) for 1 hr at RT, before being probed with 1 mg/ml of 2+1 anti-TfR bispecific antibody (R&D Systems Inc, #MAB2474) for 24 hrs at 4° C. Afterwards, membrane was washed 4× with PBST, followed by 1 hr incubation with 1:3000 dilution of HRP-conjugated goat anti-mouse IgG1 antibody (Jackson Immunoresearch, #115-035-003) at RT. After 4 washes with PBST, the membrane was developed with Supersignal West Pico reagent (ThermoFisher, #34580), imaged, and analyzed using an iBright instrument. Next, the membrane was incubated with stripping buffer (ThermoFisher, #21059) to remove the bound antibodies, before being probed with anti-human b-actin (Abcam, #ab8227) for quantification purposes. The TfR band density of each antibody was normalized to their corresponding b-actin band density. Then, these values were normalized to that obtained from the cells treated with human IgG1 isotype.

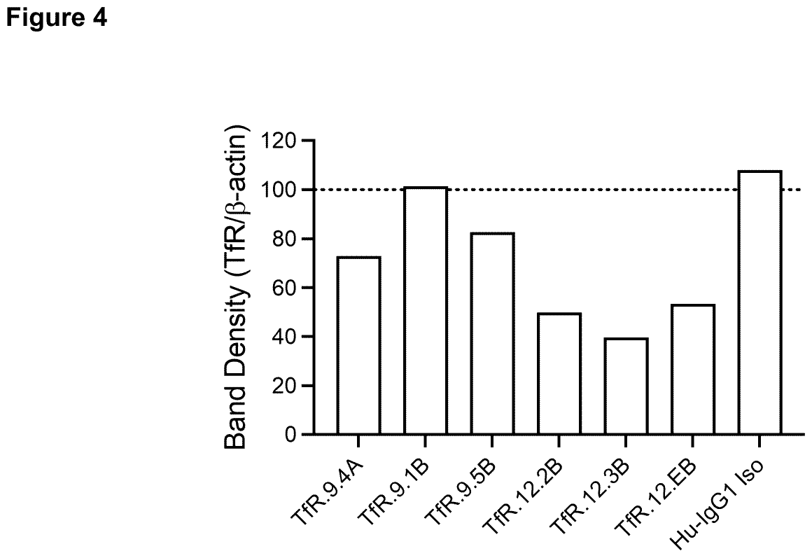

Similar to the FACS data, treatment of HCMEC/D3 cells with 2+1 anti-TfR bispecific antibodies had either minor or no effects on the total amount of TfR protein, with TfR9.1B in particular having no effect on total protein levels. In contrast, the total TfR protein in HCMEC/D3 was strongly reduced upon exposure to humanized antibodies generated from TfR12 (FIG. 4).

### Example 14: Engineering of Humanized Variants of TfR9.1B

Humanized TfR9.1B was selected for further affinity optimization. The anti-TfR scFv portion of TR9.1B was cloned into pComb3× phagemid vector (Addgene, Watertown MA) and overlap extension polymerase chain reaction (PCR) mutagenesis was used to generate randomized VH and VL libraries, consisting of a theoretical library size of 9E+07 different sequences. These were packaged into bacteriophages, each carrying and expressing a unique VH/VL combination. The library of phages was subjected to three rounds of panning using the soluble biotinylated TfR Avi-his protein with increasing stringency. Individual colonies were picked after the third round and scFv from periplasmic extracts (PPE) were screened for binding by ELISA. After the initial ELISA screening, 46 of the 9_1B scFv variants were selected to clone into the 2+1 ‘hole’ vector (described above in Example 9) in between BamHI and NotI sites which is 3′ of the 3x(G4S) linker that connect the Fc portion and the scFv portion. These 46 TfR9.1B 2+1 bispecific antibodies were expressed and purified in the Expi293 expression system as described above (Example 9) for further characterization and are listed below in Table 19.

TABLE 19
Affinity-optimized TfR9.1B variant sequences
Heavy chain and light chain sequences are
underlined.

| Variant | Anti-TfR scFv Sequence |
|---|---|
| TfR9.1B.1 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLQRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQSFGGGTKVEIK (SEQ ID NO: |
| | 228) |
| TfR9.1B.2 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RWLLRGGDHWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRESGSGSGTDFTLKISRVEAEDVGVYY |
| | CWQGTHEPQTFGGGTKVEIK (SEQ ID NO: |
| | 229) |
| TfR9.1B.3 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQSFGGGTKVEIK (SEQ ID NO: |
| | 230) |
| TfR9.1B.4 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | QSLLDSDGKIYLNWLQQRPGQSPRRLIYLVSKL |
| | DSGVPDRFSGSGSGTDFTLKISRVEAEDVGVYY |
| | CWQGTHEPQTFGGGTKVEIK (SEQ ID NO: |
| | 231) |
| TfR9.1B.5 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLLIGGDYWGQGTLVTVSSGGSEGKSSGSGSE |
| | SKSTGGSDVVMTQSPLSLPVTLGQPASISCKSS |
| | QSDLDSDGKTYLNWLQQRPGQSPRRLIYLVSKL |
| | DSGVPDRFSGSGSGTDFTLKISRVEAEDVGVYY |
| | CWQGTSFPQTFGGGTKVEIK (SEQ ID NO: |
| | 232) |
| TfR9.1B.6 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKTS |
| | QSLLDPDGKTYLNWLQQRPGQSPRRLIYLVSKL |
| | DSGVPDRESGSGSGTDFTLKISRVEAEDVGVYY |
| | CWQGTHFPQTFGGGTKVEIK (SEQ ID NO: |
| | 233) |
| TfR9.1B.7 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLQRGGDYWGQGTLVTVSSGGSEGKPSGSGSE |
| | SKSTGGSDVVMTQSPLSLPVTLGQPASISCKSS |
| | QSLKDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 234) |
| TfR9.1B.8 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | KSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 235) |
| TfR9.1B.9 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | VSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 236) |
| TfR9.1B.10 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | GWLLRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 237) |
| TfR9.1B.11 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | QSLLDSDGVTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 238) |
| TfR9.1B.12 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 239) |
| TfR9.1B.14 | QVQLVQSGAEVKKPGASVKVSCKASGYTFTEYA |
| | MHWVRQAPGQGLEWMGGINPNDGGTHYNQKFKG |
| | RVTMTVDTSTSTAYMELSSLRSEDTAVYYCASQ |
| | RWLLRGGDYWGQGTLVTVSSGGSEGKSSGSGSE |
| | SKSTGGSDVVMTQSPLSLPVTLGQPASISCKSS |
| | RSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 240) |
| TfR9.1B.15 | QVQLVQSGAEVKKPGASVKVSCKASGYTFTEYA |
| | MHWVRQAPGQGLEWMGGINPNDGGTSYSQKFKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | QSLLDKDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 241) |
| TfR9.1B.16 | QVQLVQSGAEVKKPGASVKVSCKASGYTFTEYA |
| | MHWVRQAPGQGLEWMGGINPNDSGTSYNQKEKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 242) |
| TfR9.1B.17 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSNLRSEDTAVYYCASQ |
| | RWLLKGGDYWGQGTLVTVSSGGSEGKSSGSGSE |
| | SKSTGGSDVVMTQSPLSLPVTLGQPASISCKSS |
| | QSLLDSDGITYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 243) |
| TfR9.1B.18 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | TSLLDSDGKTYLNWLQQRPGQSPRRLIYLVSKL |
| | DSGVPDRFSGSGSGTDFTLKISRVEAEDVGVYY |
| | CWQGTHEPQTFGGGTKVEIK (SEQ ID NO: |
| | 244) |
| TfR9.1B.19 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RWLQRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 245) |
| TfR9.1B.20 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLQRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTKFPQTFGGGTKVEIK (SEQ ID NO: |
| | 246) |
| TfR9.1B.21 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | QSLLDSDGRTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 247) |
| TfR9.1B.22 | QVQLVQSGAEVKKPGASVKVSCKASGYTFTEYA |
| | MHWVRQAPGQGLEWMGGINPKDGGTSYNQKFKG |
| | RVTMTVDTSTSTAYMELSSLRSEDTAVYYCASQ |
| | GWLLRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 248) |
| TfR9.1B.23 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RWLQRGGDYWGQGTLVTVSSGGSEGKSSGSGSE |
| | SKSTGGSDVVMTQSPLSLPVTLGQPASISCKSS |
| | QSLIDSDGKTYLNWLQQRPGQSPRRLIYLVSKL |
| | DSGVPDRFSGSGSGTDFTLKISRVEAEDVGVYY |
| | CWQGTHEPQTFGGGTKVEIK (SEQ ID NO: |
| | 249) |
| TfR9.1B.24 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RWLMRGGDYWGQGTLVTVSSGGSEGKSSSSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 250) |
| TfR9.1B.25 | QVQLVQSGAEVKKPGASVKVSCKASGYTFTEHA |
| | MHWVRQAPGQGLEWMGGINPNDGGTSTNQKEKG |
| | RVTMTVDTSTSTAYMELSSLRSEDTAVYYCASQ |
| | GWLLRGGDYWGQGTLVTVSSGGSEGKSSGSGSE |
| | SKSTGGSDVVMTQSPLSLPVTLGQPASISCKSS |
| | VSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID |
| | NO: 251) |
| TfR9.1B.26 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | GWLLRGGDYWGQGTLVTVSSGGSEDKSSGSGSE |
| | SKSTGGSDVVMTQSPLSLPVTLGQPASISCKSS |
| | QSLLDSDGVTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID |
| | NO: 252) |
| TfR9.1B.27 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | QSLLDEDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 253) |
| TfR9.1B.28 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RWLQRGGDYWGQGTLVTVSSGGSEGKSSGSGSE |
| | SKSTGGSDVVMTQSPLSLPVTLGQPASISCKSS |
| | QSLEDSDGKTYLNWLQQRPGQSPRRLIYLVSKL |
| | DSGVPDRFSGSGSGTDFTLKISRVEAEDVGVYY |
| | CWQGTHEPQTFGGGTKVEIK (SEQ ID NO: |
| | 254) |
| TfR9.1B.29 | QVQLVQSGAEVKKPGASVKVSCKASGYAFTEYA |
| | MHWVRQAPGQGLEWMGGINPNDGGTSYNQKFKG |
| | RVTMTVDTSTSTAYMELSSLRSEDTAVYYCASQ |
| | RWLERGGNYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRESGSGSGTDFTLKISRVEAEDVGVYY |
| | CWQGTHFPQTFGGGTKVEIK (SEQ ID NO: |
| | 255) |
| TfR9.1B.30 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RWLFRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 256) |
| TfR9.1B.31 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLQRGGDYWGQGTLVTVSSGGSEGKSSGSGSE |
| | SKSTGGSDVVMTQSPLSLPVTLGQPASISCKSS |
| | QSLQDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 257) |
| TfR9.1B.32 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RSLLRGGDYWGQGTLVTVSSGGSEGKSSGSGSE |
| | SKSTGGSDVVMTQSPLSLPVTLGQPASISCKPS |
| | QSLLDGDGKTYLNWLQQRPGQSPRRLIYLVSKL |
| | DSGVPDRESGSGSGTDFTLKISRVEAEDVGVYY |
| | CWQGTHEPQTFGGGTKVEIK (SEQ ID NO: |
| | 258) |
| TfR9.1B.33 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RWLYRGGDYWGQGTLVTVSSGGSEGKSSGSGSE |
| | SKSTGGSDVVMTQSPLSLPVTLGQPASISCKSS |
| | QSLLDEDGKTYLNWLQQRPGQSPRRLIYQVSKL |
| | DSGVPDRESGSGSGTDFTLKISRVEAEDVGVYY |
| | CWQGTHFPQTFGGGTKVEIK (SEQ ID NO: |
| | 259) |
| TfR9.1B.34 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKTS |
| | QSLLDPDGKTYLNWLQQRPGQSPRRLIYLVSKL |
| | DSGVPDRFSGSGSGTDFTLKISRVEAEDVGVYY |
| | CWQSTHEPQTFGGGTKVEIK (SEQ ID NO: |
| | 261) |
| TfR9.1B.35 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | GWLLRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYH |
| | CWQGTHFPQSFGGGTKVEIK (SEQ ID NO: |
| | 262) |
| TfR9.1B.36 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RWLERGGDYWGQGTLVTVSSGGSEGKSSGSGSE |
| | SKSTGGSDVVMTQSPLSLPVTLGQPASISCKSS |
| | QSLLDSDGVTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 263) |
| TfR9.1B.37 | QVQLVQSGAEVKKPGASVKVSCKASGYTFTEYA |
| | MHWVRQAPGQGLEWMGGINPNDGGTFYNQKFKG |
| | RVTMTVDTSTSTAYMELSSLRSEDTAVYYCASQ |
| | GWLLRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 264) |
| TfR9.1B.38 | QVQLVQSGAEVKKPGASVKVSCKASGYTFTEYA |
| | MHWVRQAPGQGLEWMGGINPNDGGTSYAQKEKG |
| | RVTMTVDTSTSTAYMELSSLRSEDTAVYYCASQ |
| | RWLNRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 265) |
| TfR9.1B.39 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | GWLLRGGDYWGQGTLVTVSSGGSEGKSSGSGSE |
| | SKSTGGSDVVMTQSPLSLPVTLGQPASISCKAS |
| | QSLLDEDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 266) |
| TfR9.1B.40 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RWLDRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTQFPQTFGGGTKVEIK (SEQ ID NO: |
| | 267) |
| TfR9.1B.41 | QVQLVQSGAEVKKPGASVKVSCKASGYTFTEYA |
| | MHWVRQAPGQGLEWMGGINPNDGGTHYNQKFKG |
| | RVTMTVDTSTSTAYMELSSLRSEDTAVYYCASQ |
| | RWLLRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 268) |
| TfR9.1B.42 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | RELLRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHEPQTFGGGTKVEIK (SEQ ID NO: |
| | 269) |
| TfR9.1B.43 | QVQLVQSGAEVKKPGASVKVSCKASGYTFTEYA |
| | MHWVRQAPGQGLEWMGGINPNDGGTSYLQKEKG |
| | RVTMTVDTSTSTAYMELSSLRSEDTAVYYCASQ |
| | RWLLRGGDYWGQGTLVTVSSGGSEGKSSGSGSE |
| | SKSTGGSDVVMTQSPLSLPVTLGQPASISCKSS |
| | QSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 270) |
| TfR9.1B.44 | QVQLVQSGAEVKKPGASVKVSCKASGYTFTEYA |
| | MHWVRQAPGQGLEWMGGINPNDGGTSYNQKEKG |
| | RVTMTVDTSTSTAYMELSSLRSEDTAVYYCASQ |
| | RWLLRGGDYWGQGTLVTVSSGGSEGKSSGSGSE |
| | SKSTGGSDVVMTQSPLSLPVTLGQPASISCKSS |
| | QSLLDSIGKTYLNWLQQRPGQSPRRLIYLVSKL |
| | DSGVPDRFSGSGSGTDFTLKISRVEAEDVGVYY |
| | CWQGTHEPQTFGGGTKVEIK (SEQ ID NO: |
| | 271) |
| TfR9.1B.45 | QVQLVQSGAEVKKPGASVKVSCKASGYTFTEYA |
| | MHWVRQAPGQGLEWMGGINPNDGGTSYNQKFKG |
| | RVTMTVDTSTSTAYMELSSLRSEDTAVYYCASQ |
| | GWLQRGGDYWGQGTLVTVSSGGSEGKSSGSGSE |
| | SKSTGGSDVVMTQSPLSLPVTLGQPASISCKSS |
| | VSLLDSDGKTYLNWLQQRPGQSPRRLIYLVSKL |
| | DSGVPDRFSGSGSGTDFTLKISRVEAEDVGVYY |
| | CWQGTHFPQTFGGGTKVEIK (SEQ ID NO: |
| | 272) |
| TfR9.1B.46 | QVQLVQSGAEVKKPGASVKVSCKASGYTFTEYA |
| | MHWVRQAPGQGLEWMGGINPNDGGTSYLQKFKG |
| | RVTMTVDTSTSTAYMELSSLRSEDTAVYYCASQ |
| | RWLLRGGDYWGQGTLVTVSSGGSEGKSSGSGSE |
| | SKSTGGSDVVMTQSPLSLPVTLGQPASISCKSS |
| | QSLLDSIGKTYLNWLQQRPGQSPRRLIYLVSKL |
| | DSGVPDRESGSGSGTDFTLKISRVEAEDVGVYY |
| | CWQGTHFPQTFGGGTKVEIK (SEQ ID NO: |
| | 273) |

TABLE 20
Variable heavy and variable light chain domains for
affinity-optimized TfR9.1B variants

| Antibody | VH sequence | VL sequence |
|---|---|---|
| TfR9.1B.1 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQS |
| | LRSEDTAVYYCASQRWLQRGG | FGGGTKVEIK (SEQ ID NO: 347) |
| | DYWGQGTLVTVSS (SEQ ID | |
| | NO: 346) | |
| TfR9.1B.2 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLLRGG | FGGGTKVEIK (SEQ ID NO: 316) |
| | DHWGQGTLVTVSS (SEQ ID | |
| | NO: 348) | |
| TfR9.1B.3 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQS |
| | LRSEDTAVYYCASQRWLERGG | FGGGTKVEIK (SEQ ID NO: 347) |
| | DYWGQGTLVTVSS (SEQ ID | |
| | NO: 349) | |
| TfR9.1B.4 | QVQLVQSGAEVKKPGASVKVS | DVVMTQSPLSLPVTLGQPASISCKSS |
| | CKASGYTFTEYAMHWVRQAPG | QSLLDSDGKIYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLERGG | FGGGTKVEIK (SEQ ID NO: 350) |
| | DYWGQGTLVTVSS (SEQ ID | |
| | NO: 349) | |
| TfR9.1B.5 | QVQLVQSGAEVKKPGASVKVS | DVVMTQSPLSLPVTLGQPASISCKSS |
| | CKASGYTFTEYAMHWVRQAPG | QSDLDSDGKTYLNWLQQRPGQSPR |
| | QGLEWMGGINPNDGGTSYNQK | RLIYLVSKLDSGVPDRFSGSGSGTDF |
| | FKGRVTMTVDTSTSTAYMELSS | TLKISRVEAEDVGVYYCWQGTSFPQ |
| | LRSEDTAVYYCASQRWLLIGGD | TFGGGTKVEIK (SEQ ID NO: 352) |
| | YWGQGTLVTVSS (SEQ ID | |
| | NO: 351) | |
| TfR9.1B.6 | QVQLVQSGAEVKKPGASVKVS | DVVMTQSPLSLPVTLGQPASISCKTS |
| | CKASGYTFTEYAMHWVRQAPG | QSLLDPDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLERGG | FGGGTKVEIK (SEQ ID NO: 353) |
| | DYWGQGTLVTVSS (SEQ ID | |
| | NO: 349) | |
| TfR9.1B.7 | QVQLVQSGAEVKKPGASVKVS | DVVMTQSPLSLPVTLGQPASISCKSS |
| | CKASGYTFTEYAMHWVRQAPG | QSLKDSDGKTYLNWLQQRPGQSPR |
| | QGLEWMGGINPNDGGTSYNQK | RLIYLVSKLDSGVPDRFSGSGSGTDF |
| | FKGRVTMTVDTSTSTAYMELSS | TLKISRVEAEDVGVYYCWQGTHFPQ |
| | LRSEDTAVYYCASQRWLQRGG | TFGGGTKVEIK (SEQ ID NO: 354) |
| | DYWGQGTLVTVSS (SEQ ID | |
| | NO: 346) | |
| TfR9.1B.8 | QVQLVQSGAEVKKPGASVKVS | DVVMTQSPLSLPVTLGQPASISCKSS |
| | CKASGYTFTEYAMHWVRQAPG | KSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLERGG | FGGGTKVEIK (SEQ ID NO: 355) |
| | DYWGQGTLVTVSS (SEQ ID | |
| | NO: 349) | |
| TfR9.1B.9 | QVQLVQSGAEVKKPGASVKVS | DVVMTQSPLSLPVTLGQPASISCKSS |
| | CKASGYTFTEYAMHWVRQAPG | VSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLERGG | FGGGTKVEIK (SEQ ID NO: 356) |
| | DYWGQGTLVTVSS (SEQ ID | |
| | NO: 349) | |
| TfR9.1B.10 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQGWLLRGG | FGGGTKVEIK (SEQ ID NO: 316) |
| | DYWGQGTLVTVSS (SEQ ID | |
| | NO: 357) | |
| TfR9.1B.11 | QVQLVQSGAEVKKPGASVKVS | DVVMTQSPLSLPVTLGQPASISCKSS |
| | CKASGYTFTEYAMHWVRQAPG | QSLLDSDGVTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | |
| | LRSEDTAVYYCASQRWLERGG | LKISRVEAEDVGVYYCWQGTHFPQT |
| | DYWGQGTLVTVSS (SEQ ID | FGGGTKVEIK (SEQ ID NO: 358) |
| | NO: 349) | |
| TfR9.1B.12 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLERGG | FGGGTKVEIK (SEQ ID NO: 316) |
| | DYWGQGTLVTVSS (SEQ ID | |
| | NO: 349) | |
| TfR9.1B.14 | QVQLVQSGAEVKKPGASVKVS | DVVMTQSPLSLPVTLGQPASISCKSS |
| | CKASGYTFTEYAMHWVRQAPG | RSLLDSDGKTYLNWLQQRPGQSPRR |
| | QGLEWMGGINPNDGGTHYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLLRGG | FGGGTKVEIK (SEQ ID NO: 360) |
| | DYWGQGTLVTVSS (SEQ ID | |
| | NO: 359) | |
| TfR9.1B.15 | QVQLVQSGAEVKKPGASVKVS | DVVMTQSPLSLPVTLGQPASISCKSS |
| | CKASGYTFTEYAMHWVRQAPG | QSLLDKDGKTYLNWLQQRPGQSPR |
| | QGLEWMGGINPNDGGTSYSQK | RLIYLVSKLDSGVPDRFSGSGSGTDF |
| | FKGRVTMTVDTSTSTAYMELSS | TLKISRVEAEDVGVYYCWQGTHFPQ |
| | LRSEDTAVYYCASQRWLERGG | TFGGGTKVEIK (SEQ ID NO: 362) |
| | DYWGQGTLVTVSS (SEQ ID | |
| | NO: 361) | |
| TfR9.1B.16 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDSGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLERGG | FGGGTKVEIK (SEQ ID NO: 316) |
| | DYWGQGTLVTVSS (SEQ ID | |
| | NO: 363) | |
| TfR9.1B.17 | QVQLVQSGAEVKKPGASVKVS | DVVMTQSPLSLPVTLGQPASISCKSS |
| | CKASGYTFTEYAMHWVRQAPG | QSLLDSDGITYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSN | LKISRVEAEDVGVYYCWQGTHFPQT |
| | LRSEDTAVYYCASQRWLLKGG | FGGGTKVEIK (SEQ ID NO: 365) |
| | DYWGQGTLVTVSS (SEQ ID | |
| | NO: 364) | |
| TfR9.1B.18 | QVQLVQSGAEVKKPGASVKVS | DVVMTQSPLSLPVTLGQPASISCKSS |
| | CKASGYTFTEYAMHWVRQAPG | TSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLERGG | FGGGTKVEIK (SEQ ID NO: 366) |
| | DYWGQGTLVTVSS (SEQ ID | |
| | NO: 349) | |
| TfR9.1B.19 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | |
| | LRSEDTAVYYCASQRWLQRGG | LKISRVEAEDVGVYYCWQGTHFPQT |
| | DYWGQGTLVTVSS (SEQ ID | FGGGTKVEIK (SEQ ID NO: 316) |
| | NO: 346) | |
| TfR9.1B.20 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTKFPQT |
| | LRSEDTAVYYCASQRWLQRGG | FGGGTKVEIK (SEQ ID NO: 367) |
| | DYWGQGTLVTVSS (SEQ ID | |
| | NO: 346) | |
| TfR9.1B.21 | QVQLVQSGAEVKKPGASVKVS | DVVMTQSPLSLPVTLGQPASISCKSS |
| | CKASGYTFTEYAMHWVRQAPG | QSLLDSDGRTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLERGG | FGGGTKVEIK (SEQ ID NO: 368) |
| | DYWGQGTLVTVSS (SEQ ID | |
| | NO: 349) | |
| TfR9.1B.22 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPKDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQGWLLRGG | FGGGTKVEIK (SEQ ID NO: 316) |
| | DYWGQGTLVTVSS (SEQ ID | |
| | NO: 369) | |
| TfR9.1B.23 | QVQLVQSGAEVKKPGASVKVS | DVVMTQSPLSLPVTLGQPASISCKSS |
| | CKASGYTFTEYAMHWVRQAPG | QSLIDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLQRGG | FGGGTKVEIK (SEQ ID NO: 370) |
| | DYWGQGTLVTVSS (SEQ ID | |
| | NO: 346) | |
| TfR9.1B.24 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLMRGG | FGGGTKVEIK (SEQ ID NO: 316) |
| | DYWGQGTLVTVSS (SEQ ID | |
| | NO: 371) | |
| TfR9.1B.25 | QVQLVQSGAEVKKPGASVKVS | DVVMTQSPLSLPVTLGQPASISCKSS |
| | CKASGYTFTEHAMHWVRQAPG | VSLLDSDGKTYLNWLQQRPGQSPRR |
| | QGLEWMGGINPNDGGTSTNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQGWLLRGG | FGGGTKVEIK (SEQ ID NO: 356) |
| | DYWGQGTLVTVSS (SEQ ID | |
| | NO: 372) | |
| TfR9.1B.26 | QVQLVQSGAEVKKPGASVKVS | DVVMTQSPLSLPVTLGQPASISCKSS |
| | CKASGYTFTEYAMHWVRQAPG | QSLLDSDGVTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | |
| | LRSEDTAVYYCASQGWLLRGG | LKISRVEAEDVGVYYCWQGTHFPQT |
| | DYWGQGTLVTVSS (SEQ ID | FGGGTKVEIK (SEQ ID NO: 358) |
| | NO: 357) | |
| TfR9.1B.27 | QVQLVQSGAEVKKPGASVKVS | DVVMTQSPLSLPVTLGQPASISCKSS |
| | CKASGYTFTEYAMHWVRQAPG | QSLLDEDGKTYLNWLQQRPGQSPR |
| | QGLEWMGGINPNDGGTSYNQK | RLIYLVSKLDSGVPDRFSGSGSGTDF |
| | FKGRVTMTVDTSTSTAYMELSS | TLKISRVEAEDVGVYYCWQGTHFPQ |
| | LRSEDTAVYYCASQRWLERGG | TFGGGTKVEIK (SEQ ID NO: 373) |
| | DYWGQGTLVTVSS (SEQ ID | |
| | NO: 349) | |
| TfR9.1B.28 | QVQLVQSGAEVKKPGASVKVS | DVVMTQSPLSLPVTLGQPASISCKSS |
| | CKASGYTFTEYAMHWVRQAPG | QSLEDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLQRGG | FGGGTKVEIK (SEQ ID NO: 374) |
| | DYWGQGTLVTVSS (SEQ ID | |
| | NO: 346) | |
| TfR9.1B.29 | QVQLVQSGAEVKKPGASVKVS | DVVMTQSPLSLPVTLGQPASISCKSS |
| | CKASGYAFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLERGG | FGGGTKVEIK (SEQ ID NO: 316) |
| | NYWGQGTLVTVSS (SEQ ID | |
| | NO: 375) | |
| TfR9.1B.30 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLFRGG | FGGGTKVEIK (SEQ ID NO: 316) |
| | DYWGQGTLVTVSS (SEQ ID | |
| | NO: 376) | |
| TfR9.1B.31 | QVQLVQSGAEVKKPGASVKVS | DVVMTQSPLSLPVTLGQPASISCKSS |
| | CKASGYTFTEYAMHWVRQAPG | QSLQDSDGKTYLNWLQQRPGQSPR |
| | QGLEWMGGINPNDGGTSYNQK | RLIYLVSKLDSGVPDRFSGSGSGTDF |
| | FKGRVTMTVDTSTSTAYMELSS | TLKISRVEAEDVGVYYCWQGTHFPQ |
| | LRSEDTAVYYCASQRWLQRGG | TFGGGTKVEIK (SEQ ID NO: 377) |
| | DYWGQGTLVTVSS (SEQ ID | |
| | NO: 346) | |
| TfR9.1B.32 | QVQLVQSGAEVKKPGASVKVS | DVVMTQSPLSLPVTLGQPASISCKPS |
| | CKASGYTFTEYAMHWVRQAPG | QSLLDGDGKTYLNWLQQRPGQSPR |
| | QGLEWMGGINPNDGGTSYNQK | RLIYLVSKLDSGVPDRFSGSGSGTDF |
| | FKGRVTMTVDTSTSTAYMELSS | TLKISRVEAEDVGVYYCWQGTHFPQ |
| | LRSEDTAVYYCASQRSLLRGGD | TFGGGTKVEIK (SEQ ID NO: 379) |
| | YWGQGTLVTVSS (SEQ ID | |
| | NO: 378) | |
| TfR9.1B.33 | QVQLVQSGAEVKKPGASVKVS | DVVMTQSPLSLPVTLGQPASISCKSS |
| | CKASGYTFTEYAMHWVRQAPG | QSLLDEDGKTYLNWLQQRPGQSPR |
| | QGLEWMGGINPNDGGTSYNQK | RLIYQVSKLDSGVPDRFSGSGSGTDF |
| | FKGRVTMTVDTSTSTAYMELSS | |
| | LRSEDTAVYYCASQRWLYRGG | TLKISRVEAEDVGVYYCWQGTHFPQ |
| | DYWGQGTLVTVSS (SEQ ID | TFGGGTKVEIK (SEQ ID NO: 381) |
| | NO: 380) | |
| TfR9.1B.34 | QVQLVQSGAEVKKPGASVKVS | DVVMTQSPLSLPVTLGQPASISCKTS |
| | CKASGYTFTEYAMHWVRQAPG | QSLLDPDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISR VEAEDVGVYYCWQSTHFPQT |
| | LRSEDTAVYYCASQRWLERGG | FGGGTKVEIK (SEQ ID NO: 382) |
| | DYWGQGTLVTVSS (SEQ ID | |
| | NO: 349) | |
| TfR9.1B.35 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYHCWQGTHFPQS |
| | LRSEDTAVYYCASQGWLLRGG | FGGGTKVEIK (SEQ ID NO: 383) |
| | DYWGQGTLVTVSS (SEQ ID | |
| | NO: 357) | |
| TfR9.1B.36 | QVQLVQSGAEVKKPGASVKVS | DVVMTQSPLSLPVTLGQPASISCKSS |
| | CKASGYTFTEYAMHWVRQAPG | QSLLDSDGVTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLERGG | FGGGTKVEIK (SEQ ID NO: 358) |
| | DYWGQGTLVTVSS (SEQ ID | |
| | NO: 349) | |
| TfR9.1B.37 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTFYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQGWLLRGG | FGGGTKVEIK (SEQ ID NO: 316) |
| | DYWGQGTLVTVSS (SEQ ID | |
| | NO: 384) | |
| TfR9.1B.38 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYAQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLNRGG | FGGGTKVEIK (SEQ ID NO: 316) |
| | DYWGQGTLVTVSS (SEQ ID | |
| | NO: 385) | |
| TfR9.1B.39 | QVQLVQSGAEVKKPGASVKVS | DVVMTQSPLSLPVTLGQPASISCKAS |
| | CKASGYTFTEYAMHWVRQAPG | QSLLDEDGKTYLNWLQQRPGQSPR |
| | QGLEWMGGINPNDGGTSYNQK | RLIYLVSKLDSGVPDRFSGSGSGTDF |
| | FKGRVTMTVDTSTSTAYMELSS | TLKISRVEAEDVGVYYCWQGTHFPQ |
| | LRSEDTAVYYCASQGWLLRGG | TFGGGTKVEIK (SEQ ID NO: 386) |
| | DYWGQGTLVTVSS (SEQ ID | |
| | NO: 357) | |
| TfR9.1B.40 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | |
| | LRSEDTAVYYCASQRWLDRGG | LKISRVEAEDVGVYYCWQGTQFPQT |
| | DYWGQGTLVTVSS (SEQ ID | FGGGTKVEIK (SEQ ID NO: 388) |
| | NO: 387) | |
| TfR9.1B.41 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTHYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLLRGG | FGGGTKVEIK (SEQ ID NO: 316) |
| | DYWGQGTLVTVSS (SEQ ID | |
| | NO: 359) | |
| TfR9.1B.42 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRELLRGGD | FGGGTKVEIK (SEQ ID NO: 316) |
| | YWGQGTLVTVSS (SEQ ID | |
| | NO: 389) | |
| TfR9.1B.43 | QVQLVQSGAEVKKPGASVKVS | DVVMTQSPLSLPVTLGQPASISCKSS |
| | CKASGYTFTEYAMHWVRQAPG | QSLLDSDGKTYLNWLQQRPGQSPRR |
| | QGLEWMGGINPNDGGTSYLQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLLRGG | FGGGTKVEIK (SEQ ID NO: 316) |
| | DYWGQGTLVTVSS (SEQ ID | |
| | NO: 390) | |
| TfR9.1B.44 | QVQLVQSGAEVKKPGASVKVS | DVVMTQSPLSLPVTLGQPASISCKSS |
| | CKASGYTFTEYAMHWVRQAPG | QSLLDSIGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLLRGG | FGGGTKVEIK (SEQ ID NO: 392) |
| | DYWGQGTLVTVSSGGSEGKSS | |
| | (SEQ ID NO: 391) | |
| TfR9.1B.45 | QVQLVQSGAEVKKPGASVKVS | DVVMTQSPLSLPVTLGQPASISCKSS |
| | CKASGYTFTEYAMHWVRQAPG | VSLLDSDGKTYLNWLQQRPGQSPRR |
| | QGLEWMGGINPNDGGTSYNQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQGWLQRGG | FGGGTKVEIK (SEQ ID NO: 356) |
| | DYWGQGTLVTVSS (SEQ ID | |
| | NO: 393) | |
| TfR9.1B.46 | QVQLVQSGAEVKKPGASVKVS | DVVMTQSPLSLPVTLGQPASISCKSS |
| | CKASGYTFTEYAMHWVRQAPG | QSLLDSIGKTYLNWLQQRPGQSPRR |
| | QGLEWMGGINPNDGGTSYLQK | LIYLVSKLDSGVPDRFSGSGSGTDFT |
| | FKGRVTMTVDTSTSTAYMELSS | LKISRVEAEDVGVYYCWQGTHFPQT |
| | LRSEDTAVYYCASQRWLLRGG | FGGGTKVEIK (SEQ ID NO: 392) |
| | DYWGQGTLVTVSS (SEQ ID | |
| | NO: 390) | |

Clone TfR9.1B.39 was chosen as the template for an additional round of random mutagenesis to remove two potential isomerization sites (1D34 in VL and D62 in VH). Using biotinylated human and cyno TfR Avi-His protein as bait for panning and ELISA for screening, seven mutants with both potential isomerization sites fixed were selected to clone into the same vector in the 2+1 bispecific format and used for further characterization (see Table 21).

TABLE 21
TfR9.1B.39 variant sequences (VH and VL sequences are underlined)

| Variant | Anti-TfR scFv Sequence |
|---|---|
| TfR9.1B.39 | QVQLVQSGAEVKKPGASVKVSCKASGYTFTEYAMHWVRQAPG |
| scFv | QGLEWMGGINPNDGGTSYNQKFKGRVTMTVDTSTSTAYMELSS |
| | LRSEDTAVYYCASQGWLLRGGDYWGQGTLVTVSSGGSEGKSSG |
| | SGSESKSTGGSDVVMTQSPLSLPVTLGQPASISCKASQSLLDEDGK |
| | TYLNWLQQRPGQSPRRLIYLVSKLDSGVPDRFSGSGSGTDFTLKIS |
| | RVEAEDVGVYYCWQGTHFPQTFGGGTKVEIK (SEQ ID NO: 274) |
| TfR9.1B.39.5 | QVQLVQSGAEVKKPGASVKVSCKASGYTFTEYAMHWVRQAPG |
| scFv | QGLEWMGGIDPRSGGTSYNQKFKGRVTMTVDTSTSTAYMELSSL |
| | RSEDTAVYYCASQGWLKRGGDYWGQGTLVTVSSGGSEGRSSGS |
| | GSESKSTGGSDVVMTQSPLSLPVTLGQPASISCKASQSLLDEEGKT |
| | YLNWLQQRPGQSPRRLIYLVSKLDSGVPDRFSGSGSGTDFTLKISR |
| | VEAEDVGVYYCWQGTHFPQTFGGGTKVEIK (SEQ ID NO: 275) |
| TfR9.1B.39.8 | QVQLVQSGAEVKKPGASVKVSCKASGYTFTEYAMHWVRQAPG |
| scFv | QGLEWMGGIDPRSGGTSYNQKFKGRVTMTVDTSTSTAYMELSSL |
| | RSEDTAVYYCASQGWLQRGGDYWGQGTLVTVSSGGSEGKSSGS |
| | GSESKSTGGSDVVMTQSPLSLPVTLGQPASISCKASQSLLDEEGKT |
| | YLNWLQQRPGQSPRRLIYLVSKLDSGVPDRFSGSGSGTDFTLKISR |
| | VEAEDVGVYYCWQGTHEPQTFGGGTKVEIK (SEQ ID NO: 276) |
| TfR9.1B.39.36 | QVQLVQSGAEVKKPGASVKVSCKASGYTFTEYAMHWVRQAPG |
| scFv | QGLEWMGGIDPNTGGTSYNQKFKGRVTMTVDTSTSTAYMELSSL |
| | RSEDTAVYYCASQGWLLRGGDYWGQGTLVTVSSGGSEGKSSGS |
| | GSESKSTGGSDVVMTQSPLSLPVTLGQPASISCKASQSLLDEEGKT |
| | YLNWLQQRPGQSPRRLIYLVSKLDSGVPDRFSGSGSGTDFTLKISR |
| | VEAEDVGVYYCWQGTHFPQTFGGGTKVEIK (SEQ ID NO: 277) |
| TfR9.1B.39.37 | QVQLVQSGAEVKKPGASVKVSCKASGYTFTEYAMHWVRQAPG |
| scFv | QGLEWMGGIAPGTGGTSYNQKFKGRVTMTVDTSTSTAYMELSSL |
| | RSEDTAVYYCASQGWLLRGGDYWGQGTLVTVSSGGSEGKSSGS |
| | GSESKSTGGSDVVMTQSPLSLPVTLGQPASISCKASQSLLDEEGKT |
| | YLNWLQQRPGQSPRRLIYLVSKLDSGVPDRFSGSGSGTDFTLKISR |
| | VEAEDVGVYYCWQGTHFPQTFGGGTKVEIK (SEQ ID NO: 278) |
| TfR9.1B.39.38 | QVQLVQSGAEVKKPGASVKVSCKASGYTFTEYAMHWVRQAPG |
| scFv | QGLEWMGGIAPNTGGTSYNQKFKGRVTMTVDTSTSTAYMELSSL |
| | RSEDTAVYYCASQGWLLRGGDYWGQGTLVTVSSGGSEGKSSGS |
| | GSESKSTGGSDVVMTQSPLSLPVTLGQPASISCKASQSLLDEGGKT |
| | YLNWLQQRPGQSPRRLIYLVSKLDSGVPDRFSGSGSGTDFTLKISR |
| | VEAEDVGVYYCWQGTHFPQTFGGGTKVEIK (SEQ ID NO: 279) |
| TfR9.1B.39.39 | QVQLVQSGAEVKKPGASVKVSCKASGYTFTEYAMHWVRQAPG |
| scFv | QGLEWMGGINPSFGGTSYNQKFKGRVTMTVDTSTSTAYMELSSL |
| | RSEDTAVYYCASQGWLLRGGDYWGQGTLVTVSSGGSEGKSSGS |
| | GSESKSTGGSDVVMTQSPLSLPVTLGQPASISCKASQSLLDEEGKT |
| | YLNWLQQRPGQSPRRLIYLVSKLDSGVPDRFSGSGSGTDFTLKISR |
| | VEAEDVGVYYCWQGTHEPQTFGGGTKVEIK (SEQ ID NO: 280) |
| TfR9.1B.39.40 | QVQLVQSGAEVKKPGASVKVSCKASGYTFTEYAMHWVRQAPG |
| scFv | QGLEWMGGINPKEGGTSYNQKFKGRVTMTVDTSTSTAYMELSSL |
| | RSEDTAVYYCASQGWLLRGGDYWGQGTLVTVSSGGSEGKSSGS |
| | GSESKSTGGSDVVMTQSPLSLPVTLGQPASISCKASQSLLDEDQKT |
| | YLNWLQQRPGQSPRRLIYLVSKLDSGVPDRFSGSGSGTDFTLKISR |
| | VEAEDVGVYYCWQGTHFPQTFGGGTKVEIK (SEQ ID NO: 281) |

TABLE 22
Variable heavy and variable light chain domains for TfR9.1B.39 variants

| Antibody | VH sequence | VL sequence |
|---|---|---|
| TfR9.1B.39 | QVQLVQSGAEVKKPGASVKV | DVVMTQSPLSLPVTLGQPASISCKAS |
| scFv | SCKASGYTFTEYAMHWVRQ | QSLLDEDGKTYLNWLQQRPGQSPR |
| | APGQGLEWMGGINPNDGGTS | RLIYLVSKLDSGVPDRFSGSGSGTDF |
| | YNQKFKGRVTMTVDTSTSTA | TLKISRVEAEDVGVYYCWQGTHFPQ |
| | YMELSSLRSEDTAVYYCASQ | TFGGGTKVEIK (SEQ ID NO: 386) |
| | GWLLRGGDYWGQGTLVTVS | |
| | S (SEQ ID NO: 357) | |
| TfR9.1B.39.5 | QVQLVQSGAEVKKPGASVKV | DVVMTQSPLSLPVTLGQPASISCKAS |
| scFv | SCKASGYTFTEYAMHWVRQ | QSLLDEEGKTYLNWLQQRPGQSPRR |
| | APGQGLEWMGGIDPRSGGTS | LIYLVSKLDSGVPDRFSGSGSGTDFT |
| | YNQKFKGRVTMTVDTSTSTA | LKISRVEAEDVGVYYCWQGTHFPQT |
| | YMELSSLRSEDTAVYYCASQ | FGGGTKVEIK (SEQ ID NO: 395) |
| | GWLKRGGDYWGQGTLVTVS | |
| | S (SEQ ID NO: 394) | |
| TfR9.1B.39.8 | QVQLVQSGAEVKKPGASVKV | DVVMTQSPLSLPVTLGQPASISCKAS |
| scFv | SCKASGYTFTEYAMHWVRQ | QSLLDEEGKTYLNWLQQRPGQSPRR |
| | APGQGLEWMGGIDPRSGGTS | LIYLVSKLDSGVPDRFSGSGSGTDFT |
| | YNQKFKGRVTMTVDTSTSTA | LKISRVEAEDVGVYYCWQGTHFPQT |
| | YMELSSLRSEDTAVYYCASQ | FGGGTKVEIK (SEQ ID NO: 395) |
| | GWLQRGGDYWGQGTLVTVS | |
| | S (SEQ ID NO: 396) | |
| TfR9.1B.39.36 | QVQLVQSGAEVKKPGASVKV | DVVMTQSPLSLPVTLGQPASISCKAS |
| scFv | SCKASGYTFTEYAMHWVRQ | QSLLDEEGKTYLNWLQQRPGQSPRR |
| | APGQGLEWMGGIDPNTGGTS | LIYLVSKLDSGVPDRFSGSGSGTDFT |
| | YNQKFKGRVTMTVDTSTSTA | LKISRVEAEDVGVYYCWQGTHFPQT |
| | YMELSSLRSEDTAVYYCASQ | FGGGTKVEIK (SEQ ID NO: 395) |
| | GWLLRGGDYWGQGTLVTVS | |
| | S (SEQ ID NO: 397) | |
| TfR9.1B.39.37 | QVQLVQSGAEVKKPGASVKV | DVVMTQSPLSLPVTLGQPASISCKAS |
| scFv | SCKASGYTFTEYAMHWVRQ | QSLLDEEGKTYLNWLQQRPGQSPRR |
| | APGQGLEWMGGIAPGTGGTS | LIYLVSKLDSGVPDRFSGSGSGTDFT |
| | YNQKFKGRVTMTVDTSTSTA | LKISRVEAEDVGVYYCWQGTHFPQT |
| | YMELSSLRSEDTAVYYCASQ | FGGGTKVEIK (SEQ ID NO: 395) |
| | GWLLRGGDYWGQGTLVTVS | |
| | S (SEQ ID NO: 398) | |
| TfR9.1B.39.38 | QVQLVQSGAEVKKPGASVKV | DVVMTQSPLSLPVTLGQPASISCKAS |
| scFv | SCKASGYTFTEYAMHWVRQ | QSLLDEGGKTYLNWLQQRPGQSPR |
| | APGQGLEWMGGIAPNTGGTS | RLIYLVSKLDSGVPDRFSGSGSGTDF |
| | YNQKFKGRVTMTVDTSTSTA | TLKISRVEAEDVGVYYCWQGTHFPQ |
| | YMELSSLRSEDTAVYYCASQ | TFGGGTKVEIK (SEQ ID NO: 400) |
| | GWLLRGGDYWGQGTLVTVS | |
| | S (SEQ ID NO: 399) | |
| TfR9.1B.39.39 | QVQLVQSGAEVKKPGASVKV | DVVMTQSPLSLPVTLGQPASISCKAS |
| scFv | SCKASGYTFTEYAMHWVRQ | QSLLDEEGKTYLNWLQQRPGQSPRR |
| | APGQGLEWMGGINPSFGGTS | LIYLVSKLDSGVPDRFSGSGSGTDFT |
| | YNQKFKGRVTMTVDTSTSTA | LKISRVEAEDVGVYYCWQGTHFPQT |
| | YMELSSLRSEDTAVYYCASQ | FGGGTKVEIK (SEQ ID NO: 395) |
| | GWLLRGGDYWGQGTLVTVS | |
| | S (SEQ ID NO: 401) | |
| TfR9.1B.39.40 | QVQLVQSGAEVKKPGASVKV | DVVMTQSPLSLPVTLGQPASISCKAS |
| scFv | SCKASGYTFTEYAMHWVRQ | QSLLDEDQKTYLNWLQQRPGQSPR |
| | APGQGLEWMGGINPKEGGTS | RLIYLVSKLDSGVPDRFSGSGSGTDF |
| | YNQKFKGRVTMTVDTSTSTA | TLKISRVEAEDVGVYYCWQGTHFPQ |
| | YMELSSLRSEDTAVYYCASQ | TFGGGTKVEIK (SEQ ID NO: 403) |
| | GWLLRGGDYWGQGTL VTVS | |
| | S (SEQ ID NO: 402) | |

TABLE 23
CDR sequences for affinity-optimized TfR9.1B.39 variants
(Kabat definition)

| Variant | CDR-H1 | CDR-H2 | CDR-H3 |
|---|---|---|---|
| TfR9.1B.39 | EYAMH (SEQ ID NO: 94) | GINPNDGGTSYNQKFKG | QGWLLRGGDY |
| | | (SEQ ID NO: 95) | (SEQ ID NO: 418) |
| TfR9.1B.39.5 | EYAMH (SEQ ID NO: 94) | GIDPRSGGTSYNQKFKG | QGWLKRGGDY |
| | | (SEQ ID NO: 412) | (SEQ ID NO: 419) |
| TfR9.1B.39.8 | EYAMH (SEQ ID NO: 94) | GIDPRSGGTSYNQKFKG | QGWLQRGGDY |
| | | (SEQ ID NO: 412) | (SEQ ID NO: 420) |
| TfR9.1B.39.36 | EYAMH (SEQ ID NO: 94) | GIDPNTGGTSYNQKFKG | QGWLLRGGDY |
| | | (SEQ ID NO: 413) | (SEQ ID NO: 418) |
| TfR9.1B.39.37 | EYAMH (SEQ ID NO: 94) | GIAPGTGGTSYNQKFKG | QGWLLRGGDY |
| | | (SEQ ID NO: 414) | (SEQ ID NO: 418) |
| TfR9.1B.39.38 | EYAMH (SEQ ID NO: 94) | GIAPNTGGTSYNQKFKG | QGWLLRGGDY |
| | | (SEQ ID NO: 415) | (SEQ ID NO: 418) |
| TfR9.1B.39.39 | EYAMH (SEQ ID NO: 94) | GINPSFGGTSYNQKFKG | QGWLLRGGDY |
| | | (SEQ ID NO: 416) | (SEQ ID NO: 418) |
| TfR9.1B.39.40 | EYAMH (SEQ ID NO: 94) | GINPKEGGTSYNQKFKG | QGWLLRGGDY |
| | | (SEQ ID NO: 417) | (SEQ ID NO: 418) |
| TfR9.1B.39 | KASQSLLDEDGKTYLN | LVSKLDS (SEQ ID | WQGTHFPQT |
| | (SEQ ID NO: 421) | NO: 150) | (SEQ ID NO: 151) |
| TfR9.1B.39.5 | KASQSLLDEEGKTYLN | LVSKLDS (SEQ ID | WQGTHFPQT |
| | (SEQ ID NO: 422) | NO: 150) | (SEQ ID NO: 151) |
| TfR9.1B.39.8 | KASQSLLDEEGKTYLN | LVSKLDS (SEQ ID | WQGTHFPQT |
| | (SEQ ID NO: 422) | NO: 150) | (SEQ ID NO: 151) |
| TfR9.1B.39.36 | KASQSLLDEEGKTYLN | LVSKLDS (SEQ ID | WQGTHFPQT |
| | (SEQ ID NO: 422) | NO: 150) | (SEQ ID NO: 151) |
| TfR9.1B.39.37 | KASQSLLDEEGKTYLN | LVSKLDS (SEQ ID | WQGTHFPQT |
| | (SEQ ID NO: 422) | NO: 150) | (SEQ ID NO: 151) |
| TfR9.1B.39.38 | KASQSLLDEGGKTYLN | LVSKLDS (SEQ ID | |
| | (SEQ ID NO: 423) | NO: 150) | (SEQ ID NO: 151) |
| TfR9.1B.39.39 | KASQSLLDEEGKTYLN | LVSKLDS (SEQ ID | WQGTHFPQT |
| | (SEQ ID NO: 422) | NO: 150) | (SEQ ID NO: 151) |
| TfR9.1B.39.40 | KASQSLLDEDQKTYLN | LVSKLDS (SEQ ID | WQGTHFPQT |
| | (SEQ ID NO: 424) | NO: 150) | (SEQ ID NO: 151) |

### Example 15: Engineering of Humanized TfR15 Antibodies

Engineering of humanized TfR15 was carried out to optimize affinity to the target and remove a potential oxidation site in the VH (Trp 38). DNA fragments of WH7, WH8, and WH12 which has Trp 38 replaced by Tyr were ordered from GeneArt and cloned into the same vector as mentioned above. Proteins were expressed and purified from Expi293 expression system and subject to further characterization. Sequences of these engineered variants are provided below in Table 24. TfR15.WH8.1.1 was optimized (via alanine scanning of the CDRs) to produce variants with a range of binding affinities (see Examples 16 and 17 below).

TABLE 24
TfR15 variants with removed liability site or reduced affinity

| Antibody | Sequence |
|---|---|
| TfR15.WH8 | QVQLVQSGAEVKKPGASVKVSCKASGYTFTSYWMHWVRQAPGQGLEWMGN |
| | IYPGSGSSNYAQKFQGRVTMTRDTSTSTVYMELSSLRSEDTAVYYCTRSSYYG |
| | NFDTMDNWGQGTTVTVSSGGSEGKSSGSGSESKSTGGSEIVLTQSPATLSLSPG |
| | ERATLSCRASSSIRYMNWYQQKPGQAPRRLIYDTSNRATGIPARFSGSGSGTDF |
| | TLTISSLEPEDFAVYYCHQRSSYPWTFGGGTKVEIK (SEQ ID NO: 223) |
| TfR15.WH7.1 | QVQLVQSGAEVKKPGASVKVSCKASGYTFTSYYMHWVRQAPGQGLEWMGN |
| | IYPGSGSSNYAQKFQGRVTMTRDTSTSTVYMELSSLRSEDTAVYYCTRSSYYG |
| | NFDTMDNWGQGTTVTVSSGGSEGKSSGSGSESKSTGGSDIQLTQSPSFLSASVG |
| | DRVTITCRASSSIRYMNWYQQKPGKAPKRLIYDTSTLQSGVPSRFSGSGSGTEF |
| | TLTISSLQPEDFATYYCHQRSSYPWTFGGGTKVEIK |
| | (SEQ ID NO: 282) |
| TfR15.WH8.1 | QVQLVQSGAEVKKPGASVKVSCKASGYTFTSYYMHWVRQAPGQGLEWMGN |
| | IYPGSGSSNYAQKFQGRVTMTRDTSTSTVYMELSSLRSEDTAVYYCTRSSYYG |
| | NFDTMDNWGQGTTVTVSSGGSEGKSSGSGSESKSTGGSEIVLTQSPATLSLSPG |
| | ERATLSCRASSSIRYMNWYQQKPGQAPRRLIYDTSNRATGIPARFSGSGSGTDF |
| | TLTISSLEPEDFAVYYCHQRSSYPWTFGGGTKVEIK |
| | (SEQ ID NO: 283) |
| TfR15.WH12.1 | QVQLVQSGAEVKKPGASVKVSCKASGYTFTSYYMHWVRQAPGQGLEWMGN |
| | IYPGSGSSNYAQKFQGRVTLTVDTSTSTVYMELSSLRSEDTAVYYCTRSSYYG |
| | NFDTMDNWGQGTTVTVSSGGSEGKSSGSGSESKSTGGSDIQMTQSPSSLSASV |
| | GDRVTITCRASSSIRYLNWYQQKPGKAPKRLIYDTSSLQSGVPSRFSGSGSGTD |
| | FTLTISSLQPEDFATYYCHQRSSYPWTFGGGTKVEIK |
| | (SEQ ID NO: 284) |
| TfR15.WH8.1.1 | QVQLVQSGAEVKKPGASVKVSCKASGYTFTSYYMHWVRQAPGQGLEWMGN |
| | IYPGSGSSNYAQKFQGRVTMTRDTSTSTVYMELSSLRSEDTAVYYCTRSSYYG |
| | NFDTMDNWGQGTTVTVSSGGSEGKSSGSGSESKSTGGSEIVLTQSPATLSLSPG |
| | ERATLSCRASSSIRAMNWYQQKPGQAPRRLIYDTSNRATGIPARFSGSGSGTDF |
| | TLTISSLEPEDFAVYYCHQRSSYPWTFGGGTKVEIK |
| | SEQ ID NO: 404) Same as TfR.15.WH8.1.42A |

### Example 16: Engineering of Humanized TfR.15. WH8.1 Variants

Engineering of humanized TfR.15.WH8.1 variants was carried out to optimize target affinity. The first step was alanine scanning of the CDRs, followed by amino acid substitution at selected positions in the CDRs. For alanine scanning of the CDRs, DNA fragments containing the variants were ordered from Integrated DNA Technologies (IDT) and cloned into an expression vector. Proteins were expressed and purified from Expi293 cells, and subjected to further characterization. From the alanine scan results (data not shown), key amino acid positions were selected for further amino acid substitution from alanine (A) to glycine (G), aspartic acid (D), asparagine (N), histidine (H), lysine (K), glutamine (Q), glutamic acid (E), or serine (S). There are 3 additional variants where, in addition to amino acid substitution, the VL and VH orientation was swapped or the linker was changed. For the WH8.1.42A.LH variant, the VH and VL orientation was swapped to VL-linker-VH. For WH8.1.42A.3x(G4S) variant, and the linker was changed to (GGGGS)x3 (SEQ ID NO: 184). For WH8.1.42A.L1 variant, the linker was changed to SPNSASHSGSAPQTSSAPGSQ (SEQ ID NO:442) (Hennecke et al., (1998) PEDS, 11:405-410). DNA fragments containing the variants were ordered from IDT and cloned into the expression vector in a 2+2 antibody format. 2+2 antibodies were expressed and purified from Expi293 expression system, as described above, and subjected to further characterization. The sequences are shown in Tables 25-27 below.

TABLE 25
Affinity-optimized TfR.15.WH8.1 variant sequences
Heavy and light chain sequences are underlined.

| Variant | Anti-TfR scFv Sequence |
|---|---|
| TFR.15.WH8.1 | QVQLVQSGAEVKKPGASVKVSCKASGYTFTSYYMHWVRQAPGQGLEWMGNI |
| | YPGSGSSNYAQKFQGRVTMTRDTSTSTVYMELSSLRSEDTAVYYCTRSSYYGN |
| | FDTMDNWGQGTTVTVSSGGSEGKSSGSGSESKSTGGSEIVLTQSPATLSLSPGER |
| | ATLSCRASSSIRYMNWYQQKPGQAPRRLIYDTSNRATGIPARFSGSGSGTDFTLT |
| | ISSLEPEDFAVYYCHQRSSYPWTFGGGTKVEIK (SEQ ID NO: 283) |
| TFR.15.WH8. | QVQLVQSGAEVKKPGASVKVSCKASGYTFTSYYMHWVRQAPGQGLEWMGNI |
| 24A1. | YPGSGSSNYAQKFQGRVTMTRDTSTSTVYMELSSLRSEDTAVYYCTRSAYYGN |
| | FDTMDNWGQGTTVTVSSGGSEGKSSGSGSESKSTGGSEIVLTQSPATLSLSPGER |
| | ATLSCRASSSIRYMNWYQQKPGQAPRRLIYDTSNRATGIPARFSGSGSGTDFTLT |
| | ISSLEPEDFAVYYCHQRSSYPWTFGGGTKVEIK (SEQ ID NO: 425) |
| TFR.15WH8. | QVQLVQSGAEVKKPGASVKVSCKASGYTFTSYYMHWVRQAPGQGLEWMGNI |
| 1.24K | YPGSGSSNYAQKFQGRVTMTRDTSTSTVYMELSSLRSEDTAVYYCTRSKYYGN |
| | FDTMDNWGQGTTVTVSSGGSEGKSSGSGSESKSTGGSEIVLTQSPATLSLSPGER |
| | ATLSCRASSSIRYMNWYQQKPGQAPRRLIYDTSNRATGIPARFSGSGSGTDFTLT |
| | ISSLEPEDFAVYYCHQRSSYPWTFGGGTKVEIK (SEQ ID NO: 426) |
| TFR.15.WH8. | QVQLVQSGAEVKKPGASVKVSCKASGYTFTSYYMHWVRQAPGQGLEWMGNI |
| 1.24D | YPGSGSSNYAQKFQGRVTMTRDTSTSTVYMELSSLRSEDTAVYYCTRSDYYGN |
| | FDTMDNWGQGTTVTVSSGGSEGKSSGSGSESKSTGGSEIVLTQSPATLSLSPGER |
| | ATLSCRASSSIRYMNWYQQKPGQAPRRLIYDTSNRATGIPARFSGSGSGTDFTLT |
| | ISSLEPEDFAVYYCHQRSSYPWTFGGGTKVEIK (SEQ ID NO: 427) |
| TFR.15.WH8. | QVQLVQSGAEVKKPGASVKVSCKASGYTFTSYYMHWVRQAPGQGLEWMGNI |
| 1.24N | YPGSGSSNYAQKFQGRVTMTRDTSTSTVYMELSSLRSEDTAVYYCTRSNYYGN |
| | FDTMDNWGQGTTVTVSSGGSEGKSSGSGSESKSTGGSEIVLTQSPATLSLSPGER |
| | ATLSCRASSSIRYMNWYQQKPGQAPRRLIYDTSNRATGIPARFSGSGSGTDFTLT |
| | ISSLEPEDFAVYYCHQRSSYPWTFGGGTKVEIK (SEQ ID NO: 428) |
| TFR.15.WH8. | QVQLVQSGAEVKKPGASVKVSCKASGYTFTSYYMHWVRQAPGQGLEWMGNI |
| 1.24H | YPGSGSSNYAQKFQGRVTMTRDTSTSTVYMELSSLRSEDTAVYYCTRSHYYGN |
| | FDTMDNWGQGTTVTVSSGGSEGKSSGSGSESKSTGGSEIVLTQSPATLSLSPGER |
| | ATLSCRASSSIRYMNWYQQKPGQAPRRLIYDTSNRATGIPARFSGSGSGTDFTLT |
| | ISSLEPEDFAVYYCHQRSSYPWTFGGGTKVEIK (SEQ ID NO: 429) |
| TFR.15.WH8. | QVQLVQSGAEVKKPGASVKVSCKASGYTFTSYYMHWVRQAPGQGLEWMGNI |
| 1.26K | YPGSGSSNYAQKFQGRVTMTRDTSTSTVYMELSSLRSEDTAVYYCTRSSYKGN |
| | FDTMDNWGQGTTVTVSSGGSEGKSSGSGSESKSTGGSEIVLTQSPATLSLSPGER |
| | ATLSCRASSSIRYMNWYQQKPGQAPRRLIYDTSNRATGIPARFSGSGSGTDFTLT |
| | ISSLEPEDFAVYYCHQRSSYPWTFGGGTKVEIK (SEQ ID NO: 430) |
| TFR.15.WH8. | QVQLVQSGAEVKKPGASVKVSCKASGYTFTSYYMHWVRQAPGQGLEWMGNI |
| 1.26Q | YPGSGSSNYAQKFQGRVTMTRDTSTSTVYMELSSLRSEDTAVYYCTRSSYQGN |
| | FDTMDNWGQGTTVTVSSGGSEGKSSGSGSESKSTGGSEIVLTQSPATLSLSPGER |
| | ATLSCRASSSIRYMNWYQQKPGQAPRRLIYDTSNRATGIPARFSGSGSGTDFTLT |
| | ISSLEPEDFAVYYCHQRSSYPWTFGGGTKVEIK (SEQ ID NO: 431) |
| TFR.15.WH8. | QVQLVQSGAEVKKPGASVKVSCKASGYTFTSYYMHWVRQAPGQGLEWMGNI |
| 1.26H | YPGSGSSNYAQKFQGRVTMTRDTSTSTVYMELSSLRSEDTAVYYCTRSSYHGN |
| | FDTMDNWGQGTTVTVSSGGSEGKSSGSGSESKSTGGSEIVLTQSPATLSLSPGER |
| | ATLSCRASSSIRYMNWYQQKPGQAPRRLIYDTSNRATGIPARFSGSGSGTDFTLT |
| | ISSLEPEDFAVYYCHQRSSYPWTFGGGTKVEIK (SEQ ID NO: 432) |
| TFR.15.WH8. | QVQLVQSGAEVKKPGASVKVSCKASGYTFTSYYMHWVRQAPGQGLEWMGNI |
| 1.26E | YPGSGSSNYAQKFQGRVTMTRDTSTSTVYMELSSLRSEDTAVYYCTRSSYEGN |
| | FDTMDNWGQGTTVTVSSGGSEGKSSGSGSESKSTGGSEIVLTQSPATLSLSPGER |
| | ATLSCRASSSIRYMNWYQQKPGQAPRRLIYDTSNRATGIPARFSGSGSGTDFTLT |
| | ISSLEPEDFAVYYCHQRSSYPWTFGGGTKVEIK (SEQ ID NO: 433) |
| TFR.15.WH8. | QVQLVQSGAEVKKPGASVKVSCKASGYTFTSYYMHWVRQAPGQGLEWMGNI |
| 1.26S | YPGSGSSNYAQKFQGRVTMTRDTSTSTVYMELSSLRSEDTAVYYCTRSSYSGNF |
| | DTMDNWGQGTTVTVSSGGSEGKSSGSGSESKSTGGSEIVLTQSPATLSLSPGER |
| | ATLSCRASSSIRYMNWYQQKPGQAPRRLIYDTSNRATGIPARFSGSGSGTDFTLT |
| | ISSLEPEDFAVYYCHQRSSYPWTFGGGTKVEIK (SEQ ID NO: 434) |
| TFR.15.WH8. | QVQLVQSGAEVKKPGASVKVSCKASGYTFTSYYMHWVRQAPGQGLEWMGNI |
| 1.42A | YPGSGSSNYAQKFQGRVTMTRDTSTSTVYMELSSLRSEDTAVYYCTRSSYYGN |
| | FDTMDNWGQGTTVTVSSGGSEGKSSGSGSESKSTGGSEIVLTQSPATLSLSPGER |
| | ATLSCRASSSIRAMNWYQQKPGQAPRRLIYDTSNRATGIPARFSGSGSGTDFTLT |
| | ISSLEPEDFAVYYCHQRSSYPWTFGGGTKVEIK (SEQ ID NO: 404) |
| TFR.15.WH8. | QVQLVQSGAEVKKPGASVKVSCKASGYTFTSYYMHWVRQAPGQGLEWMGNI |
| 1.42Q | YPGSGSSNYAQKFQGRVTMTRDTSTSTVYMELSSLRSEDTAVYYCTRSSYYGN |
| | FDTMDNWGQGTTVTVSSGGSEGKSSGSGSESKSTGGSEIVLTQSPATLSLSPGER |
| | ATLSCRASSSIRQMNWYQQKPGQAPRRLIYDTSNRATGIPARFSGSGSGTDFTLT |
| | ISSLEPEDFAVYYCHQRSSYPWTFGGGTKVEIK (SEQ ID NO: 435) |
| TFR.15.WH8. | QVQLVQSGAEVKKPGASVKVSCKASGYTFTSYYMHWVRQAPGQGLEWMGNI |
| 1.42H | YPGSGSSNYAQKFQGRVTMTRDTSTSTVYMELSSLRSEDTAVYYCTRSSYYGN |
| | FDTMDNWGQGTTVTVSSGGSEGKSSGSGSESKSTGGSEIVLTQSPATLSLSPGER |
| | ATLSCRASSSIRHMNWYQQKPGQAPRRLIYDTSNRATGIPARFSGSGSGTDFTLT |
| | ISSLEPEDFAVYYCHQRSSYPWTFGGGTKVEIK (SEQ ID NO: 436) |
| TFR.15.WH8. | QVQLVQSGAEVKKPGASVKVSCKASGYTFTSYYMHWVRQAPGQGLEWMGNI |
| 1.42D | YPGSGSSNYAQKFQGRVTMTRDTSTSTVYMELSSLRSEDTAVYYCTRSSYYGN |
| | FDTMDNWGQGTTVTVSSGGSEGKSSGSGSESKSTGGSEIVLTQSPATLSLSPGER |
| | ATLSCRASSSIRDMNWYQQKPGQAPRRLIYDTSNRATGIPARFSGSGSGTDFTLT |
| | ISSLEPEDFAVYYCHQRSSYPWTFGGGTKVEIK (SEQ ID NO: 437) |
| TFR.15.WH8. | QVQLVQSGAEVKKPGASVKVSCKASGYTFTSYYMHWVRQAPGQGLEWMGNI |
| 1.42S | YPGSGSSNYAQKFQGRVTMTRDTSTSTVYMELSSLRSEDTAVYYCTRSSYYGN |
| | FDTMDNWGQGTTVTVSSGGSEGKSSGSGSESKSTGGSEIVLTQSPATLSLSPGER |
| | ATLSCRASSSIRSMNWYQQKPGQAPRRLIYDTSNRATGIPARFSGSGSGTDFTLT |
| | ISSLEPEDFAVYYCHQRSSYPWTFGGGTKVEIK (SEQ ID NO: 438) |
| TFR.15.WH8. | QVQLVQSGAEVKKPGASVKVSCKASGYTFTSYYMHWVRQAPGQGLEWMGNI |
| 1.42K | YPGSGSSNYAQKFQGRVTMTRDTSTSTVYMELSSLRSEDTAVYYCTRSSYYGN |
| | FDTMDNWGQGTTVTVSSGGSEGKSSGSGSESKSTGGSEIVLTQSPATLSLSPGER |
| | ATLSCRASSSIRKMNWYQQKPGQAPRRLIYDTSNRATGIPARFSGSGSGTDFTLT |
| | ISSLEPEDFAVYYCHQRSSYPWTFGGGTKVEIK (SEQ ID NO: 439) |
| TFR.15.WH8. | EIVLTQSPATLSLSPGERATLSCRASSSIRAMNWYQQKPGQAPRRLIYDTSNRAT |
| 1.42A.LH | GIPARFSGSGSGTDFTLTISSLEPEDFAVYYCHQRSSYPWTFGGGTKVEIKGGSE |
| | GKSSGSGSESKSTGGSQVQLVQSGAEVKKPGASVKVSCKASGYTFTSYYMHW |
| | VRQAPGQGLEWMGNIYPGSGSSNYAQKFQGRVTMTRDTSTSTVYMELSSLRSE |
| | DTAVYYCTRSSYYGNFDTMDNWGQGTTVTVSS (SEQ ID NO: 440) |
| TFR.15.WH8. | QVQLVQSGAEVKKPGASVKVSCKASGYTFTSYYMHWVRQAPGQGLEWMGNI |
| 1.42A.3x | YPGSGSSNYAQKFQGRVTMTRDTSTSTVYMELSSLRSEDTAVYYCTRSSYYGN |
| (G4S) | FDTMDNWGQGTTVTVSSGGGGSGGGGSGGGGSEIVLTQSPATLSLSPGERATLS |
| | CRASSSIRAMNWYQQKPGQAPRRLIYDTSNRATGIPARFSGSGSGTDFTLTISSL |
| | EPEDFAVYYCHQRSSYPWTFGGGTKVEIK (SEQ ID NO: 441) |
| TFR.15.WH8. | QVQLVQSGAEVKKPGASVKVSCKASGYTFTSYYMHWVRQAPGQGLEWMGNI |
| 1.42A.L1 | YPGSGSSNYAQKFQGRVTMTRDTSTSTVYMELSSLRSEDTAVYYCTRSSYYGN |
| | FDTMDNWGQGTTVTVSSSPNSASHSGSAPQTSSAPGSQEIVLTQSPATLSLSPGE |
| | RATLSCRASSSIRAMNWYQQKPGQAPRRLIYDTSNRATGIPARFSGSGSGTDFTL |
| | TISSLEPEDFAVYYCHQRSSYPWTFGGGTKVEIK (SEQ ID NO: 442) |

TABLE 26
Variable heavy and variable light chain domains for affinity-optimized
TfR.15.WH8.1 variants

| Anti-body | VH sequence | VL sequence |
|---|---|---|
| TFR.15.WH8.1 | QVQLVQSGAEVKKPGASVKVSCKAS | EIVLTQSPATLSLSPGERATLSCRASSSI |
| | GYTFTSYYMHWVRQAPGQGLEWMG | RYMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | YGNFDTMDNWGQGTTVTVSS (SEQ | ID NO: 341) |
| | ID NO: 443) | |
| TFR.15WH8.1.24A | QVQLVQSGAEVKKPGASVKVSCKAS | EIVLTQSPATLSLSPGERATLSCRASSSI |
| | GYTFTSYYMHWVRQAPGQGLEWMG | RYMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSAY | YYCHQRSSYPWTFGGGTKVEIK (SEQ |
| | YGNFDTMDNWGQGTTVTVSS (SEQ | ID NO: 341) |
| | ID NO: 444) | |
| TFR.15.WH8.1.24K | QVQLVQSGAEVKKPGASVKVSCKAS | EIVLTQSPATLSLSPGERATLSCRASSSI |
| | GYTFTSYYMHWVRQAPGQGLEWMG | RYMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSKY | YYCHQRSSYPWTFGGGTKVEIK (SEQ |
| | YGNFDTMDNWGQGTTVTVSS (SEQ | ID NO: 341) |
| | ID NO: 445) | |
| TFR.15.WH8.1.24D | QVQLVQSGAEVKKPGASVKVSCKAS | EIVLTQSPATLSLSPGERATLSCRASSSI |
| | GYTFTSYYMHWVRQAPGQGLEWMG | RYMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSDY | YYCHQRSSYPWTFGGGTKVEIK (SEQ |
| | YGNFDTMDNWGQGTTVTVSS (SEQ | ID NO: 341) |
| | ID NO: 446) | |
| TFR.15.WH8.1.24N | QVQLVQSGAEVKKPGASVKVSCKAS | EIVLTQSPATLSLSPGERATLSCRASSSI |
| | GYTFTSYYMHWVRQAPGQGLEWMG | RYMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSNY | YYCHQRSSYPWTFGGGTKVEIK (SEQ |
| | YGNFDTMDNWGQGTTVTVSS (SEQ | ID NO: 341) |
| | ID NO: 447) | |
| TFR.15.WH8.1.24H | QVQLVQSGAEVKKPGASVKVSCKAS | EIVLTQSPATLSLSPGERATLSCRASSSI |
| | GYTFTSYYMHWVRQAPGQGLEWMG | RYMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSHY | YYCHQRSSYPWTFGGGTKVEIK |
| | YGNFDTMDNWGQGTTVTVSS (SEQ | (SEQ ID NO: 341) |
| | ID NO: 448) | |
| TFR.15.WH8.1.26K | QVQLVQSGAEVKKPGASVKVSCKAS | EIVLTQSPATLSLSPGERATLSCRASSSI |
| | GYTFTSYYMHWVRQAPGQGLEWMG | RYMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | KGNFDTMDNWGQGTTVTVSS (SEQ | ID NO: 341) |
| | ID NO: 449) | |
| TFR.15.WH8.1.26Q | QVQLVQSGAEVKKPGASVKVSCKAS | EIVLTQSPATLSLSPGERATLSCRASSSI |
| | GYTFTSYYMHWVRQAPGQGLEWMG | RYMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | QGNFDTMDNWGQGTTVTVSS (SEQ | ID NO: 341) |
| | ID NO: 450) | |
| TFR.15.WH8.1.26H | QVQLVQSGAEVKKPGASVKVSCKAS | EIVLTQSPATLSLSPGERATLSCRASSSI |
| | GYTFTSYYMHWVRQAPGQGLEWMG | RYMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | HGNFDTMDNWGQGTTVTVSS (SEQ | ID NO: 341) |
| | ID NO: 451) | |
| TFR.15.WH8.1.26E | QVQLVQSGAEVKKPGASVKVSCKAS | EIVLTQSPATLSLSPGERATLSCRASSSI |
| | GYTFTSYYMHWVRQAPGQGLEWMG | RYMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | EGNFDTMDNWGQGTTVTVSS (SEQ | ID NO: 341) |
| | ID NO: 452) | |
| TFR.15.WH8.1.26S | QVQLVQSGAEVKKPGASVKVSCKAS | EIVLTQSPATLSLSPGERATLSCRASSSI |
| | GYTFTSYYMHWVRQAPGQGLEWMG | RYMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | SGNFDTMDNWGQGTTVTVSS (SEQ | ID NO: 341) |
| | ID NO: 453) | |
| TFR.15.WH8.1.42A | QVQLVQSGAEVKKPGASVKVSCKAS | EIVLTQSPATLSLSPGERATLSCRASSSI |
| | GYTFTSYYMHWVRQAPGQGLEWMG | RAMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | YGNFDTMDNWGQGTTVTVSS (SEQ | ID NO: 454) |
| | ID NO: 443) | |
| TFR.15.WH8.1.42Q | QVQLVQSGAEVKKPGASVKVSCKAS | EIVLTQSPATLSLSPGERATLSCRASSSI |
| | GYTFTSYYMHWVRQAPGQGLEWMG | RQMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | YGNFDTMDNWGQGTTVTVSS (SEQ | ID NO: 455) |
| | ID NO: 443) | |
| TFR.15.WH8.1.42H | QVQLVQSGAEVKKPGASVKVSCKAS | EIVLTQSPATLSLSPGERATLSCRASSSI |
| | GYTFTSYYMHWVRQAPGQGLEWMG | RHMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | YGNFDTMDNWGQGTTVTVSS (SEQ | ID NO: 456) |
| | ID NO: 443) | |
| TFR.15.WH8.1.42D | QVQLVQSGAEVKKPGASVKVSCKAS | EIVLTQSPATLSLSPGERATLSCRASSSI |
| | GYTFTSYYMHWVRQAPGQGLEWMG | RDMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK |
| | YGNFDTMDNWGQGTTVTVSS (SEQ | (SEQ ID NO: 457) |
| | ID NO: 443) | |
| TFR.15.WH8.1.42S | QVQLVQSGAEVKKPGASVKVSCKAS | EIVLTQSPATLSLSPGERATLSCRASSSI |
| | GYTFTSYYMHWVRQAPGQGLEWMG | RSMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | YGNFDTMDNWGQGTTVTVSS (SEQ | ID NO: 458) |
| | ID NO: 443) | |
| TFR.15.WH8.1.42K | QVQLVQSGAEVKKPGASVKVSCKAS | EIVLTQSPATLSLSPGERATLSCRASSSI |
| | GYTFTSYYMHWVRQAPGQGLEWMG | RKMNWYQQKPGQAPRRLIYDTSNRAT |
| | NIYPGSGSSNYAQKFQGR VTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | YGNFDTMDNWGQGTTVTVSS (SEQ | ID NO: 459) |
| | ID NO: 443) | |
| TFR.15.WH8.1.42A.LH | QVQLVQSGAEVKKPGASVKVSCKAS | EIVLTQSPATLSLSPGERATLSCRASSSI |
| | GYTFTSYYMHWVRQAPGQGLEWMG | RAMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | YGNFDTMDNWGQGTTVTVSS (SEQ | ID NO: 454) |
| | ID NO: 443) | |
| TFR.15.WH8.1.42A.3x | QVQLVQSGAEVKKPGASVKVSCKAS | EIVLTQSPATLSLSPGERATLSCRASSSI |
| (G4S) | GYTFTSYYMHWVRQAPGQGLEWMG | RAMNWYQQKPGQAPRRLIYDTSNRAT |
| | NIYPGSGSSNYAQKFQGRVTMTRDTS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | YGNFDTMDNWGQGTTVTVSS (SEQ | ID NO: 454) |
| | ID NO: 443) | |
| TFR.15.WH8.1.42A.L1 | NIYPGSGSSNYAQKFQGRVTMTRDTS | EIVLTQSPATLSLSPGERATLSCRASSSI |
| | GYTFTSYYMHWVRQAPGQGLEWMG | RAMNWYQQKPGQAPRRLIYDTSNRAT |
| | QVQLVQSGAEVKKPGASVKVSCKAS | GIPARFSGSGSGTDFTLTISSLEPEDFAV |
| | TSTVYMELSSLRSEDTAVYYCTRSSY | YYCHQRSSYPWTFGGGTKVEIK (SEQ |
| | YGNFDTMDNWGQGTTVTVSS (SEQ | ID NO: 454) |
| | ID NO: 443) | |

TABLE 27
CDR sequences for affinity-optimized TfR.15.WH8.1 variants
(Kabat definition)

| Antibody | CDR-H1 | CDR-H2 | CDR-H3 |
|---|---|---|---|
| TFR.15.WH8.1 | SYYMH (SEQ ID | NIYPGSGSSNYAQKF | SSYYGNFDTMDN |
| | NO: 460) | QG (SEQ ID NO: 312) | (SEQ ID NO: 103) |
| TFR.15.WH8.1.24A | NO: 460) | QG (SEQ ID NO: 312) | (SEQ ID NO: 461) |
| | SYYMH (SEQ ID | NIYPGSGSSNYAQKF | SAYYGNFDTMDN |
| TFR. 15.WH8.1.24K | SYYMH (SEQ ID | NIYPGSGSSNYAQKF | SKYYGNFDTMDN |
| | NO: 460) | QG (SEQ ID NO: 312) | (SEQ ID NO: 462) |
| TFR.15.WH8.1.24D | SYYMH (SEQ ID | NIYPGSGSSNYAQKF | SDYYGNFDTMDN |
| | NO: 460) | QG (SEQ ID NO: 312) | (SEQ ID NO: 463) |
| TFR.15.WH8.1.24N | SYYMH (SEQ ID | NIYPGSGSSNYAQKF | SNYYGNFDTMDN |
| | NO: 460) | QG (SEQ ID NO: 312) | (SEQ ID NO: 464) |
| TFR.15.WH8.1.24H | SYYMH (SEQ ID | NIYPGSGSSNYAQKF | SHYYGNFDTMDN |
| | NO: 460) | QG (SEQ ID NO: 312) | (SEQ ID NO: 465) |
| TFR.15.WH8.1.26K | SYYMH (SEQ ID | NIYPGSGSSNYAQKF | SSYKGNFDTMDN |
| | NO: 460) | QG (SEQ ID NO: 312) | (SEQ ID NO: 466) |
| TFR.15.WH8.1.26Q | SYYMH (SEQ ID | NIYPGSGSSNYAQKF | SSYQGNFDTMDN |
| | NO: 460) | QG (SEQ ID NO: 312) | (SEQ ID NO: 467) |
| TFR.15.WH8.1.26H | SYYMH (SEQ ID | NIYPGSGSSNYAQKF | SSYHGNFDTMDN |
| | NO: 460) | QG (SEQ ID NO: 312) | (SEQ ID NO: 468) |
| TFR.15.WH8.1.26E | SYYMH (SEQ ID | NIYPGSGSSNYAQKF | SSYEGNFDTMDN |
| | NO: 460) | QG (SEQ ID NO: 312) | (SEQ ID NO: 469) |
| TFR.15.WH8.1.26S | SYYMH (SEQ ID | NIYPGSGSSNYAQKF | SSYSGNFDTMDN |
| | NO: 460) | QG (SEQ ID NO: 312) | (SEQ ID NO: 470) |
| TFR. 15.WH8.1.42A | SYYMH (SEQ ID | NIYPGSGSSNYAQKF | SSYYGNFDTMDN |
| | NO: 460) | QG (SEQ ID NO: 312) | (SEQ ID NO: 103) |
| TFR.15.WH8.1.42Q | SYYMH (SEQ ID | NIYPGSGSSNYAQKF | SSYYGNFDTMDN |
| | NO: 460) | QG (SEQ ID NO: 312) | (SEQ ID NO: 103) |
| TFR.15.WH8.1.42H | SYYMH (SEQ ID | NIYPGSGSSNYAQKF | SSYYGNFDTMDN |
| | NO: 460) | QG (SEQ ID NO: 312) | (SEQ ID NO: 103) |
| TFR.15.WH8.1.42D | SYYMH (SEQ ID | NIYPGSGSSNYAQKF | SSYYGNFDTMDN |
| | NO: 460) | QG (SEQ ID NO: 312) | (SEQ ID NO: 103) |
| TFR.15.WH8.1.42S | SYYMH (SEQ ID | NIYPGSGSSNYAQKF | SSYYGNFDTMDN |
| | NO: 460) | QG (SEQ ID NO: 312) | (SEQ ID NO: 103) |
| TFR.15.WH8.1.42K | SYYMH (SEQ ID | NIYPGSGSSNYAQKF | SSYYGNFDTMDN |
| | NO: 460) | QG (SEQ ID NO: 312) | (SEQ ID NO: 103) |
| TFR.15.WH8.1.42A.LH | SYYMH (SEQ ID | NIYPGSGSSNYAQKF | SSYYGNFDTMDN |
| | NO: 460) | QG (SEQ ID NO: 312) | (SEQ ID NO: 103) |
| TFR.15.WH8.1.42A.3x | SYYMH (SEQ ID | NIYPGSGSSNYAQKF | SSYYGNFDTMDN |
| (G4S) | NO: 460) | QG (SEQ ID NO: 312) | (SEQ ID NO: 103) |
| TFR.15.WH8.1.42A.L1 | SYYMH (SEQ ID | NIYPGSGSSNYAQKF | SSYYGNFDTMDN |
| | NO: 460) | QG (SEQ ID NO: 312) | (SEQ ID NO: 103) |
| TFR.15.WH8.1 | RASSSIRYMN | DTSNRAT (SEQ ID | HORSSYPWT (SEQ |
| | (SEQ ID NO: 307) | NO: 309) | ID NO: 157) |
| TFR. 15.WH8.1.24A | RASSSIRYMN | DTSNRAT (SEQ ID | HQRSSYPWT (SEQ |
| | (SEQ ID NO: 307) | NO: 309) | ID NO: 157) |
| TFR.15.WH8.1.24K | RASSSIRYMN | DTSNRAT (SEQ ID | HQRSSYPWT (SEQ |
| | (SEQ ID NO: 307) | NO: 309) | ID NO: 157) |
| TFR.15.WH8.1.24D | RASSSIRYMN | DTSNRAT (SEQ ID | HQRSSYPWT (SEQ |
| | (SEQ ID NO: 307) | NO: 309) | ID NO: 157) |
| TFR.15.WH8.1.24N | RASSSIRYMN | DTSNRAT (SEQ ID | HQRSSYPWT (SEQ |
| | (SEQ ID NO: 307) | NO: 309) | ID NO: 157) |
| TFR.15.WH8.1.24H | RASSSIRYMN | DTSNRAT (SEQ ID | HQRSSYPWT (SEQ |
| | (SEQ ID NO: 307) | NO: 309) | ID NO: 157) |
| TFR.15.WH8.1.26K | RASSSIRYMN | DTSNRAT (SEQ ID | HQRSSYPWT (SEQ |
| | (SEQ ID NO: 307) | NO: 309) | ID NO: 157) |
| TFR.15.WH8.1.26Q | RASSSIRYMN | DTSNRAT (SEQ ID | HQRSSYPWT (SEQ |
| | (SEQ ID NO: 307) | NO: 309) | ID NO: 157) |
| TFR.15.WH8.1.26H | RASSSIRYMN | DTSNRAT (SEQ ID | HQRSSYPWT (SEQ |
| | (SEQ ID NO: 307) | NO: 309) | ID NO: 157) |
| TFR.15.WH8.1.26E | RASSSIRYMN | DTSNRAT (SEQ ID | HQRSSYPWT (SEQ |
| | (SEQ ID NO: 307) | NO: 309) | ID NO: 157) |
| TFR.15.WH8.1.26S | RASSSIR YMN | DTSNRAT (SEQ ID | HQRSSYPWT (SEQ |
| | (SEQ ID NO: 307) | NO: 309) | ID NO: 157) |
| TFR. 15.WH8.1.42A | RASSSIRAMN | DTSNRAT (SEQ ID | HQRSSYPWT (SEQ |
| | (SEQ ID NO: 471) | NO: 309) | ID NO: 157) |
| TFR.15.WH8.1.42Q | RASSSIRQMN | DTSNRAT (SEQ ID | HQRSSYPWT (SEQ |
| | (SEQ ID NO: 472) | NO: 309) | ID NO: 157) |
| TFR.15.WH8.1.42H | RASSSIRHMN | DTSNRAT (SEQ ID | HQRSSYPWT (SEQ |
| | (SEQ ID NO: 473) | NO: 309) | ID NO: 157) |
| TFR.15.WH8.1.42D | RASSSIRDMN | DTSNRAT (SEQ ID | HQRSSYPWT (SEQ |
| | (SEQ ID NO: 474) | NO: 309) | ID NO: 157) |
| TFR.15.WH8.1.42S | RASSSIRSMN | DTSNRAT (SEQ ID | HQRSSYPWT (SEQ |
| | (SEQ ID NO: 475) | NO: 309) | ID NO: 157) |
| TFR.15.WH8.1.42K | RASSSIRKMN | DTSNRAT (SEQ ID | HQRSSYPWT (SEQ |
| | (SEQ ID NO: 476) | NO: 309) | ID NO: 157) |
| TFR.15.WH8.1.42A.LH | RASSSIRAMN | DTSNRAT (SEQ ID | HQRSSYPWT (SEQ |
| | (SEQ ID NO: 471) | NO: 309) | ID NO: 157) |
| TFR.15.WH8.1.42A.3x | RASSSIRAMN | DTSNRAT (SEQ ID | HQRSSYPWT (SEQ |
| (G4S) | (SEQ ID NO: 471) | NO: 309) | ID NO: 157) |
| TFR.1S.WH8.1.42A.LI | RASSSIRAMN | DTSNRAT (SEQ ID | HQRSSYPWT (SEQ |
| | (SEQ ID NO: 471) | NO: 309) | ID NO: 157) |

### Example 17: Binding Kinetics of TfR.15.WH8.1 Variants

Binding kinetics of 2+2 TfR.15.WH8.1 variants (Isotype IgG with 2 anti-TfR scFvs) to Avi-His tagged human and cynomolgus TfR apical domains were evaluated using a Carterra LSA instrument (Carterra, Salt Lake City, UT). A capture lawn was prepared on a HC30M sensor chip (Carterra) using a goat anti-human Fcγ polyclonal antibody (JacksonImmunoResearch) according to the instrument manufacturer's instructions. Two independent experiments were performed as follows, resulting in an N of between one and two determinations for each antibody. Spots that yielded less than 20 RU of analyte binding were excluded from further analysis.

The 2+2 anti-TfR bispecific antibodies were prepared by diluting with running buffer (HBS-EP+; Teknova) and adding 0.5 mg/mL BSA (MP Biomedicals), to give a final concentration of 15 μg/mL and captured in an array on the the goat anti-human Fcγ capture surface. The captured antibodies were tested for their ability to bind to the recombinant human and cynomolgus orthologs of TfR apical Avi-His. Estimates of affinity were generated by injecting each analyte over the entire antibody array using a single channel flow cell. TfR apical Avi-His analytes were diluted with running buffer, in a series of eight, three-fold serial dilutions starting from 2500 nM for human and cynomolgus TfR apical Avi-His, followed by two 10-fold dilutions. Analytes were injected for 5 minutes, and dissociation was followed for 15 minutes. After each analyte injection series, antibodies were regenerated with 10 mM glycine pH 2.0 buffer (Carterra). Data was processed and analyzed using NextGenKIT high-throughput kinetics analysis software (Carterra).

The equilibrium dissociation constants (K_{D}) were calculated from the fitted association and dissociation rate constants (k-on and k-off) for 2+2 anti-TfR bispecific antibodies of the present disclosure binding to human and cynomolgus TfR apical Avi-His. For measurements with N>1, means and standard deviation were calculated and measurements for all samples were combined and graphed using GraphPad Prism. The K_{D} values are summarized in Table 28 below. Some antibodies, as indicated in the table, displayed heterogeneous binding profiles that do not fit to a 1:1 binding model, thus their rate constants could not be determined in this series of experiments.

TABLE 28
Equilibrium dissociation constants (KD) for 2 + 2 anti-TfR bispecific antibodies

| | Human apical TfR | | | Cynomolgus apical TfR | | |
|---|---|---|---|---|---|---|
| Antibody | Mean K_{D} (M) | SD | N | Mean K_{D} (M) | SD | N |
| TFR.15.WH8.1 | 2.8E−08 | NA | 1 | 1.7E−08 | 4.2E−09 | 2 |
| TFR.15.WH8.1.24A | HET | NA | 1 | >2.5E−6 | 2.4E−06 | 2 |
| TFR.15.WH8.1.24D | NB | NA | 1 | NB | | 2 |
| TFR.15.WH8.1.24H | 5.6E−09 | NA | 1 | 2.6E−09 | 1.6E−09 | 2 |
| TFR.15.WH8.1.24K | NB | NA | 1 | NB | | 2 |
| TFR.15.WH8.1.24N | 1.4E−07 | NA | 1 | 4.7E−08 | 1.7E−08 | 2 |
| TFR.15.WH8.1.26E | NB | NA | 1 | NB | | 2 |
| TFR.15.WH8.1.26H | 2.8E−07 | NA | 1 | 2.2E−06 | 4.4E−07 | 2 |
| TFR.15.WH8.1.26K | NB | NA | 1 | NB | | 2 |
| TFR.15.WH8.1.26Q | NB | NA | 1 | NB | | 2 |
| TFR.15.WH8.1.26S | >2.5E−6 | NA | 1 | >2.5E−6 | 2.6E−05 | 2 |
| TFR.15.WH8.1.42A | 1.6E−07 | NA | 1 | 8.8E−08 | 3.9E−09 | 2 |
| TFR.15.WH8.1.42A.3x(G4S) | HET | NA | 1 | 7.4E−08 | 2.9E−08 | 2 |
| TFR.15.WH8.1.42D | HET | NA | 1 | NB | | 2 |
| TFR.15.WH8.1.42H | NB | NA | 1 | NB | | 2 |
| TFR.15.WH8.1.42K | NB | NA | 1 | NB | | 2 |
| TFR.15.WH8.1.42A.L1 | 1.5E−07 | NA | 1 | 1.0E−07 | 2.1E−08 | 2 |
| TFR.15.WH8.1.42A.LH | 1.9E−07 | NA | 1 | 6.0E−08 | 9.0E−09 | 2 |
| TFR.15.WH8.1.42Q | 6.2E−08 | NA | 1 | 4.0E−08 | 2.1E−08 | 2 |
| TFR.15.WH8.1.42S | 5.1E−07 | NA | 1 | 6.9E−07 | 2.3E−07 | 2 |

N = number of determinations; NT = not tested; NA = not applicable; LB = low binding (below limit of measurement); NB = no binding detected; HET = heterogeneous binding.

These results illustrate that 2+2 anti-TfR bispecific antibodies exhibit a range of affinities from approximately 3 nM to >2.5 M for TfR apical domain binding. In particular, the affinity of 2+2 anti-TfR bispecific antibodies for binding to human TfR Avi-His ranged from 6 nM to >2.5 μM; and affinity of anti-TfR antibodies of the present disclosure for binding to cynomolgus TfR Avi-His ranged from 3 nM to >2.5 μM.

### Example 18: Binding Kinetics of 2+1 Anti-TfR Bispecific Antibodies

Binding kinetics of humanized 2+1 anti-TfR bispecific antibodies to human and cyno TfR Avi-His were evaluated using a Carterra LSA instrument (Carterra, Salt Lake City, UT). Briefly, 2+1 anti-TfR bispecific antibodies were prepared in duplicates by diluting 20 to 180-fold into 10 mM Acetate, pH 4.5 (Carterra), to give final concentration of 30 μg/mL. An HC30M sensor chip (Carterra) was activated using the single channel flow cell with a 7-minute injection of a 1:1:1 mixture of 100 mM MES pH 5.5, 100 mM sulfo-NHS, 400 mM EDC (all reconstituted in MES pH 5.5; 100 μl of each mixed in vial immediately before running assay). After switching to the multi-channel array flow cell, the antibodies were injected over the activated chip in four 96-spot arrays for 15 minutes each. The remaining unconjugated active groups on the chip were then blocked by injecting 1M Ethanolamine pH 8.5 (Carterra) for 7 minutes using the single channel flow cell. The resulting sensor chip contained eight spots for each antibody, at four different densities. Two independent experiments were performed as follows, resulting in an N of between one and eight determinations for each antibody. Spots that yielded less than 20 RU of analyte binding were excluded from further analysis.

After priming with running buffer HBS-EP+(Teknova) with 0.5 mg/ml BSA (MP Biomedicals), the immobilized anti-TfR 2+1 antibodies were tested for their ability to bind to two forms of TfR Avi-His, which are human and cyno orthologs described above. Estimates of affinity were generated by injecting each analyte over the entire antibody array using the single channel flow cell. TfR Avi-His analytes were diluted with running buffer, in a series of eight, three-fold serial dilutions starting from 1 μM for human and cyno TfR Avi-His. Analytes were injected for 5 minutes, and dissociation was followed for 10 minutes. After each analyte injection, antibodies were regenerated with Pierce™ IgG Elution Buffer (ThermoScientific). Data were processed and analyzed using NextGenKIT high-throughput kinetics analysis software (Carterra).

The equilibrium dissociation constants (K_{D}) were calculated from the fitted association and dissociation rate constants (k-on and k-off) for anti-TfR Avi-His antibodies of the present disclosure. The values were combined, means and standard deviation calculated, and graphs prepared using GraphPad Prism. The K_{D} values are summarized in Table 29 below. Some antibodies, as indicated in the table, displayed heterogeneous binding profiles that do not fit to a 1:1 binding model, thus their rate constants could not be determined.

TABLE 29
Equilibrium dissociation constants (K_{D}) for anti-TfR 2 + 1 bispecific antibodies

| | huTfR Avi-His | | | cyno TfR Avi-His | | |
|---|---|---|---|---|---|---|
| Antibody | Mean K_{D} (M) | SD | N | Mean K_{D} (M) | SD | N |
| TfR9.1B.9 | 6.7E−09 | NA | 1 | heterogeneous | Heterogeneous | NA |
| TfR9.1B.10 | heterogeneous | heterogeneous | NA | heterogeneous | Heterogeneous | NA |
| TfR9.1B.11 | heterogeneous | heterogeneous | NA | heterogeneous | Heterogeneous | NA |
| TfR9.1B.13 | 2.9E−08 | 4.9E−09 | 2 | 8.3E−07 | NA | 1 |
| TfR9.1B.14 | 3.4E−08 | NA | 1 | 8.7E−07 | NA | 1 |
| TfR9.1B.20 | heterogeneous | heterogeneous | NA | heterogeneous | Heterogeneous | NA |
| TfR9.1B.22 | 2.6E−07 | NA | 1 | heterogeneous | Heterogeneous | NA |
| TfR9.1B.24 | heterogeneous | heterogeneous | NA | heterogeneous | Heterogeneous | NA |
| TfR9.1B.26 | heterogeneous | heterogeneous | NA | heterogeneous | Heterogeneous | NA |
| TfR9.1B.27 | heterogeneous | heterogeneous | NA | heterogeneous | Heterogeneous | NA |
| TfR9.1B.28 | 1.0E−08 | 1.9E−09 | 3 | heterogeneous | Heterogeneous | NA |
| TfR9.1B.33 | 2.5E−08 | NA | 1 | heterogeneous | Heterogeneous | NA |
| TfR9.1B.35 | heterogeneous | heterogeneous | NA | heterogeneous | Heterogeneous | NA |
| TfR9.1B.36 | 1.3E−08 | 1.2E−09 | 4 | heterogeneous | Heterogeneous | NA |
| TfR9.1B.38 | 1.5E−08 | 1.9E−09 | 3 | heterogeneous | Heterogeneous | NA |
| TfR9.1B.39 | 8.7E−08 | NA | 1 | heterogeneous | Heterogeneous | NA |
| TfR9.1B.40 | 8.2E−09 | 9.7E−10 | 3 | heterogeneous | Heterogeneous | NA |
| TfR9.1B.41 | 3.4E−07 | NA | 1 | heterogeneous | Heterogeneous | NA |
| TfR9.1B.43 | NB | NB | NA | heterogeneous | Heterogeneous | NA |
| TfR9.1B.45 | 1.3E−08 | NA | 1 | heterogeneous | Heterogeneous | NA |
| TfR15.WH7 | 4.3E−08 | 1.7E−08 | 2 | 5.3E−08 | 7.8E−09 | 2 |
| TfR15.WH9 | 1.1E−07 | NA | 1 | 2.2E−07 | NA | 1 |
| TfR15.WH10 | 4.8E−08 | 1.6E−09 | 2 | 3.9E−08 | 1.0E−08 | 2 |
| TfR15.WH12 | NB | NB | NA | heterogeneous | Heterogeneous | NA |
| TfR15.WH11a | 1.1E−07 | NA | 1 | 9.2E−08 | NA | 1 |
| TfR15.WH2 | NB | NB | NA | heterogeneous | Heterogeneous | NA |
| TfR15.WH8 | 2.6E−08 | NA | 1 | 1.8E−08 | NA | 1 |
| TfR15.EB6 | heterogeneous | heterogeneous | NA | 3.0E−08 | 5.0E−09 | 4 |
| TfR9.1B | 3.5E−08 | NA | 1 | heterogeneous | Heterogeneous | NA |
| TfR15 parental | 8.7E−09 | 3.8E−09 | 4 | heterogeneous | Heterogeneous | NA |

N = number of determinations; NT = not tested; NA = not applicable; LB = low binding (below limit of measurement); NB = no binding detected

These results illustrate that 2+1 anti-TfR bispecific antibodies exhibit a range of affinities from approximately 6 nM to 900 nM for TfR Avi-His. In particular, affinity of 2+1 anti-TfR bispecific antibodies of the present disclosure for binding to huTfR Avi-His ranged from 6.7 nM to 340 nM. Affinity of anti-TfR antibodies of the present disclosure for binding to cyno TfR Avi-His ranged from 18 nM to 870 nM.

### Example 19: PK of 2+1 Anti-TfR Bispecific Antibodies in huTfR ECD KI Mice

2+1 anti-TfR Bispecific Antibody Production: 2+1 anti-TfR bispecific antibodies for in-vivo PK studies were expressed and purified on a 2+1 backbone with an isotype control (non-binding) Fab arm.

Antibodies for in-vivo PK studies were generated via transient transfection of ExpiCHO cells (Invitrogen) performed according to manufacturer's instruction. For 400 mL culture, 320 μg of total DNA consisting of 3 expression plasmids (heavy chain-hole-scFv, heavy chain-knob, and light chain) were used. The molar ratio of the 3 plasmids were optimized to achieve high purity of 2+1 anti-Tfr bispecific antibodies. The cell culture supernatant was harvested at 10 days post transfection. The antibody was purified using protein A affinity chromatography followed by ion exchange chromatography on an AKTA Avant 25 (Cytiva) to remove product-related impurities. The purified antibodies were dialyzed into PBS. Analytical characterization was performed by absorbance at 280 nm, CE-SDS, size-exclusion chromatography, and endotoxin measurement.

Study design: To establish in vivo proof of concept for brain penetration of 2+1 anti-TfR bispecific antibodies, a heterozygous knock-in (KI) mouse was generated in which the ectodomain region of mouse TfR was replaced with that of human in one of the two alleles. This was because the 2+1 anti-Tfr bispecific antibodies specifically bind to primate but not mouse TfR. Notably, the total amount of TfR protein in resulting TfRmu/hu KI mouse was indistinguishable from that observed in the wild-type (WT) animals (data not shown). A two-dose strategy was used to determine both the brain uptake as well as peripheral clearance of injected Abs over time. A group of WT and hu-TfRmu/hu mice were dosed with 10 mg/kg of either isotype IgG or 2+1 anti-TfR bispecific antibodies at day 1 and 14. Blood samples were collected from antibody treated animals at different timepoints in serum separator tubes, which are then allowed to clot at room temperature (RT) before centrifugation. The resulting supernatants, representing sera, were subsequently transferred into new tubes, and stored at −80° C. until analysis.

Sample preparation: Brain tissues were collected from antibody treated mice on day 15 (i.e., 24 hrs after the second antibody injection). Prior to this, mice were anesthetized and underwent cardiac perfusion with 15 ml PBS to clear blood vessels. Brain tissues were then minced and homogenized in 1 ml HBSS buffer (MilliporeSigma #55037C) containing 10 mM HEPES (#15630130, Gibco) using a hand-operated grinder. Subsequently, samples were centrifuged at 1000 g for 5 min to pellet the vessel portion. The supernatants, representing the parenchyma, were then transferred into new tubes, and mixed with 10×RIPA buffer (final concentration 1×) including protease and phosphatase inhibitor cocktail (cOmplete™, Mini Protease Inhibitor Cocktail, Roche, #11836153001). After 20 min incubation on a nutator at 4° C., samples were frozen on dry ice and stored at −80° C. until analysis. Vessel fractions were cleared from myelin debris via centrifugation through %18 Dextran (70 kDa, #31390, Sigma-Aldrich), followed by 2 washes in HBSS buffer. Afterwards, samples were lysed in RIPA buffer (R0278, Sigma-Aldrich) and stored at −80° C. until analysis. The validity of this method was confirmed using WB analysis, in which markers of brain endothelial cells (e.g., CD31 and Claudin-5) were absent in the parenchymal portions (data not shown).

Pharmacokinetic (PK) analysis: Antibody concentrations in serum and brain samples were measured using MSD (Meso Scale Discovery) method. Briefly, 50 μl/well of goat anti-human IgG1 Ab (Jackson ImmunoResearch, #109-005-097) was added to MSD plates at 1 g/ml in PBS and incubated overnight at 4° C. Plates were then washed 3× with wash buffer (0.05% Tween-20 in 1×PBS), followed by incubation with blocking buffer (wash buffer containing 3% BSA) for 1 hr at RT. Subsequently, serum (1:10000 dilution in PBS) and brain (1:2 dilutions in PBS) samples were added in duplicates to the plates and incubated for 1 hr on a shaker at 500 RPM. A human IgG1 isotype Ab with known concentration was included in the assay to create standard curve. Next, plates were washed 3× with wash buffer, followed by addition of 30 μl/well of Sulfo-Tag goat anti-human Ab (R32AJ-5, Meso Scale Discovery) at 0.2 g/ml in PBS and 1 hr incubation on shaker at 500 RPM. Finally, after 3× washes with wash buffer, 150 μl/well of 1×READ buffer (R92PC-2, Meso Scale Discovery) was added to the plates, before they are being read using a Sector Imager S600 instrument.

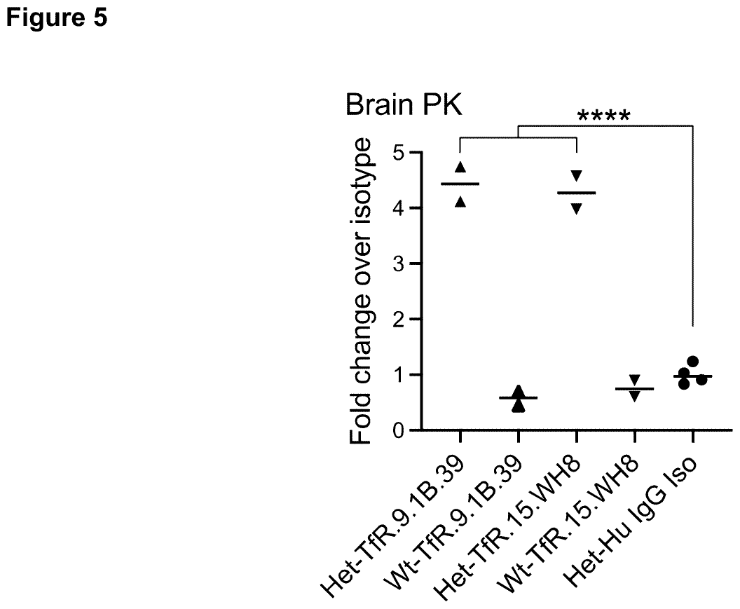

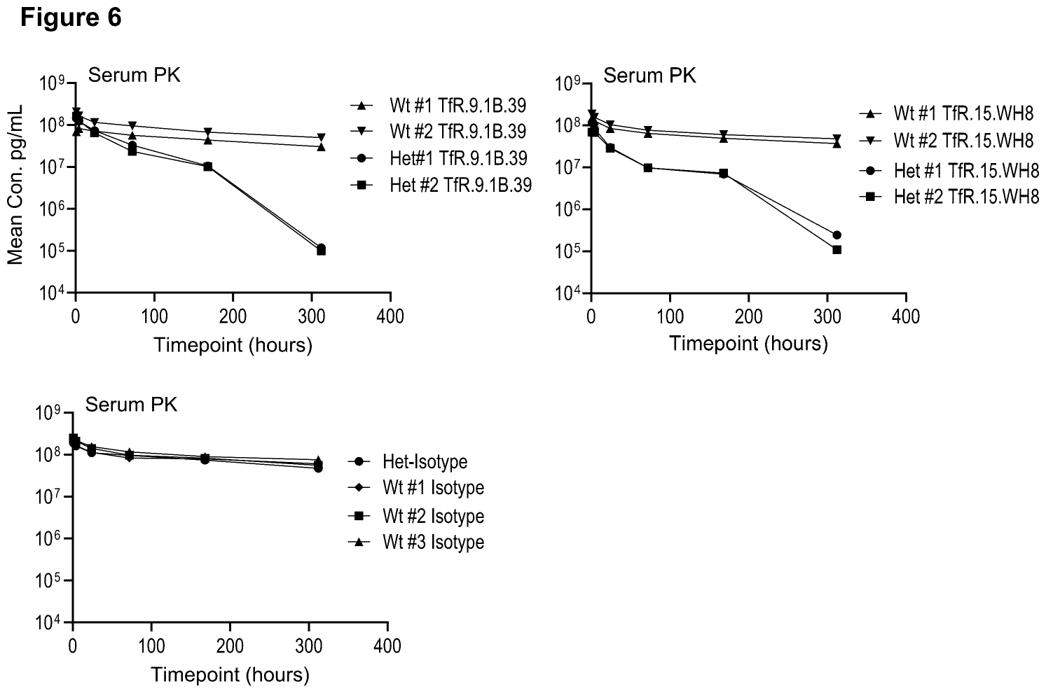

By measuring antibody concentrations in vessel depleted brains, levels of 2+1 anti-TfR bispecific antibodies were 4-5 fold higher than isotype IgG in hu-TfRmu/hu mice (FIG. 5). In contrast, brain uptake of 2+1 anti-TfR bispecific antibodies in WT animals, which do not express target receptor, was similar to that observed for isotype IgG. Together, these results indicate that the 2+1 anti-TfR bispecific antibodies have enhanced ability for brain entry mediated by the transferrin receptor. Peripheral PK was determined by measuring antibody concentrations in plasma samples of antibody treated mice. In WT mice, plasma levels of 2+1 anti-TfR bispecific antibodies were characterized by a slow half-life and were indistinguishable from isotype antibody (FIG. 6). In contrast, both 2+1 anti-TfR bispecific antibodies exhibited higher plasma clearance in hu-TfR^{mu/hu} mice compared to isotype IgG, which is consistent with TfR-mediated disposition in the periphery.

### Example 20: Effect of 2+1 Anti-TfR Bispecific Antibodies on TfR Expression on Brain Endothelial Cells In Vivo

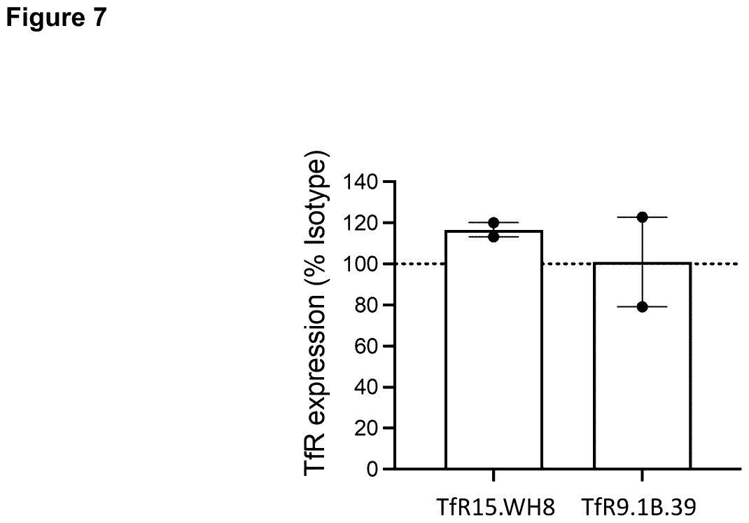

To determine if 2+1 anti-Tfr bispecific antibodies dysregulate receptor recycling in target cells in vivo, the total amount of TfR protein was quantified in the vessel portion of brain tissues, collected from antibody treated animals. To this end, isolated vessels were lysed in RIPA buffer and processed by WB as described in Example 13. Using quantitative analysis, no significant differences in TfR band densities were observed between samples obtained from hu-TfR^{+/-} KI mice that were injected with anti-TfR antibodies (i.e., TfR-9.1B.39 and TfR-15.WH8) compared to those from isotype treated animals (FIG. 7). Notably, in this assay, the membrane was probed with an anti-human TfR antibody containing no cross-reactivity to the mouse TfR (R&D Systems Inc, #MAB2474). As a result, no TfR band was detected in vessels obtained from WT animals. Together, these observations indicate that 2+1 anti-TfR bispecific antibodies do not alter TfR recycling at the blood brain barrier (BBB) in vivo.

### Example 21: In Vivo Reticulocyte Depletion Upon 2+1 Anti-TfR Bispecific Antibody Treatment in Mice

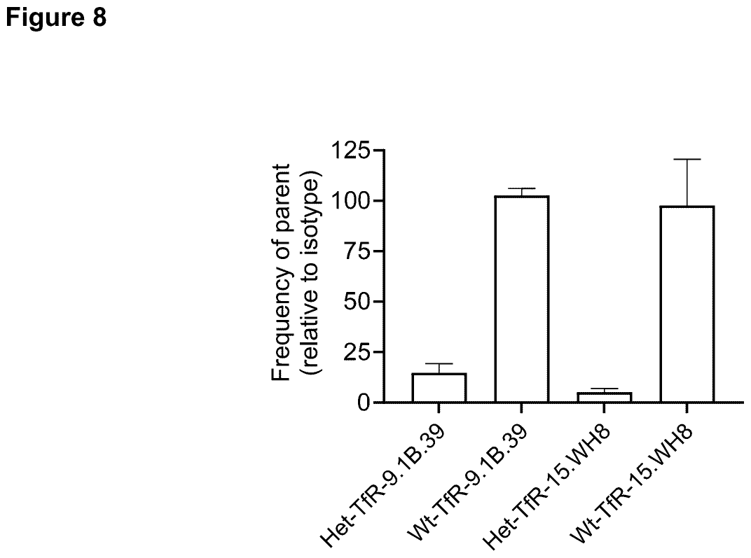

Anti-TfR antibodies are known to induce cell death in reticulocyte cell population in vivo. To examine this, a group of WT and hu-TfR^{+/-} KI mice were injected with either 2+1 anti-TfR bispecific antibodies (TfR-9.1B.39 and TfR-15.WH8), a human IgG1 isotype or the previously published anti-ms TfR antibody 8D3 (Boado et al, Biotechnol Bioeng. 2009 Mar. 1; 102(4): 1251-1258), which is known to drive strong depletion of reticulocytes in mice. The latter two antibodies were included in the experiment as negative and positive controls, respectively. All antibodies tested were on a WT human IgG1 backbone which can drive ADCC, ADCP and CDC responses in vivo. Blood samples were collected from antibody treated animals at 24 hrs post injection and analyzed by FACS. Reticulocytes can be distinguished from mature red blood cells (RBCs) by staining the cells with anti-CD71 (Invitrogen, #Catalog #17-0719-42), Thiazole Orange (MilliporeSigma, #390062) and anti-mouse CD45 for excluding circulating white blood cells (Invitrogen, #12-0451-83). Thiazole Orange binds residual ribosomal RNA molecules, which are present in reticulocytes but not in mature RBCs. Compared to RBCs, reticulocytes express high levels of transferrin receptor (CD71). As expected, injection of 8D3 antibody resulted in a complete loss of reticulocytes in WT animals (data not shown). In contrast, this cell population was not altered in isotype treated animals, regardless of genotype. Similarly, no changes in reticulocytes numbers were observed in WT mice receiving anti-TfR 2+1 bispecific antibodies, which makes sense because these animals do not express the target receptor (FIG. 8). In contrast, the hu-TfR^{+/-} KI mice treated with TfR-9.1B.39 and TfR-15.WH8 displayed 80% to 90% reduction in reticulocytes numbers, respectively (FIG. 8). Based on published results (Yamamoto et al, Molecular Genetics and Metabolism Reports, Volume 27, June 2021, 100758), reticulocyte depletion seen here is likely mediated through induction of ADCC (antibody-dependent cellular cytotoxicity) responses, which was further evaluated in subsequent examples.

### Example 22: Evaluation of In Vitro Effector Function (CDC and ADCC)

To investigate the mechanism of reticulocyte depletion seen in 2+1 anti-TfR bispecific antibodies, the ability of these same antibodies was evaluated to elicit in vitro effector responses, such as complement dependent cytotoxicity (CDC) and antibody-dependent cellular cytotoxicity (ADCC). In addition, given that anti-TfR antibodies on wt huIgG1 backbone have been shown to elicit ADCC responses, alternative Fc formats were tested, such as N325S/L328F (NSLF) and L234A/L235A/P331S (LALAPS), which have strongly decreased binding to activating FcgRs (in particular, FcgRIIIa which is the key driver of ADCC).

CDC method and results: The ability of 2+1 anti-TfR bispecific antibodies to drive complement deposition was measured on the hCMEC/D3 cell line, which expresses high levels of both receptors. Target cells were detached, washed 1× in PBS, and diluted to 2×10⁶ cells/mL in RPMI 1640 media. 50 μL of target cells were aliquoted per well (1×105 cells per well) in round-bottom 96 well plates (Falcon #351177). To these cells was added 25 μL of 4× antibody diluted in the same media. Cells and antibody were incubated at 37° C. for 15 min, then 25 μL of pooled complement human serum (Innovative Research, IPLA-CSER) was added per well as a complement source and the plates incubated for a further 2 hrs at 37° C. Afterwards, cells were washed 2× with FACS buffer (PBS+2% FBS+1 mM EDTA) and resuspended in 50 μL of FACS buffer+0.25 μL/well of propidium iodide (Fischer Scientific, BD 556463) prior to analysis on an iQue flow cytometer (IntelliCyt). CDC was analyzed as the proportion of PI+single cells in each well. In this assay none of the 2+1 anti-TfR bispecific antibodies induced a significant CDC response (data not shown).

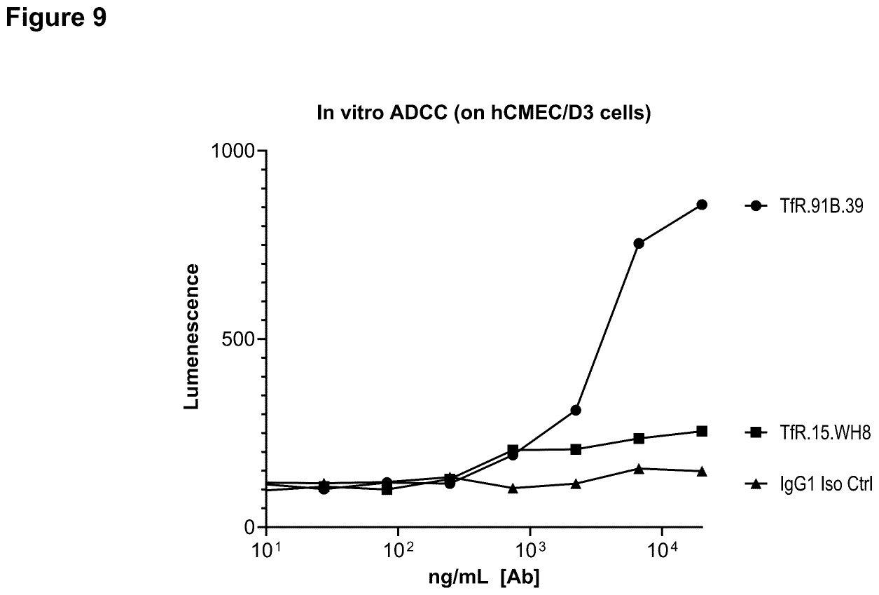

ADCC method and results: The ability of 2+1 bispecific antibodies to cause antibody-dependent cellular cytotoxicity (ADCC) was evaluated using an ADCC Reporter Bioassay system from Promega (#G7010). This system relies on an engineered Jurkat T cell line stably expressing the FcgRIIIa receptor (V158 variant) and an NFAT response element driving expression of firefly luciferase. Target cells were diluted in assay buffer (RPMI+4% low IgG Serum) at a concentration of 1.2×10⁶ per mL and 25 μL of cells (30,000 per well) were aliquoted to the appropriate wells of a 96-well white assay plate. To wells containing cells was added 25 μL of 3× antibody, also diluted in assay buffer. Following addition of antibody to target cells, the provided effector cells (frozen at 2×10⁷ per mL) were thawed at 37° C., and 630 uL added to 3.6 mL of warmed (37° C.) assay buffer, gently mixed, and 25 μL of effector cells (75000 per well, for an E:T ratio of 2.5) immediately added to the wells containing target cells and antibody. The plate was then incubated at 37° C. and 5% CO₂ for 6 hrs to allow for receptor cell activation and luciferase expression. After 6 hrs, the plate was equilibrated to room temperature (15 minutes), while the Bio-Glo Luciferase Assay Reagent was prepared. 75 μL of the Luciferase Assay Reagent were added to each well, the plate was incubated for 10 minutes, and luminescence was measured on a BioTek plate reader. In the wildtype human IgG1 format, some 2+1 bispecific antibodies against TfR were able to drive significant ADCC signal (FIG. 9). Further experiments were carried out to test whether these responses could be ameliorated by ablating binding to various FcRs.

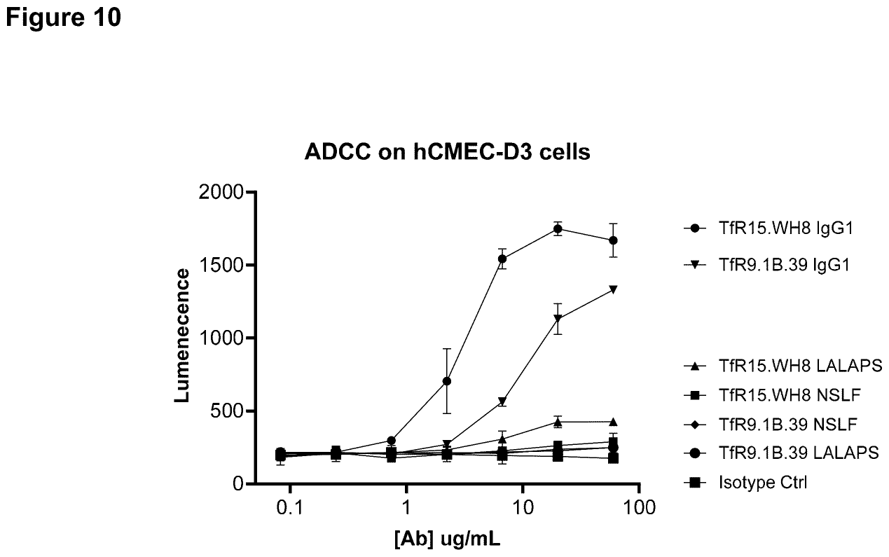

Fc Engineering and ADCC results: Two of the 2+1 anti-TfR bispecific antibodies, which elicited ADCC signal (TfR9.1B.39 and TfR15.WH8) were re-expressed in a variety of Fc formats, including wildtype hIgG1, hIgG1 NSLF and hIgG1 LALAPS and retested in the same ADCC assay format described previously. Both NSLF and LALAPS have been previously described to strongly decrease binding to FcgRIIIA (Leoh et al, Molecular Immunology, 29 Jul. 2015, 67(2 Pt B):407-415, Shang et al, J Biol Chem. 2014; 289:15309-15318), which is the primary driver of ADCC activity. In this assay, both NSLF and LALAPS were able to reduce the ADCC signal significantly, with NSLF showing >90% reduction of signal at the highest concentration tested for both 2+1 anti-Tfr bispecific antibodies tested (FIG. 10).

### Example 23: Binding Kinetics of 2+1 Anti-TfR Bispecific Antibody Affinity and Liability Optimized Clones

Binding kinetics of 2+1 anti-TfR bispecific antibodies to human TfR Avi-His were evaluated using a GatorBio BLI instrument (GatorBio, Palo Alto, CA). Briefly, 2+1 anti-TfR bispecific antibodies were diluted with running buffer (HBS-EP+, Teknova) with 0.5 mg/ml BSA (MP Biomedicals) in a series of seven, five, three-fold serial dilutions, and then two 10-fold dilutions starting at 4 μM concentration for Tf9.1B.39.36, TfR9.1B.39.37, TfR9.1B.39.38, and WH8 parental and starting at 2 μM concentration for TfR9.1B.39, TfR9.1B.39.05, TfR9.1B.39.39, TfR9.1B.39.40, TfR15.WH7.1, TfR15.WH8.1, and TfR15.WH12.1. Biotinylated TfR Avi-His was diluted with running buffer to 5 μg/mL. Streptavidin probes (Gator) were dipped into biotinylated TfR Avi-His solution, for five seconds to load protein onto the tips. The probes were then dipped into respective antibody concentrations for three minutes of association, followed by three minutes of dissociation in running buffer. Wash steps in running buffer were performed in between loading and association steps. Probes with biotinylated TfR Avi-His were regenerated with 10 mM glycine-HCl, pH 2.5 buffer. Biotinylated TfR Avi-His was replenished for five seconds at the beginning of each binding cycle. Data was double-referenced with reference probes and buffer (blank) wells, and were processed and analyzed using Gator Analysis Software (GatorBio).

The equilibrium dissociation constants (KD) were calculated from the fitted association and dissociation rate constants (k-on and k-off) for 2+1 anti-Tfr bispecific antibodies. The values were combined, means and standard deviation calculated, and graphs prepared using GraphPad Prism. Some antibodies, as indicated in the table, displayed heterogeneous binding profiles that do not fit to a 1:1 binding model, thus their rate constants could not be determined. The K_{D} values are summarized in Table 30 below.

TABLE 30
Equilibrium dissociation constants (K_{D}) for anti-TfR 2 + 1 antibodies

| | huTfR Avi-His | | | Cyno TfR Avi-His | | |
|---|---|---|---|---|---|---|
| Antibody | Mean K_{D} (M) | SD | N | Mean K_{D} (M) | SD | N |
| TfR9.1B.39 | 7.5E−07 | 3.2E−08 | 2 | 1.3E−06 | 5.1E−07 | 4 |
| TfR9.1B.39.05 | Heterogeneous | Heterogeneous | 1 | Heterogeneous | Heterogeneous | 1 |
| Tf9.1B.39.36 | 2.3E−06 | 2.8E−07 | 2 | 2.3E−06 | NA | 1 |
| TfR9.1B.39.37 | 5.5E−07 | 1.1E−07 | 2 | 1.3E−06 | 1.2E−06 | 2 |
| TfR9.1B.39.38 | 3.5E−06 | 7.2E−07 | 2 | 1.5E−06 | NA | 1 |
| TfR9.1B.39.39 | Heterogeneous | Heterogeneous | 1 | Heterogeneous | Heterogeneous | 1 |
| TfR9.1B.39.40 | LB | LB | 1 | 5.7E−07 | NA | 1 |
| TfR15.WH7.1 | 1.2E−07 | NA | 1 | 1.0E−07 | NA | 1 |
| TfR15.WH8.1 | 6.5E−08 | NA | 1 | 3.7E−08 | NA | 1 |
| TfR15.WH12.1 | Heterogeneous | Heterogeneous | 1 | Heterogeneous | Heterogeneous | 1 |

N = number of determinations; NT = not tested; NA = not applicable; LB = low binding (below limit of measurement); NB = no binding detected.

These results illustrate that 2+1 anti-TfR bispecific antibodies exhibit a range of affinities from approximately 30 nM to 4 μM for TfR Avi-His. Affinity of 2+1 anti-TfR bispecific antibodies for binding to human TfR Avi-His ranged from 65 nM to 3.5 μM; affinity of 2+1 anti-TfR bispecific antibodies for binding to cyno TfR Avi-His ranged from 38 nM to 2.3 μM.

### Example 24: TD-TfR Bi-Specific Antibody In Vitro sTREM2 Assay

In order to evaluate the pharmacokinetics and pharmacyodynamics of 2+1 anti-TfR antibodies, a 2+1 anti-TfR bi-specific antibody was generated with: (i) TfR9.1B.39.38 scFv; and (ii) a monoclonal antibody specific for human MS4A4A (referred to as “TD1”) in a 2+1 (hole) format (referred to as “TD1-TfR”) (described in Example 25). To confirm that TD1-TfR retains the function of the parental TD1 antibody, a soluble TREM2 (sTREM2) assay was conducted using human monocyte-derived macrophages. These cells would be expected to show an increased production of sTREM2 upon antibody treatment with TD1.

Human monocytes were isolated from whole blood using RosetteSep Human monocyte enrichment cocktail (Stemcell technologies) and Ficoll centrifugation per manufacturer protocols. After lysing red blood cells with ACK lysing buffer, monocytes were resuspended in complete media (RPMI, 10% FBS, Pen/Strep, L-glutamine, HEPES, non-essential amino acid, and sodium pyruvate). To obtain macrophages from these isolated monocytes, 50 ng/ml human M-CSF and 8% v/v human serum were added to the cells for 5-7 days.

10⁵ macrophages per well were stimulated with 1 ug/mL of tested antibody (huIgG1, TD1, iso-TfR (isotype control IgG antibody linked to TfR9.1B.39.38), or TD1-TfR) for 48 hours. Duplicate wells were treated as technical replicates. Supernatants were tested for sTREM2 by MSD platform.

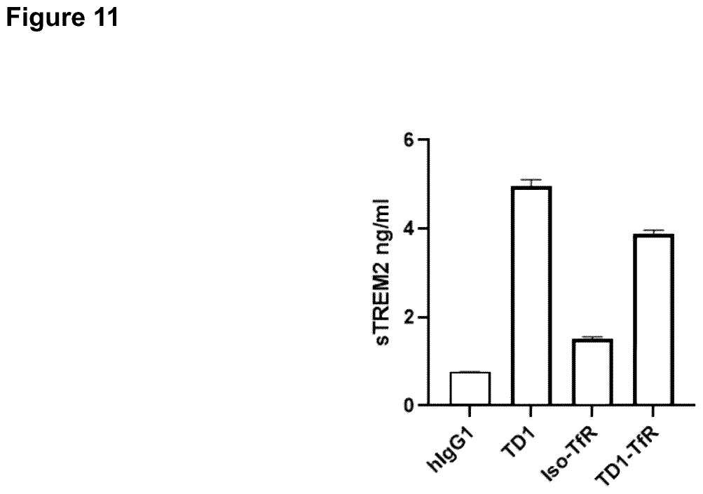

As illustrated in FIG. 11, TD1-TfR showed activity by increasing sTREM2 levels in an in vitro assay, as compared to hIgG1 and Iso-TfR. TD-1 and TD1-TfR showed similar increased sTREM2 levels in the in vitro assay. This data indicates that addition of the anti-TfR scFv to the TD1 antibody did not impair function of TD1.

### Example 25: TD1-TfR Bi-Specific Antibody and Method for Intravenous Administration to Non-Human Primates

In order to evaluate the pharmacokinetics and pharmacodynamics of 2+1 anti-TfR antibodies disclosed herein, a 2+1 anti-TfR bispecific antibody was generated with: (i) TfR9.1B.39.38 scFV; and (ii) an antibody specific for human MS4A4A (TD1), in a 2+1 (hole) format (referred to as “TD1-TfR”). TD1 has been shown to increase several biomarkers in the brains of non-human primates including soluble TREM2 (sTREM2) and Colony Stimulating Factor 1 (CSF1)—data not shown. The full amino acid sequence of TD1-TfR is below:

```
TD1 heavy chain hIgG1 NSLF (N325S, L328F) knob (T366W)
  (SEQ ID NO: 405)
QVQLVQSGSELKKPGASVKVSCKASGYAFTSYGLSWVRQAPGQGLEWMGWINTYSGV
PTYAQGFTGRFVFSLDTSVSTAYLQISSLKAEDTAVYYCARTMADYWGQGTLVTVSSA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSSKAFPAPIEKTISKAKGQPREPQVYTLPPSR
DELTKNQVSLWCLVKGFYPSDIAVEWESNGQPENNYKTTPPVLDSDGSFFLYSKLTVDK
SRWQQGNVFSCSVMHEALHNHYTQKSLSLSPGK.
TD1 heavy chain hIgG1 NSLF (N325S, L328F) hole scFv
(T366S, L368A, Y407V)-(G4S)3 linker-VH-20AA linker-VL with
mutations (H435R, Y436F)
  (SEQ ID NO: 406)
QVQLVQSGSELKKPGASVKVSCKASGYAFTSYGLSWVRQAPGQGLEWMGWINTYSGV
PTYAQGFTGRFVFSLDTSVSTAYLQISSLKAEDTAVYYCARTMADYWGQGTLVTVSSA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSSKAFPAPIEKTISKAKGQPREPQVYTLPPSR
DELTKNQVSLSCAVKGFYPSDIAVEWESNGQPENNYKTTPPVLDSDGSFFLVSKLTVDK
SRWQQGNVFSCSVMHEALHNRFTQKSLSLSPGKGGGGSGGGGSGGGGSQVQLVQSGA
EVKKPGASVKVSCKASGYTFTEYAMHWVRQAPGQGLEWMGGIAPNTGGTSYNQKFK
GRVTMTVDTSTSTAYMELSSLRSEDTAVYYCASQGWLLRGGDYWGQGTLVTVSSGGS
EGKSSGSGSESKSTGGSDVVMTQSPLSLPVTLGQPASISCKASQSLLDEGGKTYLNWLQ
QRPGQSPRRLIYLVSKLDSGVPDRFSGSGSGTDFTLKISRVEAEDVGVYYCWQGTHFPQ
TFGGGTKVEIK.
TD1 light chain
  (SEQ ID NO: 407)
DVVMTQSPLSLPVTLGQPASISCKSSRSLLYSAGKTYLSWFQQRPGQSPRRLIYLVSKLD
SGVPDRFSGSGSGTDFTLKISRVEAEDVGVYYCWQGIDFHQTFGGGTKVEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC.
```

Further, a bispecific antibody with (i) a TfR specific scFv and (ii) IgG1 NSLF in a 2+1 (hole) format was generated for control purposes (referred to as “Iso-TfR”). The full amino acid sequence of Iso-Tfr is below:

```
Isotype control heavy chain hIgG1 NSLF (N325S, L328F) knob (T366W)
  (SEQ ID NO: 408)
EVRLLESGGGLVQPGGSLRLSCAASGFTFSNYAMGWVRQAPGKGLEWVSAISGSGGST
YYADSVKGRFTTSRDDSKNALYLQMNSLRAEDTAVYYCARGGPGWYAADVWGQGTT
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSSKAFPAPIEKTISKAKGQPREPQVYT
LPPSRDELTKNQVSLWCLVKGFYPSDIAVEWESNGQPENNYKTTPPVLDSDGSFFLYSK
LTVDKSRWQQGNVFSCSVMHEALHNHYTQKSLSLSPGK.
Isotype control heavy chain hIgGI NSLF (N325S, L328F) hole scFv
(T366S, L368A, Y407V)-(G4S)3 linker-VH-20AA linker-VL with
mutations (H435R, Y436F)
  (SEQ ID NO: 409)
EVRLLESGGGLVQPGGSLRLSCAASGFTFSNYAMGWVRQAPGKGLEWVSAISGSGGST
YYADSVKGRFTTSRDDSKNALYLQMNSLRAEDTAVYYCARGGPGWYAADVWGQGTT
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSSKAFPAPIEKTISKAKGQPREPQVYT
LPPSRDELTKNQVSLSCAVKGFYPSDIAVEWESNGQPENNYKTTPPVLDSDGSFFLVSKL
TVDKSRWQQGNVFSCSVMHEALHNRFTQKSLSLSPGKGGGGSGGGGSGGGGSQVQLV
QSGAEVKKPGASVKVSCKASGYTFTEYAMHWVRQAPGQGLEWMGGIAPNTGGTSYN
QKFKGRVTMTVDTSTSTAYMELSSLRSEDTAVYYCASQGWLLRGGDYWGQGTLVTVS
SGGSEGKSSGSGSESKSTGGSDVVMTQSPLSLPVTLGQPASISCKASQSLLDEGGKTYLN
WLQQRPGQSPRRLIYLVSKLDSGVPDRFSGSGSGTDFTLKISRVEAEDVGVYYCWQGTH
FPQTFGGGTKVEIK.
Isotype control light chain
  (SEQ ID NO: 410)
DIQMTQSPSSLSASVGDRVTITCRASQSISSYLNWYQQKPGKAPKLLIYAASSLQSGVPSR
FSGSGSGTDFTLTISSLQPEDFATYYCQQADLPAFAFGGGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC.
```

The full amino acid sequence of the antibody specific for human MSA4A alone (TD1) is below:

TD1 heavy chain hIgG1 NSLF (N325S, L328F)
(SEQ ID NO: 411)
QVQLVQSGSELKKPGASVKVSCKASGYAFTSYGLSWVRQAPGQGLEWMGWINTYSGV
PTYAQGFTGRFVFSLDTSVSTAYLQISSLKAEDTAVYYCARTMADYWGQGTLVTVSSA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSSKAFPAPIEKTISKAKGQPREPQVYTLPPSR
DELTKNQVSLTCLVKGFYPSDIAVEWESNGQPENNYKTTPPVLDSDGSFFLYSKLTVDK
SRWQQGNVFSCSVMHEALHNHYTQKSLSLSPGK.
TD1 light chain
(SEQ ID NO: 407)
DVVMTQSPLSLPVTLGQPASISCKSSRSLLYSAGKTYLSWFQQRPGQSPRRLIYLVSKLD
SGVPDRFSGSGSGTDFTLKISRVEAEDVGVYYCWQGIDFHQTFGGGTKVEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC

A PK/PD study was conducted in naive male cynomolgus monkeys (n=12) following intravenous (iv) dosing of 20 mg/kg of each of the 4 antibodies in Table 31 (n=3) on Day 1 and 29 (total of 2 administrations). This dose level of TD1 has been shown to generate an incomplete PD response in NHPs, owing to the poor brain penetration of the antibody. The goal of the study was to look for increased antibody level in the brain, and a concurrent increase in the associated PD biomarkers.

TABLE 31
In vivo administration of anti-TfR bispecific antibodies

| | Number of | | |
|---|---|---|---|
| | Animals | | |
| | (male | Test | Dosing |
| Group | only) | Article | Route |
| 1 | 3 | Iso-TfR | IV |
| 2 | 3 | TD1-TfR | IV |
| 3 | 3 | TD1 | IV |
| 4 | 3 | huIgG1 | IV |
| | | (Isotype | |
| | | Control) | |

The intravenous doses were administered via a saphenous vein as slow push injection. The target dose level (mg/kg), target dose concentration (mg/mL), and target dose volume (mL/kg) were consistent between Groups 1-4.

### Example 26: Hematological Parameters Following Intravenous Administration of TD1-TfR Bi-Specific Antibody to Non-Human Primates

At pre-dose and following dose administration on days 3, 8, 15, 29, and 31, blood samples (˜2 mL) for hematology were collected from the femoral vein into tubes containing potassium EDTA.

TABLE 32
Hematology Parameters tested following intravenous
administration of anti-TfR Bi-specific Antibodies

| Red blood cell (erythrocyte) count | Platelet count |
|---|---|
| Hemoglobin | White blood cell (leukocyte) count |
| Hematocrit | Differential blood cell count (percent |
| Mean corpuscular volume | & absolute) |
| Mean corpuscular hemoglobin | Red cell distribution width |
| Mean corpuscular hemoglobin | Blood smear |
| concentration | Reticulocyte count |

Testing facility normal range for reticulocyte count is 25.7-122.3 10³/μl.

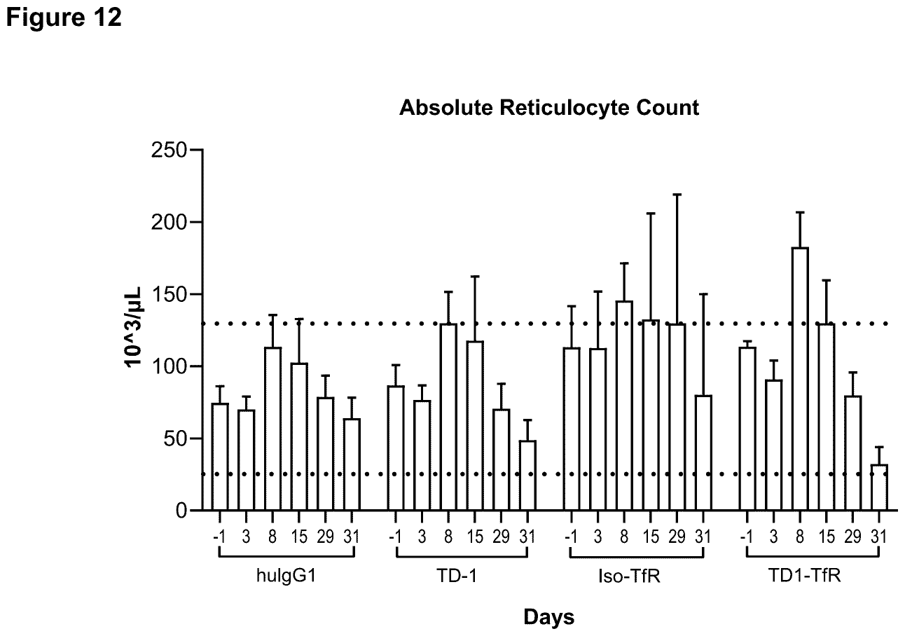

FIG. 12 illustrates absolute reticulocyte counts over the duration of the study. The normal range is indicated by dashed lines. Overall, no notable changes were observed in any hematology parameters, and no consistent reduction in reticulocytes was observed over the course of the study.

### Example 27: CSF and Serum Levels of TD1-TfR Bi-Specific Antibody Following IV Administration in Non-Human Primates

Cerebrospinal fluid (CSF) and serum were collected from the NHPs at various times following the first and second administrations as described in Example 25. The serum and CSF were tested for antibody levels as described below.

Sandwich ELISA method: 100 μL/well of goat anti-human IgG, Monkey adsorbed-BIOTIN (Southern Biotech; catalog #2049-08) working solution (0.5 μg/mL) was added to a 96-well Pierce™ streptavidin coated high binding plate with SuperBlock™ blocking buffer (ThermoScientific; Reference #15500) and incubated for 1 hour±10 minutes at Room Temperature (RT) on a plate shaker at 350 revolutions per minute (RPM). The serum or CSF quality controls (QC) and test samples were diluted to the minimum required dilution (MRD) in assay buffer (1×TBS, 0.05% Tween-20, and 0.1% BSA) prior to loading onto the assay plate while the standard calibration curve and assay buffer QCs (AB-QCs) were added directly to the assay plate. After the incubation was complete, the plate was washed 3 times with wash buffer (1×PBS and 0.05% Tween-20) using a plate washer and 100 μL/well of the calibration standards, QCs, and MRD-diluted Serum or CSF QCs and test samples were then added to the plate and incubated for 2 hours±15 minutes at room temperature on a plate shaker at 350 RPM. The plate was then washed, and 100 μL/well of goat anti-human IgG, Monkey adsorbed-TRP (Southern Biotech; catalog #2049-05) working solution (0.08 μg/mL) was added to the plate and incubated for 1 hour±10 minutes at room temperature on a plate shaker at 350 RPM. After the HRP incubation, the plate was washed and 100 μL/well of tetramethylbenzidine (TMB) substrate solution (Surmodics Product No. TMBS-1000-01) was added and incubated for 15±10 minutes at room temperature (RT) shielded from light by covering in aluminum foil or placed in a drawer. After the incubation was complete, 100 μL/well Stop Solution (Surmodics Product No. NSTP-1000-01) was added to the plate to stop the reaction. The plate was then placed on a plate shaker at 350 RPM for at least 2 minutes at RT. The plate was then read on a Molecular Devices SpectraMax M5 instrument and a raw optical density (OD450-540) signal was produced from each well. The concentration of the analyte in each unknown sample was determined by interpolation of the concentration using the calibration standard curve. The standard regression was performed by SoftMax Pro (Molecular Devices/Version 7.1) using a 4-Parameter Logistic (4-PL) model with a weighting factor of 1/Y².

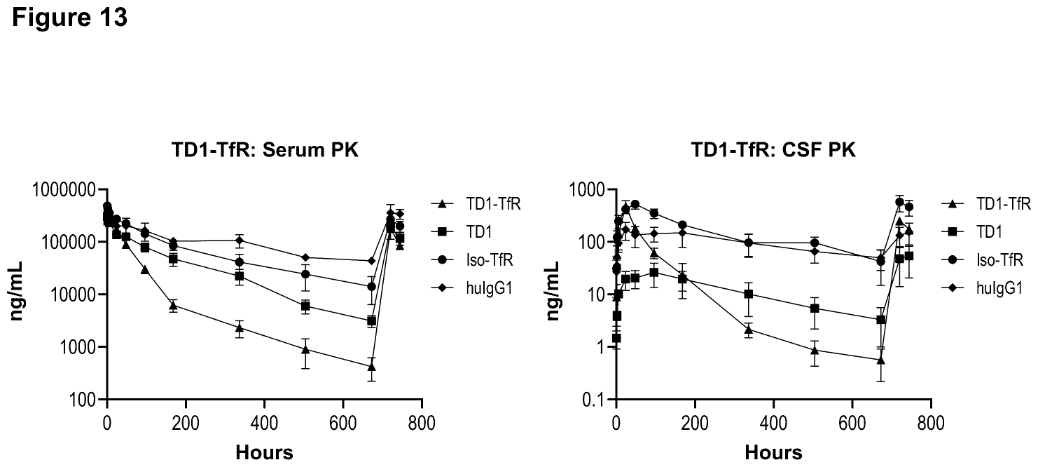

FIG. 13 illustrates that an increased CSF Cmax was seen with TD1-TfR, when compared to TD1. An increased CSF Cmax was also seen with Iso-TfR when compared to isotype control. Further, as illustrated in FIG. 13, TD1-TfR demonstrated faster serum clearance in NHPs as compared to either Iso-TfR or TD1 alone.

### Example 28: Effect of TfR Binding Arm on Brain Uptake and Brain Pharmacodynamics in Non-Human Primates

Antibody levels were also assessed directly in multiple NHP brain regions (frontal corex, entorhinal cortex and hippocampus) using vessel-depleted brain lysates.

Brain Sample preparation: Brain tissues were collected from the antibody treated cynomolgus monkeys on day 30 (i.e., 48 hours after the second antibody administration). Prior to this, animals were anaesthetized and underwent cardiac perfusion with PBS to clear blood vessels. See Example 25 for dosing details. Brain tissues were then minced and homogenized in HBSS buffer (MilliporeSigma #55037C) containing 10 mM HEPES (#15630130, Gibco) using a hand-operated grinder. Subsequently, samples were centrifuged at 1000 g for 5 minutes to pellet the vessel portion. The supernatants, representing the parenchyma, were then transferred into new tubes, and mixed with 10×RIPA buffer (final concentration 1×) including protease and phosphatase inhibitor cocktail (cOmplete™, Mini Protease Inhibitor Cocktail, Roche, #11836153001). After 45 minutes incubation on a nutator at 4° C., samples were frozen on dry ice and stored at −80° C. until analysis. Vessel fractions were cleared from myelin debris via centrifugation through 18% Dextran (70 kDa, #31390, Sigma-Aldrich), followed by 2 washes in HBSS buffer. Afterwards, samples were lysed in RIPA buffer (R0278, Sigma-Aldrich) and stored at −80° C. until analysis. The validity of this method has previously been confirmed using Western Blot analysis, in which markers of brain endothelial cells (e.g., CD31 and Claudin-5) were absent in the parenchymal portions (data not shown).

Pharmacokinetic (PK) analysis: Antibody concentrations in brain samples were measured using MSD (Meso Scale Discovery) method. Briefly, 50 μl/well of goat anti-human IgG (monkey absorbed) antibody was added to MSD plates at 1 μg/ml in PBS and incubated overnight on a shaker at 500 RPM at 4° C. Plates were then washed 3× with wash buffer (0.05% Tween-20 in 1×PBS), followed by incubation with blocking buffer (wash buffer containing 3% BSA) for 1 hour at RT. Subsequently, brain (diluted in PBS) samples were added in duplicates to the plates and incubated for 2 hours on a shaker at 500 RPM at room temperature. Plates were then washed 3× with wash buffer, followed by addition of 40 μl/well of Sulfo-Tag goat anti-human Ab (R32AJ-5, Meso Scale Discovery) at 0.5 μg/ml in PBS and 1 hr incubation on shaker at 500 RPM. Finally, after 3× washes with wash buffer, 150 μl/well of 1×READ buffer (R92PC-2, Meso Scale Discovery) was added to the plates, before they are read using a Sector Imager S600 instrument. The standard curve for each treatment was generated using eleven 2-fold serial dilutions (from 100-0.019 ng/ml) of their respective antibody. The curves were fitted using a four-parameter logistic regression for calculating antibody concentrations within samples.

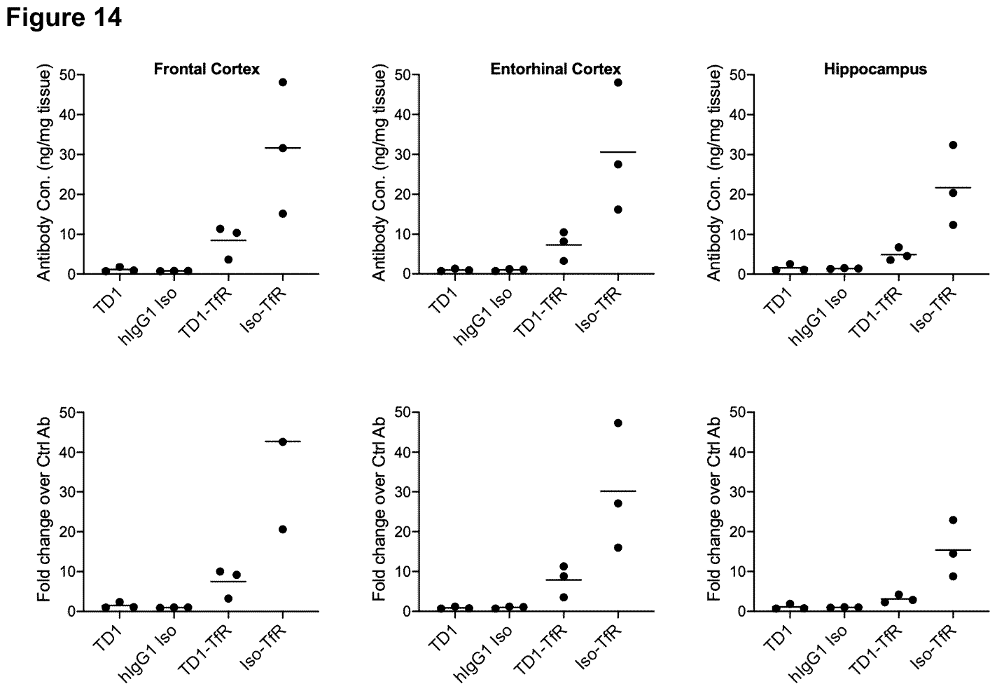

FIG. 14 illustrates that the TfR BBB targeting arm was able to increase brain uptake of (i) TD1-TfR compared to TD1 in all 3 brain regions tested (ranging from a 2 to 11 fold increase) and (ii) Iso-TfR compared to isotype parental antibody in all 3 brain regions tested (ranging from an 8 to 64 fold increase).

### Example 29: Pharmacodynamic Analysis of TD1-TfR Bi-Specific Antibody in NHP Samples

CSF and serum from NHP test subjects, as described in Example 25, and NHP brain lysates, as described in Example 28, were further tested for downstream biomarkers of TD1 function, including sTREM2 and CSF1R, as described below.

### CSF1R Method Summary (Brain Lysate)

An Enzyme-linked Immunosorbent Assay (ELISA) kit from R&D Systems (Catalog No. DY329) was used to measure the concentration of Colony Stimulating Factor 1 Receptor (CSF1R) in cynomolgus monkey brain lysates. The mouse anti-Human M-CSF R Capture Antibody (R&D Systems, Part #841246) was diluted in 1×PBS (Corning; REF 21-040-CM) to a working concentration of 4 μg/mL and coated at 100 μL/well on a 96-well microplate (Nunc-Immuno Maxisorp; ThermoScientific Catalog No. 446612) and incubated overnight at room temperature without shaking. The next day, the plate was washed 3× with wash buffer (1×PBS and 0.05% Tween-20), blocked with 300 μL of reagent diluent (R&D Systems, Part #841380) and incubated for at least 1 hour without shaking. Human M-CSF R Standards and QCs (R&D Systems, Part #841248) were prepared in Reagent Diluent. Test samples and QCs were diluted at the minimum required dilution (MRD) in Assay Buffer (1×PBS, 1% BSA and 0.05% Tween-20). The plate was then washed, 100 μL/well of Human M-CSF1R Standards, diluted QCs and diluted samples in Assay Buffer were added to the appropriate wells and incubated for 2 hours at room temperature. The plate was washed and 100 μL/well of the 100 ng/mL biotinylated Goat anti-Human M-CSF R detection antibody (R&D Systems, Part #841247) working solution in reagent diluent was added and incubated for 2 hours at room temperature. After the incubation was complete, the plate was washed and 1:200 dilution of Streptavidin-HRP (R&D Systems, Part #890803) in reagent diluent was then added and incubated for 20 minutes at room temperature. The plate was washed for a final time and 100 μL/well of substrate solution (R&D Systems, Parts #895000 and 895001) was added and incubated for 20 minutes at room temperature. After the incubation was complete, 50 μL/well of stop solution (R&D Systems, Part #895032) was added to the plate and mixed thoroughly. The plate was then read on a Molecular Devices SpectraMax M5 instrument and a raw optical density (OD₄₅₀₋₅₄₀) signal was produced from each well. The concentration of CSF1R in each unknown sample was determined by interpolation against the calibration standard curve. The standard regression was performed by SoftMax Pro (Molecular Devices, version 7.1) using a 4-Parameter Logistic (4-PL) model with a weighting factor of 1/Y².

### CSF1 Method Summary (CSF)

An Electrochemiluminescence (ECL) Assay kit from Meso Scale Discovery (MSD, Catalog #K151XRK-4) was used to measure the concentrations of Colony Stimulating Factor 1 (CSF1) in Cynomolgus Monkey Cerebral Spinal Fluid (CSF) samples. A 96-well MSD GOLD Small Spot Streptavidin Plate (MSD, Catalog #L45SA-1) was coated with 25 μL/well of the biotinylated anti-human CSF1 Antibody (MSD, Catalog #C21XR-3) diluted 1:17.5 in Diluent 100 (MSD, Catalog #R50AA-4) and incubated for 1 hour at room temperature on a plate shaker set at 700 revolutions per minute (RPM).

Standards were prepared in Diluent 43 (MSD, Catalog #R50AG-2) and were further diluted 1:1 in assay buffer prior to loading onto the plate. QCs were prepared in Diluent 43 and were diluted at the MRD in assay buffer. Diluted QCs were further diluted 1:1 with Diluent 43 prior to loading onto the plate. Test samples were diluted at the MRD in assay buffer. Diluted samples were further diluted 1:1 in Diluent 43 prior to loading onto the plate.

After washing, 50 μL of Standards, QCs and test samples diluted in Diluent 43 were added to the appropriate wells and incubated for 1 hour at room temperature on a shaker set at 700 RPM. The plate was then washed and 50p of the detection antibody (SULFO-TAG Anti-Human CSF1 Antibody; MSD, Catalog #D21XR-3) diluted 1:100 in Diluent 3 (MSD, Catalog #R50AP-2) was added to each well and incubated for 1 hour at room temperature on a plate shaker set at 700 RPM. After the incubation was complete, the plates were washed and 150 L/well of MSD GOLD Read Buffer B (MSD, Catalog #R60AM-2) was then added and plates were read on an MSD Sector 600 Imager. In the presence of read buffer, ruthenium produced a chemiluminescent signal when a voltage was applied. The intensity of the signal was proportional to the concentration of CSF1 present in the sample. The CSF1 levels were quantified according to the standard curve utilizing a four-parameter logistic (4-PL) curve fit equation with 1/y² weighting. Data was analyzed using Microsoft Excel and GraphPad Prism 9.0.

sTREM2 Method Summary (Serum, CSF and Brain Lysates)

An Electrochemiluminescence (ECL) Assay on the MSD platform was used to measure the concentration of Triggering Receptor Expressed on Myeloid Cells 2 (TREM2) in Cynomolgus Monkey Serum, Cerebral Spinal Fluid (CSF), and Brain Lysates. A 96-well standard MSD plate (Catalog #L15XA) was coated with 50 μL/well of capture antibody T2-8F11 (generated at Alector), in 1×PBS (Corning; REF 21-040-CM) at 2-8° C. overnight on a plate shaker set at 500 revolutions per minute (RPM). After washing with wash buffer (1×PBS, 0.05% Tween-20), the plate was blocked with 150 μL/well of blocking buffer (1% heat-inactivated high-grade BSA in PBS). The plate was then washed and 50 μL/well of Cynomolgus TREM2-Fc Standards (generated at Adimab LLC), QCs and diluted samples in assay buffer (1×PBS, 0.05% Tween-20, and 1% BSA) were added to the appropriate wells and incubated for 1 hour at room temperature (RT) on a shaker set at 500 RPM. TREM2 present in the Standards, QCs and samples were bound by the immobilized T2-8F11 capture antibody. After washing away any unbound substances, 50 μL/well of biotinylated goat anti-human TREM2 polyclonal antibody (R&D Systems, Catalog No. BAF1828) diluted in assay buffer at 100 ng/mL were added to wells of the plate and incubated for 1 hour at RT on a shaker set at 500 RPM. After washing the plates, 50 μL of Sulfo-Tag streptavidin (MSD, Catalog No. R32AD-1) solution (0.2 g/mL) in assay buffer was added and incubated for 30 minutes at RT on a shaker set at 500 RPM. After the incubation was complete, the plates were washed and 150 L/well of 1× Read buffer (prepared by diluting 4× Read Buffer T with Surfactant (MSD, Catalog #R92TC-1) with deionized water) was added and plates were read on an MSD Sector 600 Imager. In the presence of read buffer, ruthenium produced a chemiluminescent signal when a voltage was applied. The intensity of the signal was proportional to the concentration of TREM2 present in the sample. The standard regression was performed by SoftMax Pro (Molecular Devices, version 7.1) using a 4-Parameter Logistic (4-PL) model with a weighting factor of 1/Y².

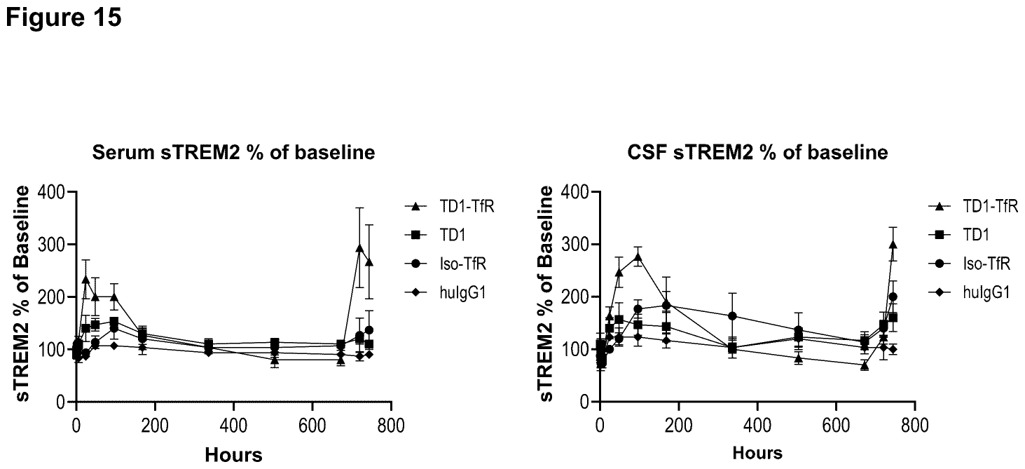

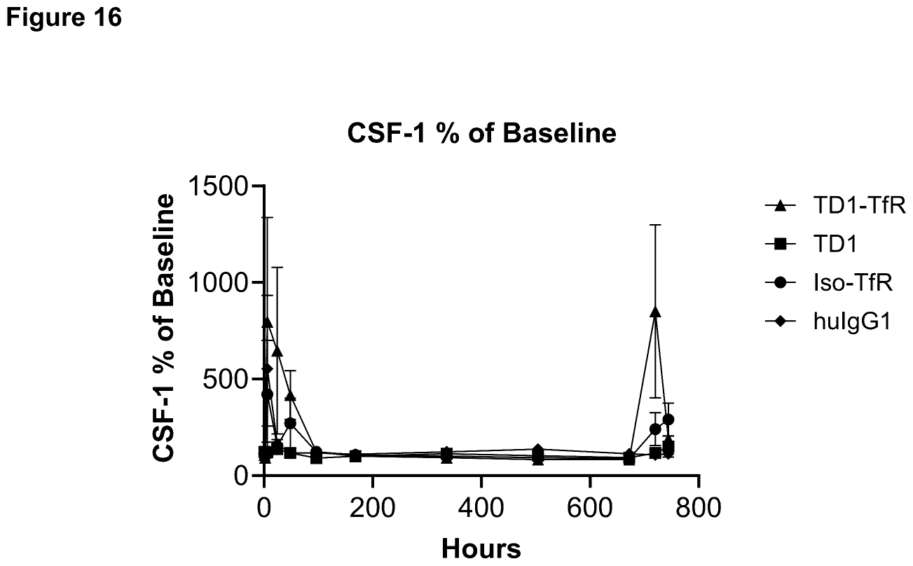

FIG. 15 illustrates that serum levels of soluble TREM2 (sTREM2) were increased in NHPs treated with TD1-TfR as compared to TD1 alone, Iso-TfR or hIgG1 following administration of the first and the second dose. FIG. 16 illustrates that CSF-1 levels were increased in CSF from NHPs treated with TD1-TfR as compared to TD1 alone, Iso-Tfr or hIgG1. These data show that the enhanced brain penetration seen in Example 28 also led to a stronger pharmacodynamic response.

### Example 30: Measuring TfR Levels in Cynomolgus Brain after Administration of 2+1 Anti-TfR Bispecific Antibodies

Anti-TfR antibodies have been shown to be able to degrade the receptor (Niewoehner et al, Neuron 2014 8:49-60), which is an undesirable side effect of a BBB-penetrating anti-TfR molecule. In order to test for this effect, we collected brain tissue samples from the non-human primates dosed with 2+1 anti-TfR and control antibodies (Example 25) and quantified total TfR levels by an MSD method.

Sample preparation: Brain tissues were collected from the antibody treated cynomolgus monkeys on day 31 (i.e., 48 hrs after the second Ab injection) and subsequently frozen. Prior to this, animals were anesthetized and underwent whole body perfusion with heparinized 0.9% saline to clear blood vessels. Thawed brain sections were then minced and homogenized at a concentration of 140 mg/mL in HBSS buffer (MilliporeSigma #55037C) containing 10 mM HEPES (#15630130, Gibco) using a hand-operated grinder. Subsequently, samples were mixed with 10× Radioimmunoprecipitation assay buffer (RIPA, 1× final concentration) containing Halt Protease Inhibitor Cocktail (Thermo Scientific #78438). After incubating on a nutator at 4° C. for 45 minutes, the samples were centrifuged at 15,000 RPM for 20 minutes at 4° C. The resulting supernatants were then transferred to new tubes and stored at −80° C. until further analysis.

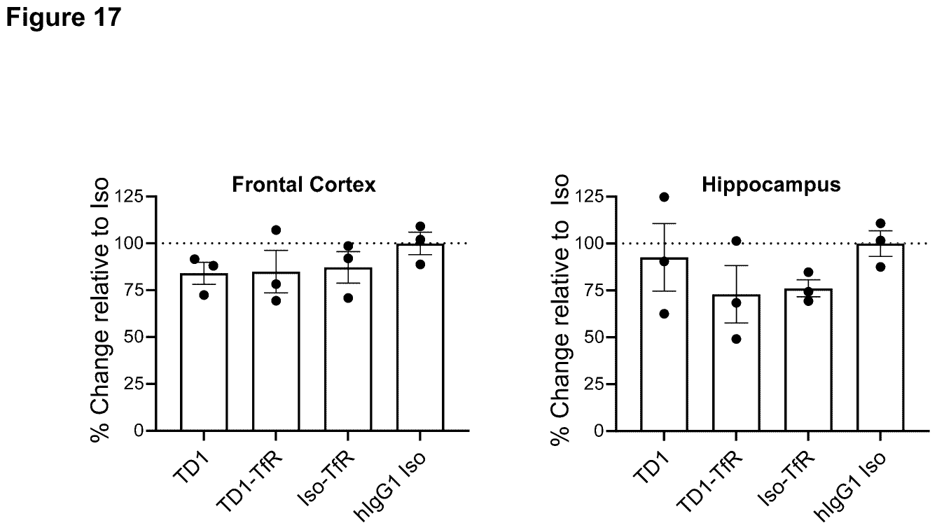

The expression levels of TfR in the NHP brain samples were determined using the MSD (Meso Scale Discovery) method. Briefly, a volume of 50 mL/well of a mouse anti-cynomolgus TfR IgG1 Ab (Invitrogen, #MA5-16644) was added to MSD plates at a concentration of 1 mg/mL in PBS. The plates were then incubated overnight at 4° C. on a shaker at 500 RPM. Afterward, the plates were washed three times with wash buffer (0.05% Tween-20 in 1×PBS) and incubated with 150 mL/well of blocking buffer (wash buffer containing 3% BSA) for 1 hour at room temperature. To generate a standard curve, eleven 3-fold serial dilutions of a cynomolgus-TfR ECD protein (produced in-house), starting at 1 mg/mL, were prepared in PBS containing RIPA buffer. Standard dilutions and brain samples (diluted 1:6 in PBS) were then added to the plates at a volume of 50 mL/well. Notably, the final concentration of RIPA buffer was kept consistent for both the standard dilutions and brain samples. Subsequently, plates were incubated for 2 hrs on a shaker at 500 RPM, followed by three washes with wash buffer. Next, 30 mL/well of a Sulfo-Tag rabbit anti-cynomolgus TfR Ab (SinoBilogical, Cat #90253-T16) was added to the plates at a concentration of 0.5 mg/mL in assay buffer (wash buffer containing 1% BSA). The plates were then incubated on a shaker at 500 RPM for 1 hour. After another round of washing, 150 mL/well of 1×READ buffer (R92PC-2, Meso Scale Discovery) was added to the plates, and then plates were immediately read using a Sector Imager S600 instrument. Data processing and analysis were performed using SoftMax Pro 7.1 and Prism 9.0 software, respectively. The brain expression levels of TfR in all animals were normalized against the average TfR levels in the control group (treated with hIgG1 isotype control antibody). As shown in FIG. 17, our quantitative data analysis revealed a slight decline in TfR expression levels in the brain tissues of animals treated with 2+1 anti-TfR bispecific antibodies compared to those injected with either hIgG1 Isotype control antibody or TD1 antibody alone that was not statistically significant. These results show that 2+1 anti-TfR bispecific antibodies disclosed herein do not have a significant impact on TfR expression in vivo.

1. An antigen-binding domain that specifically binds to the apical domain of the human transferrin receptor (TfR), wherein the antigen binding domain localizes to brain parenchyma in a subject following peripheral injection, and wherein the antigen-binding domain is not within a modified CH3 domain.

2. An antigen-binding domain that specifically binds to human transferrin receptor (TfR), wherein the antigen-binding domain comprises heavy chain variable region (VH) complementarity determining region (CDR) 1, VH CDR2, VH CDR3 and light chain variable region (VL) CDR1, CDR2, and CDR3 sequences comprising the amino acid sequences of:
(i) SEQ ID NOs:94, 415, 418, 423, 150, and 151, respectively;
(ii) SEQ ID NOs:74-76 and 135-137, respectively;
(iii) SEQ ID NOs:77-79 and 138-140, respectively;
(iv) SEQ ID NOs:80-82 and 135-137, respectively;
(v) SEQ ID NOs:83-85 and 141-143, respectively;
(vi) SEQ ID NOs:74, 86, 76 and 135-137, respectively;
(vii) SEQ ID NOs:87-89, and 143-145, respectively;
(viii) SEQ ID NOs:90-92 and 146-148, respectively;
(ix) SEQ ID NOs:83, 93, 85 and 141-143, respectively;
(x) SEQ ID NOs:94-96 and 149-151, respectively;
(xi) SEQ ID NOs:97, 86, 98, and 135-137, respectively;
(xii) SEQ ID NOs:99, 100, 101, and 152-154, respectively;
(xiii) SEQ ID NOs:99, 102, 103, and 155-157, respectively;
(xiv) SEQ ID NOs:104-106, 138, 158, and 159, respectively;
(xv) SEQ ID NOs:107-109 and 160-162, respectively;
(xvi) SEQ ID NOs:99, 110, 103, and 155-157, respectively;
(xvii) SEQ ID NOs:99, 111, 112, 152, 163, and 164, respectively;
(xviii) SEQ ID NOs:90, 113, 114, 165, 166, and 157, respectively;
(xix) SEQ ID NOs:83, 115, 85, 141, 142, and 167, respectively;
(xx) SEQ ID NOs:116, 110, 103, and 155-157, respectively;
(xxi) SEQ ID NOs:117-119 and 168-170, respectively;
(xxii) SEQ ID NOs:116, 110, 103, and 168-170, respectively;
(xxiii) SEQ ID NOs:90, 120, 114, 165, 166, and 157, respectively;
(xxiv) SEQ ID NOs:90, 113, 114, 171, 166, and 157, respectively;
(xxv) SEQ ID NOs:97, 121, 122, 135, 136, and 172, respectively;
(xxvi) SEQ ID NOs:74, 123, 124, 173, 136, and 137, respectively;
(xxvii) SEQ ID NOs:74, 125, 126, 174, 136, and 137, respectively;
(xxviii) SEQ ID NOs:99, 127, 128, 155, 175, and 176, respectively;
(xxix) SEQ ID NOs:90, 129, 130, 165, 166, and 157, respectively;
(xxx) SEQ ID NOs:131, 132, 133, 177, 166, and 178, respectively;
(xxxi) SEQ ID NOs:131, 132, 133, and 179-181, respectively;
(xxxii) SEQ ID NOs:90, 134, 114, 165, 166, and 157, respectively;
(xxxiii) SEQ ID NOs:83, 302, 303, 141, 142, and 182, respectively;
(xxxiv) SEQ ID NOs:99, 305, 103, 307, 308, and 157, respectively;
(xxxv) SEQ ID NOs:99, 306, 103, 307, 308, and 157, respectively;
(xxxvi) SEQ ID NOs:99, 306, 103, 307, 309, and 157, respectively;
(xxxvii) SEQ ID NOs:99, 306, 103, 310, 311, and 157, respectively;
(xxxviii) SEQ ID NOs:99, 305, 103, 310, 311, and 157, respectively;
(xxxix) SEQ ID NOs:99, 312, 103, 307, 308, and 157, respectively;
(xl) SEQ ID NOs:99, 312, 103, 307, 309, and 157, respectively;
(xli) SEQ ID NOs:99, 312, 103, 310, 311, and 157, respectively
(xlii) SEQ ID NOs:94, 95, 418, 421, 150, and 151, respectively;
(xliii) SEQ ID NOs:94, 412, 419, 422, 150, and 151, respectively;
(xliv) SEQ ID NOs:94, 412, 420, 422, 150, and 151, respectively;
(xlv) SEQ ID NOs:94, 413, 418, 422, 150, and 151, respectively;
(xlvi) SEQ ID NOs:94, 414, 418, 422, 150, and 151, respectively;
(xlvii) SEQ ID NOs:94, 416, 418, 422, 150, and 151, respectively;
(xlviii) SEQ ID NOs:94, 417, 418, 424, 150, and 151, respectively;
(xlix) SEQ ID NOs:460, 312, 103, 307, 309, and 157, respectively;
(l) SEQ ID NOs:460, 312, 461, 307, 309, and 157, respectively;
(li) SEQ ID NOs:460, 312, 462, 307, 309, and 157, respectively;
(lii) SEQ ID NOs:460, 312, 463, 307, 309, and 157, respectively;
(liii) SEQ ID NOs:460, 312, 464, 307, 309, and 157, respectively;
(liv) SEQ ID NOs:460, 312, 465, 307, 309, and 157, respectively;
(lv) SEQ ID NOs:460, 312, 466, 307, 309, and 157, respectively;
(lvi) SEQ ID NOs:460, 312, 467, 307, 309, and 157, respectively;
(lvii) SEQ ID NOs:460, 312, 468, 307, 309, and 157, respectively;
(lviii) SEQ ID NOs:460, 312, 469, 307, 309, and 157, respectively;
(lix) SEQ ID NOs:460, 312, 470, 307, 309, and 157, respectively;
(lx) SEQ ID NOs:460, 312, 103, 471, 309, and 157, respectively;
(lxi) SEQ ID NOs:460, 312, 103, 472, 309, and 157, respectively;
(lxii) SEQ ID NOs:460, 312, 103, 473, 309, and 157, respectively;
(lxiii) SEQ ID NOs:460, 312, 103, 474, 309, and 157, respectively;
(lxiv) SEQ ID NOs:460, 312, 103, 475, 309, and 157, respectively;
(lxv) SEQ ID NOs:460, 312, 103, 476, 309, and 157, respectively; or
(lxvi) SEQ ID NOs:460, 312, 103, 471, 309, and 157, respectively.

3. The antigen-binding domain of claim 2, wherein the antigen-binding domain comprises a VH and a VL, wherein the VH and VL comprise amino acid sequences at least 80%, at least 85%, at least 90%, at least 95%, or at least 99% identical to the amino acid sequences of:
(i) SEQ ID NOs:399 and 400, respectively;
(ii) SEQ ID NOs:10 and 11, respectively;
(iii) SEQ ID NOs:12 and 13, respectively;
(iv) SEQ ID NOs:14 and 15, respectively;
(v) SEQ ID NOs:16 and 17, respectively;
(vi) SEQ ID NOs:18 and 19, respectively;
(vii) SEQ ID NOs:20 and 21, respectively;
(viii) SEQ ID NOs:22 and 23, respectively;
(ix) SEQ ID NOs:24 and 25, respectively;
(x) SEQ ID NOs:26 and 27, respectively;
(xi) SEQ ID NOs:28 and 29, respectively;
(xii) SEQ ID NOs:30 and 31, respectively;
(xiii) SEQ ID NOs:32 and 33, respectively;
(xiv) SEQ ID NOs:34 and 35, respectively;
(xv) SEQ ID NOs:36 and 37, respectively;
(xvi) SEQ ID NOs:38 and 39, respectively;
(xvii) SEQ ID NOs:40 and 41, respectively;
(xviii) SEQ ID NOs:42 and 43, respectively;
(xix) SEQ ID NOs:44 and 45, respectively;
(xx) SEQ ID NOs:46 and 47, respectively;
(xxi) SEQ ID NOs:48 and 49, respectively;
(xxii) SEQ ID NOs:50 and 51, respectively;
(xxiii) SEQ ID NOs:52 and 53, respectively;
(xxiv) SEQ ID NOs:54 and 55, respectively;
(xxv) SEQ ID NOs:56 and 57, respectively;
(xxvi) SEQ ID NOs:58 and 59, respectively;
(xxvii) SEQ ID NOs:60 and 61, respectively;
(xxviii) SEQ ID NOs:62 and 63, respectively;
(xxix) SEQ ID NOs:64 and 65, respectively;
(xxx) SEQ ID NOs:66 and 67, respectively;
(xxxi) SEQ ID NOs:68 and 69, respectively;
(xxxii) SEQ ID NOs:70 and 71, respectively;
(xxxiii) SEQ ID NOs:72 and 73, respectively;
(xxxiv) SEQ ID NOs:313 and 314, respectively;
(xxxv) SEQ ID NOs:315 and 316, respectively;
(xxxvi) SEQ ID NOs:317 and 314, respectively;
(xxxvii) SEQ ID NOs:318 and 316, respectively;
(xxxviii) SEQ ID NOs:319 and 314, respectively;
(xxxix) SEQ ID NOs:320 and 316, respectively;
(xl) SEQ ID NOs:321 and 314, respectively;
(xli) SEQ ID NOs:322 and 316, respectively;
(xlii) SEQ ID NOs:323 and 314, respectively;
(xliii) SEQ ID NOs:324 and 316, respectively;
(xliv) SEQ ID NOs:325 and 326, respectively;
(xlv) SEQ ID NOs:327 and 328, respectively;
(xlvi) SEQ ID NOs:325 and 329, respectively;
(xlvii) SEQ ID NOs:330 and 331, respectively;
(xlviii) SEQ ID NOs:325 and 332, respectively;
(xlix) SEQ ID NOs:330 and 333, respectively;
(l) SEQ ID NOs:334 and 332, respectively;
(li) SEQ ID NOs:335 and 336, respectively;
(lii) SEQ ID NOs:337 and 338, respectively;
(liii) SEQ ID NOs:38 and 33, respectively;
(liv) SEQ ID NOs:339 and 340, respectively;
(lv) SEQ ID NOs:339 and 341, respectively;
(lvi) SEQ ID NOs:339 and 342, respectively;
(lvii) SEQ ID NOs:343 and 340, respectively;
(lviii) SEQ ID NOs:343 and 342, respectively;
(lix) SEQ ID NOs:344 and 340, respectively;
(lx) SEQ ID NOs:344 and 341, respectively;
(lxi) SEQ ID NOs:344 and 342, respectively;
(lxii) SEQ ID NOs:345 and 340, respectively;
(lxiii) SEQ ID NOs:345 and 341, respectively;
(lxiv) SEQ ID NOs:345 and 342, respectively;
(lxv) SEQ ID NOs:346 and 347, respectively;
(lxvi) SEQ ID NOs:348 and 316, respectively;
(lxvii) SEQ ID NOs:349 and 347, respectively;
(lxviii) SEQ ID NOs:349 and 350, respectively;
(lxix) SEQ ID NOs:351 and 352, respectively;
(lxx) SEQ ID NOs:349 and 353, respectively;
(lxxi) SEQ ID NOs:346 and 354, respectively;
(lxxii) SEQ ID NOs:349 and 355, respectively;
(lxxiii) SEQ ID NOs:349 and 356, respectively;
(lxxiv) SEQ ID NOs:357 and 316, respectively;
(lxxv) SEQ ID NOs:349 and 358, respectively;
(lxxvi) SEQ ID NOs:349 and 316, respectively;
(lxxvii) SEQ ID NOs:359 and 360, respectively;
(lxxviii) SEQ ID NOs:361 and 362, respectively;
(lxxix) SEQ ID NOs:363 and 316, respectively;
(lxxx) SEQ ID NOs:364 and 365, respectively;
(lxxxi) SEQ ID NOs:349 and 366, respectively;
(lxxxii) SEQ ID NOs:346 and 316, respectively;
(lxxxiii) SEQ ID NOs:346 and 367, respectively;
(lxxxiv) SEQ ID NOs:349 and 368, respectively;
(lxxxv) SEQ ID NOs:369 and 316, respectively;
(lxxxvi) SEQ ID NOs:346 and 370, respectively;
(lxxxvii) SEQ ID NOs:371 and 316, respectively;
(lxxxviii) SEQ ID NOs:372 and 356, respectively;
(lxxxix) SEQ ID NOs:357 and 358, respectively;
(xc) SEQ ID NOs:349 and 373, respectively;
(xci) SEQ ID NOs:346 and 374, respectively;
(xcii) SEQ ID NOs:375 and 316, respectively;
(xciii) SEQ ID NOs:376 and 316, respectively;
(xciv) SEQ ID NOs:346 and 377, respectively;
(xcv) SEQ ID NOs:378 and 379, respectively;
(xcvi) SEQ ID NOs:380 and 381, respectively;
(xcvii) SEQ ID NOs:349 and 382, respectively;
(xcviii) SEQ ID NOs:357 and 383, respectively;
(xcix) SEQ ID NOs:349 and 358, respectively;
(c) SEQ ID NOs:384 and 316, respectively;
(ci) SEQ ID NOs:385 and 316, respectively;
(cii) SEQ ID NOs:357 and 386, respectively;
(ciii) SEQ ID NOs:387 and 388, respectively;
(civ) SEQ ID NOs:359 and 316, respectively;
(cv) SEQ ID NOs:389 and 316, respectively;
(cvi) SEQ ID NOs:390 and 316, respectively;
(cvii) SEQ ID NOs:391 and 392, respectively;
(cviii) SEQ ID NOs:393 and 356, respectively;
(cix) SEQ ID NOs:390 and 392, respectively;
(cx) SEQ ID NOs:357 and 386, respectively;
(cxi) SEQ ID NOs:394 and 395, respectively;
(cxii) SEQ ID NOs:396 and 395, respectively;
(cxiii) SEQ ID NOs:397 and 395, respectively;
(cxiv) SEQ ID NOs:398 and 395, respectively;
(cxv) SEQ ID NOs:401 and 395, respectively;
(cxvi) SEQ ID NOs:402 and 403, respectively;
(cxvii) SEQ ID NOs:443 and 341, respectively;
(cxviii) SEQ ID NOs:444 and 341, respectively;
(cxix) SEQ ID NOs:445 and 341, respectively;
(cxx) SEQ ID NOs:446 and 341, respectively;
(cxxi) SEQ ID NOs:447 and 341, respectively;
(cxxii) SEQ ID NOs:448 and 341, respectively;
(cxxiii) SEQ ID NOs:449 and 341, respectively;
(cxxiv) SEQ ID NOs:450 and 341, respectively;
(cxxv) SEQ ID NOs:451 and 341, respectively;
(cxxvi) SEQ ID NOs:452 and 341, respectively;
(cxxvii) SEQ ID NOs:453 and 341, respectively;
(cxxviii) SEQ ID NOs:443 and 454, respectively;
(cxxix) SEQ ID NOs:443 and 455, respectively;
(cxxx) SEQ ID NOs:443 and 456, respectively;
(cxxxi) SEQ ID NOs:443 and 457, respectively;
(cxxxii) SEQ ID NOs:443 and 458, respectively; or
(cxxxiii) SEQ ID NOs:443 and 459, respectively;
or wherein the antigen-binding domain comprises a VH and VL comprising the amino acid sequences of:
(i) SEQ ID NOs:399 and 400, respectively;
(ii) SEQ ID NOs:10 and 11, respectively;
(iii) SEQ ID NOs:12 and 13, respectively;
(iv) SEQ ID NOs:14 and 15, respectively;
(v) SEQ ID NOs:16 and 17, respectively;
(vi) SEQ ID NOs:18 and 19, respectively;
(vii) SEQ ID NOs:20 and 21, respectively;
(viii) SEQ ID NOs:22 and 23, respectively;
(ix) SEQ ID NOs:24 and 25, respectively;
(x) SEQ ID NOs:26 and 27, respectively;
(xi) SEQ ID NOs:28 and 29, respectively;
(xii) SEQ ID NOs:30 and 31, respectively;
(xiii) SEQ ID NOs:32 and 33, respectively;
(xiv) SEQ ID NOs:34 and 35, respectively;
(xv) SEQ ID NOs:36 and 37, respectively;
(xvi) SEQ ID NOs:38 and 39, respectively;
(xvii) SEQ ID NOs:40 and 41, respectively;
(xviii) SEQ ID NOs:42 and 43, respectively;
(xix) SEQ ID NOs:44 and 45, respectively;
(xx) SEQ ID NOs:46 and 47, respectively;
(xxi) SEQ ID NOs:48 and 49, respectively;
(xxii) SEQ ID NOs:50 and 51, respectively;
(xxiii) SEQ ID NOs:52 and 53, respectively;
(xxiv) SEQ ID NOs:54 and 55, respectively;
(xxv) SEQ ID NOs:56 and 57, respectively;
(xxvi) SEQ ID NOs:58 and 59, respectively;
(xxvii) SEQ ID NOs:60 and 61, respectively;
(xxviii) SEQ ID NOs:62 and 63, respectively;
(xxix) SEQ ID NOs:64 and 65, respectively;
(xxx) SEQ ID NOs:66 and 67, respectively;
(xxxi) SEQ ID NOs:68 and 69, respectively;
(xxxii) SEQ ID NOs:70 and 71, respectively;
(xxxiii) SEQ ID NOs:72 and 73, respectively;
(xxxiv) SEQ ID NOs:313 and 314, respectively;
(xxxv) SEQ ID NOs:315 and 316, respectively;
(xxxvi) SEQ ID NOs:317 and 314, respectively;
(xxxvii) SEQ ID NOs:318 and 316, respectively;
(xxxviii) SEQ ID NOs:319 and 314, respectively;
(xxxix) SEQ ID NOs:320 and 316, respectively;
(xl) SEQ ID NOs:321 and 314, respectively;
(xli) SEQ ID NOs:322 and 316, respectively;
(xlii) SEQ ID NOs:323 and 314, respectively;
(xliii) SEQ ID NOs:324 and 316, respectively;
(xliv) SEQ ID NOs:325 and 326, respectively;
(xlv) SEQ ID NOs:327 and 328, respectively;
(xlvi) SEQ ID NOs:325 and 329, respectively;
(xlvii) SEQ ID NOs:330 and 331, respectively;
(xlviii) SEQ ID NOs:325 and 332, respectively;
(xlix) SEQ ID NOs:330 and 333, respectively;
(l) SEQ ID NOs:334 and 332, respectively;
(li) SEQ ID NOs:335 and 336, respectively;
(lii) SEQ ID NOs:337 and 338, respectively;
(liii) SEQ ID NOs:38 and 33, respectively;
(liv) SEQ ID NOs:339 and 340, respectively;
(lv) SEQ ID NOs:339 and 341, respectively;
(lvi) SEQ ID NOs:339 and 342, respectively;
(lvii) SEQ ID NOs:343 and 340, respectively;
(lviii) SEQ ID NOs:343 and 342, respectively;
(lix) SEQ ID NOs:344 and 340, respectively;
(lx) SEQ ID NOs:344 and 341, respectively;
(lxi) SEQ ID NOs:344 and 342, respectively;
(lxii) SEQ ID NOs:345 and 340, respectively;
(lxiii) SEQ ID NOs:345 and 341, respectively;
(lxiv) SEQ ID NOs:345 and 342, respectively;
(lxv) SEQ ID NOs:346 and 347, respectively;
(lxvi) SEQ ID NOs:348 and 316, respectively;
(lxvii) SEQ ID NOs:349 and 347, respectively;
(lxviii) SEQ ID NOs:349 and 350, respectively;
(lxix) SEQ ID NOs:351 and 352, respectively;
(lxx) SEQ ID NOs:349 and 353, respectively;
(lxxi) SEQ ID NOs:346 and 354, respectively;
(lxxii) SEQ ID NOs:349 and 355, respectively;
(lxxiii) SEQ ID NOs:349 and 356, respectively;
(lxxiv) SEQ ID NOs:357 and 316, respectively;
(lxxv) SEQ ID NOs:349 and 358, respectively;
(lxxvi) SEQ ID NOs:349 and 316, respectively;
(lxxvii) SEQ ID NOs:359 and 360, respectively;
(lxxviii) SEQ ID NOs:361 and 362, respectively;
(lxxix) SEQ ID NOs:363 and 316, respectively;
(lxx) SEQ ID NOs:364 and 365, respectively;
(lxxxi) SEQ ID NOs:349 and 366, respectively;
(lxxxii) SEQ ID NOs:346 and 316, respectively;
(lxxxiiii) SEQ ID NOs:346 and 367, respectively;
(lxxxiv) SEQ ID NOs:349 and 368, respectively;
(lxxxv) SEQ ID NOs:369 and 316, respectively;
(lxxxvi) SEQ ID NOs:346 and 370, respectively;
(lxxxvii) SEQ ID NOs:371 and 316, respectively;
(lxxxviii) SEQ ID NOs:372 and 356, respectively;
(lxxxix) SEQ ID NOs:357 and 358, respectively;
(xc) SEQ ID NOs:349 and 373, respectively;
(xci) SEQ ID NOs:346 and 374, respectively;
(xcii) SEQ ID NOs:375 and 316, respectively;
(xciii) SEQ ID NOs:376 and 316, respectively;
(xciv) SEQ ID NOs:346 and 377, respectively;
(xcv) SEQ ID NOs:378 and 379, respectively;
(xcvi) SEQ ID NOs:380 and 381, respectively;
(xcvii) SEQ ID NOs:349 and 382, respectively;
(xcviii) SEQ ID NOs:357 and 383, respectively;
(xcix) SEQ ID NOs:349 and 358, respectively;
(c) SEQ ID NOs:384 and 316, respectively;
(ci) SEQ ID NOs:385 and 316, respectively;
(cii) SEQ ID NOs:357 and 386, respectively;
(ciii) SEQ ID NOs:387 and 388, respectively;
(civ) SEQ ID NOs:359 and 316, respectively;
(cv) SEQ ID NOs:389 and 316, respectively;
(cvi) SEQ ID NOs:390 and 316, respectively;
(cvii) SEQ ID NOs:391 and 392, respectively;
(cviii) SEQ ID NOs:393 and 356, respectively;
(cix) SEQ ID NOs:390 and 392, respectively;
(cx) SEQ ID NOs:357 and 386, respectively;
(cxi) SEQ ID NOs:394 and 395, respectively;
(cxii) SEQ ID NOs:396 and 395, respectively;
(cxiii) SEQ ID NOs:397 and 395, respectively;
(cxiv) SEQ ID NOs:398 and 395, respectively;
(cxv) SEQ ID NOs:401 and 395, respectively;
(cxvi) SEQ ID NOs:402 and 403, respectively;
(cxvii) SEQ ID NOs:443 and 341, respectively;
(cxviii) SEQ ID NOs:444 and 341, respectively;
(cxix) SEQ ID NOs:445 and 341, respectively;
(cxx) SEQ ID NOs:446 and 341, respectively;
(cxxi) SEQ ID NOs:447 and 341, respectively;
(cxxii) SEQ ID NOs:448 and 341, respectively;
(cxxiii) SEQ ID NOs:449 and 341, respectively;
(cxxiv) SEQ ID NOs:450 and 341, respectively;
(cxxv) SEQ ID NOs:451 and 341, respectively;
(cxxvi) SEQ ID NOs:452 and 341, respectively;
(cxxvii) SEQ ID NOs:453 and 341, respectively;
(cxxviii) SEQ ID NOs:443 and 454, respectively;
(cxxix) SEQ ID NOs:443 and 455, respectively;
(cxxx) SEQ ID NOs:443 and 456, respectively;
(cxxxi) SEQ ID NOs:443 and 457, respectively;
(cxxxii) SEQ ID NOs:443 and 458, respectively; or
(cxxxiii) SEQ ID NOs:443 and 459, respectively.

4-6. (canceled)

7. The antigen-binding domain of claim 2, wherein the antigen-binding domain is capable of crossing the blood brain barrier (BBB).

8-35. (canceled)

36. The antigen-binding domain of claim 2, wherein the antigen-binding domain comprises the amino acid sequence of any one of SEQ ID NOs:404, 185, 186, 189, 190, 192, 193, 195-259, and 261-284.

37-38. (canceled)

39. An antigen-binding domain that specifically binds to human TfR, wherein the antigen-binding domain is a VHH comprising the VH CDR1, VH CDR2, and VH CDR3 of:
(i) SEQ ID NOs:94, 415, and 418, respectively,
(ii) SEQ ID NOs:74-76, respectively;
(iii) SEQ ID NOs:77-79, respectively;
(iv) SEQ ID NOs:80-82, respectively;
(v) SEQ ID NOs:83-85, respectively;
(vi) SEQ ID NOs:74, 86, and 76, respectively;
(vii) SEQ ID NOs:87-89, respectively;
(viii) SEQ ID NOs:90-92, respectively;
(ix) SEQ ID NOs:83, 93, 85, respectively;
(x) SEQ ID NOs:94-96, respectively;
(xi) SEQ ID NOs:97, 86, and 98, respectively;
(xii) SEQ ID NOs:99, 100, and 101, respectively;
(xiii) SEQ ID NOs:99, 102, and 103, respectively;
(xiv) SEQ ID NOs:104-106, respectively;
(xv) SEQ ID NOs:107-109, respectively;
(xi) SEQ ID NOs:99, 110, and 103, respectively;
(xvii) SEQ ID NOs:99, 111, and 112, respectively;
(xviii) SEQ ID NOs:90, 113, and 114, respectively;
(x) SEQ ID NOs:83, 115, and 85, respectively;
(xx) SEQ ID NOs:116, 110, and 103, respectively;
(xxi) SEQ ID NOs:117-119, respectively;
(xxii) SEQ ID NOs:116, 110, and 103, respectively;
(xxiii) SEQ ID NOs:90, 120, and 114, respectively;
(xxiv) SEQ ID NOs:90, 113, and 114, respectively;
(xxv) SEQ ID NOs:97, 121, and 122, respectively;
(xxvi) SEQ ID NOs:74, 123, and 124, respectively;
(xxvii) SEQ ID NOs:74, 125, and 126, respectively;
(xxviii SEQ ID NOs:99, 127, and 128, respectively;
(xxix) SEQ ID NOs:90, 129, and 130, respectively;
(xxx) SEQ ID NOs:131, 132, and 133, respectively;
(xxxi) SEQ ID NOs:131, 132, and 133, respectively;
(xxxii) SEQ ID NOs:90, 134, and 114, respectively;
(xxxiii) SEQ ID NOs:83, 302, and 303, respectively;
(xxxiv) SEQ ID NOs:99, 305, and 103, respectively;
(xxxv) SEQ ID NOs:99, 306, and 103, respectively;
(xxxvi) SEQ ID NOs:99, 306, and 103, respectively;
(xxxvii) SEQ ID NOs:99, 306, and 103, respectively;
(xxxviii) SEQ ID NOs:99, 305, and 103, respectively;
(xxxix) SEQ ID NOs:99, 312, and 103, respectively;
(xl) SEQ ID NOs:99, 312, and 103, respectively;
(xli) SEQ ID NOs:99, 312, and 103, respectively
(xlii) SEQ ID NOs:94, 95, and 418, respectively;
(xliii SEQ ID NOs:94, 412, and 419, respectively;
(xliv) SEQ ID NOs:94, 412, and 420, respectively;
(xlv) SEQ ID NOs:94, 413, and 418, respectively;
(xlvi) SEQ ID NOs:94, 414, and 418, respectively;
(xlvii SEQ ID NOs:94, 416, and 418, respectively;
(xlviii) SEQ ID NOs:94, 417, and 418, respectively;
(xlix) SEQ ID NOs:460, 312, and 103, respectively;
(l) SEQ ID NOs:460, 312, and 461, respectively;
(li) SEQ ID NOs:460, 312, and 462, respectively;
(lii) SEQ ID NOs:460, 312, and 463, respectively;
(liii) SEQ ID NOs:460, 312, and 464, respectively;
(liv) SEQ ID NOs:460, 312, and 465, respectively;
(lv) SEQ ID NOs:460, 312, and 466, respectively;
(lvi) SEQ ID NOs:460, 312, and 467, respectively;
(lvii) SEQ ID NOs:460, 312, and 468, respectively;
(lviii) SEQ ID NOs:460, 312, and 469, respectively;
(lix) SEQ ID NOs:460, 312, and 470, respectively;
(lx) SEQ ID NOs:460, 312, and 103, respectively;
(lxi) SEQ ID NOs:460, 312, and 103, respectively;
(lxi) SEQ ID NOs:460, 312, and 103, respectively;
(lxiii SEQ ID NOs:460, 312, and 103, respectively;
(lxiv) SEQ ID NOs:460, 312, and 103, respectively;
(lxv) SEQ ID NOs:460, 312, and 103, respectively: or
(lxvi) SEQ ID NOs:460, 312, and 103, respectively.

40. A fusion protein comprising the antigen-binding domain of claim 2 and a heterologous protein or peptide.

41. The fusion protein of claim 40, wherein the heterologous protein or peptide comprises the amino acid sequence of beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (GCase or GBA), progranulin (PGRN), Prosaposin (PSAP), Glycoprotein nonmetastatic protein B (GPNMB), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33 or sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), Paired immunoglobin like type 2 receptor alpha (PILRA), Membrane Spanning 4-Domains A4A (MS4A4A), Membrane Spanning 4-Domains A 6A (MS4A6A), or Transmembrane Protein 106B (TMEM106b), clusterin (APOJ), Reelin, ubiquitin protein ligase E3A (UBE3A), Tripeptidyl Peptidase 1 (CLN2/TPP1), Alpha-L-Iduronidase (IDUA), Iduronate 2-Sulfatase (IDS), glucosamine (N-acetyl)-6-sulfatase (GNS), heparan-alpha-glucosaminide N-acetyltransferase (HGSNAT), and N-acetyl-alpha-glucosaminidase (NAGLU), N-sulfoglucosamine sulfohydrolase (SGSH), or a portion thereof.

42-57. (canceled)

58. An antibody comprising the antigen-binding domain of claim 2.

59. An antibody or antigen-binding fragment thereof that binds to the same human TfR epitope as the antigen-binding domain of claim 2.

60. An antibody or antigen-binding fragment thereof that competitively inhibits binding of the antigen-binding domain of claim 2 to human TfR.

61. A multi-specific protein comprising a first antigen-binding domain that is the antigen-binding domain of claim 2 linked to (a) second antigen-binding domain, optionally wherein the second antigen-binding domain specifically binds to a CNS antigen or (b) an antibody or antigen-binding fragment thereof, optionally wherein the antibody or antigen-binding fragment thereof specifically binds to a CNS antigen.

62-101. (canceled)

102. The multi-specific protein of claim 61, wherein the CNS antigen is beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (GCase or GBA), progranulin (PGRN), Prosaposin (PSAP), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), sialic acid binding Ig-like lectin 11 (Siglec11), glycoprotein nonmetastatic melanoma protein B (GPNMB), Paired immunoglobin like type 2 receptor alpha (PILRA), Membrane Spanning 4-Domains A4A (MS4A4A), Membrane Spanning 4-Domains A 6A (MS4A6A), MS4A4E, Transmembrane Protein 106B (TMEM106b), ubiquitin protein ligase E3A (UBE3A), CR1, ABCA1, ABCA7, HLA-DR1, HLA-DR5, IL1RAP, TREML2, IL-34, SORL1, or ADAM1.

103-105. (canceled)

106. The fusion protein of claim 40, wherein the fusion protein is linked to an imaging agent.

107. (canceled)

108. A composition comprising a first polynucleotide, a second polynucleotide, and a third polynucleotide, wherein the first, second, and third polynucleotides encode the multi-specific protein of claim 61, wherein the first polynucleotide encodes a first heavy chain, the second polynucleotide encodes a second heavy chain and the antigen-binding domain that specifically binds to human TfR, and the third polynucleotide encodes a light chain.

109. A composition comprising a first polynucleotide, a second polynucleotide, and a third polynucleotide, wherein the first, second, and third polynucleotides encode the multi-specific protein of claim 61, wherein the first polynucleotide encodes a first heavy chain and a first antigen-binding domain that specifically binds to human TfR, the second polynucleotide encodes a second heavy chain and a second antigen-binding domain that specifically binds to human TfR, and the third polynucleotide encodes a light chain, optionally wherein the first and second antigen-binding domains that bind to human TfR comprise the same amino acid sequence.

110-112. (canceled)

113. A composition comprising a first polynucleotide and a second polynucleotide, wherein the first and second polynucleotides encode the multi-specific protein of claim 61, wherein the first polynucleotide encodes a heavy chain and the antigen-binding domain that bind to human TfR, and wherein the second polynucleotide encodes a light chain.

114. A host cell comprising the composition of claim 108.

115. An isolated polynucleotide comprising a nucleic acid molecule encoding the heavy chain variable region of the antigen-binding domain of claim 2 or a nucleic acid molecule encoding the light chain variable region of the antigen-binding domain of claim 2.

116. (canceled)

117. An isolated vector comprising the polynucleotide of claim 115.

118-120. (canceled)

121. A method of producing a multi-specific protein comprising culturing the host cell of claim 114 so that the multi-specific protein is produced, optionally wherein the method further comprises isolating the multi-specific protein from the culture.

122. (canceled)

123. A pharmaceutical composition comprising (i) the antigen-binding domain of claim 2 and (ii) a pharmaceutically acceptable carrier.

124-125. (canceled)

126. A method of treating a neurological disease or disorder in a subject comprising administering (i) the fusion protein of claim 40 or (ii) a pharmaceutical composition comprising the fusion protein of claim 40 and a pharmaceutically acceptable carrier to the subject.

127-137. (canceled)

138. A method of treating a lysosomal storage disease in a subject comprising administering (i) the fusion protein of claim 40 or (ii) a pharmaceutical composition comprising the fusion protein of claim 40 and a pharmaceutically acceptable carrier to the subject.

139. (canceled)

140. A method of transporting a fusion protein across the BBB of a subject, comprising administering to the subject the fusion protein of claim 40.

141-144. (canceled)

145. A method of increasing the concentration of a CNS binding antigen in the CSF of a subject, comprising administering the multi-specific protein of claim 61, wherein the concentration of the CNS binding antigen is increased as compared to administering the CNS binding antigen alone to the subject.

146. A method of imaging a CNS antigen within a subject, comprising administering to the subject the fusion protein protein of claim 106 and locating the imaging agent within the subject.

147. A method of detecting a CNS antigen in vitro, comprising contacting an in vitro sample with the fusion of claim 106 and locating the imaging agent within the sample.

148-149. (canceled)

150. A method of treating a neurological disease or disorder in a subject comprising administering (i) the multi-specific protein of claim 61 or (ii) a pharmaceutical composition comprising the multi-specific protein of claim 61 and a pharmaceutically acceptable carrier.

151. A method of treating a lysosomal storage disease in a subject comprising administering (i) the multi-specific protein of claim 61 or (ii) a pharmaceutical composition comprising the multi-specific protein of claim 61 and a pharmaceutically acceptable carrier.

152. A method of transporting an antibody across the BBB of a subject, comprising administering to the subject the antibody of claim 58.

153. A method of transporting a multi-specific protein across the BBB of a subject, comprising administering to the subject the multi-specific protein of claim 61.
